(19) United States
(12) Patent Application Publication
(10) Pub. No.: US 2026/0345632 A1
(43) Pub. Date: Nov. 5, 2026

(54) USE OF DIAZOXIDE IN TREATMENT

(71) Applicant: Soleno Therapeutics, Inc., Redwood City, CA (US)

(72) Inventors: Neil M. Cowen, Redwood City, CA (US); Anish Bhatnagar, Redwood City, CA (US); Julie S. Perry, Redwood City, CA (US); Michael F. Huang, Redwood City, CA (US)

(21) Appl. No.: 19/699,504

(22) Filed: Jun. 5, 2026

Related U.S. Application Data
(63) Continuation of application No. PCT/IB2026/052852, filed on Mar. 24, 2026.
(60) Provisional application No. 63/777,646, filed on Mar. 25, 2025.
(60) Provisional application No. 63/948,490, filed on Dec. 24, 2025.

(51) Int. Cl. A61K 31/549; A61P 3/04

(52) U.S. Cl. CPC A61K 31/549; A61P 3/04

(57) ABSTRACT

Provided herein are methods of treating a subject in need thereof wherein diazoxide, or a pharmaceutically acceptable salt thereof, is administered concomitantly with other pharmaceuticals.

### CROSS-REFERENCE TO RELATED APPLICATIONS

This application claims priority to U.S. Provisional Application Ser. No. 63/948,490, filed Dec. 24, 2025, U.S. patent application Ser. No. 19/256,001, filed Jun. 30, 2025, and U.S. Provisional Application Ser. No. 63/777,646, filed Mar. 25, 2025, each of which is herein incorporated by reference in its entirety for all purposes.

### BACKGROUND

Prader-Willi syndrome (“PWS”) is a rare, complex genetic neurobehavioral/metabolic disorder with an estimated birth incidence of 1:15,000 to 1:20,000. PWS arises from lack of expression of paternally inherited imprinted genes on chromosome 15q11-q13 caused by a paternal deletion, maternal uniparental disomy 15 or an imprinting center defect, resulting in hypothalamic dysfunction. Clinical features of PWS include hypotonia and feeding difficulties in infancy and sustained accumulation of excess body fat beginning in early childhood. Hyperphagia, a life-threatening cardinal clinical feature of PWS, presents as food obsession, aggressive food seeking, and lack of satiety, with progression to severe obesity if energy intake is not restricted. PWS is also associated with intellectual disability, low muscle mass, neuroendocrine abnormalities, including growth hormone and gonadotropin deficiency, behavioral problems, including aggression, anxiety and compulsivity, and elevated incidence of early mortality.

It has been found that certain metabolic signs or comorbidities of subjects with PWS and other syndromic obesities can be alleviated with K_{ATP} channel openers such as diazoxide, or its pharmaceutically acceptable salts. For example, diazoxide, or a pharmaceutically acceptable salt such as diazoxide choline, can be formulated as an extended-release tablet formulation and used to treat subjects with PWS. Additional uses are described in the following: International Application Pub. No. 2016/077629, U.S. Pat. Nos. 9,757,384, 10,058,557, 10,456,408, 10,874,676, 12,109,216, and 12,178,823, each of which are incorporated by reference in their entirety herein. However, the effect of concomitant use of diazoxide, or a pharmaceutically acceptable salt thereof, in conjunction with other pharmaceuticals is highly unpredictable. At this time, there is no known manner of adjusting the dosing schedule for diazoxide, or its pharmaceutically acceptable salts, to account for the effects of concomitant administration.

### SUMMARY

Disclosed herein are methods of treating a subject having Prader-Willi Syndrome, including the comorbidities of PWS such as those described herein, treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, and (b) monitoring the patient for signs and symptoms of hyperglycemia and/or hyperuricemia.

Also disclosed herein are methods of treating a subject having Prader-Willi Syndrome, including the comorbidities of PWS such as those described herein, treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, and (b) monitoring serum levels of diphenylhydantoin in the patient.

Also disclosed herein are methods of treating a subject having Prader-Willi Syndrome, including the comorbidities of PWS such as those described herein, treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, and (b) monitoring international normalized ratio (INR) of the patient.

Also disclosed herein are methods of treating a subject having Prader-Willi Syndrome, including the comorbidities of PWS such as those described herein (e.g., physiological, behavioral, and cognitive comorbidities, including those described herein), treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, wherein the subject is concomitantly administered one or more strong CYP3A4 inhibitors, (b) monitoring the subject for adverse reactions to diazoxide, or a pharmaceutically acceptable salt thereof; and (c) if the subject exhibits adverse reactions, adjusting the therapeutically effective amount of diazoxide, or a pharmaceutically acceptable salt thereof, administered to the subject.

Also disclosed herein are methods of treating a subject having Smith-Magenis Syndrome, including the comorbidities of SMS such as those described herein, treating hyperphagia or hypotonia in a subject having Smith-Magenis Syndrome, treating a subject with Smith-Magenis Syndrome having hyperphagia or hypotonia, methods of increasing the lean body mass in a subject with SMS, methods of increasing the lean body mass/fat mass ratio in a subject with SMS, methods of increasing the lower limb muscle mass in a subject with SMS, and methods of reducing aggressive behaviors in a subject with SMS. In some of the methods disclosed herein, the method comprises (a) administering to the subject a therapeutically effective amount of diazoxide, or a pharmaceutically acceptable salt thereof, wherein the subject is concomitantly administered one or more strong CYP3A4 inhibitors, (b) monitoring the subject for adverse reactions to diazoxide, or a pharmaceutically acceptable salt thereof, and (c) if the subject exhibits adverse reactions, adjusting the therapeutically effective amount of diazoxide, or a pharmaceutically acceptable salt thereof, administered to the subject.

Also disclosed herein are methods of treating a subject having Glycogen Storage Disease Type 1 (GSD1), including the comorbidities of GSD1 such as those described herein, treating hypoglycemia in a subject having GSD1, treating a subject with GSD1 having hypoglycemia, treating lactic acidosis, hyperuricemia, hypercholesterolemia, or hypertriglyceridemia in a subject having GSD1, treating a subject with GSD1 having lactic acidosis, hyperuricemia, hypercholesterolemia, or hypertriglyceridemia, treating hepatomegaly, xanthoma or epistaxis in a subject having GSD1, treating a subject with GSD1 having hepatomegaly, xanthoma or epistaxis, treating neutropenia in a subject having GSD1, treating a subject with GSD1 having neutropenia, treating hyperphagia or hypotonia in a subject having GSD1, treating a subject with GSD1 having hyperphagia or hypotonia, methods of increasing the lean body mass in a subject with GSD1, methods of increasing the lean body mass/fat mass ratio in a subject with GSD1, methods of increasing the lower limb muscle mass in a subject with GSD1, and methods of reducing aggressive behaviors or food related behaviors in a subject with GSD1. In some of the methods disclosed herein, the method comprises (a) administering to the subject a therapeutically effective amount of diazoxide, or a pharmaceutically acceptable salt thereof, wherein the subject is concomitantly administered one or more strong CYP3A4 inhibitors, (b) monitoring the subject for adverse reactions to diazoxide, or a pharmaceutically acceptable salt thereof, and (c) if the subject exhibits adverse reactions, adjusting the therapeutically effective amount of diazoxide, or a pharmaceutically acceptable salt thereof, administered to the subject.

Also disclosed herein are methods of treating a subject having Glucose Transporter Type 1 Deficiency Syndrome (Glut1DS), including the comorbidities of Glut1DS such as those described herein, treating neuroglycopenia in a subject having Glut1DS, treating a subject with Glut1DS having neuroglycopenia, treating epilepsy in a subject having Glut1DS, treating a subject with Glut1DS having epilepsy, treating hypotonia in a subject having Glut1DS, treating a subject with Glut1DS having hypotonia, methods of increasing the lean body mass in a subject with Glut1DS, methods of increasing the lean body mass/fat mass ratio in a subject with Glut1DS, methods of increasing the lower limb muscle mass in a subject with Glut1DS, and methods of reducing aggressive behaviors or sleep related behaviors in a subject with Glut1DS. In some of the methods disclosed herein, the method comprises (a) administering to the subject a therapeutically effective amount of diazoxide, or a pharmaceutically acceptable salt thereof, wherein the subject is concomitantly administered one or more strong CYP3A4 inhibitors, (b) monitoring the subject for adverse reactions to diazoxide, or a pharmaceutically acceptable salt thereof, and (c) if the subject exhibits adverse reactions, adjusting the therapeutically effective amount of diazoxide, or a pharmaceutically acceptable salt thereof, administered to the subject.

Also disclosed herein are methods of treating a subject in need thereof. In some of the methods disclosed herein, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some of the methods disclosed herein, the subject is a subject having Glycogen Storage Disease Type 1 or a subject having Glucose Transporter Type 1 Deficiency Syndrome. In some of the methods disclosed herein, the method comprises (a) administering to the subject a therapeutically effective amount of diazoxide, or a pharmaceutically acceptable salt thereof, wherein the subject is concomitantly administered one or more strong CYP3A4 inhibitors, (b) monitoring the subject for adverse reactions to diazoxide, or a pharmaceutically acceptable salt thereof; and (c) if the subject exhibits adverse reactions, adjusting the therapeutically effective amount of diazoxide, or a pharmaceutically acceptable salt thereof, administered to the subject.

In some embodiments, the therapeutically effective amount of diazoxide, or a pharmaceutically acceptable salt thereof, is reduced relative to the therapeutically effective amount for the subject if not concomitantly administered one or more strong CYP3A4 inhibitors.

In some embodiments, the therapeutically effective amount of diazoxide, or a pharmaceutically acceptable salt thereof, is reduced by between about 15.0%-30.0%.

Also disclosed herein are methods of treating a subject having Prader-Willi Syndrome, including the comorbidities of PWS such as those described herein, treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, wherein the subject is concomitantly administered one or more strong CYP1A2 inhibitors; and (b) adjusting the therapeutically effective amount of diazoxide, or a pharmaceutically acceptable salt thereof, administered to the subject.

Also disclosed herein are methods of treating a subject having Smith-Magenis Syndrome, including the comorbidities of SMS such as those described herein, treating hyperphagia or hypotonia in a subject having Smith-Magenis Syndrome, treating a subject with Smith-Magenis Syndrome having hyperphagia or hypotonia, methods of increasing the lean body mass in a subject with SMS, methods of increasing the lean body mass/fat mass ratio in a subject with SMS, methods of increasing the lower limb muscle mass in a subject with SMS, and methods of reducing aggressive behaviors in a subject with SMS. In some of the methods disclosed herein, the method comprises (a) administering to the subject a therapeutically effective amount of diazoxide, or a pharmaceutically acceptable salt thereof, wherein the subject is concomitantly administered one or more strong CYP1A2 inhibitors; and (b) adjusting the therapeutically effective amount of diazoxide, or a pharmaceutically acceptable salt thereof, administered to the subject.

Also disclosed herein are methods of treating a subject having Glycogen Storage Disease Type 1, including the comorbidities of GSD1 such as those described herein, treating hypoglycemia in a subject having GSD1, treating a subject with GSD1 having hypoglycemia, treating lactic acidosis, hyperuricemia, hypercholesterolemia, or hypertriglyceridemia in a subject having GSD1, treating a subject with GSD1 having lactic acidosis, hyperuricemia, hypercholesterolemia, or hypertriglyceridemia, treating hepatomegaly, xanthoma or epistaxis in a subject having GSD1, treating a subject with GSD1 having hepatomegaly, xanthoma or epistaxis, treating neutropenia in a subject having GSD1, treating a subject with GSD1 having neutropenia, treating hyperphagia or hypotonia in a subject having GSD1, treating a subject with GSD1 having hyperphagia or hypotonia, methods of increasing the lean body mass in a subject with GSD1, methods of increasing the lean body mass/fat mass ratio in a subject with GSD1, methods of increasing the lower limb muscle mass in a subject with GSD1, and methods of reducing aggressive behaviors in a subject with GSD1. In some of the methods disclosed herein, the method comprises (a) administering to the subject a therapeutically effective amount of diazoxide, or a pharmaceutically acceptable salt thereof, wherein the subject is concomitantly administered one or more strong CYP1A2 inhibitors; and (b) adjusting the therapeutically effective amount of diazoxide, or a pharmaceutically acceptable salt thereof, administered to the subject.

Also disclosed herein are methods of treating a subject having Glucose Transporter Type 1 Deficiency Syndrome, including the comorbidities of Glut1DS such as those described herein, treating neuroglycopenia in a subject having Glut1DS, treating a subject with Glut1DS having neuroglycopenia, treating epilepsy in a subject having Glut1DS, treating a subject with Glut1DS having epilepsy, treating hyperphagia or hypotonia in a subject having Glut1DS, treating a subject with Glut1DS having hyperphagia or hypotonia, methods of increasing the lean body mass in a subject with Glut1DS, methods of increasing the lean body mass/fat mass ratio in a subject with Glut1DS, methods of increasing the lower limb muscle mass in a subject with Glut1DS, and methods of reducing aggressive behaviors or food related behaviors in a subject with Glut1DS. In some of the methods disclosed herein, the method comprises (a) administering to the subject a therapeutically effective amount of diazoxide, or a pharmaceutically acceptable salt thereof, wherein the subject is concomitantly administered one or more strong CYP1A2 inhibitors; and (b) adjusting the therapeutically effective amount of diazoxide, or a pharmaceutically acceptable salt thereof, administered to the subject.

Also disclosed herein are methods of treating a subject in need thereof. In some of the methods disclosed herein, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some of the methods disclosed herein, the subject is a subject having Glycogen Storage Disease Type 1 or a subject having Glucose Transporter Type 1 Deficiency Syndrome. In some of the methods disclosed herein, the method comprises (a) administering to the subject a therapeutically effective amount of diazoxide, or a pharmaceutically acceptable salt thereof, wherein the subject is concomitantly administered one or more strong CYP1A2 inhibitors; and (b) adjusting the therapeutically effective amount of diazoxide, or a pharmaceutically acceptable salt thereof, administered to the subject.

In some embodiments, the therapeutically effective amount of diazoxide is reduced relative to the therapeutically effective amount for the subject if not concomitantly administered one or more strong CYP1A2 inhibitors.

Also disclosed herein are methods of treating a subject having Prader-Willi Syndrome, including the comorbidities of PWS such as those described herein, treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, wherein the subject is concomitantly administered one or more strong OAT1 inhibitors; and (b) adjusting the therapeutically effective amount of diazoxide, or a pharmaceutically acceptable salt thereof, administered to the subject.

Also disclosed herein are methods of treating a subject in need thereof. In some of the methods disclosed herein, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some of the methods disclosed herein, the subject is a subject having Glycogen Storage Disease Type 1 or a subject having Glucose Transporter Type 1 Deficiency Syndrome. In some of the methods disclosed herein, the method comprises (a) administering to the subject a therapeutically effective amount of diazoxide, or a pharmaceutically acceptable salt thereof, wherein the subject is concomitantly administered one or more strong OAT1 inhibitors; and (b) adjusting the therapeutically effective amount of diazoxide, or a pharmaceutically acceptable salt thereof, administered to the subject.

In some embodiments, the therapeutically effective amount of diazoxide is reduced relative to the therapeutically effective amount for the subject if not concomitantly administered one or more strong OAT1 inhibitors.

Also disclosed herein are methods of treating a subject having Prader-Willi Syndrome, including the comorbidities of PWS such as those described herein, treating hyperphagia or hypotonia in a subject having Prader-Willi Syndrome, treating a subject with Prader-Willi Syndrome having hyperphagia or hypotonia, methods of increasing the lean body mass in a subject with PWS, methods of increasing the lean body mass/fat mass ratio in a subject with PWS, methods of increasing the lower limb muscle mass in a subject with PWS, and methods of reducing aggressive behaviors in a subject with PWS. Also disclosed herein are methods of reducing food-related behaviors in a subject with PWS. In some of the methods disclosed herein, the method comprises (a) administering to the subject a therapeutically effective amount of diazoxide, or a pharmaceutically acceptable salt thereof, wherein the subject is concomitantly administered one or more strong OAT3 inhibitors; and (b) adjusting the therapeutically effective amount of diazoxide, or a pharmaceutically acceptable salt thereof, administered to the subject.

Also disclosed herein are methods of treating a subject in need thereof. In some of the methods disclosed herein, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some of the methods disclosed herein, the subject is a subject having Glycogen Storage Disease Type 1 or a subject having Glucose Transporter Type 1 Deficiency Syndrome. In some of the methods disclosed herein, the method comprises (a) administering to the subject a therapeutically effective amount of diazoxide, or a pharmaceutically acceptable salt thereof, wherein the subject is concomitantly administered one or more strong OAT3 inhibitors; and (b) adjusting the therapeutically effective amount of diazoxide, or a pharmaceutically acceptable salt thereof, administered to the subject.

In some embodiments, the therapeutically effective amount of diazoxide is reduced relative to the therapeutically effective amount for the subject if not concomitantly administered one or more strong OAT3 inhibitors.

In some embodiments, the pharmaceutically acceptable salt is diazoxide choline. In some embodiments, the administration of diazoxide choline is initiated at a reduced starting dose of diazoxide choline and then increased each 2-3 weeks to reach a reduced target maintenance dose of diazoxide choline.

In some embodiments, the subject has a weight less than about 150 kg, including a weight between about 3 kg and about 40 kg.

In some embodiments, the subject is less than or equal to 10 years of age.

In some embodiments, one or more comorbidities of PWS are treated. In some embodiments, one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, accumulation of excess body fat, elevated inflammatory markers, etc.) comorbidities associated with PWS are improved after treatment.

In some embodiments, one or more comorbidities of SMS are treated. In some embodiments, one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, accumulation of excess body fat, elevated inflammatory markers, etc.) comorbidities associated with SMS are improved after treatment.

In some embodiments, one or more comorbidities of GSD1 are treated. In some embodiments, one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, accumulation of excess body fat, elevated inflammatory markers, etc.) comorbidities associated with GSD1 are improved after treatment.

In some embodiments, one or more comorbidities of Glut1DS are treated. In some embodiments, one or more of the behavioral and cognitive (e.g., aggressive behaviors, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, accumulation of excess body fat, elevated inflammatory markers, etc.) comorbidities associated with Glut1DS are improved after treatment.

In some embodiments, the subject's hyperphagia is reduced after treatment. In some embodiments, the subject's food preoccupation is decreased after treatment.

In some embodiments, the subject's hypotonia is reduced after treatment. In some embodiments, the subject's lean body mass is increased after treatment. In some embodiments, the subject's lean body mass/fat mass ratio is increased after treatment. In some embodiments, the subject's lower limb muscle mass is increased after treatment. In some embodiments, the subject's muscle mass is increased after treatment. In some embodiments, the subject's muscle strength is increased after treatment. In some embodiments, the subject's lean mass index (LMI) is increased after treatment. In some embodiments, the subject's fat mass index (FMI) is decreased after treatment. In some embodiments, one or more of the subject's elevated inflammatory markers are decreased after treatment. In some embodiments, the subject's amount of circulating C-Reactive Protein (CRP) is decreased after treatment. In some embodiments, the subject's amount of excess body fat is decreased after treatment.

In some embodiments, the subject's aggressive behaviors are reduced after treatment. In some embodiments, one or more of the subject's anxiety, compulsivity, or rigidity is reduced after treatment. In some embodiments, the subject's cognitive impairment is reduced after treatment. In some embodiments, the subject's learning disability is reduced after treatment. In some embodiments, the subject's speech impairment is reduced after treatment.

In some embodiments, the subject's elevated triglyceride level is reduced after treatment. In some embodiments, the subject's elevated non-HDL cholesterol level is reduced after treatment. In some embodiments, weight loss is promoted in the obese subject. In some embodiments, the subject's reduced fasting glucose level is increased after treatment. In some embodiments, the subject's elevated fasting insulin level is reduced after treatment. In some embodiments, the subject's insulin resistance is improved after treatment.

In some embodiments, the diazoxide or pharmaceutically acceptable salt thereof is administered orally. In some embodiments, the diazoxide or pharmaceutically acceptable salt thereof is administered one or more times per day (e.g., 1, 2, 3, or 4 times per day). In some embodiments, the diazoxide or pharmaceutically acceptable salt thereof is administered once per day. In some embodiments, the diazoxide or pharmaceutically acceptable salt thereof is administered orally once per day. In some embodiments, the pharmaceutically acceptable salt is diazoxide choline.

In some embodiments, the reduced starting dose of diazoxide choline is between about 3-100 mg/day of diazoxide choline.

In some embodiments, the reduced target maintenance dose of diazoxide choline is between about 1.1-4.2 mg/kg/day of diazoxide choline. In some embodiments, the reduced target maintenance dose of diazoxide choline is about 3.0 mg/kg/day of diazoxide choline. In some embodiments, the reduced target maintenance dose of diazoxide choline is between about 9-325 mg/day of diazoxide choline. In some embodiments, the reduced target maintenance dose of diazoxide choline is reduced by between about 15%-30%.

Also provided are uses, compounds for use in treatment, and compounds for use in the preparation of medicaments for the treatment of the indications disclosed herein, consistent with the description provided for the methods of treatment described herein. For example, the present disclosure also provides use of diazoxide or a pharmaceutically acceptable salt thereof (e.g., diazoxide choline) in the preparation of medicament(s) for the treatment of a subject having Prader-Willi Syndrome, including the comorbidities of PWS described herein, treating hyperphagia or hypotonia in a subject having Prader-Willi Syndrome, treating a subject with Prader-Willi Syndrome having hyperphagia or hypotonia, increasing the lean body mass in a subject with PWS, increasing the lean body mass/fat mass ratio in a subject with PWS, increasing the lower limb muscle mass in a subject with PWS, or reducing aggressive behaviors in a subject with PWS. Also for example, the present disclosure also provides use of diazoxide or a pharmaceutically acceptable salt thereof (e.g., diazoxide choline) in the preparation of medicament(s) for the treatment of a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. Also for example, the present disclosure also provides use of diazoxide or a pharmaceutically acceptable salt thereof (e.g., diazoxide choline) in the preparation of medicament(s) for the treatment of a subject having Glycogen Storage Disease Type 1 or a subject having Glucose Transporter Type 1 Deficiency Syndrome.

Also for example, the present disclosure also provides use of diazoxide or a pharmaceutically acceptable salt thereof (e.g., diazoxide choline) for the treatment of a subject having Prader-Willi Syndrome, including the comorbidities of PWS described herein, treating hyperphagia or hypotonia in a subject having Prader-Willi Syndrome, treating a subject with Prader-Willi Syndrome having hyperphagia or hypotonia, increasing the lean body mass in a subject with PWS, increasing the lean body mass/fat mass ratio in a subject with PWS, increasing the lower limb muscle mass in a subject with PWS, or reducing aggressive behaviors in a subject with PWS. Also for example, the present disclosure also provides use of diazoxide or a pharmaceutically acceptable salt thereof (e.g., diazoxide choline) for the treatment of a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. Also for example, the present disclosure also provides use of diazoxide or a pharmaceutically acceptable salt thereof (e.g., diazoxide choline) for the treatment of a subject having Glycogen Storage Disease Type 1 or a subject having Glucose Transporter Type 1 Deficiency Syndrome.

Also for example, the present disclosure also provides diazoxide or a pharmaceutically acceptable salt thereof (e.g., diazoxide choline) for use in the treatment of a subject having Prader-Willi Syndrome, including the comorbidities of PWS described herein, treating hyperphagia or hypotonia in a subject having Prader-Willi Syndrome, treating a subject with Prader-Willi Syndrome having hyperphagia or hypotonia, increasing the lean body mass in a subject with PWS, increasing the lean body mass/fat mass ratio in a subject with PWS, increasing the lower limb muscle mass in a subject with PWS, or reducing aggressive behaviors in a subject with PWS. Also for example, the present disclosure also provides diazoxide or a pharmaceutically acceptable salt thereof (e.g., diazoxide choline) for use in the treatment of a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. Also for example, the present disclosure also provides diazoxide or a pharmaceutically acceptable salt thereof (e.g., diazoxide choline) for use in the treatment of a subject having Glycogen Storage Disease Type 1 or a subject having Glucose Transporter Type 1 Deficiency Syndrome.

Additional embodiments, features, and advantages of the invention will be apparent from the following detailed description and through practice of the invention.

### BRIEF DESCRIPTION OF THE FIGURES

FIG. 1 summarizes the change in mean plasma concentration of diazoxide over time (168 hours) for the pharmacokinetic concentration population, as described in Example 3.

### DETAILED DESCRIPTION

As previously noted, Prader-Willi syndrome (“PWS”) is a rare, complex genetic neurobehavioral/metabolic disorder with an estimated birth incidence of 1:15,000 to 1:20,000. PWS arises from lack of expression of paternally inherited imprinted genes on chromosome 15q1-q13 caused by a paternal deletion, maternal uniparental disomy 15 or an imprinting center defect, resulting in hypothalamic dysfunction.

While Prader-Willi syndrome is a genetic condition, it occurs de-novo in nearly all cases. As such, individuals with PWS can be thought of as being sampled from the larger population without regard to genetic background, sex, or ethnicity. It has been found that certain metabolic signs or comorbidities of subjects with PWS and other syndromic obesities can be alleviated with K_{ATP} channel openers, such as diazoxide or its pharmaceutically acceptable salts. Descriptions of the use of diazoxide in the treatment of PWS and Smith Magenis syndrome (SMS) subjects can be found in, for example, in U.S. Pat. Nos. 9,782,416, 7,572,789, 7,799,777, 9,381,202, 10,085,998, 11,045,478, 11,786,536, US Pub. No. 2024/0252507, International Application Pub. No. 2009/006483, U.S. Pat. No. 9,765,043, International Application Pub. No. 2016/077629, U.S. Pat. Nos. 9,757,384, 10,058,557, 10,456,408, 10,874,676, 12,109,216, 12,178,823, International App. No. PCT/IB2024/058904, and VYKAT™ XR Package Insert [Soleno Therapeutics, Inc. (2025)], each of which is incorporated by reference herein.

However, the relationship between concomitantly administered pharmaceuticals is complex.

Although there exist diazoxide dosing regiments for the treatment of PWS subjects, there is no known manner of adjusting said regiments to account for the concomitant administration of other pharmaceuticals, which is shown here to significantly impact the exposure of both diazoxide and the other administered pharmaceutical(s). Due to the potential for increased exposure to diazoxide, or its pharmaceutically acceptable salts, the typical dosing schedule may therefore result in adverse effects. It would therefore be beneficial to be able to modify the dosing schedule for subjects taking other pharmaceuticals.

Unexpectedly, as shown in the present disclosure, it has been found that reducing the administered dose of diazoxide to a subject concomitantly taking one or more select pharmaceuticals (e.g., diuretics, thiazides, diphenylhydantoin, coumarin (or a derivative thereof), strong CYP3A4 inhibitors, strong CYP1A2 inhibitors, strong OAT1 inhibitors, strong OAT3 inhibitors, etc.) results in effective treatments of PWS, including improvements in comorbidities associated with PWS, for example, treatment of a subject's hypotonia, hyperphagia, and other behavioral, cognitive and physiological characteristics of PWS (e.g., as described herein). Accordingly, disclosed herein are methods of treating a subject having Prader-Willi Syndrome that incorporate a modified diazoxide dosing schedule in their treatment. Behavioral and cognitive characteristics of PWS, also referred to as behavioral and cognitive comorbidities related to PWS include, but are not limited to, hyperphagia, aggressive behaviors, food preoccupation behaviors, anxiety, compulsivity, rigidity or resistance to change, cognitive impairment, learning disabilities, and speech impairment.

Physiological characteristics assessed for subjects with PWS, also referred to as physiological comorbidities related to PWS include, but are not limited to, hypotonia, amount of lean body mass, ratio of lean body mass to fat mass, (also referred to as “lean body mass/fat mass ratio”), lower limb muscle mass, muscle strength, accumulation of excess body fat, inflammatory markers, C-reactive protein levels, leptin levels, adiponectin levels, the ratio of adiponectin to leptin, insulin resistance, etc. Various means of measuring such characteristics as described herein and known to the skilled artisan. In some embodiments, the physiological characteristics assessed for subjects with PWS, also referred to as physiological comorbidities related to PWS include, but are not limited to, hypotonia, amount of lean body mass, ratio of lean body mass to fat mass, (also referred to as “lean body mass/fat mass ratio”), lower limb muscle mass, muscle strength, accumulation of excess body fat, inflammatory markers, C-reactive protein levels, etc., and various means of measuring such characteristics as described herein.

As used herein, concomitant administration refers to where a subject is administered diazoxide choline and is further being administered a second course of treatment with an additional therapeutic agent. For example, a CYP1A2 inhibitor or a CYP3A4 inhibitor. In other words, the subject is receiving two courses of therapy over the same period of time, with the administration regimen of each of diazoxide choline and the one or more additional therapeutic agents each being administered according to the recommended dosing regimen for the given therapeutic agent. For example, diazoxide choline can be administered as described herein to, e.g., a subject with PWS, and that same subject may be also be administered a CYP1A2 inhibitor according to the recommended dosing regimen according to the weight, age, and other factors related to optimizing CYP1A2 inhibitor dosing for a given subject. For example, the subject may be administered diazoxide choline and also be administered fluvoxamine to treat depression or obsessive-compulsive disorder. Concomitant administration does not require that administration be at the same time or within a certain window of time (e.g., within minutes or a few hours of each other), but instead both diazoxide choline and the additional therapeutic agent are both dosed over a period of time (e.g., days, weeks, or longer periods of time) consistent with the individual dosing regimens of each and, for diazoxide choline, as taught herein. For illustrative purposes and the avoidance of doubt, a dosing regimen where diazoxide choline is administered daily in the morning and the additional agent is also administered in the morning is concomitant administration. Likewise, where diazoxide choline is administered daily in the morning and the additional agent is administered once daily at night is also concomitant administration. Similarly while diazoxide choline is being administered daily, for example at a similar time each day, the other agent may be administered once, twice, three times or more daily in accordance with the recommended dosing schedule for the indication and subject being treated by the additional therapeutic agent.

The term “treat” or “treatment,” as used herein, refers to any act of managing, alleviating, reducing, delaying progression, eliminating PWS, or improving one or more comorbidities related to PWS (e.g., physiological and behavioral and cognitive comorbidities described herein), in a subject. In some embodiments, treatment may include improving all or substantially all comorbidities of PWS. Treatment may involve the administration of a diazoxide or a pharmaceutically acceptable salt thereof, lifestyle modification, or any combination thereof, with the goal of improving clinical outcomes, enhancing quality of life, or improving physiological function. The term encompasses both curative and palliative approaches, as well as measures intended to avoid worsening of a comorbidity.

Treatment of a subject with PWS may also include the improvement of a comorbidity related to PWS. For example, “improvement” of lean body mass, muscle strengths, or lower limb muscle mass in a PWS subject would be an increase in lean body mass, muscle strength or lower limb muscle mass, respectively, versus prior to treatment. Similarly, an improvement, also considered effective treatment, in the lean body mass/fat mass ratio in a subject with PWS would be an increase in the lean body mass/fat mass ratio. With respect to preventing or delaying progression of PWS, the lean body mass/fat mass ratio, for example, could be where the lean body mass/fat mass ratio is maintained or increased. With respect to, for example, hypotonia, hyperphagia, and aggressive behaviors, an improvement would be a reduction in hypotonia, hyperphagia, or aggressive behaviors, respectively. Similarly, an improvement in, for example, anxiety, compulsivity, rigidity, cognitive impairment, learning disabilities, or speech impairment, would be a reduction (also interchangeably herein referred to as a decrease), in these comorbidities.

As used herein, the terms “including,” “containing,” and “comprising” are used in their open, non-limiting sense.

The articles “a” and “an,” as used herein, refer to one or more than one (i.e., to at least one) of the grammatical object of the article. By way of example, “an element” refers to one element or more than one element.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### Diazoxide and its Pharmaceutically Acceptable Salts

Diazoxide is a potent activator of the K_{ATP} channel and is capable of crossing the blood-brain barrier. Activating the K_{ATP} channel in neuropeptide Y(NPY)/Agouti Related-Protein (AgRP) neurons in the hypothalamus results in reduced secretion of NPY and AgRP, potent endogenous appetite stimulatory neuropeptides, potentially contributing to a reduction in hyperphagia. These actions of the drug are complemented by activating the K_{ATP} channel in the dorsal motor nucleus of the vagus nerve, pancreatic β-cells, and adipocytes to reduce hyperinsulinemia and excess body fat, and improve insulin and leptin resistance, and satiety. These effects have been confirmed in animal models of hyperphagic obesity including a model of PWS, the Magel-2 null mouse (Cowen and Bhatnagar Genes 2020; 11(4):450. Bischof and Wevrick Mol. Genet. Metab. 2018; 123(4):511-517). Diazoxide choline has also shown benefits in the treatment of PWS subjects 4 years old and older in phase 2 and phase 3 clinical trials (Miller et al. J. Clin. Endocrinol. Metab. 2023; 108(7):1676-1685. Strong et al. J. Neurodev. Disord. 2024; 16(1):22. Miller et al. Obesity (Silver Spring) 2024; 32(2):252-261. Kimonis et al. PLoS ONE 2019; 14(9):e0221615).

Diazoxide choline is also known as 7-Chloro-3-methyl-1λ6, 2,4-benzothiadiazin-2-ide 1,1-dioxide 2-hydroxyethyl(trimethyl)azanium, and has the structural formula:

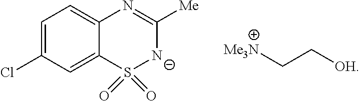

In some embodiments, the diazoxide or pharmaceutically acceptable salt thereof may be formulated as an extended-release formulation. For example, an extended-release formulation designed to allow for once-per-day administration of diazoxide or a pharmaceutically acceptable salt thereof (e.g., diazoxide choline).

As used herein, an extended-release formulation may include pharmaceutical product that may provide both the delay of release of the active pharmaceutical upon administration (e.g., pH-sensitive excipients where release begins after entry into the intestines) and controlled release for sustained release over a period of time (e.g., utilizing excipients that slow release compared to immediate release formulations but where release begins shortly after ingesting the formulation). The solubility of diazoxide choline and its slow release from an extended-release once-per-day tablet formulation (e.g., also referred to as a diazoxide extended-release formulation, which may be diazoxide or a pharmaceutically acceptable salt thereof, including an extended-release formulation of diazoxide choline), facilitate the absorption of diazoxide throughout the gastrointestinal tract (e.g., a sustained extended release formulation), resulting in stable circulating concentrations. Examples of diazoxide extended-release formulations are described in U.S. Pat. No. 9,782,416 and International App. No. PCT/IB2024/058904, each of which are incorporated by reference in their entirety herein. Various references describing the preparation of diazoxide choline and additional salts include U.S. Pat. Nos. 7,572,789, 7,799,777, 9,381,202, 10,085,998, 11,045,478, 11,786,536, and US Pub. No. 2024/0252507, each of which are incorporated by reference in their entirety herein.

As used herein, the term “pharmaceutically acceptable” indicates that the identified material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

### Administration of Diazoxide to a Subject in Need Thereof

As described herein, an effective amount of diazoxide, or a corresponding amount of a pharmaceutically acceptable salt thereof, may be administered to a subject in need thereof to achieve therapeutic outcomes while reducing the incidence of adverse effects. Subjects in need thereof who can be treated include, but are not limited to, a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. Additionally, subjects in need thereof who can be treated include, but are not limited to, a subject having Glycogen Storage Disease Type 1 or a subject having Glucose Transporter Type 1 Deficiency Syndrome.

The term “effective amount,” as used herein, refers to a quantity of diazoxide, or a corresponding amount of a pharmaceutically acceptable salt thereof, or composition comprising diazoxide, or a pharmaceutically acceptable salt thereof, sufficient to produce a desired therapeutic effect in a subject. The effect may include, but is not limited to, enhancing biological function or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

The term “administer,” as used herein, refers to the act of delivering diazoxide, or a pharmaceutically acceptable salt thereof, or composition comprising diazoxide, or a pharmaceutically acceptable salt thereof, to a subject by any suitable route, including but not limited to oral, intravenous, subcutaneous, intramuscular, transdermal, topical, inhalational, or intrathecal administration. The term encompasses both direct administration by a healthcare provider and self-administration by the subject or caregiver. Unless otherwise specified, “administer” includes single, repeated, or continuous dosing regimens as appropriate for the intended therapeutic purpose. In some embodiments, the administration is oral administration.

The term “subject,” as used herein, refers to a mammalian subject. In some embodiments, the subject is a human subject. In some embodiments, the subject is a nonhuman primate. In some embodiments, the subject is a dog, a cat, or other mammal.

In some embodiments, administration is initiated at a starting dose of diazoxide, or a pharmaceutically acceptable salt thereof, and then increased over a period of time to reach a target maintenance dose of diazoxide, or a pharmaceutically acceptable salt thereof.

In some embodiments, the target maintenance dose of diazoxide, or a pharmaceutically acceptable salt thereof, is reached after at least 4 weeks of treatment, including after 4-10 weeks of treatment, after 4-8 weeks of treatment, after 4-7 weeks of treatment, after 7-8 weeks of treatment, after 4 weeks of treatment, after 5 weeks of treatment, after 6 weeks of treatment, after 7 weeks of treatment, after 8 weeks of treatment, after 9 weeks of treatment, or after 10 weeks of treatment. In some embodiments, the target maintenance dose of diazoxide, or a pharmaceutically acceptable salt thereof, is reached after at least 10 weeks of treatment, including after 10-24 weeks of treatment, after 12-24 weeks of treatment, after 12-20 weeks of treatment, after 12-16 weeks of treatment, after 16-24 weeks of treatment, after 16-20 weeks of treatment, after 20-24 weeks of treatment, after 10 weeks of treatment, after 11 weeks of treatment, after 12 weeks of treatment, after 13 weeks of treatment, after 14 weeks of treatment, after 15 weeks of treatment, after 16 weeks of treatment, after 17 weeks of treatment, after 18 weeks of treatment, after 19 weeks of treatment, after 20 weeks of treatment, after 21 weeks of treatment, after 22 weeks of treatment, after 23 weeks of treatment, or after 24 weeks of treatment.

In some embodiments, the pharmaceutically acceptable salt is diazoxide choline. Diazoxide choline is also known as 7-Chloro-3-methyl-1λ6, 2,4-benzothiadiazin-2-ide 1,1-dioxide 2-hydroxyethyl(trimethyl)azanium, and has the structural formula:

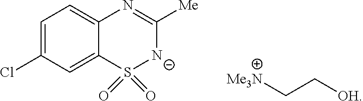

In some embodiments, the subject may be administered the free form of diazoxide or a salt other than diazoxide choline. In such cases, dosages described herein for diazoxide choline may be adjusted for the molar molecular weight of free form diazoxide or the alternative diazoxide salt being administered. The term “corresponding amount” as used herein refers to the amount of free form diazoxide or alternative diazoxide salt required to obtain the amount of diazoxide choline described in the methods, uses, and compounds for use described herein. It would be clear to one of skill in the art how to calculate the “corresponding amount” of free form or alternative salt of a compound, such as the corresponding amount of free form diazoxide or alternative salt of diazoxide choline, taking into account the difference in molecular weight between the free form of a compound and a given salt form. For example, 25.0 mg of diazoxide choline would correspond to 17.2 mg of diazoxide free form, as well as 20.0 mg of the HCl salt. As discussed herein, various references describing the preparation of diazoxide salts (e.g., diazoxide HCl, diazoxide choline, etc.) include, for example, U.S. Pat. Nos. 9,782,416, 7,572,789, 7,799,777, 9,381,202, 10,085,998, 11,045,478, 11,786,536, and US Pub. No. 2024/0252507, each of which are incorporated by reference in their entirety herein.

In some embodiments, the administration of diazoxide, or a pharmaceutically acceptable salt thereof, is initiated at a starting dose of diazoxide, or a pharmaceutically acceptable salt thereof, and then increased each 1-3 weeks to reach a target maintenance dose of diazoxide, or a pharmaceutically acceptable salt thereof. In some embodiments, the administration of diazoxide, or a pharmaceutically acceptable salt thereof, is initiated at a starting dose of diazoxide, or a pharmaceutically acceptable salt thereof, and then increased each 1-2 weeks to reach a target maintenance dose of diazoxide, or a pharmaceutically acceptable salt thereof. In some embodiments, the administration of diazoxide, or a pharmaceutically acceptable salt thereof, is initiated at a starting dose of diazoxide, or a pharmaceutically acceptable salt thereof, and then increased each 2-3 weeks to reach a target maintenance dose of diazoxide, or a pharmaceutically acceptable salt thereof. In some embodiments, the administration of diazoxide, or a pharmaceutically acceptable salt thereof, is initiated at a starting dose of diazoxide, or a pharmaceutically acceptable salt thereof, and then increased each 1-4 weeks to reach a target maintenance dose of diazoxide, or a pharmaceutically acceptable salt thereof. In some embodiments, the administration of diazoxide, or a pharmaceutically acceptable salt thereof, is initiated at a starting dose of diazoxide, or a pharmaceutically acceptable salt thereof, and then increased each 2-4 weeks to reach a target maintenance dose of diazoxide, or a pharmaceutically acceptable salt thereof. In some embodiments, the administration of diazoxide, or a pharmaceutically acceptable salt thereof, is initiated at a starting dose of diazoxide, or a pharmaceutically acceptable salt thereof, and then increased each 2-5 weeks to reach a target maintenance dose of diazoxide, or a pharmaceutically acceptable salt thereof. In some embodiments, the administration of diazoxide, or a pharmaceutically acceptable salt thereof, is initiated at a starting dose of diazoxide, or a pharmaceutically acceptable salt thereof, and then increased each 2-6 weeks to reach a target maintenance dose of diazoxide, or a pharmaceutically acceptable salt thereof.

In some embodiments, the target maintenance dose of diazoxide choline is reached after at least 4 weeks, including after 4-10 weeks, after 4-9 weeks, after 4-8 weeks, after 4-7 weeks, after 7-8 weeks, after 4 weeks, after 5 weeks, after 6 weeks, after 7 weeks, after 8 weeks, after 9 weeks, or after 10 weeks. In some embodiments, the target maintenance dose of diazoxide choline is reached after at least 10 weeks, including after 10-24 weeks, after 12-24 weeks, after 12-20 weeks, after 12-16 weeks, after 16-24 weeks, after 16-20 weeks, after 20-24 weeks, after 10 weeks, after 11 weeks, after 12 weeks, after 13 weeks, after 14 weeks, after 15 weeks, after 16 weeks, after 17 weeks, after 18 weeks, after 19 weeks, after 20 weeks, after 21 weeks, after 22 weeks, after 23 weeks, or after 24 weeks.

### Treating A Subject Having PWS

As also described herein, an effective amount of diazoxide, or a corresponding amount of a pharmaceutically acceptable salt thereof, may be administered to a subject having PWS to achieve therapeutic outcomes while reducing the incidence of adverse effects. Therapeutic outcomes may include, but are not limited to, reducing the severity or progression of PWS, alleviating comorbidities, enhancing biological function, or achieving a measurable clinical benefit. Subjects having PWS who can be treated include, but are not limited to, PWS subjects with one or more of the behavioral or cognitive and/or one more physiological characteristics of PWS, including those described herein. In some embodiments, the subject may have hyperphagia or hypotonia. In some embodiments, PWS subjects may exhibit aggressive behaviors or other behavioral characteristics that may be associated with PWS. In some embodiments, the subject may have hyperphagia.

With respect to a subject having PWS, the effect of administration may include, but is not limited to, reducing the severity or progression of PWS, alleviating comorbidities, enhancing biological function, or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, below.

TABLE 1
Example Diazoxide Choline Dosing Schedule

| | | Titration | | Titration | | Titration | | Target | |
|---|---|---|---|---|---|---|---|---|---|
| | | Step 1 | | Step 2 | | Step 3 | | Maintenance | |
| Weight | | (Weeks | | (Weeks | | (Weeks | | Dose (Week 7 | |
| Range | | 1 and 2) | | 3 and 4) | | 5 and 6) | | and After) | |
| ≥3 to <10 | kg | 1.5 | mg/kg/day | 2.4 | mg/kg/day | 3.3 | mg/kg/day | 4.2 | mg/kg/day |
| ≥10 to <14 | kg | 25 | mg/day | 50 | mg/day | 50 | mg/day | 58 | mg/day |
| ≥14 to <20 | kg | 25 | mg/day | 50 | mg/day | 75 | mg/day | 80 | mg/day |
| ≥20 to <30 | kg | 25 | mg/day | 50 | mg/day | 75 | mg/day | 100 | mg/day |
| ≥30 to <40 | kg | 75 | mg/day | 150 | mg/day | 150 | mg/day | 150 | mg/day |
| ≥40 to <65 | kg | 75 | mg/day | 150 | mg/day | 225 | mg/day | 225 | mg/day |
| ≥65 to <100 | kg | 150 | mg/day | 225 | mg/day | 300 | mg/day | 375 | mg/day |
| ≥100 to <135 | kg | 150 | mg/day | 300 | mg/day | 375 | mg/day | 450 | mg/day |
| ≥135 | kg | 150 | mg/day | 300 | mg/day | 450 | mg/day | 525 | mg/day |

In some embodiments, a subject may not initially be titrated to the target maintenance dose and instead will initially be titrated to a lower dose based on the titration steps described herein. For some subjects, after a period of time (e.g., 3 months, 4 months, 6 months, 9 months, 12 months, 18 months, or more), further dose escalations up to the target maintenance doses described herein may be undertaken based on a need for greater efficacy and a subject's response to treatment. For example, for some subjects weighing≥65 kg, it may be recommended to initially titrate to 300 mg/day (Titration Step 2 or 3) based on tolerability. Further dose escalation up to the Target Maintenance Dose may be considered based on the need for greater efficacy, balanced with the potential for adverse drug reactions. The maximum recommended dosage is 5.8 mg/kg/day or 525 mg/day.

Treating A Subject with One or More Clinically Relevant Comorbidities

As noted herein, there are circumstances under which dosing modifications and more frequent monitoring may be considered in subjects having PWS. Specifically, the present disclosure delineates example diazoxide choline dosing modifications, as well as additional monitoring protocol, for subjects with one or more clinically relevant comorbidities.

The term “clinically relevant comorbidity,” as used herein, refers to a previous or coexisting condition (e.g., a medical condition, history of a medical condition, concomitant use of one or more medications, etc.) present prior to treatment that affects the diagnosis, treatment, or prognosis of a subject's illness. For example, clinically relevant comorbidities of PWS include, but are not limited to, cardiovascular (e.g., a history of edema, edema, fluid retention, fluid overload, congestive heart failure, compromised cardiac reserve, heart disease, etc.), pulmonary (e.g., a history of asthma, asthma, chronic lung disease, Chronic Obstructive Pulmonary Disease (COPD), obstructive sleep apnea, central sleep apnea, recent pulmonary infection, etc.), and metabolic/endocrine (e.g., obesity, hyperglycemia, insulin resistance, elevated fasting glucose, elevated HbA1c, prediabetes, diabetes, concomitant use of growth hormones, corticosteroids or medications known to impact blood glucose, etc.) comorbidities, as well as concomitant use of one or more medications that could contribute to hyperglycemia, fluid retention, or weight gain.

As will be appreciated by the skilled artisan, treatment modification in view of one or more clinically relevant comorbidities is dependent on the type, frequency, severity, and number of clinically relevant comorbidities exhibited by the subject. For example, a medical practitioner may assess that treatment modification is warranted for a subject with an elevated HbA1c level greater than or equal to 6.5%, or a subject with an elevated fasting glucose level of greater than 110 mg/dL. However, a medical practitioner may assess that treatment modification is warranted at significantly lower thresholds if a subject has both an elevated HbA1c level and an elevated fasting glucose level, for instance.

Examples of modified dose titrations for subjects with one or more clinically relevant comorbidities within different weight ranges are shown in Tables 2a-2c, below.

TABLE 2a
First Example of Diazoxide Choline Dosage Modifications for
Patients with One or More Clinically Relevant Comorbidities

| Step | | | |
|---|---|---|---|
| Starting | | | |
| Dose | Step 2 | Step 3 | Target |
| Duration | | | |
| 2 to 4+ | 2 to 4+ | 2 to 4+ | Maintenance |
| Weeks | Weeks | Weeks | Therapy |

Body Weight:

| | | | | | | | | | |
|---|---|---|---|---|---|---|---|---|---|
| 20 to <30 | kg | 25 | mg | 25-50 | mg | 50-75 | mg | 50-75 mg | (max 100 mg if tolerated) |
| 30 to <40 | kg | 50-75 | mg | 75-100 | mg | 75-100 | mg | 100 mg | (max 150 mg if tolerated) |
| 40 to <65 | kg | 50-75 | mg | 75-150 | mg | 75-150 | mg | 150 mg | (max 225 mg if tolerated) |
| 65 to <100 | kg | 75-100 | mg | 150-200 | mg | 200-250 | mg | 200-250 mg | (max 375 mg if tolerated) |
| 100 to <135 | kg | 75-100 | mg | 150-225 | mg | 225-300 | mg | 300-400 mg | (max 450 mg if tolerated) |
| ≥135 | kg | 75-100 | mg | 150-225 | mg | 225-300 | mg | 300-400 mg | (max 525 mg if tolerated) |

TABLE 2b
Second Example of Diazoxide Choline Dosage Modifications for
Patients with One or More Clinically Relevant Comorbidities

| Step | | | | | | |
|---|---|---|---|---|---|---|
| Starting | | | | | | |
| Dose | Step 2 | Step 3 | Step 4 | Step 5 | Step 6 | Target |
| Duration | | | | | | |
| 2 to 4+ | 2 to 4+ | 2 to 4+ | 2 to 4+ | 2 to 4+ | 2 to 4+ | Maintenance |
| Weeks | Weeks | Weeks | Weeks | Weeks | Weeks | Therapy |

Body Weight:

| | | | | | | | | | | |
|---|---|---|---|---|---|---|---|---|---|---|
| 20 to <30 | kg | 25 mg | 50 | mg | 75 | mg | 100 mg | 100 mg | 100 mg | 100 mg |
| 30 to <40 | kg | 75 mg | 150 | mg | 150 | mg | 150 mg | 150 mg | 150 mg | 150 mg |
| 40 to <65 | kg | 75 mg | 150 | mg | 225 | mg | 225 mg | 225 mg | 225 mg | 225 mg |
| 65 to <100 | kg | 75 mg | 150 | mg | 225 | mg | 300 mg | 375 mg | 375 mg | 375 mg |
| 100 to <135 | kg | 75 mg | 150 | mg | 225 | mg | 300 mg | 375 mg | 450 mg | 450 mg |
| ≥135 | kg | 75 mg | 150 | mg | 225 | mg | 300 mg | 375 mg | 450 mg | 525 mg |

TABLE 2c
Third Example of Diazoxide Choline Dosage Modifications for
Patients with One or More Clinically Relevant Comorbidities

| | | Step 1 | Step 2 | | Step 3 | | Step 4 | Step 5 | Step 6 | Initial | Max |
|---|---|---|---|---|---|---|---|---|---|---|---|
| | | Weeks | Weeks | | Weeks | | Weeks | Weeks | Weeks | Target | Target |
| Weight | | 1-4 | 5-8 | | 9-12 | | 12-16 | 17-20 | 20-24 | Dose | Dose |
| 20 to <30 | kg | 25 mg | 50 | mg | 75 | mg | 100 mg | — | — | 100 mg | 100 mg |
| 30 to <40 | kg | 75 mg | 150 | mg | — | | — | — | — | 150 mg | 150 mg |
| 40 to <65 | kg | 75 mg | 150 | mg | 225 | mg | — | — | — | 225 mg | 225 mg |
| 65 to <100 | kg | 75 mg | 150 | mg | 225 | mg | 275 mg | — | — | 275 mg | 375 mg |
| 100 to <135 | kg | 75 mg | 150 | mg | 225 | mg | 275 mg | 325 mg | — | 325 mg | 450 mg |
| ≥135 | kg | 75 mg | 150 | mg | 225 | mg | 275 mg | 325 mg | 375 mg | 375 mg | 525 mg |

In some embodiments, certain patients may require a modified dosing schedule. Examples of said modified dosing schedules are provided in Tables 2a-2c. In some embodiments, the subject is dosed according to Tables 2a, 2b, or 2c. In some embodiments, the subject is dosed according to Table 2a. In some embodiments, the subject is dosed according to Table 2b. In some embodiments, the subject is dosed according to Table 2c.

In some embodiments, the modified starting dose of diazoxide choline is between about 25-100 mg of diazoxide choline, including between about 25-75 mg of diazoxide choline, between about 25-50 mg of diazoxide choline, between about 50-100 mg of diazoxide choline, between about 50-75 mg of diazoxide choline, or between about 75-100 mg of diazoxide choline. In some embodiments, the modified starting dose of diazoxide choline is about 25 mg of diazoxide choline, about 50 mg of diazoxide choline, about 75 mg of diazoxide choline, or about 100 mg of diazoxide choline.

In some embodiments, the modified target maintenance dose of diazoxide choline is between about 50-525 mg of diazoxide choline, including between about 50-450 mg of diazoxide choline, between about 50-400 mg of diazoxide choline, between about 50-375 mg of diazoxide choline, between about 50-300 mg of diazoxide choline, between about 50-250 mg of diazoxide choline, between about 50-225 mg of diazoxide choline, between about 50-200 mg of diazoxide choline, between about 50-150 mg of diazoxide choline, between about 50-100 mg of diazoxide choline, between about 50-75 mg of diazoxide choline, between about 100-525 mg of diazoxide choline, between about 100-450 mg of diazoxide choline, between about 100-400 mg of diazoxide choline, between about 100-375 mg of diazoxide choline, between about 100-300 mg of diazoxide choline, between about 100-250 mg of diazoxide choline, between about 100-225 mg of diazoxide choline, between about 100-200 mg of diazoxide choline, between about 100-150 mg of diazoxide choline, between about 150-525 mg of diazoxide choline, between about 150-450 mg of diazoxide choline, between about 150-400 mg of diazoxide choline, between about 150-375 mg of diazoxide choline, between about 150-300 mg of diazoxide choline, between about 150-250 mg of diazoxide choline, between about 150-225 mg of diazoxide choline, between about 150-200 mg of diazoxide choline, between about 200-525 mg of diazoxide choline, between about 200-450 mg of diazoxide choline, between about 200-400 mg of diazoxide choline, between about 200-375 mg of diazoxide choline, between about 200-300 mg of diazoxide choline, between about 200-250 mg of diazoxide choline, between about 200-225 mg of diazoxide choline, between about 225-525 mg of diazoxide choline, between about 225-450 mg of diazoxide choline, between about 225-400 mg of diazoxide choline, between about 225-375 mg of diazoxide choline, between about 225-300 mg of diazoxide choline, between about 225-250 mg of diazoxide choline, between about 300-525 mg of diazoxide choline, between about 300-450 mg of diazoxide choline, between about 300-400 mg of diazoxide choline, between about 300-375 mg of diazoxide choline, between about 375-525 mg of diazoxide choline, between about 375-450 mg of diazoxide choline, or between about 450-525 mg of diazoxide choline. In some embodiments, the modified target maintenance dose of diazoxide choline is about 50 mg of diazoxide choline, about 75 mg of diazoxide choline, about 100 mg of diazoxide choline, about 150 mg of diazoxide choline, about 200 mg of diazoxide choline, about 225 mg of diazoxide choline, about 250 mg of diazoxide choline, about 300 mg of diazoxide choline, about 375 mg of diazoxide choline, about 400 mg of diazoxide choline, about 450 mg of diazoxide choline, or about 525 mg of diazoxide choline. In some embodiments, the modified target maintenance dose of diazoxide choline is between about 50-525 mg of diazoxide choline, including between about 100-275 mg of diazoxide choline, between about 100-325 mg of diazoxide choline, between about 150-275 mg of diazoxide choline, between about 150-325 mg of diazoxide choline, between about 225-275 mg of diazoxide choline, between about 225-325 mg of diazoxide choline, between about 275-325 mg of diazoxide choline, between about 275-375 mg of diazoxide choline, or between about 325-375 mg of diazoxide choline.

In some embodiments, the modified target maintenance dose of diazoxide choline is about 275 mg of diazoxide choline or about 325 mg of diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of at least two weeks;
(b) after the initial period of at least two weeks, orally administering to the subject 25-50 mg of diazoxide choline once daily for a second period of at least two weeks;
(c) after the second period of at least two weeks, orally administering to the subject 50-75 mg of diazoxide choline once daily for a third period of at least two weeks; and
   (i) after the third period of at least two weeks, orally administering to the subject a target maintenance dose of 50-75 mg of diazoxide choline once daily; or
   (ii) after the third period of at least two weeks, orally administering to the subject a maximum target maintenance dose of 100 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of at least two weeks;
(b) after the initial period of at least two weeks, orally administering to the subject 50 mg of diazoxide choline once daily for a second period of at least two weeks;
(c) after the second period of at least two weeks, orally administering to the subject 75 mg of diazoxide choline once daily for a third period of at least two weeks; and
(d) after the third period of at least two weeks, orally administering to the subject a target maintenance dose of 100 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of at least four weeks;
(b) after the initial period of at least four weeks, orally administering to the subject 50 mg of diazoxide choline once daily for a second period of at least four weeks;
(c) after the second period of at least four weeks, orally administering to the subject 75 mg of diazoxide choline once daily for a third period of at least four weeks; and
(d) after the third period of at least four weeks, orally administering to the subject a target maintenance dose of 100 mg of diazoxide choline once daily;
wherein the subject has one or more clinically relevant comorbidities prior to treatment with diazoxide choline.

In some embodiments, the subject weighs from about 20 kg to less than 30 kg. In some embodiments, the subject weighs about 20 kg. In some embodiments, the subject weighs about 25 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50-75 mg of diazoxide choline once daily for an initial period of at least two weeks;
(b) after the initial period of at least two weeks, orally administering to the subject 75-100 mg of diazoxide choline once daily for a subsequent period of at least four weeks; and
   (i) after the subsequent period of at least four weeks, orally administering to the subject a target maintenance dose of 100 mg of diazoxide choline once daily; or
   (ii) after the subsequent period of at least four weeks, orally administering to the subject a maximum target maintenance dose of 150 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks; and
(b) after the initial period of at least two weeks, orally administering to the subject a target maintenance dose of 150 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks; and
(b) after the initial period of at least four weeks, orally administering to the subject a target maintenance dose of 150 mg of diazoxide choline once daily;
wherein the subject has one or more clinically relevant comorbidities prior to treatment with diazoxide choline.

In some embodiments, the subject weighs from 30 kg to less than 40 kg. In some embodiments, the subject weighs about 30 kg. In some embodiments, the subject weighs about 35 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50-75 mg of diazoxide choline once daily for an initial period of at least two weeks;
(b) after the initial period of at least two weeks, orally administering to the subject 75-150 mg of diazoxide choline once daily for a subsequent period of at least four weeks; and
   (i) after the subsequent period of at least four weeks, orally administering to the subject a target maintenance dose of 150 mg of diazoxide choline once daily; or
   (ii) after the subsequent period of at least four weeks, orally administering to the subject a maximum target maintenance dose of 225 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks; and
(c) after the second period of at least two weeks, orally administering to the subject a target maintenance dose of 225 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks; and
(c) after the second period of at least four weeks, orally administering to the subject a target maintenance dose of 225 mg of diazoxide choline once daily;
wherein the subject has one or more clinically relevant comorbidities prior to treatment with diazoxide choline.

In some embodiments, the subject weighs from 40 kg to less than 65 kg. In some embodiments, the subject weighs about 40 kg. In some embodiments, the subject weighs about 45 kg. In some embodiments, the subject weighs about 50 kg. In some embodiments, the subject weighs about 55 kg. In some embodiments, the subject weighs about 60 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75-100 mg of diazoxide choline once daily for an initial period of at least two weeks;
(b) after the initial period of at least two weeks, orally administering to the subject 150-200 mg of diazoxide choline once daily for a second period of at least two weeks;
(c) after the second period of at least two weeks, orally administering to the subject 200-250 mg of diazoxide choline once daily for a third period of at least two weeks; and
   (i) after the third period of at least two weeks, orally administering to the subject a target maintenance dose of 200-250 mg of diazoxide choline once daily; or
   (ii) after the third period of at least two weeks, orally administering to the subject a maximum target maintenance dose of 375 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks; and
(e) after the fourth period of at least two weeks, orally administering to the subject a target maintenance dose of 375 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 375 mg of diazoxide choline once daily;
wherein the subject has one or more clinically relevant comorbidities prior to treatment with diazoxide choline.

In some embodiments, the subject weighs from 65 kg to less than 100 kg. In some embodiments, the subject weighs about 65 kg. In some embodiments, the subject weighs about 70 kg. In some embodiments, the subject weighs about 75 kg. In some embodiments, the subject weighs about 80 kg. In some embodiments, the subject weighs about 85 kg. In some embodiments, the subject weighs about 90 kg. In some embodiments, the subject weighs about 95 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75-100 mg of diazoxide choline once daily for an initial period of at least two weeks;
(b) after the initial period of at least two weeks, orally administering to the subject 150-225 mg of diazoxide choline once daily for a second period of at least two weeks;
(c) after the second period of at least two weeks, orally administering to the subject 225-300 mg of diazoxide choline once daily for a third period of at least two weeks; and
   (i) after the third period of at least two weeks, orally administering to the subject a target maintenance dose of 300-400 mg of diazoxide choline once daily; or
   (ii) after the third period of at least two weeks, orally administering to the subject a maximum target maintenance dose of 450 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks; and
(f) after the fifth period of at least two weeks, orally administering to the subject a target maintenance dose of 450 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject an initial target dose of 325 mg of diazoxide choline once daily for a period of at least six months; and
   (i) after the period of at least six months, orally administering to the subject the initial target dose of 325 mg of diazoxide choline once daily; or
   (ii) after the period of at least six months, orally administering to the subject a maximum target dose of 450 mg of diazoxide choline once daily;
wherein the subject has one or more clinically relevant comorbidities prior to treatment with diazoxide choline.

In some embodiments, the subject weighs from 100 kg to less than 135 kg. In some embodiments, the subject weighs about 100 kg. In some embodiments, the subject weighs about 105 kg. In some embodiments, the subject weighs about 110 kg. In some embodiments, the subject weighs about 115 kg. In some embodiments, the subject weighs about 120 kg. In some embodiments, the subject weighs about 125 kg. In some embodiments, the subject weighs about 130 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75-100 mg of diazoxide choline once daily for an initial period of at least two weeks;
(b) after the initial period of at least two weeks, orally administering to the subject 150-225 mg of diazoxide choline once daily for a second period of at least two weeks;
(c) after the second period of at least two weeks, orally administering to the subject 225-300 mg of diazoxide choline once daily for a third period of at least two weeks; and
   (i) after the third period of at least two weeks, orally administering to the subject a target maintenance dose of 300-400 mg of diazoxide choline once daily; or
   (ii) after the third period of at least two weeks, orally administering to the subject a maximum target maintenance dose of 525 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks;
(f) after the fifth period of at least two weeks, orally administering to the subject 450 mg of diazoxide choline once daily for a sixth period of at least two weeks; and
(g) after the sixth period of at least two weeks, orally administering to the subject a target maintenance dose of 525 mg of diazoxide choline once daily;
wherein the subject has one or more clinically relevant comorbidities prior to treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject 325 mg of diazoxide choline once daily for a fifth period of at least four weeks;
(f) after the fifth period of at least four weeks, orally administering to the subject an initial target dose of 375 mg of diazoxide choline once daily for a period of at least six months; and
   (i) after the period of at least six months, orally administering to the subject the initial target dose of 375 mg of diazoxide choline once daily; or
   (ii) after the period of at least six months, orally administering to the subject a maximum target dose of 525 mg of diazoxide choline once daily;
wherein the subject has one or more clinically relevant comorbidities prior to treatment with diazoxide choline.

In some embodiments, the subject weighs 135 kg or more. In some embodiments, the subject weighs about 135 kg. In some embodiments, the subject weighs about 140 kg. In some embodiments, the subject weighs about 145 kg. In some embodiments, the subject weighs about 150 kg. In some embodiments, the subject weighs about 155 kg. In some embodiments, the subject weighs about 160 kg. In some embodiments, the subject weighs about 165 kg. In some embodiments, the subject weighs about 170 kg.

In some embodiments, the initial period of at least two weeks is 2-6 weeks. In some embodiments, the initial period of at least two weeks is 2-5 weeks. In some embodiments, the initial period of at least two weeks is 2-4 weeks. In some embodiments, the initial period of at least two weeks is two, three, four, five, or six weeks. In some embodiments, the initial period of at least two weeks is two, three, four, or five weeks. In some embodiments, the initial period of at least two weeks is two, three, or four weeks. In some embodiments, the initial period of at least two weeks is two weeks. In some embodiments, the initial period of at least two weeks is three weeks. In some embodiments, the initial period of at least two weeks is four weeks. In some embodiments, the initial period of at least two weeks is five weeks. In some embodiments, the initial period of at least two weeks is six weeks.

In some embodiments, the second period of at least two weeks is 2-6 weeks. In some embodiments, the second period of at least two weeks is 2-5 weeks. In some embodiments, the second period of at least two weeks is 2-4 weeks. In some embodiments, the second period of at least two weeks is two, three, four, five, or six weeks. In some embodiments, the second period of at least two weeks is two, three, four, or five weeks. In some embodiments, the second period of at least two weeks is two, three, or four weeks. In some embodiments, the second period of at least two weeks is two weeks. In some embodiments, the second period of at least two weeks is three weeks. In some embodiments, the second period of at least two weeks is four weeks. In some embodiments, the second period of at least two weeks is five weeks. In some embodiments, the second period of at least two weeks is six weeks.

In some embodiments, the third period of at least two weeks is 2-6 weeks. In some embodiments, the third period of at least two weeks is 2-5 weeks. In some embodiments, the third period of at least two weeks is 2-4 weeks. In some embodiments, the third period of at least two weeks is two, three, four, five, or six weeks.

In some embodiments, the third period of at least two weeks is two, three, four, or five weeks. In some embodiments, the third period of at least two weeks is two, three, or four weeks. In some embodiments, the third period of at least two weeks is two weeks. In some embodiments, the third period of at least two weeks is three weeks. In some embodiments, the third period of at least two weeks is four weeks. In some embodiments, the third period of at least two weeks is five weeks. In some embodiments, the third period of at least two weeks is six weeks.

In some embodiments, the fourth period of at least two weeks is 2-6 weeks. In some embodiments, the fourth period of at least two weeks is 2-5 weeks. In some embodiments, the fourth period of at least two weeks is 2-4 weeks. In some embodiments, the fourth period of at least two weeks is two, three, four, five, or six weeks. In some embodiments, the fourth period of at least two weeks is two, three, four, or five weeks. In some embodiments, the fourth period of at least two weeks is two, three, or four weeks. In some embodiments, the fourth period of at least two weeks is two weeks. In some embodiments, the fourth period of at least two weeks is three weeks. In some embodiments, the fourth period of at least two weeks is four weeks. In some embodiments, the fourth period of at least two weeks is five weeks. In some embodiments, the fourth period of at least two weeks is six weeks.

In some embodiments, the fifth period of at least two weeks is 2-6 weeks. In some embodiments, the fifth period of at least two weeks is 2-5 weeks. In some embodiments, the fifth period of at least two weeks is 2-4 weeks. In some embodiments, the fifth period of at least two weeks is two, three, four, five, or six weeks.

In some embodiments, the fifth period of at least two weeks is two, three, four, or five weeks. In some embodiments, the fifth period of at least two weeks is two, three, or four weeks. In some embodiments, the fifth period of at least two weeks is two weeks. In some embodiments, the fifth period of at least two weeks is three weeks. In some embodiments, the fifth period of at least two weeks is four weeks. In some embodiments, the fifth period of at least two weeks is five weeks. In some embodiments, the fifth period of at least two weeks is six weeks.

In some embodiments, the sixth period of at least two weeks is 2-6 weeks. In some embodiments, the sixth period of at least two weeks is 2-5 weeks. In some embodiments, the sixth period of at least two weeks is 2-4 weeks. In some embodiments, the sixth period of at least two weeks is two, three, four, five, or six weeks.

In some embodiments, the sixth period of at least two weeks is two, three, four, or five weeks. In some embodiments, the sixth period of at least two weeks is two, three, or four weeks. In some embodiments, the sixth period of at least two weeks is two weeks. In some embodiments, the sixth period of at least two weeks is three weeks. In some embodiments, the sixth period of at least two weeks is four weeks. In some embodiments, the sixth period of at least two weeks is five weeks. In some embodiments, the sixth period of at least two weeks is six weeks.

In some embodiments, the subsequent period of at least four weeks is 4-8 weeks. In some embodiments, the subsequent period of at least four weeks is 4-7 weeks. In some embodiments, the subsequent period of at least four weeks is 4-6 weeks. In some embodiments, the subsequent period of at least four weeks is four, five, six, seven, or eight weeks. In some embodiments, the subsequent period of at least four weeks is four, five, six, or seven weeks. In some embodiments, the subsequent period of at least four weeks is four, five, or six weeks. In some embodiments, the subsequent period of at least four weeks is four weeks. In some embodiments, the subsequent period of at least four weeks is five weeks. In some embodiments, the subsequent period of at least four weeks is six weeks. In some embodiments, the subsequent period of at least four weeks is seven weeks. In some embodiments, the subsequent period of at least four weeks is eight weeks.

In some embodiments, the initial period of at least four weeks is 4-8 weeks. In some embodiments, the initial period of at least four weeks is 4-7 weeks. In some embodiments, the initial period of at least four weeks is 4-6 weeks. In some embodiments, the initial period of at least four weeks is four, five, six, seven, or eight weeks. In some embodiments, the initial period of at least four weeks is four, five, six, or seven weeks. In some embodiments, the initial period of at least four weeks is four, five, or six weeks. In some embodiments, the initial period of at least four weeks is four weeks. In some embodiments, the initial period of at least four weeks is five weeks. In some embodiments, the initial period of at least four weeks is six weeks. In some embodiments, the initial period of at least four weeks is seven weeks. In some embodiments, the initial period of at least four weeks is eight weeks.

In some embodiments, the second period of at least four weeks is 4-8 weeks. In some embodiments, the second period of at least four weeks is 4-7 weeks. In some embodiments, the second period of at least four weeks is 4-6 weeks. In some embodiments, the second period of at least four weeks is four, five, six, seven, or eight weeks. In some embodiments, the second period of at least four weeks is four, five, six, or seven weeks.

In some embodiments, the second period of at least four weeks is four, five, or six weeks. In some embodiments, the second period of at least four weeks is four weeks. In some embodiments, the second period of at least four weeks is five weeks. In some embodiments, the second period of at least four weeks is six weeks. In some embodiments, the second period of at least four weeks is seven weeks. In some embodiments, the second period of at least four weeks is eight weeks.

In some embodiments, the third period of at least four weeks is 4-8 weeks. In some embodiments, the third period of at least four weeks is 4-7 weeks. In some embodiments, the third period of at least four weeks is 4-6 weeks. In some embodiments, the third period of at least four weeks is four, five, six, seven, or eight weeks. In some embodiments, the third period of at least four weeks is four, five, six, or seven weeks. In some embodiments, the third period of at least four weeks is four, five, or six weeks. In some embodiments, the third period of at least four weeks is four weeks. In some embodiments, the third period of at least four weeks is five weeks. In some embodiments, the third period of at least four weeks is six weeks. In some embodiments, the third period of at least four weeks is seven weeks. In some embodiments, the third period of at least four weeks is eight weeks.

In some embodiments, the fourth period of at least four weeks is 4-8 weeks. In some embodiments, the fourth period of at least four weeks is 4-7 weeks. In some embodiments, the fourth period of at least four weeks is 4-6 weeks. In some embodiments, the fourth period of at least four weeks is four, five, six, seven, or eight weeks. In some embodiments, the fourth period of at least four weeks is four, five, six, or seven weeks.

In some embodiments, the fourth period of at least four weeks is four, five, or six weeks. In some embodiments, the fourth period of at least four weeks is four weeks. In some embodiments, the fourth period of at least four weeks is five weeks. In some embodiments, the fourth period of at least four weeks is six weeks. In some embodiments, the fourth period of at least four weeks is seven weeks. In some embodiments, the fourth period of at least four weeks is eight weeks.

In some embodiments, the fifth period of at least four weeks is 4-8 weeks. In some embodiments, the fifth period of at least four weeks is 4-7 weeks. In some embodiments, the fifth period of at least four weeks is 4-6 weeks. In some embodiments, the fifth period of at least four weeks is four, five, six, seven, or eight weeks.

In some embodiments, the fifth period of at least four weeks is four, five, six, or seven weeks. In some embodiments, the fifth period of at least four weeks is four, five, or six weeks. In some embodiments, the fifth period of at least four weeks is four weeks. In some embodiments, the fifth period of at least four weeks is five weeks. In some embodiments, the fifth period of at least four weeks is six weeks. In some embodiments, the fifth period of at least four weeks is seven weeks. In some embodiments, the fifth period of at least four weeks is eight weeks.

In some embodiments, the period of at least six months is 6-12 months. In some embodiments, the period of at least six months is 6-11 months. In some embodiments, the period of at least six months is 6-10 months. In some embodiments, the period of at least six months is 6-9 months. In some embodiments, the period of at least six months is 6-8 months. In some embodiments, the period of at least six months is six, seven, eight, nine, ten, eleven, or twelve months. In some embodiments, the period of at least six months is six, seven, eight, nine, ten, or eleven months. In some embodiments, the period of at least six months is six, seven, eight, nine, or ten months. In some embodiments, the period of at least six months is six, seven, eight, or nine months. In some embodiments, the period of at least six months is six, seven, or eight months. In some embodiments, the period of at least six months is six months. In some embodiments, the period of at least six months is seven months. In some embodiments, the period of at least six months is eight months. In some embodiments, the period of at least six months is nine months. In some embodiments, the period of at least six months is ten months. In some embodiments, the period of at least six months is eleven months. In some embodiments, the period of at least six months is twelve months.

In some embodiments, the target maintenance dose is reached after at least 10 weeks of treatment. In some embodiments, the target maintenance dose is reached after 10-24 weeks of treatment. In some embodiments, the target maintenance dose is reached after 12-24 weeks of treatment. In some embodiments, the target maintenance dose is reached after 12-20 weeks of treatment. In some embodiments, the target maintenance dose is reached after 12-16 weeks of treatment. In some embodiments, the target maintenance dose is reached after 16-24 weeks of treatment. In some embodiments, the target maintenance dose is reached after 16-20 weeks of treatment. In some embodiments, the target maintenance dose is reached after 20-24 weeks of treatment. In some embodiments, the target maintenance dose is reached after 10 weeks of treatment.

In some embodiments, the target maintenance dose is reached after 11 weeks of treatment. In some embodiments, the target maintenance dose is reached after 12 weeks of treatment. In some embodiments, the target maintenance dose is reached after 13 weeks of treatment. In some embodiments, the target maintenance dose is reached after 14 weeks of treatment. In some embodiments, the target maintenance dose is reached after 15 weeks of treatment. In some embodiments, the target maintenance dose is reached after 16 weeks of treatment. In some embodiments, the target maintenance dose is reached after 17 weeks of treatment. In some embodiments, the target maintenance dose is reached after 18 weeks of treatment. In some embodiments, the target maintenance dose is reached after 19 weeks of treatment. In some embodiments, the target maintenance dose is reached after 20 weeks of treatment. In some embodiments, the target maintenance dose is reached after 21 weeks of treatment. In some embodiments, the target maintenance dose is reached after 22 weeks of treatment. In some embodiments, the target maintenance dose is reached after 23 weeks of treatment. In some embodiments, the target maintenance dose is reached after 24 weeks of treatment.

In some embodiments, after the target maintenance dose is reached, the amount of diazoxide choline orally administered once daily to the subject is increased by no more than 25 mg every adjustment period, wherein every adjustment period has a duration of at least two weeks, wherein the amount of diazoxide choline orally administered once daily to the subject is no more than 5.8 mg/kg. In some embodiments, after the target maintenance dose is reached, the amount of diazoxide choline orally administered once daily to the subject is increased by no more than 75 mg every adjustment period, wherein every adjustment period has a duration of at least two weeks, wherein the amount of diazoxide choline orally administered once daily to the subject is no more than 5.8 mg/kg or 525 mg, whichever is lower.

In some embodiments, the once daily administration dose of diazoxide choline is titrated over a longer duration (e.g., for at least 4 weeks per titration step) to minimize risk of fluid overload in subjects at elevated risk based on edema, obesity, or cardiopulmonary status. In some embodiments, the once daily administration dose of diazoxide choline is titrated to a lower maintenance dose to minimize risk of fluid overload in subjects at elevated risk based on edema, obesity, or cardiopulmonary status. In some embodiments, the once daily administration dose of diazoxide choline is titrated over a longer duration (e.g., for at least 4 weeks per titration step) to a lower maintenance dose to minimize risk of fluid overload in subjects at elevated risk based on edema, obesity, or cardiopulmonary status.

In some embodiments, the methods disclosed herein further comprise additional monitoring of the subject during treatment.

In some embodiments, the one or more clinically relevant comorbidities is hyperglycemia. In some embodiments, the methods disclosed herein further comprise periodically monitoring the fasting glucose of the subject during treatment. In some embodiments, the monitoring takes place at least once a week during titration (e.g., 1, 2, 3, or 4 times per week). In some embodiments, the monitoring takes place every two weeks after titration. In some embodiments, the monitoring takes place weekly for the first two weeks of treatment, then at least once every four weeks of treatment. In some embodiments, the monitoring takes place as clinically indicated after titration.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise modifying the dosing schedule administered to the subject if said subject exhibits clinically significant glucose elevation during titration. In some embodiments, the modified dosing schedule comprises titrating the subject over longer durations relative to the unmodified dosing schedule (e.g., the dosing schedule recited in Table 1). In some embodiments, the modified dosing schedule comprises titrating the subject to a lower dosage relative to the unmodified dosing schedule (e.g., the dosing schedule recited in Table 1). In some embodiments, the modified dosing schedule may be according to Table 2a, 2b, or 2c. In some embodiments, the modified dosing schedule may be according to Table 2a. In some embodiments, the modified dosing schedule may be according to Table 2b. In some embodiments, the modified dosing schedule may be according to Table 2c.

In some embodiments, the one or more clinically relevant comorbidities is elevated HbA1c. In some embodiments, the methods disclosed herein further comprise periodically monitoring the HbA1c of the subject during treatment. In some embodiments, the monitoring takes place every 3 months. In some embodiments, the monitoring takes place as clinically indicated.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise:
(a) testing the fasting plasma glucose and HbA1c of the subject prior to treatment; and
(b) optionally optimizing the blood glucose of the subject prior to treatment.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise temporarily interrupting (e.g., for 3 days, 6 days, 7 days, 10 days, 14 days, etc.) or reducing the dose administered to the subject (e.g., reducing the dose administered to the subject to the previous titration dose, etc.) if said subject exhibits clinically significant fasting glucose or HbA1c elevation during treatment.

In some embodiments, the dose administered to the subject is restored to its original amount after the subject's glycemic parameters are appropriately managed. In some embodiments, the dose administered to the subject is restored to its original amount after the subject's clinically significant fasting glucose or HbA1c elevation is resolved. In some embodiments, the subject is re-titrated after the subject's clinically significant fasting glucose or HbA1c elevation is resolved. For example, in some embodiments, where treatment is interrupted for seven days or more, re-titration of diazoxide choline should be implemented when diazoxide choline treatment is resumed. In some embodiments, the subject is re-titrated in dosage increments of no more than 25 mg of diazoxide choline every 2 weeks. In some embodiments, the subject is re-titrated in dosage increments of no more than 75 mg of diazoxide choline every 2 weeks. In some embodiments, the subject is re-titrated over a longer duration relative to the initial titration.

In some embodiments, the one or more clinically relevant comorbidities is edema or fluid overload. In some embodiments, the one or more clinically relevant comorbidities is edema. In some embodiments, the one or more clinically relevant comorbidities is fluid overload. In some embodiments, the one or more clinically relevant comorbidities is fluid retention. In some embodiments, the methods disclosed herein further comprise periodically monitoring the body weight of the subject during treatment. In some embodiments, the methods disclosed herein further comprise periodically assessing the subject for swelling of a body part during treatment. In some embodiments, the body part is an ankle, foot, or leg. In some embodiments, the body part is an ankle. In some embodiments, the body part is a foot. In some embodiments, the body part is a leg. In some embodiments, the methods disclosed herein further comprise periodically assessing the subject for one or more respiratory symptoms during treatment. In some embodiments, the one or more respiratory symptoms is dyspnea or shortness of breath. In some embodiments, the one or more respiratory symptoms is dyspnea. In some embodiments, the one or more respiratory symptoms is shortness of breath. In some embodiments, the monitoring takes place once a week during titration. In some embodiments, the monitoring takes place at regular intervals after titration.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise temporarily interrupting (e.g., for 3 days, 6 days, 7 days, 10 days, 14 days, etc.) or reducing the dose administered to the subject (e.g., reducing the dose administered to the subject to the previous titration dose, etc.) if said subject exhibits clinically significant fluid overload during treatment. In some embodiments, the dose administered to the subject is restored to its original amount after the subject's clinically significant fluid overload is resolved. In some embodiments, the subject is re-titrated after the subject's clinically significant fluid overload is resolved. For example, in some embodiments, where treatment is interrupted for seven days or more, re-titration of diazoxide choline should be implemented when diazoxide choline treatment is resumed. In some embodiments, the subject is re-titrated in dosage increments of no more than 25 mg of diazoxide choline every 2 weeks. In some embodiments, the subject is re-titrated in dosage increments of no more than 75 mg of diazoxide choline every 2 weeks. In some embodiments, the subject is re-titrated over a longer duration relative to the initial titration.

In some embodiments, the one or more clinically relevant comorbidities is obesity. In some embodiments, the one or more clinically relevant comorbidities is severe obesity. The term “severe obesity,” as used herein, refers to an acute classification of obesity based on the Body Mass Index (BMI) of the subject (Hampl et al. “Clinical Practice Guideline for the Evaluation and Treatment of Children and Adolescents With Obesity.” Pediatrics. 2023; 151(2):e2022060641). For example, severe obesity in children and adolescents younger than 20 years old may present itself as a BMI between (i) 120% to less than 140% of the 95th percentile, or (ii) 140% or greater of the 95th percentile. Also for example, severe obesity in adults greater than or equal to 20 years old may present itself as a BMI of (i) 35.0-39.9 kg/m², or (ii) ≥40 kg/m². In some embodiments, the subject is severely obese and has one or more additional clinically relevant comorbidities.

In some embodiments, the subject is severely obese and has one or more cardiovascular comorbidities, as described herein (e.g., a history of edema, edema, fluid retention, fluid overload, congestive heart failure, compromised cardiac reserve, heart disease, etc.). In some embodiments, the subject is severely obese and has a history of one or more cardiovascular comorbidities. In some embodiments, the subject is severely obese and has one or more pulmonary comorbidities, as described herein (e.g., a history of asthma, asthma, chronic lung disease, Chronic Obstructive Pulmonary Disease (COPD), obstructive sleep apnea, central sleep apnea, recent pulmonary infection, etc.). In some embodiments, the subject is severely obese and has a history of one or more pulmonary comorbidities. Thus, in some embodiments, a subject that is severely obese with one or more additional clinically relevant comorbidities may require a modified dosing schedule, such as those recited in Table 2a, 2b, or 2c.

In some embodiments, the one or more clinically relevant comorbidities is renal impairment. In some embodiments, a subject with renal impairment may require a modified dosing schedule, such as those recited in Table 2a, 2b, or 2c. In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise discontinuing administration to the subject if said subject exhibits renal impairment.

In some embodiments, the one or more clinically relevant comorbidities is hepatic impairment. In some embodiments, a subject with hepatic impairment may require a modified dosing schedule, such as those recited in Table 2a, 2b, or 2c. In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise discontinuing administration to the subject if said subject exhibits hepatic impairment.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising orally administering to the subject diazoxide choline once daily, wherein the subject has one or more clinically relevant comorbidities prior to treatment with diazoxide choline, and wherein the amount of diazoxide choline administered to the subject is reduced by between ⅓ to ½ relative to the amount that would be administered to the subject if the subject did not have one or more clinically relevant comorbidities.

In some embodiments, the amount of diazoxide choline administered to the subject is reduced by ⅓ relative to the amount that would be administered to the subject if the subject did not have one or more clinically relevant comorbidities. In some embodiments, the amount of diazoxide choline administered to the subject is reduced by ½ relative to the amount that would be administered to the subject if the subject did not have one or more clinically relevant comorbidities.

Treating A Subject with One or More Adverse Reactions

As noted herein, there are circumstances under which dosing modifications and more frequent monitoring may be considered in subjects having PWS. Specifically, the present disclosure delineates example diazoxide choline dosing modifications, as well as additional monitoring protocol, for subjects with one or more adverse reactions during treatment with diazoxide choline.

The term “adverse reaction,” as used herein, refers to an unwanted or unintended reaction to a treatment or a combination of a treatment and a coexisting condition (e.g., a medical condition, etc.) present during treatment that affects the treatment or prognosis of a subject's illness. For example, adverse reactions to the methods of treatment disclosed herein include, but are not limited to, cardiovascular (e.g., edema, fluid retention, fluid overload, congestive heart failure, reduced cardiac reserve, heart disease, etc.), pulmonary (e.g., asthma, chronic lung disease, Chronic Obstructive Pulmonary Disease (COPD), obstructive sleep apnea, central sleep apnea, pulmonary infection, etc.), and metabolic/endocrine (e.g., obesity, hyperglycemia, insulin resistance, elevated fasting glucose, elevated HbA1c, prediabetes, diabetes, etc.) reactions.

As will be appreciated by the skilled artisan, treatment modification in view of one or more adverse reactions is dependent on the type, frequency, severity, and number of adverse reactions exhibited by the subject. For example, a medical practitioner may assess that treatment modification is warranted for a subject with an elevated HbA1c level greater than or equal to 6.5%, or a subject with an elevated fasting glucose level of greater than 110 mg/dL. However, a medical practitioner may assess that treatment modification is warranted at significantly lower thresholds if a subject has both an elevated HbA1c level and an elevated fasting glucose level, for instance. Also for example, an increase (relative to baseline) of greater than or equal to 0.4 in a subject's HbA1c level, an increase (relative to baseline) of greater than or equal to 20 mg/dL in a subject's fasting glucose level, and edema (e.g., peripheral edema) greater than or equal to Grade 3, are all examples of adverse reactions that a medical practitioner may find warrant treatment modification when taken in conjunction with other factors (e.g., one or more other adverse reactions).

An example of modified dose titrations for subjects with one or more adverse reactions within different weight ranges are shown in Tables 3a-3b, below.

TABLE 3a
First Example of Diazoxide Choline Dosage Modifications
for Patients who Experience One or More Adverse Reactions

| Step | | | | | | |
|---|---|---|---|---|---|---|
| Starting | | | | | | |
| Dose | Step 2 | Step 3 | Step 4 | Step 5 | Step 6 | Target |
| Duration | | | | | | |
| 2 to 4+ | 2 to 4+ | 2 to 4+ | 2 to 4+ | 2 to 4+ | 2 to 4+ | Maintenance |
| Weeks | Weeks | Weeks | Weeks | Weeks | Weeks | Therapy |

Body Weight:

| | | | | | | | | | | |
|---|---|---|---|---|---|---|---|---|---|---|
| 20 to <30 | kg | 25 mg | 50 | mg | 75 | mg | 100 mg | 100 mg | 100 mg | 100 mg |
| 30 to <40 | kg | 75 mg | 150 | mg | 150 | mg | 150 mg | 150 mg | 150 mg | 150 mg |
| 40 to <65 | kg | 75 mg | 150 | mg | 225 | mg | 225 mg | 225 mg | 225 mg | 225 mg |
| 65 to <100 | kg | 75 mg | 150 | mg | 225 | mg | 300 mg | 375 mg | 375 mg | 375 mg |
| 100 to <135 | kg | 75 mg | 150 | mg | 225 | mg | 300 mg | 375 mg | 450 mg | 450 mg |
| ≥135 | kg | 75 mg | 150 | mg | 225 | mg | 300 mg | 375 mg | 450 mg | 525 mg |

TABLE 3b
Second Example of Diazoxide Choline Dosage Modifications
for Patients who Experience One or More Adverse Reactions

| | | Step 1 | Step 2 | | Step 3 | | Step 4 | Step 5 | Step 6 | Initial | Max |
|---|---|---|---|---|---|---|---|---|---|---|---|
| | | Weeks | Weeks | | Weeks | | Weeks | Weeks | Weeks | Target | Target |
| Weight | | 1-4 | 5-8 | | 9-12 | | 12-16 | 17-20 | 20-24 | Dose | Dose |
| 20 to <30 | kg | 25 mg | 50 | mg | 75 | mg | 100 mg | — | — | 100 mg | 100 mg |
| 30 to <40 | kg | 75 mg | 150 | mg | — | | — | — | — | 150 mg | 150 mg |
| 40 to <65 | kg | 75 mg | 150 | mg | 225 | mg | — | — | — | 225 mg | 225 mg |
| 65 to <100 | kg | 75 mg | 150 | mg | 225 | mg | 275 mg | — | — | 275 mg | 375 mg |
| 100 to <135 | kg | 75 mg | 150 | mg | 225 | mg | 275 mg | 325 mg | — | 325 mg | 450 mg |
| ≥135 | kg | 75 mg | 150 | mg | 225 | mg | 275 mg | 325 mg | 375 mg | 375 mg | 525 mg |

In some embodiments, certain patients may require a modified dosing schedule. Examples of said modified dosing schedules are provided in Tables 3a and 3b. In some embodiments, the subject is dosed according to Tables 3a or 3b. In some embodiments, the subject is dosed according to Table 3a. In some embodiments, the subject is dosed according to Table 3b.

In some embodiments, the modified starting dose of diazoxide choline is between about 25-75 mg of diazoxide choline. In some embodiments, the modified starting dose of diazoxide choline is about 25 mg of diazoxide choline or about 75 mg of diazoxide choline.

In some embodiments, the modified target maintenance dose of diazoxide choline is between about 100-525 mg of diazoxide choline, including between about 100-450 mg of diazoxide choline, between about 100-375 mg of diazoxide choline, between about 100-225 mg of diazoxide choline, between about 100-150 mg of diazoxide choline, between about 150-525 mg of diazoxide choline, between about 150-450 mg of diazoxide choline, between about 150-375 mg of diazoxide choline, between about 150-225 mg of diazoxide choline, between about 225-525 mg of diazoxide choline, between about 225-450 mg of diazoxide choline, between about 225-375 mg of diazoxide choline, between about 375-525 mg of diazoxide choline, between about 375-450 mg of diazoxide choline, or between about 450-525 mg of diazoxide choline. In some embodiments, the modified target maintenance dose of diazoxide choline is about 100 mg of diazoxide choline, about 150 mg of diazoxide choline, about 225 mg of diazoxide choline, about 375 mg of diazoxide choline, about 450 mg of diazoxide choline, or about 525 mg of diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of at least two weeks;
(b) after the initial period of at least two weeks, orally administering to the subject 50 mg of diazoxide choline once daily for a second period of at least two weeks;
(c) after the second period of at least two weeks, orally administering to the subject 75 mg of diazoxide choline once daily for a third period of at least two weeks; and
(d) after the third period of at least two weeks, orally administering to the subject a target maintenance dose of 100 mg of diazoxide choline once daily;
wherein the subject experiences one or more adverse reactions during treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of at least four weeks;
(b) after the initial period of at least four weeks, orally administering to the subject 50 mg of diazoxide choline once daily for a second period of at least four weeks;
(c) after the second period of at least four weeks, orally administering to the subject 75 mg of diazoxide choline once daily for a third period of at least four weeks; and
(d) after the third period of at least four weeks, orally administering to the subject a target maintenance dose of 100 mg of diazoxide choline once daily;
wherein the subject experiences one or more adverse reactions during treatment with diazoxide choline.

In some embodiments, the subject weighs from about 20 kg to less than 30 kg. In some embodiments, the subject weighs about 20 kg. In some embodiments, the subject weighs about 25 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks; and
(b) after the initial period of at least two weeks, orally administering to the subject a target maintenance dose of 150 mg of diazoxide choline once daily;
wherein the subject experiences one or more adverse reactions during treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks; and
(b) after the initial period of at least four weeks, orally administering to the subject a target maintenance dose of 150 mg of diazoxide choline once daily;
wherein the subject experiences one or more adverse reactions during treatment with diazoxide choline.

In some embodiments, the subject weighs from 30 kg to less than 40 kg. In some embodiments, the subject weighs about 30 kg. In some embodiments, the subject weighs about 35 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks; and
(c) after the second period of at least two weeks, orally administering to the subject a target maintenance dose of 225 mg of diazoxide choline once daily;
wherein the subject experiences one or more adverse reactions during treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks; and
(c) after the second period of at least four weeks, orally administering to the subject a target maintenance dose of 225 mg of diazoxide choline once daily;
wherein the subject experiences one or more adverse reactions during treatment with diazoxide choline.

In some embodiments, the subject weighs from 40 kg to less than 65 kg. In some embodiments, the subject weighs about 40 kg. In some embodiments, the subject weighs about 45 kg. In some embodiments, the subject weighs about 50 kg. In some embodiments, the subject weighs about 55 kg. In some embodiments, the subject weighs about 60 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks; and
(e) after the fourth period of at least two weeks, orally administering to the subject a target maintenance dose of 375 mg of diazoxide choline once daily;
wherein the subject experiences one or more adverse reactions during treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 375 mg of diazoxide choline once daily;
wherein the subject experiences one or more adverse reactions during treatment with diazoxide choline.

In some embodiments, the subject weighs from 65 kg to less than 100 kg. In some embodiments, the subject weighs about 65 kg. In some embodiments, the subject weighs about 70 kg. In some embodiments, the subject weighs about 75 kg. In some embodiments, the subject weighs about 80 kg. In some embodiments, the subject weighs about 85 kg. In some embodiments, the subject weighs about 90 kg. In some embodiments, the subject weighs about 95 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks; and
(f) after the fifth period of at least two weeks, orally administering to the subject a target maintenance dose of 450 mg of diazoxide choline once daily;
wherein the subject experiences one or more adverse reactions during treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject an initial target dose of 325 mg of diazoxide choline once daily for a period of at least six months; and
   (i) after the period of at least six months, orally administering to the subject the initial target dose of 325 mg of diazoxide choline once daily; or
   (ii) after the period of at least six months, orally administering to the subject a maximum target dose of 450 mg of diazoxide choline once daily;
wherein the subject experiences one or more adverse reactions during treatment with diazoxide choline.

In some embodiments, the subject weighs from 100 kg to less than 135 kg. In some embodiments, the subject weighs about 100 kg. In some embodiments, the subject weighs about 105 kg. In some embodiments, the subject weighs about 110 kg. In some embodiments, the subject weighs about 115 kg. In some embodiments, the subject weighs about 120 kg. In some embodiments, the subject weighs about 125 kg. In some embodiments, the subject weighs about 130 kg.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks;
(f) after the fifth period of at least two weeks, orally administering to the subject 450 mg of diazoxide choline once daily for a sixth period of at least two weeks; and
(g) after the sixth period of at least two weeks, orally administering to the subject a target maintenance dose of 525 mg of diazoxide choline once daily;
wherein the subject experiences one or more adverse reactions during treatment with diazoxide choline.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject 325 mg of diazoxide choline once daily for a fifth period of at least four weeks;
(f) after the fifth period of at least four weeks, orally administering to the subject an initial target dose of 375 mg of diazoxide choline once daily for a period of at least six months; and
   (i) after the period of at least six months, orally administering to the subject the initial target dose of 375 mg of diazoxide choline once daily; or
   (ii) after the period of at least six months, orally administering to the subject a maximum target dose of 525 mg of diazoxide choline once daily;
wherein the subject experiences one or more adverse reactions during treatment with diazoxide choline.

In some embodiments, the subject weighs 135 kg or more. In some embodiments, the subject weighs about 135 kg. In some embodiments, the subject weighs about 140 kg. In some embodiments, the subject weighs about 145 kg. In some embodiments, the subject weighs about 150 kg. In some embodiments, the subject weighs about 155 kg. In some embodiments, the subject weighs about 160 kg. In some embodiments, the subject weighs about 165 kg. In some embodiments, the subject weighs about 170 kg.

In some embodiments, the initial period of at least two weeks is 2-6 weeks. In some embodiments, the initial period of at least two weeks is 2-5 weeks. In some embodiments, the initial period of at least two weeks is 2-4 weeks. In some embodiments, the initial period of at least two weeks is two, three, four, five, or six weeks. In some embodiments, the initial period of at least two weeks is two, three, four, or five weeks. In some embodiments, the initial period of at least two weeks is two, three, or four weeks. In some embodiments, the initial period of at least two weeks is two weeks. In some embodiments, the initial period of at least two weeks is three weeks. In some embodiments, the initial period of at least two weeks is four weeks. In some embodiments, the initial period of at least two weeks is five weeks. In some embodiments, the initial period of at least two weeks is six weeks.

In some embodiments, the second period of at least two weeks is 2-6 weeks. In some embodiments, the second period of at least two weeks is 2-5 weeks. In some embodiments, the second period of at least two weeks is 2-4 weeks. In some embodiments, the second period of at least two weeks is two, three, four, five, or six weeks. In some embodiments, the second period of at least two weeks is two, three, four, or five weeks. In some embodiments, the second period of at least two weeks is two, three, or four weeks. In some embodiments, the second period of at least two weeks is two weeks. In some embodiments, the second period of at least two weeks is three weeks. In some embodiments, the second period of at least two weeks is four weeks. In some embodiments, the second period of at least two weeks is five weeks. In some embodiments, the second period of at least two weeks is six weeks.

In some embodiments, the third period of at least two weeks is 2-6 weeks. In some embodiments, the third period of at least two weeks is 2-5 weeks. In some embodiments, the third period of at least two weeks is 2-4 weeks. In some embodiments, the third period of at least two weeks is two, three, four, five, or six weeks. In some embodiments, the third period of at least two weeks is two, three, four, or five weeks. In some embodiments, the third period of at least two weeks is two, three, or four weeks. In some embodiments, the third period of at least two weeks is two weeks. In some embodiments, the third period of at least two weeks is three weeks. In some embodiments, the third period of at least two weeks is four weeks. In some embodiments, the third period of at least two weeks is five weeks. In some embodiments, the third period of at least two weeks is six weeks.

In some embodiments, the fourth period of at least two weeks is 2-6 weeks. In some embodiments, the fourth period of at least two weeks is 2-5 weeks. In some embodiments, the fourth period of at least two weeks is 2-4 weeks. In some embodiments, the fourth period of at least two weeks is two, three, four, five, or six weeks. In some embodiments, the fourth period of at least two weeks is two, three, four, or five weeks. In some embodiments, the fourth period of at least two weeks is two, three, or four weeks. In some embodiments, the fourth period of at least two weeks is two weeks. In some embodiments, the fourth period of at least two weeks is three weeks. In some embodiments, the fourth period of at least two weeks is four weeks. In some embodiments, the fourth period of at least two weeks is five weeks. In some embodiments, the fourth period of at least two weeks is six weeks.

In some embodiments, the fifth period of at least two weeks is 2-6 weeks. In some embodiments, the fifth period of at least two weeks is 2-5 weeks. In some embodiments, the fifth period of at least two weeks is 2-4 weeks. In some embodiments, the fifth period of at least two weeks is two, three, four, five, or six weeks.

In some embodiments, the fifth period of at least two weeks is two, three, four, or five weeks. In some embodiments, the fifth period of at least two weeks is two, three, or four weeks. In some embodiments, the fifth period of at least two weeks is two weeks. In some embodiments, the fifth period of at least two weeks is three weeks. In some embodiments, the fifth period of at least two weeks is four weeks. In some embodiments, the fifth period of at least two weeks is five weeks. In some embodiments, the fifth period of at least two weeks is six weeks.

In some embodiments, the sixth period of at least two weeks is 2-6 weeks. In some embodiments, the sixth period of at least two weeks is 2-5 weeks. In some embodiments, the sixth period of at least two weeks is 2-4 weeks. In some embodiments, the sixth period of at least two weeks is two, three, four, five, or six weeks.

In some embodiments, the sixth period of at least two weeks is two, three, four, or five weeks. In some embodiments, the sixth period of at least two weeks is two, three, or four weeks. In some embodiments, the sixth period of at least two weeks is two weeks. In some embodiments, the sixth period of at least two weeks is three weeks. In some embodiments, the sixth period of at least two weeks is four weeks. In some embodiments, the sixth period of at least two weeks is five weeks. In some embodiments, the sixth period of at least two weeks is six weeks.

In some embodiments, the initial period of at least four weeks is 4-8 weeks. In some embodiments, the initial period of at least four weeks is 4-7 weeks. In some embodiments, the initial period of at least four weeks is 4-6 weeks. In some embodiments, the initial period of at least four weeks is four, five, six, seven, or eight weeks. In some embodiments, the initial period of at least four weeks is four, five, six, or seven weeks.

In some embodiments, the initial period of at least four weeks is four, five, or six weeks. In some embodiments, the initial period of at least four weeks is four weeks. In some embodiments, the initial period of at least four weeks is five weeks. In some embodiments, the initial period of at least four weeks is six weeks. In some embodiments, the initial period of at least four weeks is seven weeks. In some embodiments, the initial period of at least four weeks is eight weeks.

In some embodiments, the second period of at least four weeks is 4-8 weeks. In some embodiments, the second period of at least four weeks is 4-7 weeks. In some embodiments, the second period of at least four weeks is 4-6 weeks. In some embodiments, the second period of at least four weeks is four, five, six, seven, or eight weeks. In some embodiments, the second period of at least four weeks is four, five, six, or seven weeks.

In some embodiments, the second period of at least four weeks is four, five, or six weeks. In some embodiments, the second period of at least four weeks is four weeks. In some embodiments, the second period of at least four weeks is five weeks. In some embodiments, the second period of at least four weeks is six weeks. In some embodiments, the second period of at least four weeks is seven weeks. In some embodiments, the second period of at least four weeks is eight weeks.

In some embodiments, the third period of at least four weeks is 4-8 weeks. In some embodiments, the third period of at least four weeks is 4-7 weeks. In some embodiments, the third period of at least four weeks is 4-6 weeks. In some embodiments, the third period of at least four weeks is four, five, six, seven, or eight weeks. In some embodiments, the third period of at least four weeks is four, five, six, or seven weeks. In some embodiments, the third period of at least four weeks is four, five, or six weeks. In some embodiments, the third period of at least four weeks is four weeks. In some embodiments, the third period of at least four weeks is five weeks. In some embodiments, the third period of at least four weeks is six weeks. In some embodiments, the third period of at least four weeks is seven weeks. In some embodiments, the third period of at least four weeks is eight weeks.

In some embodiments, the fourth period of at least four weeks is 4-8 weeks. In some embodiments, the fourth period of at least four weeks is 4-7 weeks. In some embodiments, the fourth period of at least four weeks is 4-6 weeks. In some embodiments, the fourth period of at least four weeks is four, five, six, seven, or eight weeks. In some embodiments, the fourth period of at least four weeks is four, five, six, or seven weeks.

In some embodiments, the fourth period of at least four weeks is four, five, or six weeks. In some embodiments, the fourth period of at least four weeks is four weeks. In some embodiments, the fourth period of at least four weeks is five weeks. In some embodiments, the fourth period of at least four weeks is six weeks. In some embodiments, the fourth period of at least four weeks is seven weeks. In some embodiments, the fourth period of at least four weeks is eight weeks.

In some embodiments, the fifth period of at least four weeks is 4-8 weeks. In some embodiments, the fifth period of at least four weeks is 4-7 weeks. In some embodiments, the fifth period of at least four weeks is 4-6 weeks. In some embodiments, the fifth period of at least four weeks is four, five, six, seven, or eight weeks.

In some embodiments, the fifth period of at least four weeks is four, five, six, or seven weeks. In some embodiments, the fifth period of at least four weeks is four, five, or six weeks. In some embodiments, the fifth period of at least four weeks is four weeks. In some embodiments, the fifth period of at least four weeks is five weeks. In some embodiments, the fifth period of at least four weeks is six weeks. In some embodiments, the fifth period of at least four weeks is seven weeks. In some embodiments, the fifth period of at least four weeks is eight weeks.

In some embodiments, the period of at least six months is 6-12 months. In some embodiments, the period of at least six months is 6-11 months. In some embodiments, the period of at least six months is 6-10 months. In some embodiments, the period of at least six months is 6-9 months. In some embodiments, the period of at least six months is 6-8 months. In some embodiments, the period of at least six months is six, seven, eight, nine, ten, eleven, or twelve months. In some embodiments, the period of at least six months is six, seven, eight, nine, ten, or eleven months. In some embodiments, the period of at least six months is six, seven, eight, nine, or ten months. In some embodiments, the period of at least six months is six, seven, eight, or nine months. In some embodiments, the period of at least six months is six, seven, or eight months. In some embodiments, the period of at least six months is six months. In some embodiments, the period of at least six months is seven months. In some embodiments, the period of at least six months is eight months. In some embodiments, the period of at least six months is nine months. In some embodiments, the period of at least six months is ten months. In some embodiments, the period of at least six months is eleven months. In some embodiments, the period of at least six months is twelve months.

In some embodiments, the target maintenance dose is reached after at least 10 weeks of treatment. In some embodiments, the target maintenance dose is reached after 10-24 weeks of treatment. In some embodiments, the target maintenance dose is reached after 12-24 weeks of treatment. In some embodiments, the target maintenance dose is reached after 12-20 weeks of treatment. In some embodiments, the target maintenance dose is reached after 12-16 weeks of treatment. In some embodiments, the target maintenance dose is reached after 16-24 weeks of treatment. In some embodiments, the target maintenance dose is reached after 16-20 weeks of treatment. In some embodiments, the target maintenance dose is reached after 20-24 weeks of treatment. In some embodiments, the target maintenance dose is reached after 10 weeks of treatment.

In some embodiments, the target maintenance dose is reached after 11 weeks of treatment. In some embodiments, the target maintenance dose is reached after 12 weeks of treatment. In some embodiments, the target maintenance dose is reached after 13 weeks of treatment. In some embodiments, the target maintenance dose is reached after 14 weeks of treatment. In some embodiments, the target maintenance dose is reached after 15 weeks of treatment. In some embodiments, the target maintenance dose is reached after 16 weeks of treatment. In some embodiments, the target maintenance dose is reached after 17 weeks of treatment. In some embodiments, the target maintenance dose is reached after 18 weeks of treatment. In some embodiments, the target maintenance dose is reached after 19 weeks of treatment. In some embodiments, the target maintenance dose is reached after 20 weeks of treatment. In some embodiments, the target maintenance dose is reached after 21 weeks of treatment. In some embodiments, the target maintenance dose is reached after 22 weeks of treatment. In some embodiments, the target maintenance dose is reached after 23 weeks of treatment. In some embodiments, the target maintenance dose is reached after 24 weeks of treatment.

In some embodiments, after the target maintenance dose is reached, the amount of diazoxide choline orally administered once daily to the subject is increased by no more than 25 mg every adjustment period, wherein every adjustment period has a duration of at least two weeks, wherein the amount of diazoxide choline orally administered once daily to the subject is no more than 5.8 mg/kg. In some embodiments, after the target maintenance dose is reached, the amount of diazoxide choline orally administered once daily to the subject is increased by no more than 75 mg every adjustment period, wherein every adjustment period has a duration of at least two weeks, wherein the amount of diazoxide choline orally administered once daily to the subject is no more than 5.8 mg/kg or 525 mg, whichever is lower.

In some embodiments, treatment is temporarily interrupted following the development of one or more adverse reactions. In some embodiments, treatment resumes at the previous once daily administration dose of diazoxide choline following an interruption in treatment lasting less than 7 days. In some embodiments, the subject is re-titrated following an interruption in treatment lasting 7 or more days. In some embodiments, treatment resumes at the starting dose of diazoxide choline following an interruption in treatment lasting 7 or more days. In some embodiments, treatment resumes at the starting dose of diazoxide choline following an interruption in treatment lasting 14 or more days. In some embodiments, the one or more adverse reactions is hyperglycemia. In some embodiments, the one or more adverse reactions is edema. In some embodiments, the one or more adverse reactions is fluid overload.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise modifying the dosing schedule administered to the subject if said subject exhibits clinically significant glucose elevation during titration. In some embodiments, the modified dosing schedule comprises titrating the subject over longer durations relative to the unmodified dosing schedule (e.g., the dosing schedule recited in Table 1). In some embodiments, the modified dosing schedule comprises titrating the subject to a lower dosage relative to the unmodified dosing schedule (e.g., the dosing schedule recited in Table 1). In some embodiments, the modified dosing schedule may be according to Table 3a or 3b. In some embodiments, the modified dosing schedule may be according to Table 3a. In some embodiments, the modified dosing schedule may be according to Table 3b.

In some embodiments, the one or more adverse reactions is obesity. In some embodiments, the one or more adverse reactions is severe obesity. The term “severe obesity,” as used herein, refers to an acute classification of obesity based on the Body Mass Index (BMI) of the subject (Hampl et al. “Clinical Practice Guideline for the Evaluation and Treatment of Children and Adolescents With Obesity.” Pediatrics. 2023; 151(2):e2022060641). For example, severe obesity in children and adolescents younger than 20 years old may present itself as a BMI between (i) 120% to less than 140% of the 95th percentile, or (ii) 140% or greater of the 95th percentile. Also for example, severe obesity in adults greater than or equal to 20 years old may present itself as a BMI of (i) 35.0-39.9 kg/m², or (ii) >40 kg/m². In some embodiments, the subject is severely obese and has one or more additional adverse reactions. In some embodiments, the subject is severely obese and has one or more cardiovascular reactions, as described herein (e.g., edema, fluid retention, fluid overload, congestive heart failure, reduced cardiac reserve, heart disease, etc.). In some embodiments, the subject is severely obese and has a history of one or more cardiovascular reactions. In some embodiments, the subject is severely obese and has one or more pulmonary reactions, as described herein (e.g., asthma, chronic lung disease, Chronic Obstructive Pulmonary Disease (COPD), obstructive sleep apnea, central sleep apnea, pulmonary infection, etc.). In some embodiments, the subject is severely obese and has a history of one or more pulmonary reactions. Thus, in some embodiments, a subject that is severely obese with one or more additional adverse reactions may require a modified dosing schedule, such as those recited in Table 3a or 3b.

In some embodiments, the once daily administration dose of diazoxide choline is reduced following the development of one or more adverse reactions. In some embodiments, the once daily administration dose of diazoxide choline remains constant for a period of time following the development of one or more adverse reactions. In some embodiments, the once daily administration of diazoxide choline is paused for a period of time following the development of one or more adverse reactions.

In some embodiments, the once daily administration dose of diazoxide choline is titrated in increments of no more than 25 mg every 2 weeks for subjects weighing less than 30 kg following the development of one or more adverse reactions. In some embodiments, the once daily administration dose of diazoxide choline is titrated over a longer duration to a maximum dosage of 5.8 mg/kg for subjects weighing less than 30 kg following the development of one or more adverse reactions. In some embodiments, the once daily administration dose of diazoxide choline is titrated in increments of no more than 75 mg every 2 weeks for subjects weighing greater than or equal to 30 kg following the development of one or more adverse reactions.

In some embodiments, the once daily administration dose of diazoxide choline is titrated over a longer duration to a maximum dosage of 5.8 mg/kg for subjects weighing greater than or equal to 30 kg following the development of one or more adverse reactions.

In some embodiments, the subject is concomitantly administered one or more risk mitigating medications following the development of one or more adverse reactions. In some embodiments, the one or more risk mitigating medications is a glucose-lowering agent or a diuretic. In some embodiments, the one or more risk mitigating medications is a glucose-lowering agent. In some embodiments, the one or more risk mitigating medications is a diuretic.

In some embodiments, the one or more adverse reactions is edema or fluid overload. In some embodiments, the one or more adverse reactions is edema. In some embodiments, the one or more adverse reactions is fluid overload. In some embodiments, the one or more adverse reactions is fluid retention. In some embodiments, the methods disclosed herein further comprise periodically monitoring the body weight of the subject during treatment. In some embodiments, the methods disclosed herein further comprise periodically assessing the subject for swelling of a body part during treatment. In some embodiments, the body part is an ankle, foot, or leg. In some embodiments, the body part is an ankle. In some embodiments, the body part is a foot. In some embodiments, the body part is a leg. In some embodiments, the methods disclosed herein further comprise periodically assessing the subject for one or more respiratory symptoms during treatment. In some embodiments, the one or more respiratory symptoms is dyspnea or shortness of breath. In some embodiments, the one or more respiratory symptoms is dyspnea. In some embodiments, the one or more respiratory symptoms is shortness of breath. In some embodiments, the monitoring takes place once a week during titration. In some embodiments, the monitoring takes place at regular intervals after titration.

As will be appreciated by the skilled artisan, there are different forms of edema, including pitting and nonpitting edema. For a given subject, a skilled artisan's assessment of pitting or nonpitting edema will be based on a number of factors. For example, a skilled artisan's assessment of nonpitting edema may be based on the observation of one or more symptoms (e.g., tense, tender, sensation of heaviness, impaired mobility, skin thickening, ulcers, cellulitis, inability to pinch skin, etc.). Risks associated with nonpitting edema include, but are not limited to, lower responsiveness to diuretics (relative to pitting edema) and an impaired ability to identify worsening fluid retention in the subject. Thus, in some embodiments, a subject with nonpitting edema may require a modified dosing schedule, such as those recited in Table 3a or 3b. In some embodiments, a subject with nonpitting edema is dosed according to Table 3a or 3b.

In some embodiments, the one or more adverse reactions is renal impairment. In some embodiments, a subject with renal impairment may require a modified dosing schedule, such as those recited in Table 3a or 3b. In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise discontinuing administration to the subject if said subject exhibits renal impairment.

In some embodiments, the one or more adverse reactions is hepatic impairment. In some embodiments, a subject with hepatic impairment may require a modified dosing schedule, such as those recited in Table 3a or 3b. In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise discontinuing administration to the subject if said subject exhibits hepatic impairment.

### Treating A Subject Having SMS

As also described herein, an effective amount of diazoxide, or a corresponding amount of a pharmaceutically acceptable salt thereof, may be administered to a subject having SMS to achieve therapeutic outcomes while reducing the incidence of adverse effects. Therapeutic outcomes may include, but are not limited to, reducing the severity or progression of SMS, alleviating comorbidities, enhancing biological function, or achieving a measurable clinical benefit. Subjects having SMS who can be treated include, but are not limited to, SMS subjects with one or more of the behavioral or cognitive and/or one more physiological characteristics of SMS, including those described herein. In some embodiments, the subject may have hyperphagia or hypotonia. In some embodiments, SMS subjects may exhibit aggressive behaviors or other behavioral characteristics that may be associated with SMS.

With respect to a subject having SMS, the effect of administration may include, but is not limited to, reducing the severity or progression of SMS, alleviating comorbidities, enhancing biological function, or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

Smith-Magenis syndrome (SMS; OMIM #182290, *607642) is a complex, generally sporadic, genetic disorder caused by haploinsufficiency of the retinoic acid induced 1 (RAI1) gene. caused by either a 17p11.2 deletion encompassing the retinoic acid-induced 1 (RAI1) gene or a mutation of RAIL. (see, for example, Burns et al., in Human Molecular Genetics, 2010, Vol. 19, No. 20, pp. 4026-4042).

Smith-Magenis syndrome is characterized by a clinically recognizable phenotype that includes physical, developmental, neurological, and behavioral features. Short stature (<5th percentile) was observed in ~67% of young patients. Short stature resolves overtime with most individuals reaching the 10-25th percentile by adulthood (see, for example, Burns et al. supra). A majority of children with SMS also have significant early-onset obesity (see, for example, European Journal of Human Genetics (2008) 16, 412-421). Obesity in teens and adults is common, typically with broad chests and truncal obesity. RAI1 haploinsufficiency represents a single-gene model of obesity with hyperphagia, abnormal fat distribution and altered hypothalamic gene expression associated with satiety, food intake, behavior and obesity.

Hearing loss is variable and is often associated with chronic ear infections. Ophthalmologic features are present in >60% of SMS patients and include myopia, iris anomalies, strabismus, microcornea, and, rarely, retinal detachment (often resulting from violent behaviors).

Prenatal history of SMS is notable for significantly decreased fetal movement in 50% of pregnancies.

Early infancy is complicated by feeding difficulties leading to failure to thrive, marked oral sensory motor dysfunction with poor suckling reflex, gastroesophageal reflux, and hypotonia.

Most SMS individuals have mild-to-moderate mental retardation. IQ decreases as the child ages, ultimately placing the individual in the mild mental retardation range by adulthood. Delayed speech with or without hearing loss occurs in 96% of SMS patients. In addition, delayed fine/gross motor skills, problems with sensory integration, and poor adaptive function are seen. Other neurological features include pes cavus or pes planus, an abnormal ‘festinating’ gait, balance problems, and a decreased sensitivity to pain, which is often observed in association with self-injury in this disorder.

Sleep disturbance has been reported in 75-100% of SMS cases. Infants typically experience hypersomnolence during the first year of life. Sleep disturbances in older children include difficulties falling asleep, diminished REM sleep, reduced 24-h and night sleep, fragmented and shortened sleep cycles with frequent nocturnal and early-morning awakenings, and excessive daytime sleepiness.

Behavioral issues are one of the unique characteristic features of SMS. Maladaptive behaviors are a cause of major concern and include frequent outbursts/temper tantrums, attention seeking, aggression, disobedience, distraction, and self-injurious behaviors. Self-injurious behaviors include head banging, skin picking, wrist biting, onychotillomania and polyembolokoilamania. The behavioral phenotype of SMS escalates with age, typically with the onset of puberty. Age, degree of developmental delay, severity of any associated systemic disorder, and degree of sleep disturbance have a strong influence on maladaptive behaviors. Individuals also have a lack of respect for personal space during a conversation and are emotionally volatile.

SMS patients share many characteristics in common with PWS patients. These include: (1) diminished fetal activity; (2) hypotonia; (3) feeding difficulties in early infancy; (4) short stature: (5) early onset obesity which may include hyperphagia; (6) developmental delays; (7) mental retardation; (8) temper outbursts; (9) aggressive behavior; (10) self-injurious behavior; (11) hypersomnolence in infancy and similar sleep disturbances in older children and adults; (12) frequent ear infections; (13) scoliosis; and (14) strabismus. While the underlying genetic basis for these disorders is quite distinct, the presentation and natural history of the disease is very similar.

### Treating A Subject Having GSD1

As also described herein, an effective amount of diazoxide, or a corresponding amount of a pharmaceutically acceptable salt thereof, may be administered to a subject having GSD1 to achieve therapeutic outcomes while reducing the incidence of adverse effects. Therapeutic outcomes may include, but are not limited to, reducing the severity or progression of GSD1, alleviating comorbidities, enhancing biological function, or achieving a measurable clinical benefit. Subjects having GSD1 who can be treated include, but are not limited to, GSD1 subjects with one or more of the behavioral or cognitive and/or one or more physiological characteristics of GSD1, including those described herein. In some embodiments, the subject may have hyperphagia or hypotonia. In some embodiments, GSD1 subjects may exhibit aggressive behaviors or other behavioral characteristics that may be associated with GSD1.

With respect to a subject having GSD 1, the effect of administration may include, but is not limited to, reducing the severity or progression of GSD1, alleviating comorbidities, enhancing biological function, or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

Glycogen Storage Disease Type 1 (GSD1), also known as Von Gierke disease, is a rare, inherited, metabolic disorder that hinders glucose production through glycogenolysis and gluconeogenesis. It is caused by genetic mutations that affect glucose-6-phosphatase (G6Pase) activity, which ultimately result in excessive glycogen accumulation in the liver and kidneys. The primary subtypes of GSD1 include Subtype 1a (GSD1a), which is caused by G6Pase deficiency due to mutations in the G6PC gene, and Subtype 1b (GSD1b), which is caused by mutations in the SLC37A4 gene (see, for example, Nana et al. StatPearls 2025).

In general, Glycogen Storage Disease Type 1 is characterized by the inability of a subject's body to properly convert glycogen into glucose. Subjects having GSD1 exhibit a myriad of clinically recognizable physical, developmental, neurological, and behavioral features. For example, common symptoms of GSD1 include, but are not limited to, hypoglycemia, lactic acidosis, hyperuricemia, hypercholesterolemia, and hypertriglyceridemia, as well as hepatomegaly and abnormal movements such as seizures (see, for example, Kishnani et al. Genet. Med. 2014; 16(11):e1). Additional common symptoms of GSD1a include, but are not limited to, xanthoma and epistaxis, whereas additional common symptoms of GSD1b include, but are not limited to, neutropenia (see, for example, Nana et al. StatPearls 2025). Although some subjects having GSD1 exhibit symptoms in the neonatal period, they are more likely to exhibit symptoms beginning at 3 to 6 months of age.

Growth retardation is a common clinical feature in subjects having GSD1. Accordingly, subjects having GSD1 often present with short stature, particularly for subjects having GSD1a (see, for example, Wu et al. Front. Pediatr. 2022), and muscular hypotonia.

### Treating A Subject Having Glut1DS

As also described herein, an effective amount of diazoxide, or a corresponding amount of a pharmaceutically acceptable salt thereof, may be administered to a subject having Glut1DS to achieve therapeutic outcomes while reducing the incidence of adverse effects. Therapeutic outcomes may include, but are not limited to, reducing the severity or progression of Glut1DS, alleviating comorbidities, enhancing biological function, or achieving a measurable clinical benefit. Subjects having Glut1DS who can be treated include, but are not limited to, Glut1DS subjects with one or more of the behavioral or cognitive and/or one or more physiological characteristics of Glut1DS, including those described herein. In some embodiments, the subject may have hypotonia. In some embodiments, Glut1DS subjects may exhibit aggressive behaviors or other behavioral characteristics that may be associated with Glut1DS.

With respect to a subject having Glut1DS, the effect of administration may include, but is not limited to, reducing the severity or progression of Glut1DS, alleviating comorbidities, enhancing biological function, or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

Glucose Transporter Type 1 Deficiency Syndrome (Glut1DS), also known as De Vivo disease, is a rare genetic disorder that significantly impacts the brain's ability to receive adequate glucose. It is caused by genetic mutations in the SLC2A1 gene, which is crucial to produce the Glucose Transporter Type 1 (Glut1) protein that facilitates the transport of glucose across the blood-brain barrier.

Subjects having Glucose Transporter Type 1 Deficiency Syndrome exhibit a myriad of clinically recognizable physical, developmental, neurological, and behavioral features. In general, Glut1DS is characterized by a spectrum of neurological symptoms and developmental challenges, profoundly affecting the quality of life and cognitive functions of affected subjects. For example, common symptoms of Glut1DS include, but are not limited to, neuroglycopenia and epilepsy. The most common phenotype for subjects having Glut1DS accounts for 85% of cases reported, and typically presents as early onset encephalopathy, drug-resistant seizure, delayed psychomotor development, and deceleration of head growth with acquired microcephaly. In addition, motor involvement, including ataxia, spasticity, and dystonia, is often observed (see, for example, Veneruzzo et al. Arch. Argent. Pediatr. 2023; 121(1):e202202677).

### Reducing the Triglyceride Level of a Subject in Need Thereof

As also described herein, an effective amount of diazoxide, or a corresponding amount of a pharmaceutically acceptable salt thereof, may be administered to a subject with an elevated triglyceride level to achieve a reduction in said triglyceride level. In some embodiments, the subject has hypertriglyceridemia.

With respect to a subject with an elevated triglyceride level, the effect of administration may include, but is not limited to, reducing said triglyceride level or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

In some embodiments, the triglyceride level of the subject prior to treatment is ≥480 mg/dL. In some embodiments, the triglyceride level of the subject prior to treatment is ≥480 mg/dL and <1500 mg/dL.

In some embodiments, such as depicted in Table 16 of Example 4, the triglyceride level of the subject is reduced by at least about 10% relative to the triglyceride level of the subject prior to treatment. In some embodiments, the triglyceride level of the subject is reduced by between about 10%-90% relative to the triglyceride level of the subject prior to treatment.

### Reducing the Non-HDL Cholesterol Level of a Subject in Need Thereof

As also described herein, an effective amount of diazoxide, or a corresponding amount of a pharmaceutically acceptable salt thereof, may be administered to a subject with an elevated non-HDL cholesterol level to achieve a reduction in said non-HDL cholesterol level. In some embodiments, the subject has dyslipidemia.

With respect to a subject with an elevated non-HDL cholesterol level, the effect of administration may include, but is not limited to, reducing said non-HDL cholesterol level or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

In some embodiments, the non-HDL cholesterol level of the subject prior to treatment is ≥110 mg/dL.

In some embodiments, the non-HDL cholesterol level of the subject prior to treatment is ≥110 mg/dL and <430 mg/dL.

In some embodiments, such as depicted in Table 17 of Example 4, the non-HDL cholesterol level of the subject is reduced by at least about 10% relative to the non-HDL cholesterol level of the subject prior to treatment. In some embodiments, the non-HDL cholesterol level of the subject is reduced by between about 10%-80% relative to the non-HDL cholesterol level of the subject prior to treatment.

### Promoting Weight Loss in an Obese Subject in Need Thereof

As also described herein, an effective amount of diazoxide, or a corresponding amount of a pharmaceutically acceptable salt thereof, may be administered to an obese subject to promote weight loss in said obese subject.

With respect to an obese subject, the effect of administration may include, but is not limited to, promoting weight loss or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

Obesity is an elevated body weight beyond the limitation of skeletal and physical requirement, as the result of an excessive accumulation of fat in the body.

In some embodiments, the BMI of the obese subject prior to treatment is ≥27 kg/m². In some embodiments, the BMI of the obese subject prior to treatment is ≥27 kg/m² and <45 kg/m².

In some embodiments, such as depicted in Table 22 of Example 5, the weight of the obese subject is reduced by at least about 1% relative to the weight of the obese subject prior to treatment. In some embodiments, the weight of the obese subject is reduced by at least about 2% relative to the weight of the obese subject prior to treatment. In some embodiments, the weight of the obese subject is reduced by at least about 3% relative to the weight of the obese subject prior to treatment.

### Increasing the Fasting Glucose Level of a Subject in Need Thereof

As also described herein, an effective amount of diazoxide, or a corresponding amount of a pharmaceutically acceptable salt thereof, may be administered to a subject with a reduced fasting glucose level to achieve an increase in said fasting glucose level. In some embodiments, the subject is glucose transporter type 1 (Glut1) deficient.

With respect to a subject with a reduced fasting glucose level, the effect of administration may include, but is not limited to, increasing said fasting glucose level or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

In some embodiments, the fasting glucose level of the subject prior to treatment is <126 mg/dL. In some embodiments, the fasting glucose level of the subject prior to treatment is <115 mg/dL.

In some embodiments, such as depicted in Table 23 of Example 5 and Table 27 of Example 6, the fasting glucose level of the subject is increased by at least about 10% relative to the fasting glucose level of the subject prior to treatment. In some embodiments, the fasting glucose level of the subject is increased by between about 10%-60% relative to the fasting glucose level of the subject prior to treatment.

### Reducing the Fasting Insulin Level of a Subject in Need Thereof

As also described herein, an effective amount of diazoxide, or a corresponding amount of a pharmaceutically acceptable salt thereof, may be administered to a subject with an elevated fasting insulin level to achieve a reduction in said fasting insulin level. In some embodiments, the subject has hyperinsulinemia.

With respect to a subject with an elevated fasting insulin level, the effect of administration may include, but is not limited to, reducing said fasting insulin level or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

In some embodiments, the fasting insulin level of the subject prior to treatment is ≥20 μIU/mL. In some embodiments, the fasting insulin level of the subject prior to treatment is ≥20 μIU/mL and <90 μIU/mL.

In some embodiments, such as depicted in Table 18 of Example 4, Table 24 of Example 5, and Table 28 of Example 6, the fasting insulin level of the subject is reduced by at least about 25% relative to the fasting insulin level of the subject prior to treatment. In some embodiments, the fasting insulin level of the subject is reduced by between about 25%-97% relative to the fasting insulin level of the subject prior to treatment.

### Improving Insulin Resistance in a Subject in Need Thereof

As also described herein, an effective amount of diazoxide, or a corresponding amount of a pharmaceutically acceptable salt thereof, may be administered to a subject with insulin resistance to achieve an improvement in said insulin resistance.

With respect to a subject with insulin resistance, the effect of administration may include, but is not limited to, improving said insulin resistance or achieving a measurable clinical benefit. The specific amount may vary depending on factors such as the subject's condition, age, weight, disease severity, route of administration, and individual response. Unless otherwise specified, an effective amount encompasses doses that achieve the intended effect without causing unacceptable toxicity or adverse effects.

An example of dose titrations for subjects within different weight ranges are shown in Table 1, above.

Insulin resistance in a subject can be assessed by methods known to the skilled artisan, including, for example, determination of the Homeostatic Model Assessment of Insulin Resistance (HOMA-IR) score. For example, using fasting blood glucose and insulin levels. The formula is: (fasting insulin (mU/L)*fasting glucose (mmol/L))/22.5. A HOMA-IR value of 1 or less is generally considered normal, indicating good insulin sensitivity. Higher levels are indicative of insulin resistance. See e.g., Matthews D. R., et al., Diabetologia. 1985 July; 28(7):412-9.

In some embodiments, the HOMA-IR score of the subject prior to treatment is ≥4.5. In some embodiments, the HOMA-IR score of the subject prior to treatment is ≥4.5 and <27.0.

In some embodiments, such as depicted in Table 19 of Example 4 and Table 25 of Example 5, the HOMA-IR score of the subject is reduced by at least about 5% relative to the HOMA-IR score of the subject prior to treatment. In some embodiments, the HOMA-IR score of the subject is reduced by between about 5%-96% relative to the HOMA-IR score of the subject prior to treatment.

Drug Interactions—Diazoxide and other Pharmaceuticals

As noted herein, interactions between pharmaceuticals are highly unpredictable, yet significantly impact the safety (e.g., increase in severity or frequency of adverse effects) of a given treatment. Regarding diazoxide, it has unexpectedly been found that concomitant administration with other select pharmaceuticals potentially increases exposure to diazoxide, or pharmaceutically acceptable salts thereof, and thus must be taken into account when developing a dosing schedule (such that it may be altered if necessary).

Specifically, the present disclosure delineates the unexpected effect that concomitant administration of strong Cytochrome P450 1A2 (CYP1A2) inhibitors or strong Cytochrome P450 3A4 (CYP3A4) inhibitors with diazoxide, or pharmaceutically acceptable salts thereof, results in increased exposure to diazoxide. Accordingly, the suggested relationship between the doses of diazoxide choline and these inhibitors is highlighted in Table 4, below.

TABLE 4
Clinically Significant Drug Interactions with Diazoxide Choline
Strong CYP1A2 Inhibitors

| | |
|---|---|
| Prevention or | Reduce the dosage of Diazoxide Choline when concomitantly used with |
| Management | strong inhibitors of CYP1A2 [see Dosage and Administration (2.4)]. |
| Mechanism | Diazoxide Choline is a CYP1A2 substrate. Concomitant use of Diazoxide |
| and Clinical | Choline with strong CYP1A2 inhibitors increases exposure of diazoxide, |
| Effect(s) | which may increase the frequency and/or severity of adverse reactions from |
| | Diazoxide Choline [see Clinical Pharmacology (12.3)]. |

CYP1A2 Substrates

| | |
|---|---|
| Prevention or | Concomitant use of Diazoxide Choline with CYP1A2 substrates is not |
| Management | recommended. |
| Mechanism | Diazoxide Choline is an inhibitor of CYP1A2. Concomitant use of |
| and Clinical | Diazoxide Choline with CYP1A2 substrates increases exposure of these |
| Effect(s) | substrates. This may increase the frequency and/or severity of adverse |
| | reactions from such substrates. |

Strong CYP3A4 Inhibitors

| | |
|---|---|
| Prevention or | Monitor the frequency and severity of adverse reactions from Diazoxide |
| Management | Choline. A dosage reduction of Diazoxide Choline may be needed when |
| | used concomitantly with strong CYP3A4 inhibitors. |
| Mechanism | Concomitant use of Diazoxide Choline with strong CYP3A4 inhibitors |
| and Clinical | increases exposure of diazoxide, which may increase the frequency and/or |
| Effect(s) | severity of adverse reactions from Diazoxide Choline [see Clinical |
| | Pharmacology (12.3)]. |

Dual Strong CYP3A4/Moderate 1A2 Inducers

| | |
|---|---|
| Prevention or | Concomitant use of Diazoxide Choline with dual strong CYP3A4/moderate |
| Management | CYP1A2 inducers is not recommended. |
| Mechanism | Diazoxide Choline is a substrate of CYP3A4 and CYP1A2. Concomitant |
| and Clinical | use of Diazoxide Choline with strong CYP3A4/moderate 1A2 inducers |
| Effect(s) | may decrease exposure of Diazoxide Choline. This may decrease the |
| | efficacy of Diazoxide Choline [see Clinical Pharmacology (12.3)]. |

Drugs Highly Bound to Protein

| | |
|---|---|
| Prevention or | Monitor international normalized ratio (INR) in patients who use coumarin |
| Management | or its derivatives concomitantly with Diazoxide Choline. Dosage |
| | modification of coumarin or its derivatives may be needed when used |
| | concomitantly with Diazoxide Choline. |
| | Monitor diphenylhydantoin serum levels when Diazoxide Choline is used |
| | concomitantly with diphenylhydantoin. Dosage modification of |
| | diphenylhydantoin may be needed when used concomitantly with |
| | Diazoxide Choline. |
| Mechanism | Diazoxide is highly bound to serum proteins. |
| and Clinical | Diazoxide may displace other drugs which are also highly bound to protein |
| Effect(s) | resulting in higher or lower blood levels of the concomitantly used drugs. |
| | The impact of protein binding displacement is expected to be clinically |
| | important for drugs with narrow therapeutic range such as coumarin or its |
| | derivatives and diphenylhydantoin. Protein binding displacement may |
| | result in an increased risk of adverse reactions due to higher blood levels of |
| | coumarin or its derivative or loss of efficacy due to lower exposures of |
| | diphenylhydantoin. |

Thiazide or Other Diuretics

| | |
|---|---|
| Prevention or | Monitor for signs and symptoms of hyperglycemia [see Warnings and |
| Management | Precautions (5.1)] and hyperuricemia when Diazoxide Choline is used |
| | concomitantly with thiazides or other diuretics. Dosage adjustment of |
| | Diazoxide Choline or diuretics may be needed when Diazoxide Choline is |
| | concomitantly used with diuretics. |
| Mechanism | Both diazoxide and thiazides or other diuretics may produce hyperglycemia |
| and Clinical | and hyperuricemia. The concomitant use of Diazoxide Choline with |
| Effect(s) | thiazides or other diuretics may potentiate the hyperglycemic and |
| | hyperuricemic effects of diazoxide [see Adverse Reactions (6) and Clinical |
| | Pharmacology (12.2)]. |

Accordingly, disclosed herein are methods of treating a subject in need thereof, said methods comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject in need thereof, wherein:
(c) the subject is administered a therapeutically effective amount of diazoxide, or a pharmaceutically acceptable salt thereof, wherein the subject is concomitantly administered one or more strong CYP1A2 inhibitors; and
(d) the therapeutically effective amount of diazoxide, or a pharmaceutically acceptable salt thereof, administered to the subject is adjusted.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(e) administering to the subject a therapeutically effective amount of diazoxide, or a pharmaceutically acceptable salt thereof, wherein the subject is concomitantly administered one or more strong CYP1A2 inhibitors; and
(f) adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(g) the subject is administered a therapeutically effective amount of diazoxide, or a pharmaceutically acceptable salt thereof, wherein the subject is concomitantly administered one or more strong CYP1A2 inhibitors; and
(h) the therapeutically effective amount of diazoxide, or a pharmaceutically acceptable salt thereof, administered to the subject is adjusted.

In some embodiments, the one or more strong CYP1A2 inhibitors is selected from the group consisting of amiodarone, ciprofloxacin, and fluvoxamine.

In some embodiments, the therapeutically effective amount of diazoxide is reduced relative to the therapeutically effective amount for the subject if not concomitantly administered one or more strong CYP1A2 inhibitors.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, the therapeutically effective amount of diazoxide is the same as the therapeutically effective amount for the subject if concomitantly administered one or more moderate CYP1A2 inhibitors.

The present disclosure also delineates the unexpected effect that concomitant administration of strong Organic Anion Transporter 1 (OAT1) inhibitors or strong Organic Anion Transporter 3 (OAT3) inhibitors with diazoxide, or pharmaceutically acceptable salts thereof, results in increased exposure to diazoxide. Diazoxide choline is a substrate of OAT1 and OAT3 in vitro. Accordingly, transport of diazoxide choline may be reduced by strong OAT1 and OAT3 inhibitors, leading to increased plasma concentrations.

Accordingly, disclosed herein are methods of treating a subject in need thereof, said methods comprising:
(i) administering to the subject a therapeutically effective amount of diazoxide, or a pharmaceutically acceptable salt thereof, wherein the subject is concomitantly administered one or more strong OAT1 inhibitors; and
(j) adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject in need thereof, wherein:
(k) the subject is administered a therapeutically effective amount of diazoxide, or a pharmaceutically acceptable salt thereof, wherein the subject is concomitantly administered one or more strong OAT1 inhibitors; and
(l) the therapeutically effective amount of diazoxide, or a pharmaceutically acceptable salt thereof, administered to the subject is adjusted.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(m) administering to the subject a therapeutically effective amount of diazoxide, or a pharmaceutically acceptable salt thereof, wherein the subject is concomitantly administered one or more strong OAT1 inhibitors; and
(n) adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(o) the subject is administered a therapeutically effective amount of diazoxide, or a pharmaceutically acceptable salt thereof, wherein the subject is concomitantly administered one or more strong OAT1 inhibitors; and
(p) the therapeutically effective amount of diazoxide, or a pharmaceutically acceptable salt thereof, administered to the subject is adjusted.

In some embodiments, the one or more strong OAT1 inhibitors is selected from the group consisting of benzylpenicillin, methotrexate, and pravastatin. In some embodiments, the one or more strong OAT1 inhibitors is benzylpenicillin, methotrexate, or pravastatin. In some embodiments, the one or more strong OAT1 inhibitors is benzylpenicillin. In some embodiments, the one or more strong OAT1 inhibitors is methotrexate. In some embodiments, the one or more strong OAT1 inhibitors is pravastatin.

In some embodiments, the therapeutically effective amount of diazoxide is reduced relative to the therapeutically effective amount for the subject if not concomitantly administered one or more strong OAT1 inhibitors.

Also disclosed herein are methods of treating a subject in need thereof, said methods comprising:
(q) administering to the subject a therapeutically effective amount of diazoxide, or a pharmaceutically acceptable salt thereof, wherein the subject is concomitantly administered one or more strong OAT3 inhibitors; and
(r) adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject in need thereof, wherein:
(s) the subject is administered a therapeutically effective amount of diazoxide, or a pharmaceutically acceptable salt thereof, wherein the subject is concomitantly administered one or more strong OAT3 inhibitors; and
(t) the therapeutically effective amount of diazoxide, or a pharmaceutically acceptable salt thereof, administered to the subject is adjusted.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(u) administering to the subject a therapeutically effective amount of diazoxide, or a pharmaceutically acceptable salt thereof, wherein the subject is concomitantly administered one or more strong OAT3 inhibitors; and
(v) adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(w) the subject is administered a therapeutically effective amount of diazoxide, or a pharmaceutically acceptable salt thereof, wherein the subject is concomitantly administered one or more strong OAT3 inhibitors; and
(x) the therapeutically effective amount of diazoxide, or a pharmaceutically acceptable salt thereof, administered to the subject is adjusted.

In some embodiments, the one or more strong OAT3 inhibitors is selected from the group consisting of benzylpenicillin, methotrexate, and pravastatin. In some embodiments, the one or more strong OAT3 inhibitors is benzylpenicillin, methotrexate, or pravastatin. In some embodiments, the one or more strong OAT3 inhibitors is benzylpenicillin. In some embodiments, the one or more strong OAT3 inhibitors is methotrexate. In some embodiments, the one or more strong OAT3 inhibitors is pravastatin.

In some embodiments, the therapeutically effective amount of diazoxide is reduced relative to the therapeutically effective amount for the subject if not concomitantly administered one or more strong OAT3 inhibitors.

An example of reduced dose titrations for subjects, such as subjects with PWS, within different weight ranges are shown in Tables 5a-5b, below.

TABLE 5a
First Example of Diazoxide Choline Dosage Modifications for Concomitant Use with
Strong CYP1A2 Inhibitors, Strong OAT1 Inhibitors, or Strong OAT3 Inhibitors

| | Diazoxide Choline Recommended Once Daily Dosage | | | | | | | |
|---|---|---|---|---|---|---|---|---|
| | Starting | | Titration | | Titration | | | |
| | Dosage | | Dosage | | Dosage | | Target | |
| | Weeks | | Weeks | | Weeks | | Maintenance | |
| Weight | 1 and 2 | | 3 and 4 | | 5 and 6 | | Dosage | |
| ≥3 to <10 kg | 1.1 | mg/kg/day | 1.7 | mg/kg/day | 2.4 | mg/kg/day | 3.0 | mg/kg/day |
| ≥10 to <14 kg | 18 | mg/day | 36 | mg/day | 36 | mg/day | 42 | mg/day |
| ≥14 to <20 kg | 18 | mg/day | 36 | mg/day | 54 | mg/day | 58 | mg/day |
| 20 kg to <30 kg | 25 | mg/day | 25 | mg/day | 50 | mg/day | 75 | mg/day |
| 30 kg to <40 kg | 50 | mg/day | 100 | mg/day | 100 | mg/day | 100 | mg/day |
| 40 kg to <65 kg | 50 | mg/day | 100 | mg/day | 150 | mg/day | 150 | mg/day |
| 65 kg to <100 kg | 100 | mg/day | 150 | mg/day | 200 | mg/day | 250 | mg/day |
| 100 kg to <135 kg | 100 | mg/day | 200 | mg/day | 250 | mg/day | 300 | mg/day |
| ≥135 kg | 100 | mg/day | 200 | mg/day | 300 | mg/day | 325 | mg/day |

In some embodiments, a subject may not initially be titrated to the target maintenance dose and instead will initially be titrated to a lower dose based on the titration steps described herein. For some subjects, after a period of time (e.g., 3 months, 4 months, 6 months, 9 months, 12 months, 18 months, or more), further dose escalations up to the target maintenance doses described herein may be undertaken based on a need for greater efficacy and a subject's response to treatment. For example, for some subjects weighing≥65 kg, it may be recommended to initially titrate to 200 mg/day (Titration Dosage for Weeks 3-4 or Weeks 5-6) based on tolerability. Further dose escalation up to the Target Maintenance Dosage may be considered based on the need for greater efficacy, balanced with the potential for adverse drug reactions.

TABLE 5b
Second Example of Diazoxide Choline Dosage Modifications for Concomitant Use with
Strong CYP1A2 Inhibitors, Strong OAT1 Inhibitors, or Strong OAT3 Inhibitors

| | | Step 1 | Step 2 | | Step 3 | | Step 4 | | Step 5 | Step 6 | Initial | | Max | |
|---|---|---|---|---|---|---|---|---|---|---|---|---|---|---|
| | | Weeks | Weeks | | Weeks | | Weeks | | Weeks | Weeks | Target | | Target | |
| Weight | | 1-4 | 5-8 | | 9-12 | | 12-16 | | 17-20 | 20-24 | Dose | | Dose | |
| 20 to <30 | kg | 25 mg | 25 | mg | 50 | mg | 75 | mg | — | — | 75 | mg | 75 | mg |
| 30 to <40 | kg | 50 mg | 100 | mg | — | | — | | — | — | 100 | mg | 100 | mg |
| 40 to <65 | kg | 50 mg | 100 | mg | 150 | mg | — | | — | — | 150 | mg | 150 | mg |
| 65 to <100 | kg | 50 mg | 100 | mg | 150 | mg | 200 | mg | — | — | 200 | mg | 250 | mg |
| 100 to <135 | kg | 50 mg | 100 | mg | 150 | mg | 200 | mg | 225 mg | — | 225 | mg | 300 | mg |
| ≥135 | kg | 50 mg | 100 | mg | 150 | mg | 200 | mg | 225 mg | 275 mg | 275 | mg | 325 | mg |

In some embodiments, certain patients may require a modified dosing schedule. Examples of said modified dosing schedules are provided in Tables 5a and 5b. In some embodiments, the subject is dosed according to Tables 5a or 5b. In some embodiments, the subject is dosed according to Table 5a. In some embodiments, the subject is dosed according to Table 5b.

In some embodiments, a subject with one or more clinically relevant comorbidities (e.g., such as the clinically relevant comorbidities described herein) may also require concomitant administration of one or more strong CYP1A2 inhibitors, one or more strong OAT1 inhibitors, or one or more strong OAT3 inhibitors. Thus, in some embodiments, such a subject may be dosed according to Table 5b. In some embodiments, the subject in need of concomitant administration of one or more strong CYP1A2 inhibitors is also a subject having one or more clinically relevant comorbidities, as described herein. In some embodiments, the subject in need of concomitant administration of one or more strong OAT1 inhibitors is also a subject having one or more clinically relevant comorbidities, as described herein. In some embodiments, the subject in need of concomitant administration of one or more strong OAT3 inhibitors is also a subject having one or more clinically relevant comorbidities, as described herein.

In some embodiments, the reduced target maintenance dose of diazoxide choline is between about 1.1-4.2 mg/kg/day of diazoxide choline. In some embodiments, the reduced target maintenance dose of diazoxide choline is about 1.1 mg/kg/day of diazoxide choline. In some embodiments, the reduced target maintenance dose of diazoxide choline is about 3.0 mg/kg/day of diazoxide choline. In some embodiments, the reduced target maintenance dose of diazoxide choline is about 4.2 mg/kg/day of diazoxide choline.

In some embodiments, the reduced starting dose of diazoxide choline is between about 3-100 mg/day of diazoxide choline, including between about 4-100 mg/day of diazoxide choline, between about 3-18 mg/day of diazoxide choline, between about 3-25 mg/day of diazoxide choline, between about 3-50 mg/day of diazoxide choline, between about 5-18 mg/day of diazoxide choline, between about 5-50 mg/day of diazoxide choline, between about 5-100 mg/day of diazoxide choline, between about 10-18 mg/day of diazoxide choline, between about 10-50 mg/day of diazoxide choline, between about 10-100 mg/day of diazoxide choline, between about 25-50 mg/day of diazoxide choline, between about 25-100 mg/day of diazoxide choline, between about 18-25 mg/day of diazoxide choline, between about 18-50 mg/day of diazoxide choline, or between about 18-100 mg/day of diazoxide choline. In some embodiments, the reduced starting dose of diazoxide choline is about 3 mg/day, about 4 mg/day, about 5 mg/day, 10 mg/day, about 15 mg/day, about 18 mg/day, about 25 mg/day, about 50 mg/day, or about 100 mg/day of diazoxide choline.

In some embodiments, the starting dose of diazoxide, or a pharmaceutically acceptable salt thereof, is reduced relative to the starting dose for the subject if not concomitantly administered one or more strong CYP1A2 inhibitors. In some embodiments, the starting dose of diazoxide, or a pharmaceutically acceptable salt thereof, is reduced by between about 25%-35% relative to the starting dose for the subject if not concomitantly administered one or more strong CYP1A2 inhibitors. In some embodiments, the starting dose of diazoxide, or a pharmaceutically acceptable salt thereof, is reduced relative to the starting dose for the subject if not concomitantly administered one or more strong OAT1 inhibitors. In some embodiments, the starting dose of diazoxide, or a pharmaceutically acceptable salt thereof, is reduced by between about 25%-35% relative to the starting dose for the subject if not concomitantly administered one or more strong OAT1 inhibitors. In some embodiments, the starting dose of diazoxide, or a pharmaceutically acceptable salt thereof, is reduced relative to the starting dose for the subject if not concomitantly administered one or more strong OAT3 inhibitors. In some embodiments, the starting dose of diazoxide, or a pharmaceutically acceptable salt thereof, is reduced by between about 25%-35% relative to the starting dose for the subject if not concomitantly administered one or more strong OAT3 inhibitors.

In some embodiments, the reduced starting dose of diazoxide, or a pharmaceutically acceptable salt thereof, is increased to a reduced second dose of diazoxide, or a pharmaceutically acceptable salt thereof, after 2-3 weeks.

In some embodiments, the reduced second dose of diazoxide choline is between about 5-200 mg/day of diazoxide choline, including between about 5-25 mg/day of diazoxide choline, between about 5-36 mg/day of diazoxide choline, between about 5-100 mg/day of diazoxide choline, between about 5-150 mg/day of diazoxide choline, between about 25-36 mg/day of diazoxide choline, between about 25-100 mg/day of diazoxide choline, between about 25-150 mg/day of diazoxide choline, between about 25-200 mg/day of diazoxide choline, between about 100-150 mg/day of diazoxide choline, between about 100-200 mg/day of diazoxide choline, or between about 150-200 mg/day of diazoxide choline. In some embodiments, the reduced second dose of diazoxide choline is about 5 mg/day, about 25 mg/day, about 36 mg/day, about 100 mg/day, about 150 mg/day, or about 200 mg/day of diazoxide choline.

In some embodiments, the second dose of diazoxide, or a pharmaceutically acceptable salt thereof, is reduced relative to the second dose for the subject if not concomitantly administered one or more strong CYP1A2 inhibitors. In some embodiments, the second dose of diazoxide, or a pharmaceutically acceptable salt thereof, is reduced by between about 25%-55% relative to the second dose for the subject if not concomitantly administered one or more strong CYP1A2 inhibitors. In some embodiments, the second dose of diazoxide, or a pharmaceutically acceptable salt thereof, is reduced relative to the second dose for the subject if not concomitantly administered one or more strong OAT1 inhibitors. In some embodiments, the second dose of diazoxide, or a pharmaceutically acceptable salt thereof, is reduced by between about 25%-55% relative to the second dose for the subject if not concomitantly administered one or more strong OAT1 inhibitors. In some embodiments, the second dose of diazoxide, or a pharmaceutically acceptable salt thereof, is reduced relative to the second dose for the subject if not concomitantly administered one or more strong OAT3 inhibitors. In some embodiments, the second dose of diazoxide, or a pharmaceutically acceptable salt thereof, is reduced by between about 25%-55% relative to the second dose for the subject if not concomitantly administered one or more strong OAT3 inhibitors.

In some embodiments, the reduced second dose of diazoxide, or a pharmaceutically acceptable salt thereof, is increased to a reduced third dose of diazoxide, or a pharmaceutically acceptable salt thereof, after 2-3 weeks.

In some embodiments, the reduced third dose of diazoxide choline is between about 7-300 mg/day of diazoxide choline, including between about 7-36 mg/day of diazoxide choline, between about 7-50 mg/day of diazoxide choline, between about 7-54 mg/day of diazoxide choline, between about 7-100 mg/day of diazoxide choline, between about 7-150 mg/day of diazoxide choline, between about 7-200 mg/day of diazoxide choline, between about 7-250 mg/day of diazoxide choline, between about 36-50 mg/day of diazoxide choline, between about 36-54 mg/day of diazoxide choline, between about 36-100 mg/day of diazoxide choline, between about 36-150 mg/day of diazoxide choline, between about 36-200 mg/day of diazoxide choline, between about 36-250 mg/day of diazoxide choline, between about 36-300 mg/day of diazoxide choline, between about 50-54 mg/day of diazoxide choline, between about 50-100 mg/day of diazoxide choline, between about 50-150 mg/day of diazoxide choline, between about 50-200 mg/day of diazoxide choline, between about 50-250 mg/day of diazoxide choline, between about 50-300 mg/day of diazoxide choline, between about 54-100 mg/day of diazoxide choline, between about 54-150 mg/day of diazoxide choline, between about 54-200 mg/day of diazoxide choline, between about 54-250 mg/day of diazoxide choline, between about 54-300 mg/day of diazoxide choline, between about 100-150 mg/day of diazoxide choline, between about 100-200 mg/day of diazoxide choline, between about 100-250 mg/day of diazoxide choline, between about 100-300 mg/day of diazoxide choline, between about 150-200 mg/day of diazoxide choline, between about 150-250 mg/day of diazoxide choline, between about 150-300 mg/day of diazoxide choline, between about 200-250 mg/day of diazoxide choline, between about 200-300 mg/day of diazoxide choline, or between about 250-300 mg/day of diazoxide choline. In some embodiments, the reduced third dose of diazoxide choline is about 7 mg/day, about 36 mg/day, about 50 mg/day, about 54 mg/day, about 100 mg/day, about 150 mg/day, about 200 mg/day, about 250 mg/day, or about 300 mg/day of diazoxide choline.

In some embodiments, the third dose of diazoxide, or a pharmaceutically acceptable salt thereof, is reduced relative to the third dose for the subject if not concomitantly administered one or more strong CYP1A2 inhibitors. In some embodiments, the third dose of diazoxide, or a pharmaceutically acceptable salt thereof, is reduced by between about 25%-35% relative to the third dose for the subject if not concomitantly administered one or more strong CYP1A2 inhibitors. In some embodiments, the third dose of diazoxide, or a pharmaceutically acceptable salt thereof, is reduced relative to the third dose for the subject if not concomitantly administered one or more strong OAT1 inhibitors. In some embodiments, the third dose of diazoxide, or a pharmaceutically acceptable salt thereof, is reduced by between about 25%-35% relative to the third dose for the subject if not concomitantly administered one or more strong OAT1 inhibitors. In some embodiments, the third dose of diazoxide, or a pharmaceutically acceptable salt thereof, is reduced relative to the third dose for the subject if not concomitantly administered one or more strong OAT3 inhibitors. In some embodiments, the third dose of diazoxide, or a pharmaceutically acceptable salt thereof, is reduced by between about 25%-35% relative to the third dose for the subject if not concomitantly administered one or more strong OAT3 inhibitors.

In some embodiments, the reduced third dose of diazoxide, or a pharmaceutically acceptable salt thereof, is increased to the reduced target maintenance dose of diazoxide, or a pharmaceutically acceptable salt thereof, after 2-3 weeks.

In some embodiments, the reduced target maintenance dose of diazoxide choline is between about 9-325 mg/day of diazoxide choline, including between about 9-42 mg/day of diazoxide choline, between about 9-58 mg/day of diazoxide choline, between about 9-75 mg/day of diazoxide choline, between about 9-100 mg/day of diazoxide choline, between about 9-150 mg/day of diazoxide choline, between about 9-250 mg/day of diazoxide choline, between about 9-300 mg/day of diazoxide choline, between about 9-325 mg/day of diazoxide choline, between about 42-58 mg/day of diazoxide choline, between about 42-75 mg/day of diazoxide choline, between about 42-100 mg/day of diazoxide choline, between about 42-150 mg/day of diazoxide choline, between about 42-250 mg/day of diazoxide choline, between about 42-300 mg/day of diazoxide choline, between about 42-325 mg/day of diazoxide choline, between about 58-75 mg/day of diazoxide choline, between about 58-100 mg/day of diazoxide choline, between about 58-150 mg/day of diazoxide choline, between about 58-250 mg/day of diazoxide choline, between about 58-300 mg/day of diazoxide choline, between about 58-325 mg/day of diazoxide choline, between about 75-100 mg/day of diazoxide choline, between about 75-150 mg/day of diazoxide choline, between about 75-250 mg/day of diazoxide choline, between about 75-300 mg/day of diazoxide choline, between about 75-325 mg/day of diazoxide choline, between about 100-150 mg/day of diazoxide choline, between about 100-250 mg/day of diazoxide choline, between about 100-300 mg/day of diazoxide choline, between about 100-325 mg/day of diazoxide choline, between about 150-250 mg/day of diazoxide choline, between about 150-300 mg/day of diazoxide choline, between about 150-325 mg/day of diazoxide choline, between about 250-300 mg/day of diazoxide choline, between about 250-325 mg/day of diazoxide choline, or between about 300-325 mg/day of diazoxide choline. In some embodiments, the reduced target maintenance dose of diazoxide choline is about 9 mg/day, about 42 mg/day, about 58 mg/day, about 75 mg/day, about 100 mg/day, about 150 mg/day, about 250 mg/day, about 300 mg/day, or about 325 mg/day of diazoxide choline. In some embodiments, the reduced target maintenance dose of diazoxide choline is between about 9-325 mg/day of diazoxide choline, including between about 75-200 mg/day of diazoxide choline, between about 75-225 mg/day of diazoxide choline, between about 75-275 mg/day of diazoxide choline, between about 100-200 mg/day of diazoxide choline, between about 100-225 mg/day of diazoxide choline, between about 100-275 mg/day of diazoxide choline, between about 150-200 mg/day of diazoxide choline, between about 150-225 mg/day of diazoxide choline, between about 150-275 mg/day of diazoxide choline, between about 200-225 mg/day of diazoxide choline, between about 200-275 mg/day of diazoxide choline, or between about 225-275 mg/day of diazoxide choline. In some embodiments, the reduced target maintenance dose of diazoxide choline is about 200 mg/day, about 225 mg/day, or about 275 mg/day of diazoxide choline.

In some embodiments, the target maintenance dose of diazoxide, or a pharmaceutically acceptable salt thereof, is reduced relative to the target maintenance dose for the subject if not concomitantly administered one or more strong CYP1A2 inhibitors. In some embodiments, the target maintenance dose of diazoxide, or a pharmaceutically acceptable salt thereof, is reduced by between about 23%-40% relative to the target maintenance dose for the subject if not concomitantly administered one or more strong CYP1A2 inhibitors. In some embodiments, the target maintenance dose of diazoxide, or a pharmaceutically acceptable salt thereof, is reduced relative to the target maintenance dose for the subject if not concomitantly administered one or more strong OAT1 inhibitors. In some embodiments, the target maintenance dose of diazoxide, or a pharmaceutically acceptable salt thereof, is reduced by between about 23%-40% relative to the target maintenance dose for the subject if not concomitantly administered one or more strong OAT1 inhibitors. In some embodiments, the target maintenance dose of diazoxide, or a pharmaceutically acceptable salt thereof, is reduced relative to the target maintenance dose for the subject if not concomitantly administered one or more strong OAT3 inhibitors.

In some embodiments, the target maintenance dose of diazoxide, or a pharmaceutically acceptable salt thereof, is reduced by between about 23%-40% relative to the target maintenance dose for the subject if not concomitantly administered one or more strong OAT3 inhibitors.

In some embodiments, the target maintenance dose of diazoxide, or a pharmaceutically acceptable salt thereof, for a subject with moderate renal impairment is between about one-third and about one-half of the target maintenance dose of diazoxide, or a pharmaceutically acceptable salt thereof, for a subject without moderate renal impairment.

In some embodiments, treatment is temporarily interrupted. In some embodiments, treatment resumes at the starting dose of diazoxide, or a pharmaceutically acceptable salt thereof, following an interruption in treatment lasting 7 or more days. In some embodiments, treatment resumes at the starting dose of diazoxide, or a pharmaceutically acceptable salt thereof, following an interruption in treatment lasting 14 or more days.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods further comprise discontinuing treatment of the subject. In some embodiments, treatment of the subject is discontinued without tapering.

In some embodiments, the administration of diazoxide choline is in an amount from about 1.1 mg/kg/day to about 4.2 mg/kg/day. In some embodiments, the administration of diazoxide choline is in an amount of about 4.2 mg/kg/day. In some embodiments the administration of diazoxide choline is in an amount of about 1.1 mg/kg/day, about 1.5 mg/kg/day, about 2.4 mg/kg/day, about 3.3 mg/kg/day, or about 4.2 mg/kg/day.

In some embodiments, a once-daily administration of diazoxide choline is in an amount from about 3 mg to about 9 mg. In some embodiments, a once-daily administration of diazoxide choline is in an amount from about 4 mg to about 9 mg. In some embodiments, a once-daily administration of diazoxide choline is in an amount from about 18 mg to about 42 mg. In some embodiments, the once-daily administration of diazoxide choline is in an amount from about 18 mg to about 58 mg. In some embodiments, the once-daily administration of diazoxide choline is in an amount from about 25 mg to about 75 mg. In some embodiments, the once-daily administration of diazoxide choline is in an amount from about 50 mg to about 100 mg. In some embodiments, the once-daily administration of diazoxide choline is in an amount from about 50 mg to about 150 mg. In some embodiments, the once-daily administration of diazoxide choline is in an amount from about 100 mg to about 250 mg. In some embodiments, the once-daily administration of diazoxide choline is in an amount from about 100 mg to about 300 mg. In some embodiments, the once-daily administration of diazoxide choline is in an amount from about 100 mg to about 325 mg. In some embodiments, the once-daily administration of diazoxide choline is in an amount from about 50 mg to about 200 mg. In some embodiments, the once-daily administration of diazoxide choline is in an amount from about 50 mg to about 250 mg. In some embodiments, the once-daily administration of diazoxide choline is in an amount from about 50 mg to about 225 mg. In some embodiments, the once-daily administration of diazoxide choline is in an amount from about 50 mg to about 300 mg. In some embodiments, the once-daily administration of diazoxide choline is in an amount from about 50 mg to about 275 mg. In some embodiments, the once-daily administration of diazoxide choline is in an amount from about 50 mg to about 325 mg.

In some embodiments, diazoxide, or a pharmaceutically acceptable salt thereof, including diazoxide choline, may be administered to a subject in need thereof to achieve therapeutic outcomes while reducing the incidence of adverse effects. In some embodiments, diazoxide, or a pharmaceutically acceptable salt thereof, including diazoxide choline, may be administered to the subject in need thereof at a dosage including from about 3 mg to about 100 mg, about 100 mg to about 200 mg, about 200 mg to about 300 mg, about 300 mg to about 400 mg, about 400 mg to about 500 mg, about 500 mg to about 600 mg, including from about 12 mg to about 580 mg. In some embodiments, diazoxide may be administered to the subject in need thereof at a dosage including from about 3 mg to about 400 mg, including about 17 mg to about 400 mg, about 17 mg to about 350 mg, about 17 mg to about 300 mg, about 17 mg to about 250 mg, about 17 mg to about 200 mg, about 17 mg to about 150 mg, about 17 mg to about 100 mg, or about 17 mg to about 50 mg. In some embodiments, diazoxide may be administered to the subject in need thereof at a dosage from about 3 mg to about 400 mg including from about 100 mg to about 400 mg, about 150 mg to about 400 mg, about 200 mg to about 400 mg, about 250 mg to about 400 mg, or about 300 mg to about 400 mg.

In some embodiments, diazoxide, or a pharmaceutically acceptable salt thereof, including diazoxide choline, may be administered to a subject with PWS to achieve therapeutic outcomes while reducing the incidence of adverse effects. In some embodiments, diazoxide, or a pharmaceutically acceptable salt thereof, including diazoxide choline, may be administered to the subject with PWS at a dosage including from about 3 mg to about 100 mg, about 100 mg to about 200 mg, about 200 mg to about 300 mg, about 300 mg to about 400 mg, about 400 mg to about 500 mg, about 500 mg to about 600 mg, including from about 12 mg to about 580 mg. In some embodiments, diazoxide may be administered to the subject with PWS at a dosage including from about 3 mg to about 400 mg, including about 17 mg to about 400 mg, about 17 mg to about 350 mg, about 17 mg to about 300 mg, about 17 mg to about 250 mg, about 17 mg to about 200 mg, about 17 mg to about 150 mg, about 17 mg to about 100 mg, or about 17 mg to about 50 mg. In some embodiments, diazoxide may be administered to the subject with PWS at a dosage from about 3 mg to about 400 mg including from about 100 mg to about 400 mg, about 150 mg to about 400 mg, about 200 mg to about 400 mg, about 250 mg to about 400 mg, or about 300 mg to about 400 mg.

In some embodiments, diazoxide, or a pharmaceutically acceptable salt thereof, is administered one or more times per day (e.g., 1, 2, 3, 4, or more times per day). In some embodiments, diazoxide, or a pharmaceutically acceptable salt thereof, is administered once per day to the subject. In some embodiments, diazoxide, or a pharmaceutically acceptable salt thereof, is administered twice per day to the subject. In some embodiments, diazoxide, or a pharmaceutically acceptable salt thereof, is administered thrice per day to the subject. In some embodiments, diazoxide, or a pharmaceutically acceptable salt thereof, is administered four or more times per day to the subject.

Also disclosed herein are methods of treating a subject in need thereof,
said methods comprising:
   (a1) orally administering to the subject 25 mg of diazoxide choline once daily for an initial period of four weeks;
   (b1) after the initial four-week period, orally administering to the subject 50 mg of diazoxide choline once daily for a subsequent period of two weeks; and
   (c1) after the subsequent two-week period, orally administering to the subject 75 mg of diazoxide choline once daily;
or
   (a2) orally administering to the subject 50 mg of diazoxide choline once daily for an initial period of two weeks; and
   (b2) after the initial two-week period, orally administering to the subject 100 mg of diazoxide choline once daily;
or
   (a3) orally administering to the subject 50 mg of diazoxide choline once daily for an initial period of two weeks;
   (b3) after the initial two-week period, orally administering to the subject 100 mg of diazoxide choline once daily for a second two-week period; and
   (c3) after the second two-week period, orally administering to the subject 150 mg of diazoxide choline once daily;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 150 mg of diazoxide choline;
   (c4) after the second two-week period, orally administering to the subject a second titration dosage of diazoxide choline once daily for a third two-week period, wherein the second titration dosage is 200 mg of diazoxide choline; and
   (d4) after the third two-week period, orally administering to the subject a target maintenance dose of diazoxide choline once daily, wherein the target maintenance dose is 250 mg of diazoxide choline;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 200 mg of diazoxide choline;
   (c4) after the second two-week period, orally administering to the subject a second titration dosage of diazoxide choline once daily for a third two-week period, wherein the second titration dosage is 250 mg of diazoxide choline; and
   (d4) after the third two-week period, orally administering to the subject a target maintenance dose of diazoxide choline once daily, wherein the target maintenance dose is 300 mg of diazoxide choline;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 200 mg of diazoxide choline;
   (c4) after the second two-week period, orally administering to the subject a second titration dosage of diazoxide choline once daily for a third two-week period, wherein the second titration dosage is 300 mg of diazoxide choline; and
   (d4) after the third two-week period, orally administering to the subject a target maintenance dose of diazoxide choline once daily, wherein the target maintenance dose is 325 mg of diazoxide choline;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof,
wherein:
   (a1) 25 mg of diazoxide choline is orally administered to the subject once daily for an initial period of four weeks;
   (b1) after the initial four-week period, 50 mg of diazoxide choline is orally administered to the subject once daily for a subsequent period of two weeks; and
   (c1) after the subsequent two-week period, 75 mg of diazoxide choline is orally administered to the subject once daily;
or
   (a2) 50 mg of diazoxide choline is orally administered to the subject once daily for an initial period of two weeks; and
   (b2) after the initial two-week period, 100 mg of diazoxide choline is orally administered to the subject once daily;
or
   (a3) 50 mg of diazoxide choline is orally administered to the subject once daily for an initial period of two weeks;
   (b3) after the initial two-week period, 100 mg of diazoxide choline is orally administered to the subject once daily for a second two-week period; and
   (c3) after the second two-week period, 150 mg of diazoxide choline is orally administered to the subject once daily;
or
   (a4) a starting dosage of 100 mg of diazoxide choline is orally administered to the subject once daily for an initial period of two weeks;
   (b4) after the initial two-week period, a first titration dosage of diazoxide choline is orally administered to the subject once daily for a second two-week period, wherein the first titration dosage is 150 mg of diazoxide choline;
   (c4) after the second two-week period, a second titration dosage of diazoxide choline is orally administered to the subject once daily for a third two-week period, wherein the second titration dosage is 200 mg of diazoxide choline; and
   (d4) after the third two-week period, a target maintenance dose of diazoxide choline is orally administered to the subject once daily, wherein the target maintenance dose is 250 mg of diazoxide choline;
or
   (a4) a starting dosage of 100 mg of diazoxide choline is orally administered to the subject once daily for an initial period of two weeks;
   (b4) after the initial two-week period, a first titration dosage of diazoxide choline is orally administered to the subject once daily for a second two-week period, wherein the first titration dosage is 200 mg of diazoxide choline;
   (c4) after the second two-week period, a second titration dosage of diazoxide choline is orally administered to the subject once daily for a third two-week period, wherein the second titration dosage is 250 mg of diazoxide choline; and
   (d4) after the third two-week period, a target maintenance dose of diazoxide choline is orally administered to the subject once daily, wherein the target maintenance dose is 300 mg of diazoxide choline;
or
   (a4) a starting dosage of 100 mg of diazoxide choline is orally administered to the subject once daily for an initial period of two weeks;
   (b4) after the initial two-week period, a first titration dosage of diazoxide choline is orally administered to the subject once daily for a second two-week period, wherein the first titration dosage is 200 mg of diazoxide choline;
   (c4) after the second two-week period, a second titration dosage of diazoxide choline is orally administered to the subject once daily for a third two-week period, wherein the second titration dosage is 300 mg of diazoxide choline; and
   (d4) after the third two-week period, a target maintenance dose of diazoxide choline is orally administered to the subject once daily, wherein the target maintenance dose is 325 mg of diazoxide choline;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS,
said methods comprising:
   (a1) orally administering to the subject 25 mg of diazoxide choline once daily for an initial period of four weeks;
   (b1) after the initial four-week period, orally administering to the subject 50 mg of diazoxide choline once daily for a subsequent period of two weeks; and
   (c1) after the subsequent two-week period, orally administering to the subject 75 mg of diazoxide choline once daily;
or
   (a2) orally administering to the subject 50 mg of diazoxide choline once daily for an initial period of two weeks; and
   (b2) after the initial two-week period, orally administering to the subject 100 mg of diazoxide choline once daily;
or
   (a3) orally administering to the subject 50 mg of diazoxide choline once daily for an initial period of two weeks;
   (b3) after the initial two-week period, orally administering to the subject 100 mg of diazoxide choline once daily for a second two-week period; and
   (c3) after the second two-week period, orally administering to the subject 150 mg of diazoxide choline once daily;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 150 mg of diazoxide choline;
   (c4) after the second two-week period, orally administering to the subject a second titration dosage of diazoxide choline once daily for a third two-week period, wherein the second titration dosage is 200 mg of diazoxide choline; and
   (d4) after the third two-week period, orally administering to the subject a target maintenance dose of diazoxide choline once daily, wherein the target maintenance dose is 250 mg of diazoxide choline;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 200 mg of diazoxide choline;
   (c4) after the second two-week period, orally administering to the subject a second titration dosage of diazoxide choline once daily for a third two-week period, wherein the second titration dosage is 250 mg of diazoxide choline; and
   (d4) after the third two-week period, orally administering to the subject a target maintenance dose of diazoxide choline once daily, wherein the target maintenance dose is 300 mg of diazoxide choline;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 200 mg of diazoxide choline;
   (c4) after the second two-week period, orally administering to the subject a second titration dosage of diazoxide choline once daily for a third two-week period, wherein the second titration dosage is 300 mg of diazoxide choline; and
   (d4) after the third two-week period, orally administering to the subject a target maintenance dose of diazoxide choline once daily, wherein the target maintenance dose is 325 mg of diazoxide choline;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS,
wherein:
   (a1) 25 mg of diazoxide choline is orally administered to the subject once daily for an initial period of four weeks;
   (b1) after the initial four-week period, 50 mg of diazoxide choline is orally administered to the subject once daily for a subsequent period of two weeks; and
   (c1) after the subsequent two-week period, 75 mg of diazoxide choline is orally administered to the subject once daily;
or
   (a2) 50 mg of diazoxide choline is orally administered to the subject once daily for an initial period of two weeks; and
   (b2) after the initial two-week period, 100 mg of diazoxide choline is orally administered to the subject once daily;
or
   (a3) 50 mg of diazoxide choline is orally administered to the subject once daily for an initial period of two weeks;
   (b3) after the initial two-week period, 100 mg of diazoxide choline is orally administered to the subject once daily for a second two-week period; and
   (c3) after the second two-week period, 150 mg of diazoxide choline is orally administered to the subject once daily;
or
   (a4) a starting dosage of 100 mg of diazoxide choline is orally administered to the subject once daily for an initial period of two weeks;
   (b4) after the initial two-week period, a first titration dosage of diazoxide choline is orally administered to the subject once daily for a second two-week period, wherein the first titration dosage is 150 mg of diazoxide choline;
   (c4) after the second two-week period, a second titration dosage of diazoxide choline is orally administered to the subject once daily for a third two-week period, wherein the second titration dosage is 200 mg of diazoxide choline; and
   (d4) after the third two-week period, a target maintenance dose of diazoxide choline is orally administered to the subject once daily, wherein the target maintenance dose is 250 mg of diazoxide choline;
or
   (a4) a starting dosage of 100 mg of diazoxide choline is orally administered to the subject once daily for an initial period of two weeks;
   (b4) after the initial two-week period, a first titration dosage of diazoxide choline is orally administered to the subject once daily for a second two-week period, wherein the first titration dosage is 200 mg of diazoxide choline;
   (c4) after the second two-week period, a second titration dosage of diazoxide choline is orally administered to the subject once daily for a third two-week period, wherein the second titration dosage is 250 mg of diazoxide choline; and
   (d4) after the third two-week period, a target maintenance dose of diazoxide choline is orally administered to the subject once daily, wherein the target maintenance dose is 300 mg of diazoxide choline;
or
   (a4) a starting dosage of 100 mg of diazoxide choline is orally administered to the subject once daily for an initial period of two weeks;
   (b4) after the initial two-week period, a first titration dosage of diazoxide choline is orally administered to the subject once daily for a second two-week period, wherein the first titration dosage is 200 mg of diazoxide choline;
   (c4) after the second two-week period, a second titration dosage of diazoxide choline is orally administered to the subject once daily for a third two-week period, wherein the second titration dosage is 300 mg of diazoxide choline; and
   (d4) after the third two-week period, a target maintenance dose of diazoxide choline is orally administered to the subject once daily, wherein the target maintenance dose is 325 mg of diazoxide choline;
wherein the subject is concomitantly being administered one or more strong CYP1A2 inhibitors.

In some embodiments, the subject weighs 20 kg to less than 30 kg. In some embodiments, the subject weighs 30 kg to less than 40 kg. In some embodiments, the subject weighs 40 kg to less than 65 kg. In some embodiments, the subject weighs 65 kg to less than 100 kg. In some embodiments, the subject weighs 100 kg to less than 135 kg. In some embodiments, the subject weighs 135 kg or more. In some embodiments, the subject weighs 20 kg. In some embodiments, the subject weighs 25 kg. In some embodiments, the subject weighs 30 kg. In some embodiments, the subject weighs 35 kg. In some embodiments, the subject weighs 40 kg. In some embodiments, the subject weighs 45 kg. In some embodiments, the subject weighs 50 kg. In some embodiments, the subject weighs 55 kg. In some embodiments, the subject weighs 60 kg. In some embodiments, the subject weighs 65 kg. In some embodiments, the subject weighs 70 kg. In some embodiments, the subject weighs 75 kg. In some embodiments, the subject weighs 80 kg. In some embodiments, the subject weighs 85 kg. In some embodiments, the subject weighs 90 kg. In some embodiments, the subject weighs 95 kg. In some embodiments, the subject weighs 100 kg. In some embodiments, the subject weighs 105 kg. In some embodiments, the subject weighs 110 kg. In some embodiments, the subject weighs 115 kg. In some embodiments, the subject weighs 120 kg. In some embodiments, the subject weighs 125 kg. In some embodiments, the subject weighs 130 kg. In some embodiments, the subject weighs 135 kg. In some embodiments, the subject weighs 140 kg. In some embodiments, the subject weighs 145 kg. In some embodiments, the subject weighs 150 kg.

Also disclosed herein are methods of treating a subject in need thereof, said methods comprising:
(a) orally administering to the subject 25 mg of diazoxide choline once daily for an initial period of four weeks;
(b) after the initial four-week period, orally administering to the subject 50 mg of diazoxide choline once daily for a subsequent period of two weeks; and
(c) after the subsequent two-week period, orally administering to the subject 75 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 25 mg of diazoxide choline is orally administered to the subject once daily for an initial period of four weeks;
(b) after the initial four-week period, 50 mg of diazoxide choline is orally administered to the subject once daily for a subsequent period of two weeks; and
(c) after the subsequent two-week period, 75 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of four weeks;
(b) after the initial four-week period, orally administering to the subject 50 mg of diazoxide choline once daily for a subsequent period of two weeks; and
(c) after the subsequent two-week period, orally administering to the subject 75 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 25 mg of diazoxide choline is orally administered to the subject once daily for an initial period of four weeks;
(b) after the initial four-week period, 50 mg of diazoxide choline is orally administered to the subject once daily for a subsequent period of two weeks; and
(c) after the subsequent two-week period, 75 mg of diazoxide choline is orally administered to the subject once daily;
wherein the subject is concomitantly being administered one or more strong CYP1A2 inhibitors.

Also disclosed herein are methods of treating a subject in need thereof, said methods comprising:
(a) orally administering to the subject 25 mg of diazoxide choline once daily for an initial period of at least eight weeks;
(b) after the initial period of at least eight weeks, orally administering to the subject 50 mg of diazoxide choline once daily for a sequential period of at least four weeks; and
(c) after the sequential period of at least four weeks, orally administering to the subject a target dose of 75 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 25 mg of diazoxide choline is orally administered to the subject once daily for an initial period of at least eight weeks;
(b) after the initial period of at least eight weeks, 50 mg of diazoxide choline is orally administered to the subject once daily for a sequential period of at least four weeks; and
(c) after the sequential period of at least four weeks, a target dose of 75 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 25 mg of diazoxide choline once daily for an initial period of at least eight weeks;
(b) after the initial period of at least eight weeks, orally administering to the subject 50 mg of diazoxide choline once daily for a sequential period of at least four weeks; and
(c) after the sequential period of at least four weeks, orally administering to the subject a target dose of 75 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 25 mg of diazoxide choline is orally administered to the subject once daily for an initial period of at least eight weeks;
(b) after the initial period of at least eight weeks, 50 mg of diazoxide choline is orally administered to the subject once daily for a sequential period of at least four weeks; and
(c) after the sequential period of at least four weeks, a target dose of 75 mg of diazoxide choline is orally administered to the subject once daily;
wherein the subject is concomitantly being administered one or more strong CYP1A2 inhibitors.

In some embodiments, the subject weighs 20 kg to less than 30 kg. In some embodiments, the subject weighs 20 kg. In some embodiments, the subject weighs 25 kg.

In some embodiments, the subject weighs 20 kg to less than 30 kg and is orally administered 25 mg of diazoxide choline once daily for an initial period of four weeks, orally administered 50 mg of diazoxide choline once daily for a subsequent period of two weeks, and orally administered 75 mg of diazoxide choline once daily after the subsequent two-week period.

Also disclosed herein are methods of treating a subject in need thereof, said methods comprising:
(a) orally administering to the subject 50 mg of diazoxide choline once daily for an initial period of two weeks; and
(b) after the initial two-week period, orally administering to the subject 100 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of two weeks; and
(b) after the initial two-week period, 100 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of two weeks; and
(b) after the initial two-week period, orally administering to the subject 100 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of two weeks; and
(b) after the initial two-week period, 100 mg of diazoxide choline is orally administered to the subject once daily;
wherein the subject is concomitantly being administered one or more strong CYP1A2 inhibitors.

Also disclosed herein are methods of treating a subject in need thereof, said methods comprising:
(a) orally administering to the subject 50 mg of diazoxide choline once daily for an initial period of at least four weeks; and
(b) after the initial period of at least four weeks, orally administering to the subject a target dose of 100 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of at least four weeks; and
(b) after the initial period of at least four weeks, a target dose of 100 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of at least four weeks; and
(b) after the initial period of at least four weeks, orally administering to the subject a target dose of 100 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of at least four weeks; and
(b) after the initial period of at least four weeks, a target dose of 100 mg of diazoxide choline is orally administered to the subject once daily;
wherein the subject is concomitantly being administered one or more strong CYP1A2 inhibitors.

In some embodiments, the subject weighs 30 kg to less than 40 kg. In some embodiments, the subject weighs 30 kg. In some embodiments, the subject weighs 35 kg.

In some embodiments, the subject weighs 30 kg to less than 40 kg and is orally administered 50 mg of diazoxide choline once daily for an initial period of two weeks, and orally administered 100 mg of diazoxide choline once daily after the initial two-week period.

Also disclosed herein are methods of treating a subject in need thereof, said methods comprising:
(a) orally administering to the subject 50 mg of diazoxide choline once daily for an initial period of two weeks;
(b) after the initial two-week period, orally administering to the subject 100 mg of diazoxide choline once daily for a second two-week period; and
(c) after the second two-week period, orally administering to the subject 150 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of two weeks;
(b) after the initial two-week period, 100 mg of diazoxide choline is orally administered to the subject once daily for a second two-week period; and
(c) after the second two-week period, 150 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of two weeks;
(b) after the initial two-week period, orally administering to the subject 100 mg of diazoxide choline once daily for a second two-week period; and
(c) after the second two-week period, orally administering to the subject 150 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of two weeks;
(b) after the initial two-week period, 100 mg of diazoxide choline is orally administered to the subject once daily for a second two-week period; and
(c) after the second two-week period, 150 mg of diazoxide choline is orally administered to the subject once daily;
wherein the subject is concomitantly being administered one or more strong CYP1A2 inhibitors.

Also disclosed herein are methods of treating a subject in need thereof, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks; and
(c) after the second period of at least four weeks, orally administering to the subject a target dose of 150 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks; and
(c) after the second period of at least four weeks, a target dose of 150 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks; and
(c) after the second period of at least four weeks, orally administering to the subject a target dose of 150 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks; and
(c) after the second period of at least four weeks, a target dose of 150 mg of diazoxide choline is orally administered to the subject once daily;
wherein the subject is concomitantly being administered one or more strong CYP1A2 inhibitors.

In some embodiments, the subject weighs 40 kg to less than 65 kg. In some embodiments, the subject weighs 40 kg. In some embodiments, the subject weighs 45 kg. In some embodiments, the subject weighs 50 kg. In some embodiments, the subject weighs 55 kg. In some embodiments, the subject weighs 60 kg.

In some embodiments, the subject weighs 40 kg to less than 65 kg and is orally administered 50 mg of diazoxide choline once daily for an initial period of two weeks, orally administered 100 mg of diazoxide choline once daily for a subsequent period of two weeks, and orally administered 150 mg of diazoxide choline once daily after the subsequent two-week period.

Also disclosed herein are methods of treating a subject in need thereof, said methods comprising:
(a) orally administering to the subject a starting dosage of 100 mg of diazoxide choline once daily for an initial period of two weeks;
(b) after the initial two-week period, orally administering to the subject a first titration dosage of diazoxide choline once daily for a second two-week period, wherein the first titration dosage is 150 mg or 200 mg of diazoxide choline;
(c) after the second two-week period, orally administering to the subject a second titration dosage of diazoxide choline once daily for a third two-week period, wherein the second titration dosage is 200 mg, 250 mg, or 300 mg of diazoxide choline; and
(d) after the third two-week period, orally administering to the subject a target maintenance dose of diazoxide choline once daily, wherein the target maintenance dose is 250 mg, 300 mg, or 325 mg of diazoxide choline;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) a starting dosage of 100 mg of diazoxide choline is orally administered to the subject once daily for an initial period of two weeks;
(b) after the initial two-week period, a first titration dosage of diazoxide choline is orally administered to the subject once daily for a second two-week period, wherein the first titration dosage is 150 mg or 200 mg of diazoxide choline;
(c) after the second two-week period, a second titration dosage of diazoxide choline is orally administered to the subject once daily for a third two-week period, wherein the second titration dosage is 200 mg, 250 mg, or 300 mg of diazoxide choline; and
(d) after the third two-week period, a target maintenance dose of diazoxide choline is orally administered to the subject once daily, wherein the target maintenance dose is 250 mg, 300 mg, or 325 mg of diazoxide choline;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject a starting dosage of 100 mg of diazoxide choline once daily for an initial period of two weeks;
(b) after the initial two-week period, orally administering to the subject a first titration dosage of diazoxide choline once daily for a second two-week period, wherein the first titration dosage is 150 mg or 200 mg of diazoxide choline;
(c) after the second two-week period, orally administering to the subject a second titration dosage of diazoxide choline once daily for a third two-week period, wherein the second titration dosage is 200 mg, 250 mg, or 300 mg of diazoxide choline; and
(d) after the third two-week period, orally administering to the subject a target maintenance dose of diazoxide choline once daily, wherein the target maintenance dose is 250 mg, 300 mg, or 325 mg of diazoxide choline;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) a starting dosage of 100 mg of diazoxide choline is orally administered to the subject once daily for an initial period of two weeks;
(b) after the initial two-week period, a first titration dosage of diazoxide choline is orally administered to the subject once daily for a second two-week period, wherein the first titration dosage is 150 mg or 200 mg of diazoxide choline;
(c) after the second two-week period, a second titration dosage of diazoxide choline is orally administered to the subject once daily for a third two-week period, wherein the second titration dosage is 200 mg, 250 mg, or 300 mg of diazoxide choline; and
(d) after the third two-week period, a target maintenance dose of diazoxide choline is orally administered to the subject once daily, wherein the target maintenance dose is 250 mg, 300 mg, or 325 mg of diazoxide choline;
wherein the subject is concomitantly being administered one or more strong CYP1A2 inhibitors.

In some embodiments, the starting dosage is 100 mg, the first titration dosage is 150 mg, the second titration dosage is 200 mg, and the target maintenance dosage is 250 mg. In some embodiments, the subject weighs 65 kg to less than 100 kg and the starting dosage is 100 mg, the first titration dosage is 150 mg, the second titration dosage is 200 mg, and the target maintenance dosage is 250 mg.

In some embodiments, the subject weighs 65 kg to less than 100 kg. In some embodiments, the subject weighs 65 kg. In some embodiments, the subject weighs 70 kg. In some embodiments, the subject weighs 75 kg. In some embodiments, the subject weighs 80 kg. In some embodiments, the subject weighs 85 kg. In some embodiments, the subject weighs 90 kg. In some embodiments, the subject weighs 95 kg.

In some embodiments, the starting dosage is 100 mg, the first titration dosage is 200 mg, the second titration dosage is 250 mg, and the target maintenance dosage is 300 mg. In some embodiments, the subject weighs 100 kg to less than 135 kg and the starting dosage is 100 mg, the first titration dosage is 200 mg, the second titration dosage is 250 mg, and the target maintenance dosage is 300 mg.

In some embodiments, the subject weighs 100 kg to less than 135 kg. In some embodiments, the subject weighs 100 kg. In some embodiments, the subject weighs 105 kg. In some embodiments, the subject weighs 110 kg. In some embodiments, the subject weighs 115 kg. In some embodiments, the subject weighs 120 kg. In some embodiments, the subject weighs 125 kg. In some embodiments, the subject weighs 130 kg.

In some embodiments, the starting dosage is 100 mg, the first titration dosage is 200 mg, the second titration dosage is 300 mg, and the target maintenance dosage is 325 mg. In some embodiments, the subject weighs 135 kg or more and the starting dosage is 100 mg, the first titration dosage is 200 mg, the second titration dosage is 300 mg, and the target maintenance dosage is 325 mg.

In some embodiments, the subject weighs 135 kg or more. In some embodiments, the subject weighs 135 kg. In some embodiments, the subject weighs 140 kg. In some embodiments, the subject weighs 145 kg.

In some embodiments, the subject weighs 150 kg.

Also disclosed herein are methods of treating a subject in need thereof, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject an initial target dose of 200 mg of diazoxide choline once daily for a period of at least six months; and
   (i) after the period of at least six months, orally administering to the subject the initial target dose of 200 mg of diazoxide choline once daily; or
   (ii) after the period of at least six months, orally administering to the subject a maximum target dose of 250 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks;
(c) after the second period of at least four weeks, 150 mg of diazoxide choline is orally administered to the subject once daily for a third period of at least four weeks;
(d) after the third period of at least four weeks, an initial target dose of 200 mg of diazoxide choline is orally administered to the subject once daily for a period of at least six months; and
   (i) after the period of at least six months, the initial target dose of 200 mg of diazoxide choline is orally administered to the subject once daily; or
   (ii) after the period of at least six months, a maximum target dose of 250 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject an initial target dose of 200 mg of diazoxide choline once daily for a period of at least six months; and
   (i) after the period of at least six months, orally administering to the subject the initial target dose of 200 mg of diazoxide choline once daily; or
   (ii) after the period of at least six months, orally administering to the subject a maximum target dose of 250 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks;
(c) after the second period of at least four weeks, 150 mg of diazoxide choline is orally administered to the subject once daily for a third period of at least four weeks;
(d) after the third period of at least four weeks, an initial target dose of 200 mg of diazoxide choline is orally administered to the subject once daily for a period of at least six months; and
   (i) after the period of at least six months, the initial target dose of 200 mg of diazoxide choline is orally administered to the subject once daily; or
   (ii) after the period of at least six months, a maximum target dose of 250 mg of diazoxide choline is orally administered to the subject once daily;
wherein the subject is concomitantly being administered one or more strong CYP1A2 inhibitors.

In some embodiments, the subject weighs 65 kg to less than 100 kg. In some embodiments, the subject weighs 65 kg. In some embodiments, the subject weighs 70 kg. In some embodiments, the subject weighs 75 kg. In some embodiments, the subject weighs 80 kg. In some embodiments, the subject weighs 85 kg. In some embodiments, the subject weighs 90 kg. In some embodiments, the subject weighs 95 kg.

Also disclosed herein are methods of treating a subject in need thereof, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject 200 mg of diazoxide choline once daily for a fourth period of at least four weeks;
(e) after the fourth period of at least four weeks, orally administering to the subject an initial target dose of 225 mg of diazoxide choline once daily for a period of at least six months; and
   (i) after the period of at least six months, orally administering to the subject the initial target dose of 225 mg of diazoxide choline once daily; or
   (ii) after the period of at least six months, orally administering to the subject a maximum target dose of 300 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks;
(c) after the second period of at least four weeks, 150 mg of diazoxide choline is orally administered to the subject once daily for a third period of at least four weeks;
(d) after the third period of at least four weeks, 200 mg of diazoxide choline is orally administered to the subject once daily for a fourth period of at least four weeks;
(e) after the fourth period of at least four weeks, an initial target dose of 225 mg of diazoxide choline is orally administered to the subject once daily for a period of at least six months; and
   (i) after the period of at least six months, the initial target dose of 225 mg of diazoxide choline is orally administered to the subject once daily; or
   (ii) after the period of at least six months, a maximum target dose of 300 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject 200 mg of diazoxide choline once daily for a fourth period of at least four weeks;
(e) after the fourth period of at least four weeks, orally administering to the subject an initial target dose of 225 mg of diazoxide choline once daily for a period of at least six months; and
   (i) after the period of at least six months, orally administering to the subject the initial target dose of 225 mg of diazoxide choline once daily; or
   (ii) after the period of at least six months, orally administering to the subject a maximum target dose of 300 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks;
(c) after the second period of at least four weeks, 150 mg of diazoxide choline is orally administered to the subject once daily for a third period of at least four weeks;
(d) after the third period of at least four weeks, 200 mg of diazoxide choline is orally administered to the subject once daily for a fourth period of at least four weeks;
(e) after the fourth period of at least four weeks, an initial target dose of 225 mg of diazoxide choline is orally administered to the subject once daily for a period of at least six months; and
   (i) after the period of at least six months, the initial target dose of 225 mg of diazoxide choline is orally administered to the subject once daily; or
   (ii) after the period of at least six months, a maximum target dose of 300 mg of diazoxide choline is orally administered to the subject once daily;
wherein the subject is concomitantly being administered one or more strong CYP1A2 inhibitors.

In some embodiments, the subject weighs 100 kg to less than 135 kg. In some embodiments, the subject weighs 100 kg. In some embodiments, the subject weighs 105 kg. In some embodiments, the subject weighs 110 kg. In some embodiments, the subject weighs 115 kg. In some embodiments, the subject weighs 120 kg. In some embodiments, the subject weighs 125 kg. In some embodiments, the subject weighs 130 kg.

Also disclosed herein are methods of treating a subject in need thereof, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject 200 mg of diazoxide choline once daily for a fourth period of at least four weeks;
(e) after the fourth period of at least four weeks, orally administering to the subject 225 mg of diazoxide choline once daily for a fifth period of at least four weeks;
(f) after the fifth period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 325 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject in need thereof, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks;
(c) after the second period of at least four weeks, 150 mg of diazoxide choline is orally administered to the subject once daily for a third period of at least four weeks;
(d) after the third period of at least four weeks, 200 mg of diazoxide choline is orally administered to the subject once daily for a fourth period of at least four weeks;
(e) after the fourth period of at least four weeks, 225 mg of diazoxide choline is orally administered to the subject once daily for a fifth period of at least four weeks;
(f) after the fifth period of at least four weeks, an initial target dose of 275 mg of diazoxide choline is orally administered to the subject once daily for a period of at least six months; and
   (i) after the period of at least six months, the initial target dose of 275 mg of diazoxide choline is orally administered to the subject once daily; or
   (ii) after the period of at least six months, a maximum target dose of 325 mg of diazoxide choline is orally administered to the subject once daily;
wherein the subject is concomitantly being administered one or more strong CYP1A2 inhibitors.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject 200 mg of diazoxide choline once daily for a fourth period of at least four weeks;
(e) after the fourth period of at least four weeks, orally administering to the subject 225 mg of diazoxide choline once daily for a fifth period of at least four weeks;
(f) after the fifth period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 325 mg of diazoxide choline once daily;
wherein the subject is concomitantly being administered one or more strong CYP1A2 inhibitors.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(a) 50 mg of diazoxide choline is orally administered to the subject once daily for an initial period of at least four weeks;
(b) after the initial period of at least four weeks, 100 mg of diazoxide choline is orally administered to the subject once daily for a second period of at least four weeks;
(c) after the second period of at least four weeks, 150 mg of diazoxide choline is orally administered to the subject once daily for a third period of at least four weeks;
(d) after the third period of at least four weeks, 200 mg of diazoxide choline is orally administered to the subject once daily for a fourth period of at least four weeks;
(e) after the fourth period of at least four weeks, 225 mg of diazoxide choline is orally administered to the subject once daily for a fifth period of at least four weeks;
(f) after the fifth period of at least four weeks, an initial target dose of 275 mg of diazoxide choline is orally administered to the subject once daily for a period of at least six months; and
   (i) after the period of at least six months, the initial target dose of 275 mg of diazoxide choline is orally administered to the subject once daily; or
   (ii) after the period of at least six months, a maximum target dose of 325 mg of diazoxide choline is orally administered to the subject once daily;
wherein the subject is concomitantly being administered one or more strong CYP1A2 inhibitors.

In some embodiments, the subject weighs 135 kg or more. In some embodiments, the subject weighs 135 kg. In some embodiments, the subject weighs 140 kg. In some embodiments, the subject weighs 145 kg.

In some embodiments, the subject weighs 150 kg.

In some embodiments, the initial period of at least four weeks is 4-8 weeks. In some embodiments, the initial period of at least four weeks is 4-7 weeks. In some embodiments, the initial period of at least four weeks is 4-6 weeks. In some embodiments, the initial period of at least four weeks is four, five, six, seven, or eight weeks. In some embodiments, the initial period of at least four weeks is four, five, six, or seven weeks.

In some embodiments, the initial period of at least four weeks is four, five, or six weeks. In some embodiments, the initial period of at least four weeks is four weeks. In some embodiments, the initial period of at least four weeks is five weeks. In some embodiments, the initial period of at least four weeks is six weeks. In some embodiments, the initial period of at least four weeks is seven weeks. In some embodiments, the initial period of at least four weeks is eight weeks.

In some embodiments, the sequential period of at least four weeks is 4-8 weeks. In some embodiments, the sequential period of at least four weeks is 4-7 weeks. In some embodiments, the sequential period of at least four weeks is 4-6 weeks. In some embodiments, the sequential period of at least four weeks is four, five, six, seven, or eight weeks. In some embodiments, the sequential period of at least four weeks is four, five, six, or seven weeks. In some embodiments, the sequential period of at least four weeks is four, five, or six weeks. In some embodiments, the sequential period of at least four weeks is four weeks. In some embodiments, the sequential period of at least four weeks is five weeks. In some embodiments, the sequential period of at least four weeks is six weeks. In some embodiments, the sequential period of at least four weeks is seven weeks. In some embodiments, the sequential period of at least four weeks is eight weeks.

In some embodiments, the second period of at least four weeks is 4-8 weeks. In some embodiments, the second period of at least four weeks is 4-7 weeks. In some embodiments, the second period of at least four weeks is 4-6 weeks. In some embodiments, the second period of at least four weeks is four, five, six, seven, or eight weeks. In some embodiments, the second period of at least four weeks is four, five, six, or seven weeks.

In some embodiments, the second period of at least four weeks is four, five, or six weeks. In some embodiments, the second period of at least four weeks is four weeks. In some embodiments, the second period of at least four weeks is five weeks. In some embodiments, the second period of at least four weeks is six weeks. In some embodiments, the second period of at least four weeks is seven weeks. In some embodiments, the second period of at least four weeks is eight weeks.

In some embodiments, the third period of at least four weeks is 4-8 weeks. In some embodiments, the third period of at least four weeks is 4-7 weeks. In some embodiments, the third period of at least four weeks is 4-6 weeks. In some embodiments, the third period of at least four weeks is four, five, six, seven, or eight weeks. In some embodiments, the third period of at least four weeks is four, five, six, or seven weeks. In some embodiments, the third period of at least four weeks is four, five, or six weeks. In some embodiments, the third period of at least four weeks is four weeks. In some embodiments, the third period of at least four weeks is five weeks. In some embodiments, the third period of at least four weeks is six weeks. In some embodiments, the third period of at least four weeks is seven weeks. In some embodiments, the third period of at least four weeks is eight weeks.

In some embodiments, the fourth period of at least four weeks is 4-8 weeks. In some embodiments, the fourth period of at least four weeks is 4-7 weeks. In some embodiments, the fourth period of at least four weeks is 4-6 weeks. In some embodiments, the fourth period of at least four weeks is four, five, six, seven, or eight weeks. In some embodiments, the fourth period of at least four weeks is four, five, six, or seven weeks.

In some embodiments, the fourth period of at least four weeks is four, five, or six weeks. In some embodiments, the fourth period of at least four weeks is four weeks. In some embodiments, the fourth period of at least four weeks is five weeks. In some embodiments, the fourth period of at least four weeks is six weeks. In some embodiments, the fourth period of at least four weeks is seven weeks. In some embodiments, the fourth period of at least four weeks is eight weeks.

In some embodiments, the fifth period of at least four weeks is 4-8 weeks. In some embodiments, the fifth period of at least four weeks is 4-7 weeks. In some embodiments, the fifth period of at least four weeks is 4-6 weeks. In some embodiments, the fifth period of at least four weeks is four, five, six, seven, or eight weeks.

In some embodiments, the fifth period of at least four weeks is four, five, six, or seven weeks. In some embodiments, the fifth period of at least four weeks is four, five, or six weeks. In some embodiments, the fifth period of at least four weeks is four weeks. In some embodiments, the fifth period of at least four weeks is five weeks. In some embodiments, the fifth period of at least four weeks is six weeks. In some embodiments, the fifth period of at least four weeks is seven weeks. In some embodiments, the fifth period of at least four weeks is eight weeks.

In some embodiments, the initial period of at least eight weeks is 8-12 weeks. In some embodiments, the initial period of at least eight weeks is 8-11 weeks. In some embodiments, the initial period of at least eight weeks is 8-10 weeks. In some embodiments, the initial period of at least eight weeks is eight, nine, ten, eleven, or twelve weeks. In some embodiments, the initial period of at least eight weeks is eight, nine, ten, or eleven weeks. In some embodiments, the initial period of at least eight weeks is eight, nine, or ten weeks. In some embodiments, the initial period of at least eight weeks is eight weeks. In some embodiments, the initial period of at least eight weeks is nine weeks. In some embodiments, the initial period of at least eight weeks is ten weeks. In some embodiments, the initial period of at least eight weeks is eleven weeks. In some embodiments, the initial period of at least eight weeks is twelve weeks.

In some embodiments, the period of at least six months is 6-12 months. In some embodiments, the period of at least six months is 6-11 months. In some embodiments, the period of at least six months is 6-10 months. In some embodiments, the period of at least six months is 6-9 months. In some embodiments, the period of at least six months is 6-8 months. In some embodiments, the period of at least six months is six, seven, eight, nine, ten, eleven, or twelve months. In some embodiments, the period of at least six months is six, seven, eight, nine, ten, or eleven months. In some embodiments, the period of at least six months is six, seven, eight, nine, or ten months. In some embodiments, the period of at least six months is six, seven, eight, or nine months. In some embodiments, the period of at least six months is six, seven, or eight months. In some embodiments, the period of at least six months is six months. In some embodiments, the period of at least six months is seven months. In some embodiments, the period of at least six months is eight months. In some embodiments, the period of at least six months is nine months. In some embodiments, the period of at least six months is ten months. In some embodiments, the period of at least six months is eleven months. In some embodiments, the period of at least six months is twelve months.

In some embodiments, the one or more strong CYP1A2 inhibitors is amiodarone, ciprofloxacin, or fluvoxamine. In some embodiments, the one or more strong CYP1A2 inhibitors is amiodarone. In some embodiments, the one or more strong CYP1A2 inhibitors is ciprofloxacin. In some embodiments, the one or more strong CYP1A2 inhibitors is fluvoxamine.

In some embodiments, the subject in need of concomitant administration of one or more strong CYP1A2 inhibitors is also a subject having one or more clinically relevant comorbidities, as described herein.

In some embodiments, the subject has one or more clinically relevant comorbidities. In some embodiments, the one or more clinically relevant comorbidities is obesity. In some embodiments, the one or more clinically relevant comorbidities is severe obesity. In some embodiments, the subject is severely obese and has one or more additional clinically relevant comorbidities. In some embodiments, the subject is severely obese and has one or more cardiovascular comorbidities, as described herein (e.g., a history of edema, edema, fluid retention, fluid overload, congestive heart failure, compromised cardiac reserve, heart disease, etc.). In some embodiments, the subject is severely obese and has a history of one or more cardiovascular comorbidities. In some embodiments, the subject is severely obese and has one or more pulmonary comorbidities, as described herein (e.g., a history of asthma, asthma, chronic lung disease, Chronic Obstructive Pulmonary Disease (COPD), obstructive sleep apnea, central sleep apnea, recent pulmonary infection, etc.). In some embodiments, the subject is severely obese and has a history of one or more pulmonary comorbidities. Thus, in some embodiments, a subject that is severely obese with one or more additional clinically relevant comorbidities may require a modified dosing schedule, such as the one recited in Table 5b.

Also disclosed herein are methods of treating a subject in need thereof, wherein said subject is in need of concomitant administration of one or more strong CYP1A2 inhibitors and is administered a once daily oral maintenance dose of diazoxide choline, said methods comprising:
(a) orally administering a once daily reduced maintenance dose of diazoxide choline, and
(b) administering one or more CYP1A2 inhibitors;
   wherein,
      (i) if the maintenance dose is 100 mg, then the reduced maintenance dose is 75 mg,
      (ii) if the maintenance dose is 150 mg, then the reduced maintenance dose is 100 mg,
      (iii) if the maintenance dose is 225 mg, then the reduced maintenance dose is 150 mg,
      (iv) if the maintenance dose is 375 mg, then the reduced maintenance dose is 250 mg,
      (v) if the maintenance dose is 450 mg, then the reduced maintenance dose is 300 mg, or
      (vi) if the maintenance dose is 525 mg, then the reduced maintenance dose is 325 mg.

Also disclosed herein is diazoxide choline, or pharmaceutical compositions comprising diazoxide choline, for use in a method of treating a subject in need thereof, wherein said subject is in need of concomitant administration of one or more strong CYP1A2 inhibitors and is administered a once daily oral maintenance dose of diazoxide choline, wherein:
(a) a once daily reduced maintenance dose of diazoxide choline is orally administered, and
(b) one or more CYP1A2 inhibitors are administered;
   wherein,
      (i) if the maintenance dose is 100 mg, then the reduced maintenance dose is 75 mg,
      (ii) if the maintenance dose is 150 mg, then the reduced maintenance dose is 100 mg,
      (iii) if the maintenance dose is 225 mg, then the reduced maintenance dose is 150 mg,
      (iv) if the maintenance dose is 375 mg, then the reduced maintenance dose is 250 mg,
      (v) if the maintenance dose is 450 mg, then the reduced maintenance dose is 300 mg, or
      (vi) if the maintenance dose is 525 mg, then the reduced maintenance dose is 325 mg.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, wherein said subject is in need of concomitant administration of one or more strong CYP1A2 inhibitors and is administered a once daily oral maintenance dose of diazoxide choline, said methods comprising:
(a) orally administering a once daily reduced maintenance dose of diazoxide choline, and
(b) administering one or more CYP1A2 inhibitors;
   wherein,
      (i) if the maintenance dose is 100 mg, then the reduced maintenance dose is 75 mg,
      (ii) if the maintenance dose is 150 mg, then the reduced maintenance dose is 100 mg,
      (iii) if the maintenance dose is 225 mg, then the reduced maintenance dose is 150 mg,
      (iv) if the maintenance dose is 375 mg, then the reduced maintenance dose is 250 mg,
      (v) if the maintenance dose is 450 mg, then the reduced maintenance dose is 300 mg, or
      (vi) if the maintenance dose is 525 mg, then the reduced maintenance dose is 325 mg.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein said subject is in need of concomitant administration of one or more strong CYP1A2 inhibitors and is administered a once daily oral maintenance dose of diazoxide choline, wherein:
(a) a once daily reduced maintenance dose of diazoxide choline is orally administered, and
(b) one or more CYP1A2 inhibitors are administered;
   wherein,
      (i) if the maintenance dose is 100 mg, then the reduced maintenance dose is 75 mg,
      (ii) if the maintenance dose is 150 mg, then the reduced maintenance dose is 100 mg,
      (iii) if the maintenance dose is 225 mg, then the reduced maintenance dose is 150 mg,
      (iv) if the maintenance dose is 375 mg, then the reduced maintenance dose is 250 mg,
      (v) if the maintenance dose is 450 mg, then the reduced maintenance dose is 300 mg, or
      (vi) if the maintenance dose is 525 mg, then the reduced maintenance dose is 325 mg.

In some embodiments, the reduced maintenance dose is 75 mg. In some embodiments, the reduced maintenance dose is 100 mg. In some embodiments, the reduced maintenance dose is 150 mg. In some embodiments, the reduced maintenance dose is 250 mg. In some embodiments, the reduced maintenance dose is 300 mg. In some embodiments, the reduced maintenance dose is 325 mg.

In some embodiments, the subject weighs 20 kg to less than 30 kg. In some embodiments, the subject weighs 30 kg to less than 40 kg. In some embodiments, the subject weighs 40 kg to less than 65 kg. In some embodiments, the subject weighs 65 kg to less than 100 kg. In some embodiments, the subject weighs 100 kg to less than 135 kg. In some embodiments, the subject weighs 135 kg or more.

In some embodiments, the subject weighs 20 kg to less than 30 kg and the reduced maintenance dose is 75 mg. In some embodiments, the subject weighs 30 kg to less than 40 kg and the reduced maintenance dose is 100 mg. In some embodiments, the subject weighs 40 kg to less than 65 kg and the reduced maintenance dose is 150 mg. In some embodiments, the subject weighs 65 kg to less than 100 kg and the reduced maintenance dose is 250 mg. In some embodiments, the subject weighs 100 kg to less than 135 kg and the reduced maintenance dose is 300 mg. In some embodiments, the subject weighs 135 kg or more and the reduced maintenance dose is 325 mg.

In some embodiments, the one or more strong CYP1A2 inhibitors is amiodarone, ciprofloxacin, or fluvoxamine. In some embodiments, the one or more strong CYP1A2 inhibitors is amiodarone. In some embodiments, the one or more strong CYP1A2 inhibitors is ciprofloxacin. In some embodiments, the one or more strong CYP1A2 inhibitors is fluvoxamine.

Also disclosed herein are methods of treating a subject in need thereof, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 25 mg of diazoxide choline once daily for an initial period of four weeks;
   (ii) after the initial four-week period, orally administering to the subject 50 mg of diazoxide choline once daily for a subsequent period of two weeks; and
   (iii) after the subsequent two-week period, orally administering to the subject 75 mg of diazoxide choline once daily.

Also disclosed herein is diazoxide choline, or pharmaceutical compositions comprising diazoxide choline, for use in a method of treating a subject in need thereof, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) 25 mg of diazoxide choline is orally administered to the subject once daily for an initial period of four weeks;
   (ii) after the initial four-week period, 50 mg of diazoxide choline is orally administered to the subject once daily for a subsequent period of two weeks; and
   (iii) after the subsequent two-week period, 75 mg of diazoxide choline is orally administered to the subject once daily.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 25 mg of diazoxide choline once daily for an initial period of four weeks;
   (ii) after the initial four-week period, orally administering to the subject 50 mg of diazoxide choline once daily for a subsequent period of two weeks; and
   (iii) after the subsequent two-week period, orally administering to the subject 75 mg of diazoxide choline once daily.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) 25 mg of diazoxide choline is orally administered to the subject once daily for an initial period of four weeks;
   (ii) after the initial four-week period, 50 mg of diazoxide choline is orally administered to the subject once daily for a subsequent period of two weeks; and
   (iii) after the subsequent two-week period, 75 mg of diazoxide choline is orally administered to the subject once daily.

In some embodiments, the subject weighs 20 kg to less than 30 kg. In some embodiments, the subject weighs 20 kg. In some embodiments, the subject weighs 25 kg.

In some embodiments, the subject weighs 20 kg to less than 30 kg and is orally administered 25 mg of diazoxide choline once daily for an initial period of four weeks, orally administered 50 mg of diazoxide choline once daily for a subsequent period of two weeks, and orally administered 75 mg of diazoxide choline once daily after the subsequent two-week period.

Also disclosed herein are methods of treating a subject in need thereof, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 50 mg of diazoxide choline once daily for an initial period of two weeks; and
   (ii) after the initial two-week period, orally administering to the subject 100 mg of diazoxide choline once daily.

Also disclosed herein is diazoxide choline, or pharmaceutical compositions comprising diazoxide choline, for use in a method of treating a subject in need thereof, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) 50 mg of diazoxide choline is orally administered to the subject once daily for an initial period of two weeks; and
   (ii) after the initial two-week period, 100 mg of diazoxide choline is orally administered to the subject once daily.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 50 mg of diazoxide choline once daily for an initial period of two weeks; and
   (ii) after the initial two-week period, orally administering to the subject 100 mg of diazoxide choline once daily.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) 50 mg of diazoxide choline is orally administered to the subject once daily for an initial period of two weeks; and
   (ii) after the initial two-week period, 100 mg of diazoxide choline is orally administered to the subject once daily.

In some embodiments, the subject weighs 30 kg to less than 40 kg. In some embodiments, the subject weighs 30 kg. In some embodiments, the subject weighs 35 kg.

In some embodiments, the subject weighs 30 kg to less than 40 kg and is orally administered 50 mg of diazoxide choline once daily for an initial period of two weeks, and orally administered 100 mg of diazoxide choline once daily after the initial two-week period.

Also disclosed herein are methods of treating a subject in need thereof, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 50 mg of diazoxide choline once daily for an initial period of two weeks;
   (ii) after the initial two-week period, orally administering to the subject 100 mg of diazoxide choline once daily for a subsequent period of two weeks; and
   (iii) after the subsequent two-weeks period, orally administering to the subject 150 mg of diazoxide choline once daily.

Also disclosed herein is diazoxide choline, or pharmaceutical compositions comprising diazoxide choline, for use in a method of treating a subject in need thereof, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) 50 mg of diazoxide choline is orally administered to the subject once daily for an initial period of two weeks;
   (ii) after the initial two-week period, 100 mg of diazoxide choline is orally administered to the subject once daily for a subsequent period of two weeks; and
   (iii) after the subsequent two-weeks period, 150 mg of diazoxide choline is orally administered to the subject once daily.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 50 mg of diazoxide choline once daily for an initial period of two weeks;
   (ii) after the initial two-week period, orally administering to the subject 100 mg of diazoxide choline once daily for a subsequent period of two weeks; and
   (iii) after the subsequent two-weeks period, orally administering to the subject 150 mg of diazoxide choline once daily.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) 50 mg of diazoxide choline is orally administered to the subject once daily for an initial period of two weeks;
   (ii) after the initial two-week period, 100 mg of diazoxide choline is orally administered to the subject once daily for a subsequent period of two weeks; and
   (iii) after the subsequent two-weeks period, 150 mg of diazoxide choline is orally administered to the subject once daily.

In some embodiments, the subject weighs 40 kg to less than 65 kg. In some embodiments, the subject weighs 40 kg. In some embodiments, the subject weighs 45 kg. In some embodiments, the subject weighs 50 kg. In some embodiments, the subject weighs 55 kg. In some embodiments, the subject weighs 60 kg.

In some embodiments, the subject weighs 40 kg to less than 65 kg and is orally administered 50 mg of diazoxide choline once daily for an initial period of two weeks, orally administered 100 mg of diazoxide choline once daily for a subsequent period of two weeks, and orally administered 150 mg of diazoxide choline once daily after the subsequent two-week period.

Also disclosed herein are methods of treating a subject in need thereof, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject a starting dosage of 100 mg of diazoxide choline once daily for an initial period of two weeks;
   (ii) after the initial two-week period, orally administering to the subject a first titration dosage of diazoxide choline once daily for a second two-week period, wherein the first titration dosage is 150 mg or 200 mg of diazoxide choline;
   (iii) after the second two-week period, orally administering to the subject a second titration dosage of diazoxide choline once daily for a third two-week period, wherein the second titration dosage is 200 mg, 250 mg, or 300 mg of diazoxide choline; and
   (iv) after the third two-week period, orally administering to the subject a target maintenance dose of diazoxide choline once daily, wherein the target maintenance dose is 250 mg, 300 mg, or 325 mg of diazoxide choline.

Also disclosed herein is diazoxide choline, or pharmaceutical compositions comprising diazoxide choline, for use in a method of treating a subject in need thereof, wherein said subject is in need of concomitant administration of one or more strong CYP1A2 inhibitors and is administered a once daily oral maintenance dose of diazoxide choline, wherein:

(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) a starting dosage of 100 mg of diazoxide choline is orally administered to the subject once daily for an initial period of two weeks;
   (ii) after the initial two-week period, a first titration dosage of diazoxide choline is orally administered to the subject once daily for a second two-week period, wherein the first titration dosage is 150 mg or 200 mg of diazoxide choline;
   (iii) after the second two-week period, a second titration dosage of diazoxide choline is orally administered to the subject once daily for a third two-week period, wherein the second titration dosage is 200 mg, 250 mg, or 300 mg of diazoxide choline; and
   (iv) after the third two-week period, a target maintenance dose of diazoxide choline is orally administered to the subject once daily, wherein the target maintenance dose is 250 mg, 300 mg, or 325 mg of diazoxide choline.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, wherein said subject is in need of concomitant administration of one or more strong CYP1A2 inhibitors and is administered a once daily oral maintenance dose of diazoxide choline, said methods comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject a starting dosage of 100 mg of diazoxide choline once daily for an initial period of two weeks;
   (ii) after the initial two-week period, orally administering to the subject a first titration dosage of diazoxide choline once daily for a second two-week period, wherein the first titration dosage is 150 mg or 200 mg of diazoxide choline;
   (iii) after the second two-week period, orally administering to the subject a second titration dosage of diazoxide choline once daily for a third two-week period, wherein the second titration dosage is 200 mg, 250 mg, or 300 mg of diazoxide choline; and
   (iv) after the third two-week period, orally administering to the subject a target maintenance dose of diazoxide choline once daily, wherein the target maintenance dose is 250 mg, 300 mg, or 325 mg of diazoxide choline.

Also disclosed herein is diazoxide choline, or pharmaceutical compositions comprising diazoxide choline, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein said subject is in need of concomitant administration of one or more strong CYP1A2 inhibitors and is administered a once daily oral maintenance dose of diazoxide choline, wherein:
(a) the once daily oral maintenance dose of diazoxide choline is discontinued, and
(b) after the once daily oral maintenance dose of diazoxide choline is discontinued, one or more strong CYP1A2 inhibitors is administered to the subject and diazoxide choline is concomitantly administered to the subject on the following schedule:
   (i) a starting dosage of 100 mg of diazoxide choline is orally administered to the subject once daily for an initial period of two weeks;
   (ii) after the initial two-week period, a first titration dosage of diazoxide choline is orally administered to the subject once daily for a second two-week period, wherein the first titration dosage is 150 mg or 200 mg of diazoxide choline;
   (iii) after the second two-week period, a second titration dosage of diazoxide choline is orally administered to the subject once daily for a third two-week period, wherein the second titration dosage is 200 mg, 250 mg, or 300 mg of diazoxide choline; and
   (iv) after the third two-week period, a target maintenance dose of diazoxide choline is orally administered to the subject once daily, wherein the target maintenance dose is 250 mg, 300 mg, or 325 mg of diazoxide choline.

In some embodiments, the starting dosage is 100 mg, the first titration dosage is 150 mg, the second titration dosage is 200 mg, and the target maintenance dosage is 250 mg. In some embodiments, the subject weighs 65 kg to less than 100 kg and the starting dosage is 100 mg, the first titration dosage is 150 mg, the second titration dosage is 200 mg, and the target maintenance dosage is 250 mg.

In some embodiments, the subject weighs 65 kg to less than 100 kg. In some embodiments, the subject weighs 65 kg. In some embodiments, the subject weighs 70 kg. In some embodiments, the subject weighs 75 kg. In some embodiments, the subject weighs 80 kg. In some embodiments, the subject weighs 85 kg. In some embodiments, the subject weighs 90 kg. In some embodiments, the subject weighs 95 kg.

In some embodiments, the starting dosage is 100 mg, the first titration dosage is 200 mg, the second titration dosage is 250 mg, and the target maintenance dosage is 300 mg. In some embodiments, the subject weighs 100 kg to less than 135 kg and the starting dosage is 100 mg, the first titration dosage is 200 mg, the second titration dosage is 250 mg, and the target maintenance dosage is 300 mg.

In some embodiments, the subject weighs 100 kg to less than 135 kg. In some embodiments, the subject weighs 100 kg. In some embodiments, the subject weighs 105 kg. In some embodiments, the subject weighs 110 kg. In some embodiments, the subject weighs 115 kg. In some embodiments, the subject weighs 120 kg. In some embodiments, the subject weighs 125 kg. In some embodiments, the subject weighs 130 kg.

In some embodiments, the starting dosage is 100 mg, the first titration dosage is 200 mg, the second titration dosage is 300 mg, and the target maintenance dosage is 325 mg. In some embodiments, the subject weighs 135 kg or more and the starting dosage is 100 mg, the first titration dosage is 200 mg, the second titration dosage is 300 mg, and the target maintenance dosage is 325 mg.

In some embodiments, the subject weighs 135 kg or more. In some embodiments, the subject weighs 135 kg. In some embodiments, the subject weighs 140 kg. In some embodiments, the subject weighs 145 kg.

In some embodiments, the subject weighs 150 kg.

In some embodiments, the one or more strong CYP1A2 inhibitors is amiodarone, ciprofloxacin, or fluvoxamine. In some embodiments, the one or more strong CYP1A2 inhibitors is amiodarone. In some embodiments, the one or more strong CYP1A2 inhibitors is ciprofloxacin. In some embodiments, the one or more strong CYP1A2 inhibitors is fluvoxamine.

In some embodiments, the subject is in need of concomitant administration of one or more strong OAT1 inhibitors. In some embodiments, the subject is in need of concomitant administration of one or more strong OAT3 inhibitors. For subjects in need of concomitant administration of one or more strong OAT1 inhibitors or one or more strong OAT3 inhibitors, the subject can be provided modified dosing of diazoxide choline as described herein for administration of diazoxide choline with one or more strong CYP1A2 inhibitors.

As will be appreciated by the skilled artisan, administration of diazoxide, or a pharmaceutically acceptable salt thereof, to the subject in need thereof may be varied according to frequency, mechanisms of delivery, or dosage forms based on the signs or comorbidities being exhibited by the subject, the age of the subject, etc. and as guided by a skilled physician. For example, the diazoxide, or a pharmaceutically acceptable salt thereof, may be administered to the subject in need thereof using the oral dosage form of a tablet, capsule or liquid. The diazoxide or the pharmaceutically acceptable salt thereof may be administered to the subject in need thereof once daily (e.g., once every about 24 hours). In addition, the methods, uses, and compounds for use described herein may be adapted for the administration of diazoxide, or a pharmaceutically acceptable salt therefore, to be twice daily (e.g., two times per every about 24 hours), thrice daily (e.g., three times per every about 24 hours) or the like as described herein. In some embodiments, the diazoxide or the pharmaceutically acceptable salt thereof may be administered to the subjects in need thereof using various mechanisms of delivery including, but not limited to, oral delivery, sublingual delivery, intravenous delivery, or the like. In certain embodiments, the administration of diazoxide or the pharmaceutically acceptable salt thereof to the subjects in need thereof, if via oral delivery, may include types of oral delivery, such as via a capsule or tablet.

As will be appreciated by the skilled artisan, administration of diazoxide, or a pharmaceutically acceptable salt thereof, to the subject with PWS may be varied according to frequency, mechanisms of delivery, or dosage forms based on the signs or comorbidities being exhibited by the subject, the age of the subject, etc. and as guided by a skilled physician. For example, the diazoxide, or a pharmaceutically acceptable salt thereof, may be administered to the subject with PWS using the oral dosage form of a tablet, capsule or liquid. The diazoxide or the pharmaceutically acceptable salt thereof may be administered to the subject with PWS once daily (e.g., once every about 24 hours). In addition, the methods, uses, and compounds for use described herein may be adapted for the administration of diazoxide, or a pharmaceutically acceptable salt therefore, to be twice daily (e.g., two times per every about 24 hours), thrice daily (e.g., three times per every about 24 hours) or the like as described herein. In some embodiments, the diazoxide or the pharmaceutically acceptable salt thereof may be administered to the subjects with PWS using various mechanisms of delivery including, but not limited to, oral delivery, sublingual delivery, intravenous delivery, or the like. In certain embodiments, the administration of diazoxide or the pharmaceutically acceptable salt thereof to the subjects with PWS, if via oral delivery, may include types of oral delivery, such as via a capsule or tablet.

In some embodiments, the diazoxide, or a pharmaceutically acceptable salt thereof, is formulated as a pharmaceutical formulation comprising at least one excipient that extends the release of diazoxide, or a pharmaceutically acceptable salt thereof.

In some embodiments, the dosage forms may include extended-release variations. For example, diazoxide extended-release formulations may therefore result in better tolerability and efficacy at lower doses or mucoadhesive properties to slow the transit of solid oral dosage form (e.g., tablet or capsule) through the gastrointestinal tract. In some embodiments, an extended-release formulation may include pharmaceutical product that may provide both the delay of release of the pharmaceutical active upon administration (e.g., pH-sensitive excipients where release is begin after entry into the intestines) and controlled release for sustained release over a period of time (e.g., utilizing excipients that slow release compared to immediate release formulations but where release begins shortly after ingesting the formulation). Extended-release diazoxide or diazoxide choline formulations may be found in U.S. Pat. Nos. 9,782,416, 7,799,777, 7,572,789, 9,381,202, 10,085,998, 11,045,478, 9,757,384, 10,058,557, 10,456,408, and International App. No. PCT/IB2024/058904, each of which are incorporated by reference in their entirety herein.

In some embodiments of the methods described herein, comprising methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, the diazoxide extended-release formulation may not be substituted with a diazoxide oral suspension.

In some embodiments, the diazoxide, or its pharmaceutically acceptable salt, may be administered to the subject in need thereof, e.g., a PWS subject, for any length of time greater than or equal to about 1 week.

For example, the diazoxide, or its pharmaceutically acceptable salt, may be administered to the subject for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, about least 20 weeks, at least about 25 weeks, at least about 30 weeks, at least about 35 weeks, at least about 40 weeks, at least about 45 weeks, at least about 50 weeks, at least about 52 weeks, at least about 13 months, at least about 14 months, at least about 15 months or greater, including indefinitely.

Also disclosed herein are methods of treating a subject in need thereof, said methods comprising:
(y) administering to the subject a therapeutically effective amount of diazoxide, or a pharmaceutically acceptable salt thereof, wherein the subject is concomitantly administered one or more strong CYP3A4 inhibitors;
(z) monitoring the subject for adverse reactions to diazoxide, or a pharmaceutically acceptable salt thereof; and
(aa) if the subject exhibits adverse reactions, adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject in need thereof, wherein:
(bb) the subject is administered a therapeutically effective amount of diazoxide, or a pharmaceutically acceptable salt thereof, wherein the subject is concomitantly administered one or more strong CYP3A4 inhibitors;
(cc) the subject is monitored for adverse reactions to diazoxide, or a pharmaceutically acceptable salt thereof; and
(dd) if the subject exhibits adverse reactions, the therapeutically effective amount of diazoxide, or a pharmaceutically acceptable salt thereof, administered to the subject is adjusted.

In some embodiments, the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance. In some embodiments, the subject is a subject having Glycogen Storage Disease Type 1 or a subject having Glucose Transporter Type 1 Deficiency Syndrome.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(ee) administering to the subject a therapeutically effective amount of diazoxide, or a pharmaceutically acceptable salt thereof, wherein the subject is concomitantly administered one or more strong CYP3A4 inhibitors;
(ff) monitoring the subject for adverse reactions to diazoxide, or a pharmaceutically acceptable salt thereof; and
(gg) if the subject exhibits adverse reactions, adjusting the therapeutically effective amount of diazoxide, or a pharmaceutically acceptable salt thereof, administered to the subject.

Also disclosed herein is diazoxide, or a pharmaceutically acceptable salt thereof, or pharmaceutical compositions comprising diazoxide, or a pharmaceutically acceptable salt thereof, for use in a method of treating a subject having PWS, including the comorbidities of PWS, for use in a method of treating hyperphagia in a subject having PWS, for use in a method of treating a subject with PWS having hyperphagia, for use in a method of treating hypotonia in a subject having PWS, for use in a method of treating a subject with PWS having hypotonia, for use in a method of increasing the lean body mass in a subject with PWS, for use in a method of increasing the lean body mass/fat mass ratio in a subject with PWS, for use in a method of increasing the lower limb muscle mass in a subject with PWS, and for use in a method of reducing aggressive behaviors in a subject with PWS, wherein:
(hh) the subject is administered a therapeutically effective amount of diazoxide, or a pharmaceutically acceptable salt thereof, wherein the subject is concomitantly administered one or more strong CYP3A4 inhibitors;
(ii) the subject is monitored for adverse reactions to diazoxide, or a pharmaceutically acceptable salt thereof; and
(jj) if the subject exhibits adverse reactions, the therapeutically effective amount of diazoxide, or a pharmaceutically acceptable salt thereof, administered to the subject is adjusted.

In some embodiments, the therapeutically effective amount of diazoxide, or a pharmaceutically acceptable salt thereof, is reduced relative to the therapeutically effective amount for the subject if not concomitantly administered one or more strong CYP3A4 inhibitors.

In some embodiments, the therapeutically effective amount of diazoxide, or a pharmaceutically acceptable salt thereof, is reduced by between about 15.0%-30.0%, including by between about 20.0%-30.0%, and by between about 25.0%-30.0%. In some embodiments, the therapeutically effective amount of diazoxide, or a pharmaceutically acceptable salt thereof, is reduced by about 15.0%, by about 20.0%, by about 25.0%, or by about 30.0%.

In some embodiments, the one or more strong CYP3A4 inhibitors is selected from the group consisting of ceritinib, clarithromycin, delaviridone, idelalisib, indinavir, itraconazole, ketoconazole, mibefradil, nefazodone, nelfinivir, ribociclib, ritonavir, saquinavir, telaprevir, telithromycin, tucatinib, voriconazole.

In some embodiments, the adverse reactions include hyperglycemia and the risk of type 2 diabetes or ketoacidosis, peripheral edema and fluid overload, and hypertrichosis.

In some embodiments, the subject is concomitantly administered diphenylhydantoin. In some embodiments, the dosage of diphenylhydantoin is reduced.

Also disclosed herein are methods of treating a subject having PWS, including the comorbidities of PWS, methods of treating hyperphagia in a subject having PWS, methods of treating a subject with PWS having hyperphagia, methods of treating hypotonia in a subject having PWS, methods of treating a subject with PWS having hypotonia, methods of increasing the lean body mass in a subject with PWS, methods of increasing the lean body mass/fat mass ratio in a subject with PWS, methods of increasing the lower limb muscle mass in a subject with PWS, methods of reducing aggressive behaviors in a subject with PWS, and methods of reducing food-related behaviors in a subject with PWS, said methods comprising:
(kk) administering to the subject a therapeutically effective amount of diazoxide, or a pharmaceutically acceptable salt thereof, and
(ll) monitoring the patient for signs and symptoms of hyperglycemia and/or hyperuricemia.

In some embodiments, the subject is concomitantly administered a thiazide.

In some embodiments, the therapeutically effective amount of diazoxide, or a pharmaceutically acceptable salt thereof, is adjusted if the patient exhibits signs or symptoms of hyperglycemia and/or hyperuricemia.

In some embodiments, the subject is concomitantly administered a diuretic. In some embodiments, the amount of diuretic administered to the subject is reduced if the patient exhibits signs or symptoms of hyperglycemia and/or hyperuricemia.

In some embodiments, the subject, e.g., a subject with PWS, has a weight less than about 135 kg. In some embodiments, the subject has a weight between about 3 kg and about 40 kg, including between about 20 kg and about 40 kg, between about 30 kg and about 40 kg, between about 20 kg and about 30 kg, between about 3 kg and about 20 kg, between about 10 kg and about 20 kg, between about 14 kg and about 20 kg, between about 3 kg and about 14 kg, between about 10 kg and about 14 kg, between about 3 kg and about 8 kg, between about 3 kg and about 10 kg, or between about 5 kg and about 10 kg.

In some embodiments, the subject is less than or equal to 10 years of age, including less than or equal to 8 years of age, less than or equal to 4 years of age, less than or equal to 2 years of age, less than or equal to 1 year of age, from 6 months to 1 year of age, from 2 months to 6 months of age, or 2 months to 1 year of age.

In some embodiments, the subject has SMS and one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, elevated inflammatory markers, accumulation of excess body fat, etc.) comorbidities associated with SMS are improved after treatment. In some embodiments, the improvement in comorbidities associated with SMS is determined after at least 13 weeks of treatment, including after at least 26 weeks of treatment, after at least 39 weeks of treatment, or after at least 52 weeks of treatment.

In some embodiments, the subject has GSD1 and one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, elevated inflammatory markers, accumulation of excess body fat, etc.) comorbidities associated with GSD1 are improved after treatment. In some embodiments, the improvement in comorbidities associated with GSD1 is determined after at least 13 weeks of treatment, including after at least 26 weeks of treatment, after at least 39 weeks of treatment, or after at least 52 weeks of treatment.

In some embodiments, the subject has Glut1DS and one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, elevated inflammatory markers, accumulation of excess body fat, etc.) comorbidities associated with Glut1DS are improved after treatment. In some embodiments, the improvement in comorbidities associated with Glut1DS is determined after at least 13 weeks of treatment, including after at least 26 weeks of treatment, after at least 39 weeks of treatment, or after at least 52 weeks of treatment.

In some embodiments, the subject has an elevated triglyceride level. In some embodiments, the subject has an elevated triglyceride level that is reduced after treatment. In some embodiments, the reduction in the subject's triglyceride level is determined after at least 13 weeks of treatment, including after at least 26 weeks of treatment, after at least 39 weeks of treatment, or after at least 52 weeks of treatment.

In some embodiments, the subject has an elevated non-HDL cholesterol level. In some embodiments, the subject has an elevated non-HDL cholesterol level that is reduced after treatment. In some embodiments, the reduction in the subject's non-HDL cholesterol level is determined after at least 13 weeks of treatment, including after at least 26 weeks of treatment, after at least 39 weeks of treatment, or after at least 52 weeks of treatment.

In some embodiments, the subject is obese. In some embodiments, weight loss is promoted in the obese subject after treatment. In some embodiments, the weight loss in the obese subject is determined after at least 13 weeks of treatment, including after at least 26 weeks of treatment, after at least 39 weeks of treatment, or after at least 52 weeks of treatment.

In some embodiments, the subject has a reduced fasting glucose level. In some embodiments, the subject has a reduced fasting glucose level that is increased after treatment. In some embodiments, the increase in the subject's fasting glucose level is determined after at least 13 weeks of treatment, including after at least 26 weeks of treatment, after at least 39 weeks of treatment, or after at least 52 weeks of treatment.

In some embodiments, the subject has an elevated fasting insulin level. In some embodiments, the subject has an elevated fasting insulin level that is reduced after treatment. In some embodiments, the reduction in the subject's fasting insulin level is determined after at least 13 weeks of treatment, including after at least 26 weeks of treatment, after at least 39 weeks of treatment, or after at least 52 weeks of treatment.

In some embodiments, the subject has insulin resistance. In some embodiments, the subject has insulin resistance that is improved after treatment. In some embodiments, the improvement in the subject's insulin resistance is determined after at least 13 weeks of treatment, including after at least 26 weeks of treatment, after at least 39 weeks of treatment, or after at least 52 weeks of treatment.

### A Subject Having PWS

In some embodiments, the subject has PWS and one or more of the behavioral and cognitive (e.g., aggressive behaviors, hyperphagia, anxiety, compulsivity, rigidity, cognitive impairment, learning disabilities, speech impairment, etc.) or physiological (e.g., reduced lean body mass, reduced lean body mass/fat mass ratio, hypotonia, reduced lower limb muscle mass, reduced muscle strength, elevated inflammatory markers, accumulation of excess body fat, etc.) comorbidities associated with PWS are improved after treatment. In some embodiments, the improvement in comorbidities associated with PWS is determined after at least 13 weeks of treatment, including after at least 26 weeks of treatment, after at least 39 weeks of treatment, or after at least 52 weeks of treatment.

In some embodiments, one or more of the subject's behavioral and cognitive comorbidities is improved after treatment. In some embodiments, the improvement is determined after at least 13 weeks of treatment, including after at least 26 weeks of treatment, after at least 39 weeks of treatment, or after at least 52 weeks of treatment.

In some embodiments, one or more of the subject's physiological comorbidities is improved after treatment. In some embodiments, the improvement is determined after at least 13 weeks of treatment, including after at least 26 weeks of treatment, after at least 39 weeks of treatment, or after at least 52 weeks of treatment.

In some embodiments, the subject's hyperphagia is reduced after treatment. In some embodiments, the reduction is determined after at least 13 weeks of treatment, including after at least 26 weeks of treatment, after at least 39 weeks of treatment, or after at least 52 weeks of treatment. The term “hyperphagia,” as used herein, refers to the ingestion of a greater than optimal quantity of food.

In some embodiments of the methods, uses, and compounds for use described herein, the subject with PWS may have mild, moderate, or severe hyperphagia. In some embodiments of the methods, uses, and compounds for use described herein, the subject with PWS may have moderate to severe hyperphagia. For a given subject, a skilled physician's assessment of mild, moderate, or severe hyperphagia will be based on a number of factors (e.g., the various symptoms and behaviors described herein). In clinical trials, the numerical value of the subject's Hyperphagia Questionnaire for Clinical Trials (HQ-CT) total score is typically interpreted to be proportional to the overall severity of their hyperphagic and food-related behaviors. In other words, a numerically greater HQ-CT total score is indicative of a subject with a greater number and/or severity of hyperphagic and/or food-related behaviors (e.g., a subject with a HQ-CT total score of 14 would typically have less severe hyperphagic and/or food-related behaviors than a subject with a HQ-CT total score of 24).

With respect to monitoring response to treatment, a decrease in the subject's HQ-CT total score relative to the subject's HQ-CT total score prior to treatment is considered an improvement in hyperphagia. In some cases, for example, for subjects beginning treatment before hyperphagia presents itself or before it is considered clinically significant, maintaining the same HQ-CT total score over time can be indicative of effective treatment of the subject (e.g., a HQ-CT total score of 10-12 prior to treatment that remains within this range during treatment). For subjects with numerically greater HQ-CT total scores prior to treatment (e.g., those considered indicative of clinically significant hyperphagia, for example, a HQ-CT total score greater than or equal to 13, which would typically be considered moderate to severe hyperphagia), a decrease of 5-7 points in a subject's HQ-CT total score may be considered a clinically significant improvement in hyperphagia.

In some embodiments, the subject's Hyperphagia Questionnaire for Clinical Trials (HQ-CT) score is decreased compared to prior to commencement of treatment after treatment. In some embodiments, the decrease is determined after at least 13 weeks of treatment, including after at least 26 weeks of treatment, after at least 39 weeks of treatment, or after at least 52 weeks of treatment. A subject's HQ-CT score is determined by the caregiver providing answers to the 9 questions on the HQ-CT, provided below, corresponding to scores for the question of 0-4 and summing the scores across all questions (Fehnel S, Brown™, Nelson L, Chen A, Roof E, Kim D D, Dykens E M. Development of the hyperphagia questionnaire for use in Prader-Willi syndrome clinical trials. Poster presented at the 2015 ISPOR 20th Annual International Meeting; May 2015. Philadelphia, PA), the disclosure of which is incorporated by reference in its entirety.

HQ-CT Questions and Instructions: The following items refer to the person in your care and assessment of his/her food-related behavior during the past 2 weeks.
(1) During the past 2 weeks, how upset did the person generally become when denied a desired food?
   Not at all upset
   A little upset
   Moderately upset
   Very upset
   Extremely upset
(2) During the past 2 weeks, how often did the person try to bargain or manipulate to get more food at meals?
   Never
   Up to 2 times a week
   3 to 6 times a week
   Every day
   Several times a day
(3) During the past 2 weeks, how often did the person forage through trash for food?
   Never
   1 time
   2 times
   3 times
   4 or more times
(4) During the past 2 weeks, how often did the person get up at night to food seek?
   Never
   1 time
   2 times
   3 times
   4 or more times
(5) During the past 2 weeks, how persistent was the person in asking or looking for food after being told “no” or “no more” ?
   Not at all persistent
   A little persistent
   Moderately persistent
   Very persistent
   Extremely persistent
(6) During the past 2 weeks, outside of normal meal times, how much time did the person generally spend asking or talking about food?
   Less than 5 minutes a day
   5 to 15 minutes a day
   15 to 30 minutes a day
   30 minutes to 1 hour a day
   More than 1 hour a day
(7) During the past 2 weeks, how often did the person try to sneak or steal food (that you are aware of)?
   Never
   1 time
   2 times
   3 times
   4 or more times
(8) During the past 2 weeks, when others tried to stop the person from asking about food, how distressed did he or she generally appear?
   Not at all distressed
   A little distressed
   Moderately distressed
   Very distressed
   Extremely distressed
(9) During the past 2 weeks, how often did food-related behavior interfere with the person's normal daily activities, such as self-care, recreation, school, or work?
   Never
   Up to 2 times a week
   3 to 6 times a week
   Every day
   Several times a day

In some embodiments, the subject's food preoccupation is decreased after treatment. In some embodiments, the decrease is determined after at least 13 weeks of treatment, including after at least 26 weeks of treatment, after at least 39 weeks of treatment, or after at least 52 weeks of treatment. The term “food preoccupation,” as used herein, refers to excessive interest in food or eating.

In some embodiments, the subject's Food Related Problems Questionnaire (FRPQ) score is decreased compared to prior to commencement of treatment after treatment. In some embodiments, the decrease is determined after at least 13 weeks of treatment, including after at least 26 weeks of treatment, after at least 39 weeks of treatment, or after at least 52 weeks of treatment. A subject's FRPQ score is determined by summing the scores for 16 individual questions on the questionnaire, each scored on a 7-point Likert scale. The FRPQ summarizes complicated food related behaviors in 3 subscales (food preoccupation, impairment of satiety, and a composite of negative behavior). (Russell and Oliver Br. J. Clin. Psychol. 2003; 42:379-392).

A subject's FRPQ score is determined by the caregiver providing answers to the 16 questions on the FRPQ, provided below, corresponding to scores for the question of 0-6 (i.e., each question is answered using a 7-point Likert scale from 0 (never) to 6 (always) indicating the frequency of a food-related behavior). A subject's FRPQ score corresponds to the sum of the answers provided for the 16 individual questions. The FRPQ score may also be represented as summed subscores pertaining to “preoccupation with food,” “impairment of satiety,” and “composite negative behavior.” The “composite negative behavior” results can be further divided into subscores of “takes and stores food,” “inappropriate response,” and “eats inedible items” (Renzi et al. Food Nutr. Res. 2023; 67:10.29219/fnr.v67.9321) (Russell and Oliver Br. J. Clin. Psychol. 2003; 42:379-392).

The following items are questions from the FRPQ. The legend for subscores is as follows: “P”=preoccupation with food, “S”=impairment of satiety, and “N”=composite negative behavior; as noted above, the “composite negative behavior” results can be further divided into “N, TS”=negative behavior, takes and stores food; “N, IR”=negative behavior, inappropriate response; and “N, EI”=negative behavior, eats inedible items.
(1) How often does the person compare the size or content of their meal with others?(P)
(2) If given the opportunity, how often would the person ‘help themselves’ to food which they should not have?(N, TS)
(3) Is the person ever able to accept an explanation given to them if a meal is delayed?(N, IR)
(4) Does the person ever hide or hoard food?(N, TS)
(5) How often does the person talk about food?(P)
(6) If the person was denied food, how often would they respond negatively?(N, IR)
(7) Is it necessary to lock food away to stop the person from taking food?(N, TS)
(8) After an normal size meal, how often will the person say they still feel hungry?(S)
(9) If the person was tired, ill or upset, how often would this result in them going without food?(S)
(10) If it was available, would the person eat food not suitable for consumption (eg. frozen food, scraps from dustbins)?(N, EI)
(11) If a meal includes an item of food the person does not like or is not expecting, how often would this result in behavioural difficulties?(N, IR)
(12) Does the person ever eat non-edible items (e.g. dog food, leaves)?(N, EI)
(13) How frequently will the person share food with others?(S)
(14) Does the person ever describe ‘feeling full’?(S)
(15) Does the person ever associate people and/or places with specific food items or occasions involving food?(P)
(16) If given the opportunity, does the person ever eat more than a standard sized meal?(S)

In some embodiments, the subject's muscle strength is increased after treatment. In some embodiments, the increase is determined after at least 13 weeks of treatment, including after at least 26 weeks of treatment, after at least 39 weeks of treatment, or after at least 52 weeks of treatment.

In some embodiments, the subject's hypotonia is reduced after treatment. In some embodiments, the reduction is determined after at least 13 weeks of treatment, including after at least 26 weeks of treatment, after at least 39 weeks of treatment, or after at least 52 weeks of treatment. The term “hypotonia,” as used herein, refers to a condition characterized by decreased muscle tone.

In some embodiments, the subject's lean body mass/fat mass ratio is increased after treatment. In some embodiments, the increase is determined after at least 13 weeks of treatment, including after at least 26 weeks of treatment, after at least 39 weeks of treatment, or after at least 52 weeks of treatment. In some embodiments, the subject's lean body mass/fat mass ratio is increased by at least 1% compared to prior to commencement of treatment, including by at least 2% compared to prior to commencement of treatment, or by at least 3% compared to prior to commencement of treatment.

Changes in a subject's lean body mass, lean body mass/fat mass ratio, or lower limb muscle mass may be determined using a variety of methods known to the skilled clinician, including, for example, the use of Dual-energy X-ray Absorptiometry (DEXA) scans, which are non-invasive and can quickly measure body composition (e.g., fat, lean tissue and muscle mass), visceral adipose tissue, bone health, and estimate resting metabolic rate. The use of DEXA to evaluate PWS patients is described in J. Pediatr. Endocrinol. Metab. 1999 Apr; 12 Suppl 1:351-3. and J. Clin. Endocrinol. Metab. 2014 September; 99(9): E1727-31. Lean body mass includes muscle, organs, bones, and connective tissues. Lower limb muscle mass refers to the total amount of muscle tissue in the legs, including the thighs, calves, and feet.

In some embodiments, the subject's lean body mass is increased after treatment. In some embodiments, the increase is determined after at least 13 weeks of treatment, including after at least 26 weeks of treatment, after at least 39 weeks of treatment, or after at least 52 weeks of treatment. In some embodiments, the subject's lean body mass is increased by at least 1% compared to prior to commencement of treatment, including by at least 2% compared to prior to commencement of treatment, or by at least 3% compared to prior to commencement of treatment. In some embodiments, the subject's lean body mass is increased by at least 4%, (e.g., by about 5% or more) compared to prior to commencement of treatment. In some embodiments, the subject's lean body mass is increased by 7.5% or more compared to prior to commencement of treatment. In some embodiments, the subject's lean body mass is increased by at least 10% compared to prior to commencement of treatment, including at least by about 12%, at least about 12-13%.

In some embodiments, the subject's lower limb muscle mass is increased after treatment. In some embodiments, the increase is determined after at least 13 weeks of treatment, including after at least 26 weeks of treatment, after at least 39 weeks of treatment, or after at least 52 weeks of treatment.

In some embodiments, the subject's muscle mass is increased after treatment. In some embodiments, the increase is determined after at least 13 weeks of treatment, including after at least 26 weeks of treatment, after at least 39 weeks of treatment, or after at least 52 weeks of treatment.

In some embodiments, the subject's lean mass index (LMI) is increased after treatment. In some embodiments, the increase is determined after at least 13 weeks of treatment, including after at least 26 weeks of treatment, after at least 39 weeks of treatment, or after at least 52 weeks of treatment. In some embodiments, the subject's LMI is increased by at least 5% compared to prior to commencement of treatment, including by at least 10% compared to prior to commencement of treatment, or by at least 15% compared to prior to commencement of treatment.

A subject's LMI is a measurement of lean body mass relative to height (LMI=Lean Body Mass (kg)/Height² (m²)), where lean body mass is total body weight minus the weight of body fat and bone mineral content. Lean body mass is the primary determinant of energy expenditure. Thus, increasing a subject's lean body mass is expected to lead to a greater expenditure of energy over time.

In some embodiments, the subject's fat mass index (FMI) is decreased after treatment. In some embodiments, the decrease is determined after at least 13 weeks of treatment, including after at least 26 weeks of treatment, after at least 39 weeks of treatment, or after at least 52 weeks of treatment. In some embodiments, the subject's FMI is decreased by at least 5% compared to prior to commencement of treatment, including by at least 10% compared to prior to commencement of treatment, or by at least 15% compared to prior to commencement of treatment.

A subject's FMI is a measure of body fat distribution that takes into account both body fat mass and height. It is calculated as FMI=Fat Mass (kg)/Height² (m²). Higher FMI values indicate a higher proportion of body fat.

In some embodiments, the subject's amount of excess body fat is decreased after treatment. In some embodiments, the decrease is determined after at least 13 weeks of treatment, including after at least 26 weeks of treatment, after at least 39 weeks of treatment, or after at least 52 weeks of treatment.

In some embodiments, the rate of a subject's accumulation of excess body fat is decreased after treatment. In some embodiments, the decrease is determined after at least 13 weeks of treatment, including after at least 26 weeks of treatment, after at least 39 weeks of treatment, or after at least 52 weeks of treatment.

In some embodiments, one or more behavioral and cognitive comorbidities of PWS is reduced in the subject after treatment. In some embodiments, the reduction is determined after at least 13 weeks of treatment, including after at least 26 weeks of treatment, after at least 39 weeks of treatment, or after at least 52 weeks of treatment. In some embodiments, the one or more behavioral complications of PWS is selected from the group consisting of aggressive behavior, compulsive behavior, anxiety, rigidity or resistance to change, and temper tantrums.

Aggressive behaviors include temper outbursts (i.e., highly emotional or explosive episodes) in which the subject exhibits anger excessive for the situation or beyond their control, and which may include, but is not limited to, yelling, screaming, intimidation, breaking or throwing objects, or attacks on individuals.

Compulsive behaviors include any repetitive act that one feels obligated to perform, even if one is aware that these acts are not in line with one's overall goal. Compulsive behaviors include, but are not limited to, excessive cleaning, grooming, counting, hoarding, and checking (e.g., checking that doors or windows are locked). In a subject having PWS, compulsive behaviors may include insistence on sameness in certain routines, needing to know/tell/ask, collecting items of interest/hoarding, checking on collected items of interest, and repetitive rewriting, arranging, and rearranging.

Anxiety includes feelings of apprehension, worry, or fear, often about future events or situations. There is a set of explicit behaviors that appear to represent anxiety symptoms commonly expressed in certain subjects having PWS. These include repetitive questioning, especially related to schedules, food, and people; pacing; loud and/or fast talking; and excessive body and/or hand movements.

Rigidity or resistance to change includes reluctance to accept or adapt to novel ideas, processes, situations, or circumstances. Examples of rigidity in subjects having PWS include needing to have things done a certain way, including in a particular order, time, or place; difficulty with changes in schedule or routine; difficulty transitioning from one activity to another; getting stuck on a certain thought or idea; and excessively talking about a certain thought or idea.

In some embodiments, the subject's aggressive behaviors are reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's compulsive behaviors are reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's anxiety is reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's rigidity or resistance to change is reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's cognitive impairment is reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's learning disability is reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's speech impairment is reduced after treatment. In some embodiments, the reduction is determined after at least 13 weeks of treatment, including after at least 26 weeks of treatment, after at least 39 weeks of treatment, or after at least 52 weeks of treatment.

In some embodiments, the subject's cognitive ability is improved after treatment. In some embodiments, the improvement is determined after at least 13 weeks of treatment, including after at least 26 weeks of treatment, after at least 39 weeks of treatment, or after at least 52 weeks of treatment.

A subject's PWSP score is determined by the caregiver providing responses to the 58 statements (pertaining to nonfood-related behaviors) on the PWSP, provided below, corresponding to scores for the question of 0-2 and summing the scores across all questions. In the PWSP, the caregiver is asked to rate the subject's behavior using a 3-point response scale where: 0=Not True, 1=Sometimes True, and 2=Often True (Dykens et al. Orphanet J Rare Dis. 2024; 19(1):83). The PWSP score may also be represented as summed subscores pertaining to “aggressive behaviors” (items 1-9), “anxiety” (items 10-20), “rigidity/irritability” (items 21-30), “compulsivity” (items 31-40), “depression” (items 41-45), and “disordered thinking” (items 46-50 and 52). The “aggressive behaviors” results can be further divided into a subscore for “aggressive, destructive behaviors” (items 1-6).

The following items are statements from the PWSP, and pertain to different nonfood-related behaviors often observed in people with PWS (but may or may not apply to your own child, adolescent, or adult with PWS). Please respond with how much each nonfood-related behavior applies to your child, adolescent, or adult with PWS now or within the last two weeks.
(1) Physically aggressive (e.g., swings at others if too close; may throw items or destroy property).
(2) Has instances of rage (anger directed toward others or property that cause injury).
(3) Seeks to intimidate (gets close when angry, tries to appear threatening).
(4) Is verbally aggressive (e.g., argues, yells/screams, loud, demands to be heard, gets in your face, may make threatening gestures).
(5) When angry, will destroy items of personal value.
(6) Has temper tantrums contained to him/herself (e.g., cries, screams, throws self on ground, holds breath, stomps feet).
(7) Avoids taking responsibility for mistakes.
(8) Seems to finally understand something only after other people get really mad.
(9) Disagrees for the sake of disagreement.
(10) Difficulty controlling volume of their voice.
(11) Needs others to acknowledge that he/she is right or doing things correctly (e.g., overly concerned about messing up or being wrong, or overly defensive when told he/she is incorrect).
(12) Speech is intense, pressurized or fast, interrupts others to say things.
(13) Needs to be in control, is bossy, tells others how things must be done.
(14) Asks intrusive or inappropriate questions.
(15) Overly preoccupied with a certain person(s) (e.g., talks/asks about the person, needs reassurance about the person, needs to be in contact with the person).
(16) Repeatedly asks about people, events, or situations.
(17) Is stuck on specific topic(s) in conversations (e.g., starts conversations with topic, brings conversation back to topic).
(18) Becomes physically agitated when nervous (e.g., moves around, paces, rocks, rubs hands together, moves with intensity).
(19) Creates situation to avoid demands (e.g., causes problems at school to be sent home, spends time in the bathroom to avoid class, refuses to go to work/school).
(20) Seems uptight, wound up, or unable to relax.
(21) Insists his/her own opinions are correct even when facts contradict them (e.g., “You changed your hair color.” “No, it's the same color.” “No, you changed it.”).
(22) Difficulty taking other points of view (e.g., difficulty seeing that others have views, needs, or emotions that are different from their own).
(23) Rigid or concrete thinking, things are either black/white, all/none, right/wrong.
(24) Wants his/her own way all the time, unwilling to compromise.
(25) Feels that others intentionally bother or annoy them.
(26) Misinterprets or over-personalizes others' actions as negative (e.g., a stranger's frown means they are mad at him/her).
(27) Easily slighted, feels as if treated unfairly.
(28) Acts impulsively before considering the consequences.
(29) Needs to test boundaries or rules, especially with new people or in new settings.
(30) Shuts down after aggressive or upsetting episode, doesn't respond to name or redirection.
(31) Insists that daily routines (e.g., events, meals, self-care, routes to home/school/work) happen in the same way.
(32) Difficulty changing from one activity to another, gets stuck.
(33) Checks on things (e.g., items in room, backpack) to make sure they are still there and as they left them.
(34) Asks or says the same or similar things over and over again.
(35) Arranges items until they are “just right” (e.g., cards, toys, books, collections).
(36) Saves or collects items that you would likely throw away or recycle (e.g., junk mail, old school papers, boxes, wrappers, notes, pictures, old pencils, magazines).
(37) Has difficulty throwing or giving away items that he/she doesn't use (e.g., needs to keep items as “they are mine,” or just in case items are needed).
(38) Saves or collects items around a specific interest or theme (e.g., baby dolls, puzzles, Harry Potter, trains, animals, Disney characters, TV shows).
(39) Picks at skin or lips (e.g., scratches, rubs, picks).
(40) Picks or chews nails or cuticles.
(41) Has a negative outlook on life (e.g., expects that things won't go his/her way, pessimistic).
(42) Looks/seems sad or unhappy.
(43) Easily frustrated (e.g., may give up quickly or become upset with tasks that require any effort).
(44) Doesn't actively seek the company of others (e.g., unlikely to join in social activities).
(45) Prefers to spend time alone.
(46) Inflated sense of self (e.g., unrealistic expectations of their own limitations).
(47) Misinterprets others' actions as overly positive (e.g., a stranger's smile or casual greeting means they are a friend).
(48) Insists that strangers are people they know (e.g., “That girl was in my class last year.”).
(49) States that TV or other fictional characters are real (e.g., Harry Potter, Twilight, Disney).
(50) Talks to inanimate objects (e.g., books, movies).
(51) Overly involved in imaginary thinking, activities.
(52) Sees or talks to people who are not there.
(53) Rectal picking (e.g., may not directly observe this behavior but evidence may be on hands, underwear, or sheets).
(54) Makes gestures or behaves in ways that could cause serious self-harm (e.g., runs into street/traffic, opens door in moving car, injures self with intent).
(55) Runs away.
(56) Makes statements such as “I don't want to be alive,” “It would be better if I weren't here,” or “I can't do this anymore.”
(57) Creates situations to get into the hospital or attention from the police or health professionals.
(58) Reports hearing voices that others do not (e.g., could be scary or friendly voices).

In some embodiments, one or more of the subject's elevated inflammatory markers are decreased after treatment. In some embodiments, the decrease is determined after at least 13 weeks of treatment, including after at least 26 weeks of treatment, after at least 39 weeks of treatment, or after at least 52 weeks of treatment.

In some embodiments, the subject's amount of circulating C-Reactive Protein (CRP) is decreased after treatment. In some embodiments, the decrease is determined after at least 13 weeks of treatment, including after at least 26 weeks of treatment, after at least 39 weeks of treatment, or after at least 52 weeks of treatment.

Circulating CRP can be measured by methods known to the skilled artisan, including, for example, the high-sensitivity CRP (hsCRP) blood test. See e.g., Bassuk S. S., et al., Curr. Probl. Cardiol. 2004 Aug; 29(8):439-93.

In some embodiments, the subject's amount of circulating leptin is decreased after treatment. In some embodiments, the decrease is determined after at least 13 weeks of treatment, including after at least 26 weeks of treatment, after at least 39 weeks of treatment, or after at least 52 weeks of treatment.

In some embodiments, the subject's amount of circulating adiponectin is increased after treatment. In some embodiments, the increase is determined after at least 13 weeks of treatment, including after at least 26 weeks of treatment, after at least 39 weeks of treatment, or after at least 52 weeks of treatment.

In some embodiments, the subject's ratio of circulating adiponectin to circulating leptin is increased after treatment. In some embodiments, the increase is determined after at least 13 weeks of treatment, including after at least 26 weeks of treatment, after at least 39 weeks of treatment, or after at least 52 weeks of treatment.

Circulating leptin or circulating adiponectin can be measured in serum or plasma by methods known to the skilled artisan, including, for example, through the use of enzyme-linked immunosorbent assays (ELISAs).

In some embodiments, the subject's insulin resistance is improved after treatment. In some embodiments, the improvement is determined after at least 13 weeks of treatment, including after at least 26 weeks of treatment, after at least 39 weeks of treatment, or after at least 52 weeks of treatment.

### ENUMERATED EMBODIMENTS

Embodiment I-1. A method of treating a subject having Prader-Willi Syndrome, the method comprising:
(a) administering to the subject a therapeutically effective amount of diazoxide, or a pharmaceutically acceptable salt thereof, and
(b) monitoring the patient for signs and symptoms of hyperglycemia and/or hyperuricemia.

Embodiment I-2. The method of embodiment I-1, wherein the subject is concomitantly administered a diuretic.

Embodiment I-3. The method of embodiment 1-2, wherein the amount of diuretic administered to the subject is reduced if the patient exhibits signs or symptoms of hyperglycemia and/or hyperuicemia.

Embodiment I-4. The method of any one of embodiments I-1 to 1-3, wherein the subject is concomitantly administered a thiazide.

Embodiment I-5. The method of any one of embodiments I-1 to 1-4, wherein the therapeutically effective amount of diazoxide, or a pharmaceutically acceptable salt thereof, is adjusted if the patient exhibits signs or symptoms of hyperglycemia and/or hyperuricemia.

Embodiment I-6. The method of embodiment 1-5, wherein the therapeutically effective amount of diazoxide, or a pharmaceutically acceptable salt thereof, is reduced.

Embodiment I-7. A method of treating a subject having Prader-Willi Syndrome, the method comprising:
(a) administering to the subject a therapeutically effective amount of diazoxide, or a pharmaceutically acceptable salt thereof, and
(b) monitoring serum levels of diphenylhydantoin in the patient.

Embodiment I-8. The method of embodiment I-7, wherein the subject is concomitantly administered diphenylhydantoin.

Embodiment I-9. The method of embodiment I-8, wherein the dosage of diphenylhydantoin is reduced.

Embodiment I-10. A method of treating a subject having Prader-Willi Syndrome, the method comprising:
(a) administering to the subject a therapeutically effective amount of diazoxide, or a pharmaceutically acceptable salt thereof, and
(b) monitoring international normalized ratio (INR) of the patient.

Embodiment I-11. The method of embodiment I-10, wherein the subject is concomitantly administered coumarin or a derivative thereof.

Embodiment I-12. The method of embodiment I-11, wherein the dosage of coumarin, or a derivative thereof, is reduced.

Embodiment I-13. A method of treating a subject having Prader-Willi Syndrome, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment I-14. The method of embodiment I-13, wherein the therapeutically effective amount of diazoxide, or a pharmaceutically acceptable salt thereof, is reduced relative to the therapeutically effective amount for the subject if not concomitantly administered one or more strong CYP3A4 inhibitors.

Embodiment I-15. The method of embodiment I-13 or I-14, wherein the therapeutically effective amount of diazoxide, or a pharmaceutically acceptable salt thereof, is reduced by between about 15.0%-30.0%.

Embodiment I-16. The method of any one of embodiments I-13 to I-15, wherein the therapeutically effective amount of diazoxide, or a pharmaceutically acceptable salt thereof, is reduced by about 15.0%.

Embodiment I-17. The method of any one of embodiments I-13 to I-16, wherein the therapeutically effective amount of diazoxide, or a pharmaceutically acceptable salt thereof, is reduced by about 30.0%.

Embodiment I-18. The method of anyone of embodiments I-13 to I-17, wherein the adverse reactions include hyperglycemia and the risk of type 2 diabetes or ketoacidosis, peripheral edema and fluid overload, and hypertrichosis.

Embodiment I-19. The method of any one of embodiments I-13 to I-18, wherein the one or more strong CYP3A4 inhibitors is selected from the group consisting of ceritinib, clarithromycin, delaviridone, idelalisib, indinavir, itraconazole, ketoconazole, mibefradil, nefazodone, nelfinivir, ribociclib, ritonavir, saquinavir, telaprevir, telithromycin, tucatinib, voriconazole.

Embodiment I-20. A method of treating a subject having Prader-Willi Syndrome, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment I-21. The method of embodiment I-20, wherein the one or more strong CYP1A2 inhibitors is selected from the group consisting of amiodarone, ciprofloxacin, and fluvoxamine.

Embodiment I-22. The method of embodiment I-20 or I-21, wherein the therapeutically effective amount of diazoxide is reduced relative to the therapeutically effective amount for the subject if not concomitantly administered one or more strong CYP1A2 inhibitors.

Embodiment I-23. The method of any one of embodiments I-1 to I-19, wherein the diazoxide, or a pharmaceutically acceptable salt thereof, is formulated as a pharmaceutical formulation comprising at least one excipient that extends the release of diazoxide, or a pharmaceutically acceptable salt thereof.

Embodiment I-24. The method of any one of embodiments I-1 to I-23, wherein the pharmaceutically acceptable salt is diazoxide choline.

Embodiment I-25. The method of embodiment I-24, wherein the administration of diazoxide choline is initiated at a reduced starting dose of diazoxide choline and then increased each 2-3 weeks to reach a reduced target maintenance dose of diazoxide choline.

Embodiment I-26. The method of embodiment I-25, wherein the reduced target maintenance dose of diazoxide choline is reached after 4-10 weeks.

Embodiment I-27. The method of embodiment I-25 or I-26, wherein the reduced target maintenance dose of diazoxide choline is reached after 4-8 weeks.

Embodiment I-28. The method of any one of embodiments I-25 to I-27, wherein the reduced target maintenance dose of diazoxide choline is reached after 7-8 weeks.

Embodiment I-29. The method of any one of embodiments I-24 to I-28, wherein the reduced target maintenance dose of diazoxide choline is between about 1.1-4.2 mg/kg/day of diazoxide choline.

Embodiment I-30. The method of any one of embodiments I-24 to I-29, wherein the reduced target maintenance dose of diazoxide choline is about 3.0 mg/kg/day of diazoxide choline.

Embodiment I-31. The method of any one of embodiments I-24 to I-30, wherein the reduced starting dose of diazoxide choline is between about 3-100 mg/day of diazoxide choline.

Embodiment I-32. The method of embodiment I-31, wherein the reduced starting dose of diazoxide choline is between about 3-18 mg/day of diazoxide choline.

Embodiment I-33. The method of embodiment I-32, wherein the reduced starting dose of diazoxide choline is about 3 mg/day of diazoxide choline.

Embodiment I-34. The method of embodiment I-32, wherein the reduced starting dose of diazoxide choline is about 18 mg/day of diazoxide choline.

Embodiment I-35. The method of any one of embodiments I-24 to I-31, wherein the reduced starting dose of diazoxide choline is between about 25-100 mg/day of diazoxide choline.

Embodiment I-36. The method of embodiment I-35, wherein the reduced starting dose of diazoxide choline is about 25 mg/day of diazoxide choline.

Embodiment I-37. The method of embodiment I-35, wherein the reduced starting dose of diazoxide choline is about 100 mg/day of diazoxide choline.

Embodiment I-38. The method of any one of embodiments I-24 to I-37, wherein after 2-3 weeks the reduced starting dose of diazoxide choline is increased to a reduced second dose of diazoxide choline.

Embodiment I-39. The method of embodiment I-38, wherein the reduced second dose of diazoxide choline is between about 5-200 mg/day of diazoxide choline.

Embodiment I-40. The method of embodiment I-39, wherein the reduced second dose of diazoxide choline is between about 5-36 mg/day of diazoxide choline.

Embodiment I-41. The method of embodiment I-40, wherein the reduced second dose of diazoxide choline is about 5 mg/day of diazoxide choline.

Embodiment I-42. The method of embodiment I-40, wherein the reduced second dose of diazoxide choline is about 36 mg/day of diazoxide choline.

Embodiment I-43. The method of embodiment I-38 or I-39, wherein the reduced second dose of diazoxide choline is between about 25-200 mg/day of diazoxide choline.

Embodiment I-44. The method of embodiment I-43, wherein the reduced second dose of diazoxide choline is about 25 mg/day of diazoxide choline.

Embodiment I-45. The method of embodiment I-43, wherein the reduced second dose of diazoxide choline is about 200 mg/day of diazoxide choline.

Embodiment I-46. The method of any one of embodiments I-38 to I-45, wherein after 2-3 weeks the reduced second dose of diazoxide choline is increased to a reduced third dose of diazoxide choline.

Embodiment I-47. The method of embodiment I-46, wherein the reduced third dose of diazoxide choline is between about 7-300 mg/day of diazoxide choline.

Embodiment I-48. The method of embodiment I-47, wherein the reduced third dose of diazoxide choline is between about 7-54 mg/day of diazoxide choline.

Embodiment I-49. The method of embodiment I-48, wherein the reduced third dose of diazoxide choline is about 7 mg/day of diazoxide choline.

Embodiment I-50. The method of embodiment I-48, wherein the reduced third dose of diazoxide choline is about 54 mg/day of diazoxide choline.

Embodiment I-51. The method of embodiment I-46 or I-47, wherein the reduced third dose of diazoxide choline is between about 50-300 mg/day of diazoxide choline.

Embodiment I-52. The method of embodiment I-51, wherein the reduced third dose of diazoxide choline is about 50 mg/day of diazoxide choline.

Embodiment I-53. The method of embodiment I-51, wherein the reduced third dose of diazoxide choline is about 300 mg/day of diazoxide choline.

Embodiment I-54. The method of any one of embodiments I-46 to I-53, wherein after 2-3 weeks the reduced third dose of diazoxide choline is increased to the reduced target maintenance dose of diazoxide choline.

Embodiment I-55. The method of any one of embodiments I-24 to I-54, wherein the reduced target maintenance dose of diazoxide choline is between about 9-325 mg/day of diazoxide choline.

Embodiment I-56. The method of embodiment I-55, wherein the reduced target maintenance dose of diazoxide choline is between about 9-58 mg/day of diazoxide choline.

Embodiment I-57. The method of embodiment I-56, wherein the reduced target maintenance dose of diazoxide choline is about 9 mg/day of diazoxide choline.

Embodiment I-58. The method of embodiment I-56, wherein the reduced target maintenance dose of diazoxide choline is about 58 mg/day of diazoxide choline.

Embodiment I-59. The method of any one of embodiments I-24 to I-55, wherein the reduced target maintenance dose of diazoxide choline is between about 75-325 mg/day of diazoxide choline.

Embodiment I-60. The method of embodiment I-59, wherein the reduced target maintenance dose of diazoxide choline is about 75 mg/day of diazoxide choline.

Embodiment I-61. The method of embodiment I-59, wherein the reduced target maintenance dose of diazoxide choline is about 325 mg/day of diazoxide choline.

Embodiment I-62. The method of embodiment I-24, wherein the administration of diazoxide choline is in an amount from about 1.1 mg/kg/day to about 4.2 mg/kg/day.

Embodiment I-63. The method of embodiment I-24 or I-62, wherein the administration of diazoxide choline is in an amount of about 3.0 mg/kg/day.

Embodiment I-64. The method of embodiment I-24, wherein the once-daily administration of diazoxide choline is in an amount from about 18 mg to about 42 mg.

Embodiment I-65. The method of embodiment I-24, wherein the once-daily administration of diazoxide choline is in an amount from about 18 mg to about 58 mg.

Embodiment I-66. The method of embodiment I-24, wherein the once-daily administration of diazoxide choline is in an amount from about 25 mg to about 75 mg.

Embodiment I-67. The method of embodiment I-24, wherein the once-daily administration of diazoxide choline is in an amount from about 50 mg to about 100 mg.

Embodiment I-68. The method of embodiment I-24, wherein the once-daily administration of diazoxide choline is in an amount from about 50 mg to about 150 mg.

Embodiment I-69. The method of embodiment I-24, wherein the once-daily administration of diazoxide choline is in an amount from about 100 mg to about 250 mg.

Embodiment I-70. The method of embodiment I-24, wherein the once-daily administration of diazoxide choline is in an amount from about 100 mg to about 300 mg.

Embodiment I-71. The method of embodiment I-24, wherein the once-daily administration of diazoxide choline is in an amount from about 100 mg to about 325 mg.

Embodiment I-72. The method of any one of embodiments I-1 to I-71, wherein the subject has a weight less than about 135 kg.

Embodiment I-73. The method of embodiment I-72, wherein the subject has a weight between about 3 kg and about 40 kg.

Embodiment I-74. The method of embodiment I-73, wherein the subject has a weight between about 20 kg and about 40 kg.

Embodiment I-75. The method of embodiment I-74, wherein the subject has a weight between about 30 kg and about 40 kg.

Embodiment I-76. The method of embodiment I-74, wherein the subject has a weight between about 20 kg and about 30 kg.

Embodiment I-77. The method of embodiment I-73, wherein the subject has a weight between about 3 kg and about 20 kg.

Embodiment I-78. The method of embodiment I-77, wherein the subject has a weight between about 10 kg and about 20 kg.

Embodiment I-79. The method of embodiment I-77, wherein the subject has a weight between about 3 kg and about 10 kg.

Embodiment I-80. The method of any one of embodiments I-1 to I-79, wherein the subject is less than or equal to 8 years of age.

Embodiment I-81. The method of embodiment I-80, wherein the subject is less than or equal to 4 years of age.

Embodiment I-82. The method of embodiment I-81, wherein the subject is less than or equal to 1 year of age.

Embodiment I-83. The method of any one of embodiments I-1 to I-82, wherein after at least 13 weeks of treatment, the subject's hypotonia is reduced.

Embodiment I-84. The method of any one of embodiments I-1 to I-83, wherein after at least 13 weeks of treatment, the subject's lean body mass/fat mass ratio is increased.

Embodiment I-85. The method of embodiment I-84, wherein the subject's lean body mass/fat mass ratio is increased by at least 3% compared to prior to commencement of treatment.

Embodiment I-86. The method of any one of embodiments I-1 to I-85, wherein after at least 13 weeks of treatment, the subject's lean body mass is increased.

Embodiment I-87. The method of embodiment I-86, wherein the subject's lean body mass is increased by at least 3% compared to prior to commencement of treatment.

Embodiment I-88. The method of any one of embodiments I-1 to I-87, wherein after at least 13 weeks of treatment, the subject's lower limb muscle mass is increased.

Embodiment I-89. The method of any one of embodiments I-1 to I-88, wherein after at least 13 weeks of treatment, the subject's food preoccupation is decreased.

Embodiment I-90. The method of embodiment I-89, wherein the subject's Food Related Problems Questionnaire (FRPQ) score is decreased compared to prior to commencement of treatment.

Embodiment I-91. The method of any one of embodiments I-1 to I-90, wherein after at least 13 weeks of treatment, the subject's muscle mass is increased.

Embodiment I-92. The method of any one of embodiments I-1 to I-91, wherein after at least 13 weeks of treatment, the subject's lean mass index (LMI) is increased.

Embodiment I-93. The method of embodiment I-92, wherein the subject's LMI is increased by at least 10% compared to prior to commencement of treatment.

Embodiment I-94. The method of any one of embodiments I-1 to I-93, wherein after at least 13 weeks of treatment, the subject's fat mass index (FMI) is decreased.

Embodiment I-95. The method of embodiment I-94, wherein the subject's FMI is decreased by at least 5% compared to prior to commencement of treatment.

Embodiment I-96. The method of any one of embodiments I-1 to I-95, wherein after at least 13 weeks of treatment, one or more behavioral or cognitive complications of PWS is reduced in the subject.

Embodiment I-97. The method of embodiment I-96, wherein the one or more behavioral complications of PWS is selected from the group consisting of aggressive behavior, compulsive behavior, anxiety, rigidity or resistance to change, and temper tantrums.

Embodiment I-98. The method of any one of embodiments I-1 to I-97, wherein after at least 13 weeks of treatment, the subject's cognitive ability is improved.

Embodiment I-99. The method of any one of embodiments I-1 to I-98, wherein after at least 13 weeks of treatment, the subject's amount of circulating C-Reactive Protein (CRP) is decreased.

Embodiment II-1. A method of treating a subject having Prader-Willi Syndrome, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-2. The method of embodiment II-1, wherein the diazoxide, or a pharmaceutically acceptable salt thereof, is formulated as a pharmaceutical formulation comprising at least one excipient that extends the release of diazoxide, or a pharmaceutically acceptable salt thereof.

Embodiment II-3. The method of embodiments II-1 or II-2, wherein the pharmaceutically acceptable salt is diazoxide choline.

Embodiment II-4. The method of embodiment II-3, wherein the administration of diazoxide choline is initiated at a reduced starting dose of diazoxide choline and then increased each 2-3 weeks to reach a reduced target maintenance dose of diazoxide choline.

Embodiment II-5. The method of embodiment II-4, wherein the reduced target maintenance dose of diazoxide choline is reached after 4-10 weeks.

Embodiment II-6. The method of embodiments II-4 or II-5, wherein the reduced target maintenance dose of diazoxide choline is reached after 4-8 weeks.

Embodiment II-7. The method of any one of embodiments II-4 to II-6, wherein the reduced target maintenance dose of diazoxide choline is reached after 7-8 weeks.

Embodiment II-8. The method of any one of embodiments II-4 to II-7, wherein the reduced target maintenance dose of diazoxide choline is between about 1.1-4.2 mg/kg/day of diazoxide choline.

Embodiment II-9. The method of any one of embodiments II-4 to II-8, wherein the reduced target maintenance dose of diazoxide choline is about 3.0 mg/kg/day of diazoxide choline.

Embodiment II-10. The method of any one of embodiments II-4 to II-9, wherein the reduced starting dose of diazoxide choline is between about 3-100 mg/day of diazoxide choline.

Embodiment II-11. The method of embodiment II-10, wherein the reduced starting dose of diazoxide choline is between about 3-18 mg/day of diazoxide choline.

Embodiment II-12. The method of embodiment II-11, wherein the reduced starting dose of diazoxide choline is about 3 mg/day of diazoxide choline.

Embodiment II-13. The method of embodiment II-11, wherein the reduced starting dose of diazoxide choline is about 18 mg/day of diazoxide choline.

Embodiment II-14. The method of embodiment II-10, wherein the reduced starting dose of diazoxide choline is between about 25-100 mg/day of diazoxide choline.

Embodiment II-15. The method of embodiment II-14, wherein the reduced starting dose of diazoxide choline is about 25 mg/day of diazoxide choline.

Embodiment II-16. The method of embodiment II-14, wherein the reduced starting dose of diazoxide choline is about 100 mg/day of diazoxide choline.

Embodiment II-17. The method of any one of embodiments II-4 to II-16, wherein after 2-3 weeks the reduced starting dose of diazoxide choline is increased to a reduced second dose of diazoxide choline.

Embodiment II-18. The method of embodiment II-17, wherein the reduced second dose of diazoxide choline is between about 5-200 mg/day of diazoxide choline.

Embodiment II-19. The method of embodiment II-18, wherein the reduced second dose of diazoxide choline is between about 5-36 mg/day of diazoxide choline.

Embodiment II-20. The method of embodiment II-19, wherein the reduced second dose of diazoxide choline is about 5 mg/day of diazoxide choline.

Embodiment II-21. The method of embodiment II-19, wherein the reduced second dose of diazoxide choline is about 36 mg/day of diazoxide choline.

Embodiment II-22. The method of embodiment II-18, wherein the reduced second dose of diazoxide choline is between about 25-200 mg/day of diazoxide choline.

Embodiment II-23. The method of embodiment II-22, wherein the reduced second dose of diazoxide choline is about 25 mg/day of diazoxide choline.

Embodiment II-24. The method of embodiment II-22, wherein the reduced second dose of diazoxide choline is about 200 mg/day of diazoxide choline.

Embodiment II-25. The method of any one of embodiments II-17 to II-24, wherein after 2-3 weeks the reduced second dose of diazoxide choline is increased to a reduced third dose of diazoxide choline.

Embodiment II-26. The method of embodiment II-25, wherein the reduced third dose of diazoxide choline is between about 7-300 mg/day of diazoxide choline.

Embodiment II-27. The method of embodiment II-26, wherein the reduced third dose of diazoxide choline is between about 7-54 mg/day of diazoxide choline.

Embodiment II-28. The method of embodiment II-27, wherein the reduced third dose of diazoxide choline is about 7 mg/day of diazoxide choline.

Embodiment II-29. The method of embodiment II-27, wherein the reduced third dose of diazoxide choline is about 54 mg/day of diazoxide choline.

Embodiment II-30. The method of embodiment II-26, wherein the reduced third dose of diazoxide choline is between about 50-300 mg/day of diazoxide choline.

Embodiment II-31. The method of embodiment II-30, wherein the reduced third dose of diazoxide choline is about 50 mg/day of diazoxide choline.

Embodiment II-32. The method of embodiment II-30, wherein the reduced third dose of diazoxide choline is about 300 mg/day of diazoxide choline.

Embodiment II-33. The method of any one of embodiments II-25 to II-32, wherein after 2-3 weeks the reduced third dose of diazoxide choline is increased to the reduced target maintenance dose of diazoxide choline.

Embodiment II-34. The method of any one of embodiments II-4 to II-33, wherein the reduced target maintenance dose of diazoxide choline is between about 9-325 mg/day of diazoxide choline.

Embodiment II-35. The method of embodiment II-34, wherein the reduced target maintenance dose of diazoxide choline is between about 9-58 mg/day of diazoxide choline.

Embodiment II-36. The method of embodiment II-35, wherein the reduced target maintenance dose of diazoxide choline is about 9 mg/day of diazoxide choline.

Embodiment II-37. The method of embodiment II-35, wherein the reduced target maintenance dose of diazoxide choline is about 58 mg/day of diazoxide choline.

Embodiment II-38. The method of embodiment II-34, wherein the reduced target maintenance dose of diazoxide choline is between about 75-325 mg/day of diazoxide choline.

Embodiment II-39. The method of embodiment II-38, wherein the reduced target maintenance dose of diazoxide choline is about 75 mg/day of diazoxide choline.

Embodiment II-40. The method of embodiment II-38, wherein the reduced target maintenance dose of diazoxide choline is about 325 mg/day of diazoxide choline.

Embodiment II-41. The method of embodiment II-3, wherein the administration of diazoxide choline is in an amount from about 1.1 mg/kg/day to about 4.2 mg/kg/day.

Embodiment II-42. The method of embodiments II-3 or II-41, wherein the administration of diazoxide choline is in an amount of about 3.0 mg/kg/day.

Embodiment II-43. The method of embodiment II-3, wherein the once-daily administration of diazoxide choline is in an amount from about 3 mg to about 30 mg.

Embodiment II-44. The method of embodiment II-3, wherein the once-daily administration of diazoxide choline is in an amount from about 18 mg to about 42 mg.

Embodiment II-45. The method of embodiment II-3, wherein the once-daily administration of diazoxide choline is in an amount from about 18 mg to about 58 mg.

Embodiment II-46. The method of embodiment II-3, wherein the once-daily administration of diazoxide choline is in an amount from about 25 mg to about 75 mg.

Embodiment II-47. The method of embodiment II-3, wherein the once-daily administration of diazoxide choline is in an amount from about 50 mg to about 100 mg.

Embodiment II-48. The method of embodiment II-3, wherein the once-daily administration of diazoxide choline is in an amount from about 50 mg to about 150 mg.

Embodiment II-49. The method of embodiment II-3, wherein the once-daily administration of diazoxide choline is in an amount from about 100 mg to about 250 mg.

Embodiment II-50. The method of embodiment II-3, wherein the once-daily administration of diazoxide choline is in an amount from about 100 mg to about 300 mg.

Embodiment II-51. The method of embodiment II-3, wherein the once-daily administration of diazoxide choline is in an amount from about 100 mg to about 325 mg.

Embodiment II-52. The method of embodiment II-3, wherein the therapeutically effective amount of diazoxide choline is reduced such that it is an amount from about 1.1 mg/kg/day to about 3.0 mg/kg/day.

Embodiment II-53. A method of treating a subject having Smith-Magenis Syndrome, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-54. The method of embodiment II-53, wherein the pharmaceutically acceptable salt is diazoxide choline, and wherein the therapeutically effective amount of diazoxide choline is reduced such that it is an amount from about 1.1 mg/kg/day to about 3.0 mg/kg/day.

Embodiment II-55. A method of reducing the triglyceride level of a subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-56. The method of embodiment II-55, wherein the pharmaceutically acceptable salt is diazoxide choline, and wherein the therapeutically effective amount of diazoxide choline is reduced such that it is an amount from about 1.4 mg/kg/day to about 3.0 mg/kg/day.

Embodiment II-57. A method of reducing the non-HDL cholesterol level of a subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-58. The method of embodiment II-57, wherein the pharmaceutically acceptable salt is diazoxide choline, and wherein the therapeutically effective amount of diazoxide choline is reduced such that it is an amount from about 1.4 mg/kg/day to about 3.0 mg/kg/day.

Embodiment II-59. A method of promoting weight loss in an obese subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-60. The method of embodiment II-59, wherein the pharmaceutically acceptable salt is diazoxide choline, and wherein the therapeutically effective amount of diazoxide choline is reduced such that it is an amount from about 2.1 mg/kg/day to about 4.7 mg/kg/day.

Embodiment II-61. A method of increasing the fasting glucose level of a subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-62. The method of embodiment II-61, wherein the pharmaceutically acceptable salt is diazoxide choline, and wherein the therapeutically effective amount of diazoxide choline is reduced such that it is an amount from about 2.0 mg/kg/day to about 7.7 mg/kg/day.

Embodiment II-63. A method of reducing the fasting insulin level of a subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-64. The method of embodiment II-63, wherein the pharmaceutically acceptable salt is diazoxide choline, and wherein the therapeutically effective amount of diazoxide choline is reduced such that it is an amount from about 0.8 mg/kg/day to about 4.3 mg/kg/day.

Embodiment II-65. A method of improving insulin resistance in a subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-66. The method of embodiment II-65, wherein the pharmaceutically acceptable salt is diazoxide choline, and wherein the therapeutically effective amount of diazoxide choline is reduced such that it is an amount from about 1.6 mg/kg/day to about 4.4 mg/kg/day.

Embodiment II-67. A method of treating a subject in need thereof, the method comprising:
(a) administering to the subject a therapeutically effective amount of diazoxide, or a pharmaceutically acceptable salt thereof, wherein the subject is concomitantly administered one or more strong CYP1A2 inhibitors; and
(b) adjusting the therapeutically effective amount of diazoxide, or a pharmaceutically acceptable salt thereof, administered to the subject.

Embodiment II-68. The method of embodiment II-67, wherein the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance.

Embodiment II-69. The method of any one of embodiments II-1 to II-68, wherein the one or more strong CYP1A2 inhibitors is selected from the group consisting of amiodarone, ciprofloxacin, and fluvoxamine.

Embodiment II-70. The method of any one of embodiments II-1 to II-69, wherein the therapeutically effective amount of diazoxide is reduced relative to the therapeutically effective amount for the subject if not concomitantly administered one or more strong CYP1A2 inhibitors.

Embodiment II-71. A method of treating a subject having Prader-Willi Syndrome, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-72. The method of embodiment II-71, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-73. The method of embodiment II-72, wherein the therapeutically effective amount of diazoxide, or a pharmaceutically acceptable salt thereof, is reduced by about 15%.

Embodiment II-74. The method of embodiment II-72, wherein the therapeutically effective amount of diazoxide, or a pharmaceutically acceptable salt thereof, is reduced by about 30%.

Embodiment II-75. A method of treating a subject having Smith-Magenis Syndrome, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-76. The method of embodiment II-71, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-77. The method of embodiment II-76, wherein the therapeutically effective amount of diazoxide, or a pharmaceutically acceptable salt thereof, is reduced by about 15%.

Embodiment II-78. The method of embodiment II-76, wherein the therapeutically effective amount of diazoxide, or a pharmaceutically acceptable salt thereof, is reduced by about 30%.

Embodiment II-79. A method of reducing the triglyceride level of a subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-80. The method of embodiment II-71, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-81. The method of embodiment II-80, wherein the therapeutically effective amount of diazoxide, or a pharmaceutically acceptable salt thereof, is reduced by about 15%.

Embodiment II-82. The method of embodiment II-80, wherein the therapeutically effective amount of diazoxide, or a pharmaceutically acceptable salt thereof, is reduced by about 30%.

Embodiment II-83. A method of reducing the non-HDL cholesterol level of a subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-84. The method of embodiment II-71, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-85. The method of embodiment II-84, wherein the therapeutically effective amount of diazoxide, or a pharmaceutically acceptable salt thereof, is reduced by about 15%.

Embodiment II-86. The method of embodiment II-84, wherein the therapeutically effective amount of diazoxide, or a pharmaceutically acceptable salt thereof, is reduced by about 30%.

Embodiment II-87. A method of promoting weight loss in an obese subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-88. The method of embodiment II-71, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-89. The method of embodiment II-88, wherein the therapeutically effective amount of diazoxide, or a pharmaceutically acceptable salt thereof, is reduced by about 15%.

Embodiment II-90. The method of embodiment II-88, wherein the therapeutically effective amount of diazoxide, or a pharmaceutically acceptable salt thereof, is reduced by about 30%.

Embodiment II-91. A method of increasing the fasting glucose level of a subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-92. The method of embodiment II-91, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-93. The method of embodiment II-92, wherein the therapeutically effective amount of diazoxide, or a pharmaceutically acceptable salt thereof, is reduced by about 15%.

Embodiment II-94. The method of embodiment II-92, wherein the therapeutically effective amount of diazoxide, or a pharmaceutically acceptable salt thereof, is reduced by about 30%.

Embodiment II-95. A method of reducing the fasting insulin level of a subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-96. The method of embodiment II-95, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-97. The method of embodiment II-96, wherein the therapeutically effective amount of diazoxide, or a pharmaceutically acceptable salt thereof, is reduced by about 15%.

Embodiment II-98. The method of embodiment II-96, wherein the therapeutically effective amount of diazoxide, or a pharmaceutically acceptable salt thereof, is reduced by about 30%.

Embodiment II-99. A method of improving insulin resistance in a subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-100. The method of embodiment II-99, wherein the therapeutically effective amount of diazoxide, or a pharmaceutically acceptable salt thereof, is reduced by between about 15%-30% relative to the therapeutically effective amount for the subject if not concomitantly administered one or more strong CYP3A4 inhibitors.

Embodiment II-101. The method of embodiment II-100, wherein the therapeutically effective amount of diazoxide, or a pharmaceutically acceptable salt thereof, is reduced by about 15%.

Embodiment II-102. The method of embodiment II-100, wherein the therapeutically effective amount of diazoxide, or a pharmaceutically acceptable salt thereof, is reduced by about 30%.

Embodiment II-103. A method of treating a subject in need thereof, the method comprising:
(a) administering to the subject a therapeutically effective amount of diazoxide, or a pharmaceutically acceptable salt thereof, wherein the subject is concomitantly administered one or more strong CYP3A4 inhibitors;
(b) monitoring the subject for adverse reactions to diazoxide, or a pharmaceutically acceptable salt thereof; and
(c) if the subject exhibits adverse reactions, adjusting the therapeutically effective amount of diazoxide, or a pharmaceutically acceptable salt thereof, administered to the subject.

Embodiment II-104. The method of embodiment II-103, wherein the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance.

Embodiment II-105. The method of any one of embodiments II-71 to II-104, wherein the adverse reactions include hyperglycemia and the risk of type 2 diabetes or ketoacidosis, peripheral edema and fluid overload, and hypertrichosis.

Embodiment II-106. The method of any one of embodiments II-71 to II-105, wherein the one or more strong CYP3A4 inhibitors is selected from the group consisting of ceritinib, clarithromycin, delaviridone, idelalisib, indinavir, itraconazole, ketoconazole, mibefradil, nefazodone, nelfinivir, ribociclib, ritonavir, saquinavir, telaprevir, telithromycin, tucatinib, voriconazole.

Embodiment II-107. The method of any one of embodiments II-71 to II-70, wherein the subject has a weight less than about 135 kg.

Embodiment II-108. The method of embodiment II-107, wherein the subject has a weight between about 3 kg and about 40 kg.

Embodiment II-109. The method of embodiment II-108, wherein the subject has a weight between about 20 kg and about 40 kg.

Embodiment II-110. The method of embodiment II-109, wherein the subject has a weight between about 30 kg and about 40 kg.

Embodiment II-111. The method of embodiment II-109, wherein the subject has a weight between about 20 kg and about 30 kg.

Embodiment II-112. The method of embodiment II-108, wherein the subject has a weight between about 3 kg and about 20 kg.

Embodiment II-113. The method of embodiment II-112, wherein the subject has a weight between about 10 kg and about 20 kg.

Embodiment II-114. The method of embodiment II-112, wherein the subject has a weight between about 3 kg and about 10 kg.

Embodiment II-115. The method of any one of embodiments II-71 to II-114, wherein the subject is less than or equal to 8 years of age.

Embodiment II-116. The method of embodiment II-115, wherein the subject is less than or equal to 4 years of age.

Embodiment II-117. The method of embodiment II-116, wherein the subject is less than or equal to 1 year of age.

Embodiment II-118. The method of any one of embodiments II-71 to II-117, wherein after at least 13 weeks of treatment, the subject's hypotonia is reduced.

Embodiment II-119. The method of any one of embodiments II-71 to II-118, wherein after at least 13 weeks of treatment, the subject's lean body mass/fat mass ratio is increased.

Embodiment II-120. The method of embodiment II-119, wherein the subject's lean body mass/fat mass ratio is increased by at least 3% compared to prior to commencement of treatment.

Embodiment II-121. The method of any one of embodiments II-71 to II-120, wherein after at least 13 weeks of treatment, the subject's lean body mass is increased.

Embodiment II-122. The method of embodiment II-121, wherein the subject's lean body mass is increased by at least 3% compared to prior to commencement of treatment.

Embodiment II-123. The method of any one of embodiments II-71 to II-122, wherein after at least 13 weeks of treatment, the subject's lower limb muscle mass is increased.

Embodiment II-124. The method of any one of embodiments II-71 to II-123, wherein after at least 13 weeks of treatment, the subject's food preoccupation is decreased.

Embodiment II-125. The method of embodiment II-124, wherein the subject's Food Related Problems Questionnaire (FRPQ) score is decreased compared to prior to commencement of treatment.

Embodiment II-126. The method of any one of embodiments II-71 to II-125, wherein after at least 13 weeks of treatment, the subject's muscle mass is increased.

Embodiment II-127. The method of any one of embodiments II-71 to II-126, wherein after at least 13 weeks of treatment, the subject's lean mass index (LMI) is increased.

Embodiment II-128. The method of embodiment II-127, wherein the subject's LMI is increased by at least 10% compared to prior to commencement of treatment.

Embodiment II-129. The method of any one of embodiments II-71 to II-128, wherein after at least 13 weeks of treatment, the subject's fat mass index (FMI) is decreased.

Embodiment II-130. The method of embodiment II-129, wherein the subject's FMI is decreased by at least 5% compared to prior to commencement of treatment.

Embodiment II-131. The method of any one of embodiments II-71 to II-130, wherein after at least 13 weeks of treatment, one or more behavioral or cognitive complications of PWS is reduced in the subject.

Embodiment II-132. The method of embodiment II-131, wherein the one or more behavioral complications of PWS is selected from the group consisting of aggressive behavior, compulsive behavior, anxiety, rigidity or resistance to change, and temper tantrums.

Embodiment II-133. The method of any one of embodiments II-71 to II-132, wherein after at least 13 weeks of treatment, the subject's cognitive ability is improved.

Embodiment II-134. The method of any one of embodiments II-71 to II-133, wherein after at least 13 weeks of treatment, the subject's amount of circulating C-Reactive Protein (CRP) is decreased.

Embodiment II-135. A method of treating hyperphagia in a subject having Prader-Willi Syndrome, the method comprising:
(a) administering to the subject a therapeutically effective amount of diazoxide choline, wherein the subject is concomitantly administered one or more strong CYP1A2 inhibitors; and
(b) adjusting the therapeutically effective amount of diazoxide choline administered to the subject.

Embodiment II-136. The method of embodiment II-135, wherein the administration of diazoxide choline is initiated at a reduced starting dose of diazoxide choline and then increased each 2-3 weeks to reach a reduced target maintenance dose of diazoxide choline.

Embodiment II-137. The method of embodiment II-136, wherein the reduced target maintenance dose of diazoxide choline is reached after 4-10 weeks.

Embodiment II-138. The method of embodiment II-136, wherein the reduced starting dose of diazoxide choline is between about 3-100 mg/day of diazoxide choline.

Embodiment II-139. The method of embodiment II-138, wherein the reduced starting dose of diazoxide choline is between about 3-18 mg/day of diazoxide choline.

Embodiment II-140. The method of embodiment II-138, wherein the reduced starting dose of diazoxide choline is between about 25-100 mg/day of diazoxide choline.

Embodiment II-141. The method of embodiment II-136, wherein after 2-3 weeks the reduced starting dose of diazoxide choline is increased to a reduced second dose of diazoxide choline.

Embodiment II-142. The method of embodiment II-141, wherein the reduced second dose of diazoxide choline is between about 5-200 mg/day of diazoxide choline.

Embodiment II-143. The method of embodiment II-142, wherein the reduced second dose of diazoxide choline is between about 5-36 mg/day of diazoxide choline.

Embodiment II-144. The method of embodiment II-142, wherein the reduced second dose of diazoxide choline is between about 25-200 mg/day of diazoxide choline.

Embodiment II-145. The method of embodiment II-141, wherein after 2-3 weeks the reduced second dose of diazoxide choline is increased to a reduced third dose of diazoxide choline.

Embodiment II-146. The method of embodiment II-145, wherein the reduced third dose of diazoxide choline is between about 7-300 mg/day of diazoxide choline.

Embodiment II-147. The method of embodiment II-146, wherein the reduced third dose of diazoxide choline is between about 50-300 mg/day of diazoxide choline.

Embodiment II-148. The method of embodiment II-145, wherein after 2-3 weeks the reduced third dose of diazoxide choline is increased to the reduced target maintenance dose of diazoxide choline.

Embodiment II-149. The method of embodiment II-148, wherein the reduced target maintenance dose of diazoxide choline is between about 9-325 mg/day of diazoxide choline.

Embodiment II-150. The method of embodiment II-149, wherein the reduced target maintenance dose of diazoxide choline is between about 75-325 mg/day of diazoxide choline.

Embodiment II-151. The method of embodiment II-135, wherein the once-daily administration of diazoxide choline is in an amount from about 25 mg to about 75 mg.

Embodiment II-152. The method of embodiment II-135, wherein the once-daily administration of diazoxide choline is in an amount from about 50 mg to about 100 mg.

Embodiment II-153. The method of embodiment II-135, wherein the once-daily administration of diazoxide choline is in an amount from about 50 mg to about 150 mg.

Embodiment II-154. The method of embodiment II-135, wherein the once-daily administration of diazoxide choline is in an amount from about 100 mg to about 250 mg.

Embodiment II-155. The method of embodiment II-135, wherein the once-daily administration of diazoxide choline is in an amount from about 100 mg to about 300 mg.

Embodiment II-156. The method of embodiment II-135, wherein the once-daily administration of diazoxide choline is in an amount from about 100 mg to about 325 mg.

Embodiment II-157. The method of embodiment II-135, wherein the one or more strong CYP1A2 inhibitors is selected from the group consisting of amiodarone, ciprofloxacin, and fluvoxamine.

Embodiment II-158. The method of embodiment II-135, wherein the therapeutically effective amount of diazoxide is reduced relative to the therapeutically effective amount for the subject if not concomitantly administered one or more strong CYP1A2 inhibitors.

Embodiment II-159. A method of treating a subject in need thereof, the method comprising:
(a) administering to the subject a therapeutically effective amount of diazoxide choline wherein the subject is concomitantly administered one or more strong CYP1A2 inhibitors; and
(b) adjusting the therapeutically effective amount of diazoxide choline administered to the subject;
wherein the subject is a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance.

Embodiment II-160. A method of treating hyperphagia in a subject having Prader-Willi Syndrome, the method comprising:
(a) administering to the subject a therapeutically effective amount of diazoxide choline, wherein the subject is concomitantly administered one or more strong CYP3A4 inhibitors;
(b) monitoring the subject for adverse reactions to diazoxide choline; and
(c) if the subject exhibits adverse reactions, adjusting the therapeutically effective amount of diazoxide choline administered to the subject.

Embodiment II-161. The method of embodiment II-160, wherein the therapeutically effective amount of diazoxide choline is reduced by between about 15%-30% relative to the therapeutically effective amount for the subject if not concomitantly administered one or more strong CYP3A4 inhibitors.

Embodiment II-162. The method of embodiment II-160, wherein the adverse reactions include hyperglycemia and the risk of type 2 diabetes or ketoacidosis, peripheral edema and fluid overload, and hypertrichosis.

Embodiment II-163. The method of embodiment II-160, wherein the one or more strong CYP3A4 inhibitors is selected from the group consisting of ceritinib, clarithromycin, delaviridone, idelalisib, indinavir, itraconazole, ketoconazole, mibefradil, nefazodone, nelfinivir, ribociclib, ritonavir, saquinavir, telaprevir, telithromycin, tucatinib, voriconazole.

Embodiment II-164. A method of treating a subject in need thereof, the method comprising:
(a) administering to the subject a therapeutically effective amount of diazoxide choline wherein the subject is concomitantly administered one or more strong CYP3A4 inhibitors;
(b) monitoring the subject for adverse reactions to diazoxide choline; and
(c) if the subject exhibits adverse reactions, adjusting the therapeutically effective amount of diazoxide choline administered to the subject;
wherein the subject is a subject having Prader-Willi Syndrome, a subject having Smith-Magenis Syndrome, a subject with an elevated triglyceride level, a subject with an elevated non-HDL cholesterol level, a subject that is obese, a subject with a reduced fasting glucose level, a subject with an elevated fasting insulin level, or a subject with insulin resistance.

Embodiment II-165. A method of treating hyperphagia in a subject having Prader-Willi Syndrome, the method comprising:
(a) orally administering to the subject 25 mg of diazoxide choline once daily for an initial period of four weeks;
(b) after the initial four-week period, orally administering to the subject 50 mg of diazoxide choline once daily for a subsequent period of two weeks; and
(c) after the subsequent two-week period, orally administering to the subject 75 mg of diazoxide choline once daily;
wherein the subject is concomitantly being administered one or more strong CYP1A2 inhibitors.

Embodiment II-166. The method of embodiment II-165, wherein the subject weighs 20 kg to less than 30 kg.

Embodiment II-167. A method of treating hyperphagia in a subject having Prader-Willi Syndrome, the method comprising:
(a) orally administering to the subject 50 mg of diazoxide choline once daily for an initial period of two weeks; and
(b) after the initial two-week period, orally administering to the subject 100 mg of diazoxide choline once daily;
wherein the subject is concomitantly being administered one or more strong CYP1A2 inhibitors.

Embodiment II-168. The method of embodiment II-167, wherein the subject weighs 30 kg to less than 40 kg.

Embodiment II-169. A method of treating hyperphagia in a subject having Prader-Willi Syndrome, the method comprising:
(a) orally administering to the subject 50 mg of diazoxide choline once daily for an initial period of two weeks;
(b) after the initial two-week period, orally administering to the subject 100 mg of diazoxide choline once daily for a second two-week period; and
(c) after the second two-week period, orally administering to the subject 150 mg of diazoxide choline once daily;
wherein the subject is concomitantly being administered one or more strong CYP1A2 inhibitors.

Embodiment II-170. The method of embodiment II-169, wherein the subject weighs 40 kg to less than 65 kg.

Embodiment II-171. A method of treating hyperphagia in a subject having Prader-Willi Syndrome, the method comprising:
(a) orally administering to the subject a starting dosage of 100 mg of diazoxide choline once daily for an initial period of two weeks;
(b) after the initial two-week period, orally administering to the subject a first titration dosage of diazoxide choline once daily for a second two-week period, wherein the first titration dosage is 150 mg or 200 mg of diazoxide choline;
(c) after the second two-week period, orally administering to the subject a second titration dosage of diazoxide choline once daily for a third two-week period, wherein the second titration dosage is 200 mg, 250 mg, or 300 mg of diazoxide choline; and
(d) after the third two-week period, orally administering to the subject a target maintenance dose of diazoxide choline once daily, wherein the target maintenance dose is 250 mg, 300 mg, or 325 mg of diazoxide choline;
wherein the subject is concomitantly being administered one or more strong CYP1A2 inhibitors.

Embodiment II-172. The method of embodiment II-171, wherein the starting dosage is 100 mg, the first titration dosage is 150 mg, the second titration dosage is 200 mg, and the target maintenance dosage is 250 mg.

Embodiment II-173. The method of embodiment II-171, wherein the starting dosage is 100 mg, the first titration dosage is 200 mg, the second titration dosage is 250 mg, and the target maintenance dosage is 300 mg.

Embodiment II-174. The method of embodiment II-171, wherein the starting dosage is 100 mg, the first titration dosage is 200 mg, the second titration dosage is 300 mg, and the target maintenance dosage is 325 mg.

Embodiment II-175. The method of embodiment II-172, wherein the subject weighs 65 kg to less than 100 kg.

Embodiment II-176. The method of embodiment II-173, wherein the subject weighs 100 kg to less than 135 kg.

Embodiment II-177. The method of embodiment II-174, wherein the subject weighs 135 kg or more.

Embodiment II-178. The method of embodiment II-165, wherein the one or more strong CYP1A2 inhibitors is amiodarone, ciprofloxacin, or fluvoxamine.

Embodiment II-179. The method of embodiment II-165, wherein the strong CYP1A2 inhibitor is fluvoxamine.

Embodiment II-180. The method of embodiment II-167, wherein the one or more strong CYP1A2 inhibitors is amiodarone, ciprofloxacin, or fluvoxamine.

Embodiment II-181. The method of embodiment II-167, wherein the strong CYP1A2 inhibitor is fluvoxamine.

Embodiment II-182. The method of embodiment II-169, wherein the one or more strong CYP1A2 inhibitors is amiodarone, ciprofloxacin, or fluvoxamine.

Embodiment II-183. The method of embodiment II-169, wherein the strong CYP1A2 inhibitor is fluvoxamine.

Embodiment II-184. The method of embodiment II-171, wherein the one or more strong CYP1A2 inhibitors is amiodarone, ciprofloxacin, or fluvoxamine.

Embodiment II-185. The method of embodiment II-171, wherein the strong CYP1A2 inhibitor is fluvoxamine.

Embodiment II-186. The method of embodiment II-172, wherein the one or more strong CYP1A2 inhibitors is amiodarone, ciprofloxacin, or fluvoxamine.

Embodiment II-187. The method of embodiment II-172, wherein the strong CYP1A2 inhibitor is fluvoxamine.

Embodiment II-188. The method of embodiment II-173, wherein the one or more strong CYP1A2 inhibitors is amiodarone, ciprofloxacin, or fluvoxamine.

Embodiment II-189. The method of embodiment II-173, wherein the strong CYP1A2 inhibitor is fluvoxamine.

Embodiment II-190. The method of embodiment II-174, wherein the one or more strong CYP1A2 inhibitors is amiodarone, ciprofloxacin, or fluvoxamine.

Embodiment II-191. The method of embodiment II-174, wherein the strong CYP1A2 inhibitor is fluvoxamine.

Embodiment II-192. A method of treating hyperphagia in a subject having Prader-Willi Syndrome, wherein said subject is in need of concomitant administration of one or more strong CYP1A2 inhibitors and is administered a once daily oral maintenance dose of diazoxide choline, the method comprising:
(a) orally administering a once daily reduced maintenance dose of diazoxide choline, and
(b) administering one or more CYP1A2 inhibitors;
wherein,
   (i) if the maintenance dose is 100 mg, then the reduced maintenance dose is 75 mg,
   (ii) if the maintenance dose is 150 mg, then the reduced maintenance dose is 100 mg,
   (iii) if the maintenance dose is 225 mg, then the reduced maintenance dose is 150 mg,
   (iv) if the maintenance dose is 375 mg, then the reduced maintenance dose is 250 mg,
   (v) if the maintenance dose is 450 mg, then the reduced maintenance dose is 300 mg, or
   (vi) if the maintenance dose is 525 mg, then the reduced maintenance dose is 325 mg.

Embodiment II-193. A method of treating hyperphagia in a subject having Prader-Willi Syndrome, wherein said subject is in need of concomitant administration of one or more strong CYP1A2 inhibitors and is administered a once daily oral maintenance dose of diazoxide choline, the method comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 25 mg of diazoxide choline once daily for an initial period of four weeks;
   (ii) after the initial four-week period, orally administering to the subject 50 mg of diazoxide choline once daily for a subsequent period of two weeks; and
   (iii) after the subsequent two-week period, orally administering to the subject 75 mg of diazoxide choline once daily.

Embodiment II-194. The method of embodiment II-193, wherein the subject weighs 20 kg to less than 30 kg.

Embodiment II-195. A method of treating hyperphagia in a subject having Prader-Willi Syndrome, wherein said subject is in need of concomitant administration of one or more strong CYP1A2 inhibitors and is administered a once daily oral maintenance dose of diazoxide choline, the method comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 50 mg of diazoxide choline once daily for an initial period of two weeks; and
   (ii) after the initial two-week period, orally administering to the subject 100 mg of diazoxide choline once daily.

Embodiment II-196. The method of embodiment II-195, wherein the subject weighs 30 kg to less than 40 kg.

Embodiment II-197. A method of treating hyperphagia in a subject having Prader-Willi Syndrome, wherein said subject is in need of concomitant administration of one or more strong CYP1A2 inhibitors and is administered a once daily oral maintenance dose of diazoxide choline, the method comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject 50 mg of diazoxide choline once daily for an initial period of two weeks;
   (ii) after the initial two-week period, orally administering to the subject 100 mg of diazoxide choline once daily for a subsequent period of two weeks; and
   (iii) after the subsequent two-weeks period, orally administering to the subject 150 mg of diazoxide choline once daily.

Embodiment II-198. The method of embodiment II-197, wherein the subject weighs 40 kg to less than 65 kg.

Embodiment II-199. A method of treating hyperphagia in a subject having Prader-Willi Syndrome, wherein said subject is in need of concomitant administration of one or more strong CYP1A2 inhibitors and is administered a once daily oral maintenance dose of diazoxide choline, the method comprising:
(a) discontinuing the once daily oral maintenance dose of diazoxide choline, and
(b) after discontinuing the once daily oral maintenance dose of diazoxide choline, administering one or more strong CYP1A2 inhibitors to the subject and concomitantly administering diazoxide choline to the subject on the following schedule:
   (i) orally administering to the subject a starting dosage of 100 mg of diazoxide choline once daily for an initial period of two weeks;
   (ii) after the initial two-week period, orally administering to the subject a first titration dosage of diazoxide choline once daily for a second two-week period, wherein the first titration dosage is 150 mg or 200 mg of diazoxide choline;
   (iii) after the second two-week period, orally administering to the subject a second titration dosage of diazoxide choline once daily for a third two-week period, wherein the second titration dosage is 200 mg, 250 mg, or 300 mg of diazoxide choline; and
   (iv) after the third two-week period, orally administering to the subject a target maintenance dose of diazoxide choline once daily, wherein the target maintenance dose is 250 mg, 300 mg, or 325 mg of diazoxide choline.

Embodiment II-200. The method of embodiment II-199, wherein the starting dosage is 100 mg, the first titration dosage is 150 mg, the second titration dosage is 200 mg, and the target maintenance dosage is 250 mg.

Embodiment II-201. The method of embodiment II-199, wherein the starting dosage is 100 mg, the first titration dosage is 200 mg, the second titration dosage is 250 mg, and the target maintenance dosage is 300 mg.

Embodiment II-202. The method of embodiment II-199, wherein the starting dosage is 100 mg, the first titration dosage is 200 mg, the second titration dosage is 300 mg, and the target maintenance dosage is 325 mg.

Embodiment II-203. The method of embodiment II-200, wherein the subject weighs 65 kg to less than 100 kg.

Embodiment II-204. The method of embodiment II-201, wherein the subject weighs 100 kg to less than 135 kg.

Embodiment II-205. The method of embodiment II-202, wherein the subject weighs 135 kg or more.

Embodiment III-1. A method of treating hyperphagia in a subject having Prader-Willi Syndrome, the method comprising:
(a) orally administering to the subject 25 mg of diazoxide choline once daily for an initial period of at least two weeks;
(b) after the initial period of at least two weeks, orally administering to the subject 25-50 mg of diazoxide choline once daily for a second period of at least two weeks;
(c) after the second period of at least two weeks, orally administering to the subject 50-75 mg of diazoxide choline once daily for a third period of at least two weeks; and
   (i) after the third period of at least two weeks, orally administering to the subject a target maintenance dose of 50-75 mg of diazoxide choline once daily; or
   (ii) after the third period of at least two weeks, orally administering to the subject a maximum target maintenance dose of 100 mg of diazoxide choline once daily;
wherein the subject has one or more clinically relevant comorbidities prior to treatment with diazoxide choline.

Embodiment III-2. A method of treating hyperphagia in a subject having Prader-Willi Syndrome, the method comprising:
(a) orally administering to the subject 25 mg of diazoxide choline once daily for an initial period of at least two weeks;
(b) after the initial period of at least two weeks, orally administering to the subject 50 mg of diazoxide choline once daily for a second period of at least two weeks;
(c) after the second period of at least two weeks, orally administering to the subject 75 mg of diazoxide choline once daily for a third period of at least two weeks; and
(d) after the third period of at least two weeks, orally administering to the subject a target maintenance dose of 100 mg of diazoxide choline once daily;
wherein the subject has one or more clinically relevant comorbidities prior to treatment with diazoxide choline.

Embodiment III-3. The method of embodiment III-1 or III-2, wherein the subject weighs from about 20 kg to less than 30 kg.

Embodiment III-4. A method of treating hyperphagia in a subject having Prader-Willi Syndrome, the method comprising:
(a) orally administering to the subject 50-75 mg of diazoxide choline once daily for an initial period of at least two weeks;
(b) after the initial period of at least two weeks, orally administering to the subject 75-100 mg of diazoxide choline once daily for a subsequent period of at least four weeks; and
   (i) after the subsequent period of at least four weeks, orally administering to the subject a target maintenance dose of 100 mg of diazoxide choline once daily; or
   (ii) after the subsequent period of at least four weeks, orally administering to the subject a maximum target maintenance dose of 150 mg of diazoxide choline once daily;
wherein the subject has one or more clinically relevant comorbidities prior to treatment with diazoxide choline.

Embodiment III-5. A method of treating hyperphagia in a subject having Prader-Willi Syndrome, the method comprising:
(a) orally administering to the subject 75 mg of diazoxide choline once daily for an initial period of at least two weeks; and
(b) after the initial period of at least two weeks, orally administering to the subject a target maintenance dose of 150 mg of diazoxide choline once daily;
wherein the subject has one or more clinically relevant comorbidities prior to treatment with diazoxide choline.

Embodiment III-6. The method of embodiment III-4 or III-5, wherein the subject weighs from 30 kg to less than 40 kg.

Embodiment III-7. A method of treating hyperphagia in a subject having Prader-Willi Syndrome, the method comprising:
(a) orally administering to the subject 50-75 mg of diazoxide choline once daily for an initial period of at least two weeks;
(b) after the initial period of at least two weeks, orally administering to the subject 75-150 mg of diazoxide choline once daily for a subsequent period of at least four weeks; and
   (i) after the subsequent period of at least four weeks, orally administering to the subject a target maintenance dose of 150 mg of diazoxide choline once daily; or
   (ii) after the subsequent period of at least four weeks, orally administering to the subject a maximum target maintenance dose of 225 mg of diazoxide choline once daily;
wherein the subject has one or more clinically relevant comorbidities prior to treatment with diazoxide choline.

Embodiment III-8. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks; and
(c) after the second period of at least two weeks, orally administering to the subject a target maintenance dose of 225 mg of diazoxide choline once daily;
wherein the subject has one or more clinically relevant comorbidities prior to treatment with diazoxide choline.

Embodiment III-9. The method of embodiment III-7 or III-8, wherein the subject weighs from 40 kg to less than 65 kg.

Embodiment III-10. A method of treating hyperphagia in a subject having Prader-Willi Syndrome, the method comprising:
(a) orally administering to the subject 75-100 mg of diazoxide choline once daily for an initial period of at least two weeks;
(b) after the initial period of at least two weeks, orally administering to the subject 150-200 mg of diazoxide choline once daily for a second period of at least two weeks;
(c) after the second period of at least two weeks, orally administering to the subject 200-250 mg of diazoxide choline once daily for a third period of at least two weeks; and
   (i) after the third period of at least two weeks, orally administering to the subject a target maintenance dose of 200-250 mg of diazoxide choline once daily; or
   (ii) after the third period of at least two weeks, orally administering to the subject a maximum target maintenance dose of 375 mg of diazoxide choline once daily;
wherein the subject has one or more clinically relevant comorbidities prior to treatment with diazoxide choline.

Embodiment III-11. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks; and
(e) after the fourth period of at least two weeks, orally administering to the subject a target maintenance dose of 375 mg of diazoxide choline once daily;
wherein the subject has one or more clinically relevant comorbidities prior to treatment with diazoxide choline.

Embodiment III-12. The method of embodiment III-10 or III-11, wherein the subject weighs from 65 kg to less than 100 kg.

Embodiment III-13. A method of treating hyperphagia in a subject having Prader-Willi Syndrome, the method comprising:
(a) orally administering to the subject 75-100 mg of diazoxide choline once daily for an initial period of at least two weeks;
(b) after the initial period of at least two weeks, orally administering to the subject 150-225 mg of diazoxide choline once daily for a second period of at least two weeks;
(c) after the second period of at least two weeks, orally administering to the subject 225-300 mg of diazoxide choline once daily for a third period of at least two weeks; and
   (i) after the third period of at least two weeks, orally administering to the subject a target maintenance dose of 300-400 mg of diazoxide choline once daily; or
   (ii) after the third period of at least two weeks, orally administering to the subject a maximum target maintenance dose of 450 mg of diazoxide choline once daily;
wherein the subject has one or more clinically relevant comorbidities prior to treatment with diazoxide choline.

Embodiment III-14. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks; and
(f) after the fifth period of at least two weeks, orally administering to the subject a target maintenance dose of 450 mg of diazoxide choline once daily;
wherein the subject has one or more clinically relevant comorbidities prior to treatment with diazoxide choline.

Embodiment III-15. The method of embodiment III-13 or III-14, wherein the subject weighs from 100 kg to less than 135 kg.

Embodiment III-16. A method of treating hyperphagia in a subject having Prader-Willi Syndrome, the method comprising:
(a) orally administering to the subject 75-100 mg of diazoxide choline once daily for an initial period of at least two weeks;
(b) after the initial period of at least two weeks, orally administering to the subject 150-225 mg of diazoxide choline once daily for a second period of at least two weeks;
(c) after the second period of at least two weeks, orally administering to the subject 225-300 mg of diazoxide choline once daily for a third period of at least two weeks; and
   (i) after the third period of at least two weeks, orally administering to the subject a target maintenance dose of 300-400 mg of diazoxide choline once daily; or
   (ii) after the third period of at least two weeks, orally administering to the subject a maximum target maintenance dose of 525 mg of diazoxide choline once daily;
wherein the subject has one or more clinically relevant comorbidities prior to treatment with diazoxide choline.

Embodiment III-17. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks;
(f) after the fifth period of at least two weeks, orally administering to the subject 450 mg of diazoxide choline once daily for a sixth period of at least two weeks; and
(g) after the sixth period of at least two weeks, orally administering to the subject a target maintenance dose of 525 mg of diazoxide choline once daily;
wherein the subject has one or more clinically relevant comorbidities prior to treatment with diazoxide choline.

Embodiment III-18. The method of embodiment III-16 or III-17, wherein the subject weighs 135 kg or more.

Embodiment III-19. The method of any one of embodiments III-1 to III-18, wherein the one or more clinically relevant comorbidities is a cardiovascular, pulmonary, or metabolic/endocrine comorbidity.

Embodiment III-20. The method of any one of embodiments III-1 to III-19, wherein the one or more clinically relevant comorbidities is a history of edema, edema, fluid retention, fluid overload, congestive heart failure, compromised cardiac reserve, or heart disease.

Embodiment III-21. The method of any one of embodiments III-1 to III-19, wherein the one or more clinically relevant comorbidities is a history of asthma, asthma, Chronic Obstructive Pulmonary Disease (COPD), obstructive sleep apnea, or central sleep apnea.

Embodiment III-22. The method of any one of embodiments III-1 to III-19, wherein the one or more clinically relevant comorbidities is obesity, hyperglycemia, elevated fasting glucose, elevated HbA1c, prediabetes, diabetes, or concomitant use of corticosteroids or medications known to impact blood glucose.

Embodiment III-23. The method of any one of embodiments III-1 to III-18, wherein the one or more clinically relevant comorbidities is concomitant use of one or more medications that could contribute to hyperglycemia, fluid retention, or weight gain.

Embodiment III-24. The method of any one of embodiments III-1 to III-19 or III-22, wherein the one or more clinically relevant comorbidities is hyperglycemia.

Embodiment III-25. The method of embodiment III-24, wherein the method further comprises periodically monitoring fasting glucose during treatment.

Embodiment III-26. The method of embodiment III-25, wherein the monitoring takes place at least once a week during titration.

Embodiment III-27. The method of embodiment III-25, wherein the monitoring takes place every two weeks after titration.

Embodiment III-28. The method of embodiment III-25, wherein the monitoring takes place as clinically indicated after titration.

Embodiment III-29. The method of any one of embodiments III-1 to III-19 or III-22, wherein the one or more clinically relevant comorbidities is elevated HbA1c.

Embodiment III-30. The method of embodiment III-29, wherein the method further comprises periodically monitoring HbA1c during treatment.

Embodiment III-31. The method of embodiment III-30, wherein the monitoring takes place every 3 months.

Embodiment III-32. The method of embodiment III-30, wherein the monitoring takes place as clinically indicated.

Embodiment III-33. The method of any one of embodiments III-1 to III-20, wherein the one or more clinically relevant comorbidities is edema or fluid overload.

Embodiment III-34. The method of embodiment III-33, wherein the method further comprises periodically monitoring body weight during treatment.

Embodiment III-35. The method of embodiment III-33 or III-34, wherein the method further comprises periodically assessing the subject for swelling of a body part during treatment.

Embodiment III-36. The method of embodiment III-35, wherein the body part is an ankle, foot, or leg.

Embodiment III-37. The method of any one of embodiments III-33 to III-36, wherein the method further comprises periodically assessing the subject for one or more respiratory symptoms during treatment.

Embodiment III-38. The method of embodiment III-37, wherein the one or more respiratory symptoms is dyspnea or shortness of breath.

Embodiment III-39. The method of any one of embodiments III-34 to III-38, wherein the monitoring takes place once a week during titration.

Embodiment III-40. The method of any one of embodiments III-34 to III-38, wherein the monitoring takes place at regular intervals after titration.

Embodiment III-41. A method of treating hyperphagia in a subject having Prader-Willi Syndrome, the method comprising orally administering to the subject diazoxide choline once daily, wherein the subject has one or more clinically relevant comorbidities prior to treatment with diazoxide choline, and wherein the amount of diazoxide choline administered to the subject is reduced by between ⅓ to ½ relative to the amount that would be administered to the subject if the subject did not have one or more clinically relevant comorbidities.

Embodiment III-42. The method of embodiment III-41, wherein the amount of diazoxide choline administered to the subject is reduced by ⅓ relative to the amount that would be administered to the subject if the subject did not have one or more clinically relevant comorbidities.

Embodiment III-43. The method of embodiment III-41, wherein the amount of diazoxide choline administered to the subject is reduced by ½ relative to the amount that would be administered to the subject if the subject did not have one or more clinically relevant comorbidities.

Embodiment III-44. A method of treating hyperphagia in a subject having Prader-Willi Syndrome, the method comprising:
(a) orally administering to the subject 25 mg of diazoxide choline once daily for an initial period of at least two weeks;
(b) after the initial period of at least two weeks, orally administering to the subject 50 mg of diazoxide choline once daily for a second period of at least two weeks;
(c) after the second period of at least two weeks, orally administering to the subject 75 mg of diazoxide choline once daily for a third period of at least two weeks; and
(d) after the third period of at least two weeks, orally administering to the subject a target maintenance dose of 100 mg of diazoxide choline once daily;
wherein the subject experiences one or more adverse reactions during treatment with diazoxide choline.

Embodiment III-45. The method of embodiment III-44, wherein the subject weighs from about 20 kg to less than 30 kg.

Embodiment III-46. A method of treating hyperphagia in a subject having Prader-Willi Syndrome, the method comprising:
(a) orally administering to the subject 75 mg of diazoxide choline once daily for an initial period of at least two weeks; and
(b) after the initial period of at least two weeks, orally administering to the subject a target maintenance dose of 150 mg of diazoxide choline once daily;
wherein the subject experiences one or more adverse reactions during treatment with diazoxide choline.

Embodiment III-47. The method of embodiment III-46, wherein the subject weighs from 30 kg to less than 40 kg.

Embodiment III-48. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks; and
(c) after the second period of at least two weeks, orally administering to the subject a target maintenance dose of 225 mg of diazoxide choline once daily;
wherein the subject experiences one or more adverse reactions during treatment with diazoxide choline.

Embodiment III-49. The method of embodiment III-48, wherein the subject weighs from 40 kg to less than 65 kg.

Embodiment III-50. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks; and
(e) after the fourth period of at least two weeks, orally administering to the subject a target maintenance dose of 375 mg of diazoxide choline once daily;
wherein the subject experiences one or more adverse reactions during treatment with diazoxide choline.

Embodiment III-51. The method of embodiment III-50, wherein the subject weighs from 65 kg to less than 100 kg.

Embodiment III-52. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks; and
(f) after the fifth period of at least two weeks, orally administering to the subject a target maintenance dose of 450 mg of diazoxide choline once daily;
wherein the subject experiences one or more adverse reactions during treatment with diazoxide choline.

Embodiment III-53. The method of embodiment III-52, wherein the subject weighs from 100 kg to less than 135 kg.

Embodiment III-54. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks;
(f) after the fifth period of at least two weeks, orally administering to the subject 450 mg of diazoxide choline once daily for a sixth period of at least two weeks; and
(g) after the sixth period of at least two weeks, orally administering to the subject a target maintenance dose of 525 mg of diazoxide choline once daily;
wherein the subject experiences one or more adverse reactions during treatment with diazoxide choline.

Embodiment III-55. The method of embodiment III-54, wherein the subject weighs 135 kg or more.

Embodiment III-56. The method of any one of embodiments III-44 to III-55, wherein the once daily administration of diazoxide choline is paused for a period of time following the development of one or more adverse reactions.

Embodiment III-57. The method of any one of embodiments III-44 to III-55, wherein the once daily administration dose of diazoxide choline is reduced following the development of one or more adverse reactions.

Embodiment III-58. The method of any one of embodiments III-44 to III-57, wherein the subject is concomitantly administered one or more risk mitigating medications following the development of one or more adverse reactions.

Embodiment III-59. The method of embodiment III-58, wherein the one or more risk mitigating medications is a glucose-lowering agent or a diuretic.

Embodiment III-60. The method of any one of embodiments III-44 to III-59, wherein the one or more adverse reactions is a cardiovascular, pulmonary, or metabolic/endocrine adverse reaction.

Embodiment III-61. The method of any one of embodiments III-44 to III-60, wherein the one or more adverse reactions is edema, fluid retention, fluid overload, congestive heart failure, reduced cardiac reserve, or heart disease.

Embodiment III-62. The method of any one of embodiments III-44 to III-60, wherein the one or more adverse reactions is asthma, Chronic Obstructive Pulmonary Disease (COPD), obstructive sleep apnea, or central sleep apnea.

Embodiment III-63. The method of any one of embodiments III-44 to III-60, wherein the one or more adverse reactions is obesity, hyperglycemia, elevated fasting glucose, elevated HbA1c, prediabetes, or diabetes.

Embodiment III-64. The method of any one of embodiments III-1 to III-40 or III-44 to III-63, wherein the initial period of at least two weeks is two, three, or four weeks.

Embodiment III-65. The method of any one of embodiments III-1 to III-3, III-8 to 111-40, 111-44 to III-45, or III-48 to III-64, wherein the second period of at least two weeks is two, three, or four weeks.

Embodiment III-66. The method of any one of embodiments III-1 to III-3, III-10 to 111-40, 111-44 to III-45, or III-50 to III-65, wherein the third period of at least two weeks is two, three, or four weeks.

Embodiment III-67. The method of any one of embodiments III-11 to III-12, III-14 to 111-15, 111-17 to III-40, or III-50 to III-66, wherein the fourth period of at least two weeks is two, three, or four weeks.

Embodiment III-68. The method of any one of embodiments III-14 to III-15, III-17 to III-40, or III-52 to III-67, wherein the fifth period of at least two weeks is two, three, or four weeks.

Embodiment III-69. The method of any one of embodiments III-17 to III-40 or III-54 to III-68, wherein the sixth period of at least two weeks is two, three, or four weeks.

Embodiment III-70. The method of any one of embodiments 111-4, 111-6 to 111-7, 111-9, 111-19 to III-40, or III-64, wherein the subsequent period of at least four weeks is four, five, six, seven, or eight weeks.

Embodiment III-71. The method of any one of embodiments III-1 to III-40 or III-44 to III-70, wherein the target maintenance dose is reached after 12 weeks of treatment.

Embodiment III-72. The method of any one of embodiments III-1 to III-40 or III-44 to III-70, wherein the target maintenance dose is reached after 14 weeks of treatment.

Embodiment III-73. The method of any one of embodiments III-1 to III-40 or III-44 to III-70, wherein the target maintenance dose is reached after 16 weeks of treatment.

Embodiment III-74. The method of any one of embodiments III-1 to III-40 or III-44 to III-70, wherein the target maintenance dose is reached after 20 weeks of treatment.

Embodiment III-75. The method of any one of embodiments III-1 to III-40 or III-44 to III-70, wherein the target maintenance dose is reached after 24 weeks of treatment.

Embodiment III-76. The method of any one of embodiments III-1 to III-3, III-19 to III-40, III-44 to III-45, III-56 to III-66, or III-71 to III-75, wherein after the target maintenance dose is reached, the amount of diazoxide choline orally administered once daily to the subject is increased by no more than 25 mg every adjustment period, wherein every adjustment period has a duration of at least two weeks, wherein the amount of diazoxide choline orally administered once daily to the subject is no more than 5.8 mg/kg.

Embodiment III-77. The method of any one of embodiments III-4 to III-40 or III-46 to III-75, wherein after the target maintenance dose is reached, the amount of diazoxide choline orally administered once daily to the subject is increased by no more than 75 mg every adjustment period, wherein every adjustment period has a duration of at least two weeks, wherein the amount of diazoxide choline orally administered once daily to the subject is no more than 5.8 mg/kg or 525 mg, whichever is lower.

Embodiment IV-1. A method of treating hyperphagia in a subject having Prader-Willi Syndrome,
the method comprising:
   (a1) orally administering to the subject 25 mg of diazoxide choline once daily for an initial period of four weeks;
   (b1) after the initial four-week period, orally administering to the subject 50 mg of diazoxide choline once daily for a subsequent period of two weeks; and
   (c1) after the subsequent two-week period, orally administering to the subject 75 mg of diazoxide choline once daily;
or
   (a2) orally administering to the subject 50 mg of diazoxide choline once daily for an initial period of two weeks; and
   (b2) after the initial two-week period, orally administering to the subject 100 mg of diazoxide choline once daily;
or
   (a3) orally administering to the subject 50 mg of diazoxide choline once daily for an initial period of two weeks;
   (b3) after the initial two-week period, orally administering to the subject 100 mg of diazoxide choline once daily for a second two-week period; and
   (c3) after the second two-week period, orally administering to the subject 150 mg of diazoxide choline once daily;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 150 mg of diazoxide choline;
   (c4) after the second two-week period, orally administering to the subject a second titration dosage of diazoxide choline once daily for a third two-week period, wherein the second titration dosage is 200 mg of diazoxide choline; and
   (d4) after the third two-week period, orally administering to the subject a target maintenance dose of diazoxide choline once daily, wherein the target maintenance dose is 250 mg of diazoxide choline;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 200 mg of diazoxide choline;
   (c4) after the second two-week period, orally administering to the subject a second titration dosage of diazoxide choline once daily for a third two-week period, wherein the second titration dosage is 250 mg of diazoxide choline; and
   (d4) after the third two-week period, orally administering to the subject a target maintenance dose of diazoxide choline once daily, wherein the target maintenance dose is 300 mg of diazoxide choline;
or
   (a4) orally administering to the subject a starting dosage of 100 mg of diazoxide choline once daily for an initial period of two weeks;
   (b4) after the initial two-week period, orally administering to the subject a first titration dosage of diazoxide choline once daily for a second two-week period, wherein the first titration dosage is 200 mg of diazoxide choline;
   (c4) after the second two-week period, orally administering to the subject a second titration dosage of diazoxide choline once daily for a third two-week period, wherein the second titration dosage is 300 mg of diazoxide choline; and
   (d4) after the third two-week period, orally administering to the subject a target maintenance dose of diazoxide choline once daily, wherein the target maintenance dose is 325 mg of diazoxide choline;
wherein the subject is concomitantly being administered one or more strong CYP1A2 inhibitors.

Embodiment IV-2. The method of embodiment IV-1, wherein the method comprises:
(a1) orally administering to the subject 25 mg of diazoxide choline once daily for an initial period of four weeks;
(b1) after the initial four-week period, orally administering to the subject 50 mg of diazoxide choline once daily for a subsequent period of two weeks; and
(c1) after the subsequent two-week period, orally administering to the subject 75 mg of diazoxide choline once daily;
wherein the subject is concomitantly being administered one or more strong CYP1A2 inhibitors.

Embodiment IV-3. The method of embodiment IV-2, wherein the subject weighs 20 kg to less than 30 kg.

Embodiment IV-4. The method of embodiment IV-1, wherein the method comprises:
(a2) orally administering to the subject 50 mg of diazoxide choline once daily for an initial period of two weeks; and
(b2) after the initial two-week period, orally administering to the subject 100 mg of diazoxide choline once daily;
wherein the subject is concomitantly being administered one or more strong CYP1A2 inhibitors.

Embodiment IV-5. The method of embodiment IV-4, wherein the subject weighs 30 kg to less than 40 kg.

Embodiment IV-6. The method of embodiment IV-1, wherein the method comprises:
(a3) orally administering to the subject 50 mg of diazoxide choline once daily for an initial period of two weeks;
(b3) after the initial two-week period, orally administering to the subject 100 mg of diazoxide choline once daily for a second two-week period; and
(c3) after the second two-week period, orally administering to the subject 150 mg of diazoxide choline once daily;
wherein the subject is concomitantly being administered one or more strong CYP1A2 inhibitors.

Embodiment IV-7. The method of embodiment IV-6, wherein the subject weighs 40 kg to less than 65 kg.

Embodiment IV-8. The method of embodiment IV-1, wherein the method comprises:
(a4) orally administering to the subject a starting dosage of 100 mg of diazoxide choline once daily for an initial period of two weeks;
(b4) after the initial two-week period, orally administering to the subject a first titration dosage of diazoxide choline once daily for a second two-week period, wherein the first titration dosage is 150 mg or 200 mg of diazoxide choline;
(c4) after the second two-week period, orally administering to the subject a second titration dosage of diazoxide choline once daily for a third two-week period, wherein the second titration dosage is 200 mg, 250 mg, or 300 mg of diazoxide choline; and
(d4) after the third two-week period, orally administering to the subject a target maintenance dose of diazoxide choline once daily, wherein the target maintenance dose is 250 mg, 300 mg, or 325 mg of diazoxide choline;
wherein the subject is concomitantly being administered one or more strong CYP1A2 inhibitors.

Embodiment IV-9. The method of embodiment IV-8, wherein the starting dosage is 100 mg, the first titration dosage is 150 mg, the second titration dosage is 200 mg, and the target maintenance dosage is 250 mg.

Embodiment IV-10. The method of embodiment IV-8, wherein the starting dosage is 100 mg, the first titration dosage is 200 mg, the second titration dosage is 250 mg, and the target maintenance dosage is 300 mg.

Embodiment IV-11. The method of embodiment IV-8, wherein the starting dosage is 100 mg, the first titration dosage is 200 mg, the second titration dosage is 300 mg, and the target maintenance dosage is 325 mg.

Embodiment IV-12. The method of embodiment IV-9, wherein the subject weighs 65 kg to less than 100 kg.

Embodiment IV-13. The method of embodiment IV-10, wherein the subject weighs 100 kg to less than 135 kg.

Embodiment IV-14. The method of embodiment IV-11, wherein the subject weighs 135 kg or more.

Embodiment IV-15. The method of embodiment IV-2, wherein the one or more strong CYP1A2 inhibitors is amiodarone, ciprofloxacin, or fluvoxamine.

Embodiment IV-16. The method of embodiment IV-2, wherein the strong CYP1A2 inhibitor is fluvoxamine.

Embodiment IV-17. The method of embodiment IV-4, wherein the one or more strong CYP1A2 inhibitors is amiodarone, ciprofloxacin, or fluvoxamine.

Embodiment IV-18. The method of embodiment IV-4, wherein the strong CYP1A2 inhibitor is fluvoxamine.

Embodiment IV-19. The method of embodiment IV-6, wherein the one or more strong CYP1A2 inhibitors is amiodarone, ciprofloxacin, or fluvoxamine.

Embodiment IV-20. The method of embodiment IV-6, wherein the strong CYP1A2 inhibitor is fluvoxamine.

Embodiment IV-21. The method of embodiment IV-8, wherein the one or more strong CYP1A2 inhibitors is amiodarone, ciprofloxacin, or fluvoxamine.

Embodiment IV-22. The method of embodiment IV-8, wherein the strong CYP1A2 inhibitor is fluvoxamine.

Embodiment IV-23. The method of embodiment IV-9, wherein the one or more strong CYP1A2 inhibitors is amiodarone, ciprofloxacin, or fluvoxamine.

Embodiment IV-24. The method of embodiment IV-9, wherein the strong CYP1A2 inhibitor is fluvoxamine.

Embodiment IV-25. The method of embodiment IV-10, wherein the one or more strong CYP1A2 inhibitors is amiodarone, ciprofloxacin, or fluvoxamine.

Embodiment IV-26. The method of embodiment IV-10, wherein the strong CYP1A2 inhibitor is fluvoxamine.

Embodiment IV-27. The method of embodiment IV-11, wherein the one or more strong CYP1A2 inhibitors is amiodarone, ciprofloxacin, or fluvoxamine.

Embodiment IV-28. The method of embodiment IV-11, wherein the strong CYP1A2 inhibitor is fluvoxamine.

Embodiment IV-29. A method of treating hyperphagia in a subject having Prader-Willi Syndrome, the method comprising:
(a) orally administering to the subject 25 mg of diazoxide choline once daily for an initial period of at least two weeks;
(b) after the initial period of at least two weeks, orally administering to the subject 25-50 mg of diazoxide choline once daily for a second period of at least two weeks;
(c) after the second period of at least two weeks, orally administering to the subject 50-75 mg of diazoxide choline once daily for a third period of at least two weeks; and
   (i) after the third period of at least two weeks, orally administering to the subject a target maintenance dose of 50-75 mg of diazoxide choline once daily; or
   (ii) after the third period of at least two weeks, orally administering to the subject a maximum target maintenance dose of 100 mg of diazoxide choline once daily;
wherein the subject has one or more clinically relevant comorbidities prior to treatment with diazoxide choline.

Embodiment IV-30. A method of treating hyperphagia in a subject having Prader-Willi Syndrome, the method comprising:
(a) orally administering to the subject 25 mg of diazoxide choline once daily for an initial period of at least two weeks;
(b) after the initial period of at least two weeks, orally administering to the subject 50 mg of diazoxide choline once daily for a second period of at least two weeks;
(c) after the second period of at least two weeks, orally administering to the subject 75 mg of diazoxide choline once daily for a third period of at least two weeks; and
(d) after the third period of at least two weeks, orally administering to the subject a target maintenance dose of 100 mg of diazoxide choline once daily;
wherein the subject has one or more clinically relevant comorbidities prior to treatment with diazoxide choline.

Embodiment IV-31. The method of embodiment IV-29 or IV-30, wherein the subject weighs from about 20 kg to less than 30 kg.

Embodiment IV-32. A method of treating hyperphagia in a subject having Prader-Willi Syndrome, the method comprising:
(a) orally administering to the subject 50-75 mg of diazoxide choline once daily for an initial period of at least two weeks;
(b) after the initial period of at least two weeks, orally administering to the subject 75-100 mg of diazoxide choline once daily for a subsequent period of at least four weeks; and
   (i) after the subsequent period of at least four weeks, orally administering to the subject a target maintenance dose of 100 mg of diazoxide choline once daily; or
   (ii) after the subsequent period of at least four weeks, orally administering to the subject a maximum target maintenance dose of 150 mg of diazoxide choline once daily;
wherein the subject has one or more clinically relevant comorbidities prior to treatment with diazoxide choline.

Embodiment IV-33. A method of treating hyperphagia in a subject having Prader-Willi Syndrome, the method comprising:
(a) orally administering to the subject 75 mg of diazoxide choline once daily for an initial period of at least two weeks; and
(b) after the initial period of at least two weeks, orally administering to the subject a target maintenance dose of 150 mg of diazoxide choline once daily;
wherein the subject has one or more clinically relevant comorbidities prior to treatment with diazoxide choline.

Embodiment IV-34. The method of embodiment IV-32 or IV-33, wherein the subject weighs from 30 kg to less than 40 kg.

Embodiment IV-35. A method of treating hyperphagia in a subject having Prader-Willi Syndrome, the method comprising:
(a) orally administering to the subject 50-75 mg of diazoxide choline once daily for an initial period of at least two weeks;
(b) after the initial period of at least two weeks, orally administering to the subject 75-150 mg of diazoxide choline once daily for a subsequent period of at least four weeks; and
   (i) after the subsequent period of at least four weeks, orally administering to the subject a target maintenance dose of 150 mg of diazoxide choline once daily; or
   (ii) after the subsequent period of at least four weeks, orally administering to the subject a maximum target maintenance dose of 225 mg of diazoxide choline once daily;
wherein the subject has one or more clinically relevant comorbidities prior to treatment with diazoxide choline.

Embodiment IV-36. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks; and
(c) after the second period of at least two weeks, orally administering to the subject a target maintenance dose of 225 mg of diazoxide choline once daily;
wherein the subject has one or more clinically relevant comorbidities prior to treatment with diazoxide choline.

Embodiment IV-37. The method of embodiment IV-35 or IV-36, wherein the subject weighs from 40 kg to less than 65 kg.

Embodiment IV-38. A method of treating hyperphagia in a subject having Prader-Willi Syndrome, the method comprising:
(a) orally administering to the subject 75-100 mg of diazoxide choline once daily for an initial period of at least two weeks;
(b) after the initial period of at least two weeks, orally administering to the subject 150-200 mg of diazoxide choline once daily for a second period of at least two weeks;
(c) after the second period of at least two weeks, orally administering to the subject 200-250 mg of diazoxide choline once daily for a third period of at least two weeks; and
   (i) after the third period of at least two weeks, orally administering to the subject a target maintenance dose of 200-250 mg of diazoxide choline once daily; or
   (ii) after the third period of at least two weeks, orally administering to the subject a maximum target maintenance dose of 375 mg of diazoxide choline once daily;
wherein the subject has one or more clinically relevant comorbidities prior to treatment with diazoxide choline.

Embodiment IV-39. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks; and
(e) after the fourth period of at least two weeks, orally administering to the subject a target maintenance dose of 375 mg of diazoxide choline once daily;
wherein the subject has one or more clinically relevant comorbidities prior to treatment with diazoxide choline.

Embodiment IV-40. A method of treating hyperphagia in a subject having Prader-Willi Syndrome, the method comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 375 mg of diazoxide choline once daily;
wherein the subject has one or more clinically relevant comorbidities prior to treatment with diazoxide choline.

Embodiment IV-41. The method of any one of embodiments IV-38 to IV-40, wherein the subject weighs from 65 kg to less than 100 kg.

Embodiment IV-42. A method of treating hyperphagia in a subject having Prader-Willi Syndrome, the method comprising:
(a) orally administering to the subject 75-100 mg of diazoxide choline once daily for an initial period of at least two weeks;
(b) after the initial period of at least two weeks, orally administering to the subject 150-225 mg of diazoxide choline once daily for a second period of at least two weeks;
(c) after the second period of at least two weeks, orally administering to the subject 225-300 mg of diazoxide choline once daily for a third period of at least two weeks; and
   (i) after the third period of at least two weeks, orally administering to the subject a target maintenance dose of 300-400 mg of diazoxide choline once daily; or
   (ii) after the third period of at least two weeks, orally administering to the subject a maximum target maintenance dose of 450 mg of diazoxide choline once daily;
wherein the subject has one or more clinically relevant comorbidities prior to treatment with diazoxide choline.

Embodiment IV-43. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks; and
(f) after the fifth period of at least two weeks, orally administering to the subject a target maintenance dose of 450 mg of diazoxide choline once daily;
wherein the subject has one or more clinically relevant comorbidities prior to treatment with diazoxide choline.

Embodiment IV-44. A method of treating hyperphagia in a subject having Prader-Willi Syndrome, the method comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject an initial target dose of 325 mg of diazoxide choline once daily for a period of at least six months; and
   (i) after the period of at least six months, orally administering to the subject the initial target dose of 325 mg of diazoxide choline once daily; or
   (ii) after the period of at least six months, orally administering to the subject a maximum target dose of 450 mg of diazoxide choline once daily;
wherein the subject has one or more clinically relevant comorbidities prior to treatment with diazoxide choline.

Embodiment IV-45. The method of any one of embodiments IV-42 to IV-44, wherein the subject weighs from 100 kg to less than 135 kg.

Embodiment IV-46. A method of treating hyperphagia in a subject having Prader-Willi Syndrome, the method comprising:
(a) orally administering to the subject 75-100 mg of diazoxide choline once daily for an initial period of at least two weeks;
(b) after the initial period of at least two weeks, orally administering to the subject 150-225 mg of diazoxide choline once daily for a second period of at least two weeks;
(c) after the second period of at least two weeks, orally administering to the subject 225-300 mg of diazoxide choline once daily for a third period of at least two weeks; and
   (i) after the third period of at least two weeks, orally administering to the subject a target maintenance dose of 300-400 mg of diazoxide choline once daily; or
   (ii) after the third period of at least two weeks, orally administering to the subject a maximum target maintenance dose of 525 mg of diazoxide choline once daily;
wherein the subject has one or more clinically relevant comorbidities prior to treatment with diazoxide choline.

Embodiment IV-47. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks;
(f) after the fifth period of at least two weeks, orally administering to the subject 450 mg of diazoxide choline once daily for a sixth period of at least two weeks; and
(g) after the sixth period of at least two weeks, orally administering to the subject a target maintenance dose of 525 mg of diazoxide choline once daily;
wherein the subject has one or more clinically relevant comorbidities prior to treatment with diazoxide choline.

Embodiment IV-48. A method of treating hyperphagia in a subject having Prader-Willi Syndrome, the method comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject 325 mg of diazoxide choline once daily for a fifth period of at least four weeks;
(f) after the fifth period of at least four weeks, orally administering to the subject an initial target dose of 375 mg of diazoxide choline once daily for a period of at least six months; and
   (i) after the period of at least six months, orally administering to the subject the initial target dose of 375 mg of diazoxide choline once daily; or
   (ii) after the period of at least six months, orally administering to the subject a maximum target dose of 525 mg of diazoxide choline once daily;
wherein the subject has one or more clinically relevant comorbidities prior to treatment with diazoxide choline.

Embodiment IV-49. The method of any one of embodiments IV-46 to IV-48, wherein the subject weighs 135 kg or more.

Embodiment IV-50. The method of any one of embodiments IV-29 to IV-49, wherein the one or more clinically relevant comorbidities is a cardiovascular, pulmonary, or metabolic/endocrine comorbidity.

Embodiment IV-51. The method of any one of embodiments IV-29 to IV-50, wherein the one or more clinically relevant comorbidities is a history of edema, edema, fluid retention, fluid overload, congestive heart failure, compromised cardiac reserve, or heart disease.

Embodiment IV-52. The method of any one of embodiments IV-29 to IV-50, wherein the one or more clinically relevant comorbidities is a history of asthma, asthma, Chronic Obstructive Pulmonary Disease (COPD), obstructive sleep apnea, or central sleep apnea.

Embodiment IV-53. The method of any one of embodiments IV-29 to IV-50, wherein the one or more clinically relevant comorbidities is obesity, hyperglycemia, elevated fasting glucose, elevated HbA1c, prediabetes, diabetes, or concomitant use of corticosteroids or medications known to impact blood glucose.

Embodiment IV-54. The method of any one of embodiments IV-29 to IV-50 or IV-53, wherein the one or more clinically relevant comorbidities is obesity.

Embodiment IV-55. The method of any one of embodiments IV-29 to IV-50 or IV-53 to IV-54, wherein the one or more clinically relevant comorbidities is severe obesity.

Embodiment IV-56. The method of any one of embodiments IV-29 to IV-49, wherein the one or more clinically relevant comorbidities is concomitant use of one or more medications that could contribute to hyperglycemia, fluid retention, or weight gain.

Embodiment IV-57. The method of any one of embodiments IV-29 to IV-50 or IV-53, wherein the one or more clinically relevant comorbidities is hyperglycemia.

Embodiment IV-58. The method of embodiment IV-57, wherein the method further comprises periodically monitoring fasting glucose during treatment.

Embodiment IV-59. The method of embodiment IV-58, wherein the monitoring takes place at least once a week during titration.

Embodiment IV-60. The method of embodiment IV-58, wherein the monitoring takes place every two weeks after titration.

Embodiment IV-61. The method of embodiment IV-58, wherein the monitoring takes place as clinically indicated after titration.

Embodiment IV-62. The method of any one of embodiments IV-29 to IV-50 or IV-53, wherein the one or more clinically relevant comorbidities is elevated HbA1c.

Embodiment IV-63. The method of embodiment IV-62, wherein the method further comprises periodically monitoring HbA1c during treatment.

Embodiment IV-64. The method of embodiment IV-63, wherein the monitoring takes place every 3 months.

Embodiment IV-65. The method of embodiment IV-63, wherein the monitoring takes place as clinically indicated.

Embodiment IV-66. The method of any one of embodiments IV-29 to IV-51, wherein the one or more clinically relevant comorbidities is edema or fluid overload.

Embodiment IV-67. The method of embodiment IV-66, wherein the method further comprises periodically monitoring body weight during treatment.

Embodiment IV-68. The method of embodiment IV-66 or IV-67, wherein the method further comprises periodically assessing the subject for swelling of a body part during treatment.

Embodiment IV-69. The method of embodiment IV-68, wherein the body part is an ankle, foot, or leg.

Embodiment IV-70. The method of any one of embodiments IV-66 to IV-69, wherein the method further comprises periodically assessing the subject for one or more respiratory symptoms during treatment.

Embodiment IV-71. The method of embodiment IV-70, wherein the one or more respiratory symptoms is dyspnea or shortness of breath.

Embodiment IV-72. The method of any one of embodiments IV-67 to IV-71, wherein the monitoring takes place once a week during titration.

Embodiment IV-73. The method of any one of embodiments IV-67 to IV-71, wherein the monitoring takes place at regular intervals after titration.

Embodiment IV-74. A method of treating hyperphagia in a subject having Prader-Willi Syndrome, the method comprising orally administering to the subject diazoxide choline once daily, wherein the subject has one or more clinically relevant comorbidities prior to treatment with diazoxide choline, and wherein the amount of diazoxide choline administered to the subject is reduced by between ⅓ to ½ relative to the amount that would be administered to the subject if the subject did not have one or more clinically relevant comorbidities.

Embodiment IV-75. The method of embodiment IV-74, wherein the amount of diazoxide choline administered to the subject is reduced by ⅓ relative to the amount that would be administered to the subject if the subject did not have one or more clinically relevant comorbidities.

Embodiment IV-76. The method of embodiment IV-74, wherein the amount of diazoxide choline administered to the subject is reduced by ½ relative to the amount that would be administered to the subject if the subject did not have one or more clinically relevant comorbidities.

Embodiment IV-77. A method of treating hyperphagia in a subject having Prader-Willi Syndrome, the method comprising:
(a) orally administering to the subject 25 mg of diazoxide choline once daily for an initial period of at least two weeks;
(b) after the initial period of at least two weeks, orally administering to the subject 50 mg of diazoxide choline once daily for a second period of at least two weeks;
(c) after the second period of at least two weeks, orally administering to the subject 75 mg of diazoxide choline once daily for a third period of at least two weeks; and
(d) after the third period of at least two weeks, orally administering to the subject a target maintenance dose of 100 mg of diazoxide choline once daily;
wherein the subject experiences one or more adverse reactions during treatment with diazoxide choline.

Embodiment IV-78. The method of embodiment IV-77, wherein the subject weighs from about 20 kg to less than 30 kg.

Embodiment IV-79. A method of treating hyperphagia in a subject having Prader-Willi Syndrome, the method comprising:
(a) orally administering to the subject 75 mg of diazoxide choline once daily for an initial period of at least two weeks; and
(b) after the initial period of at least two weeks, orally administering to the subject a target maintenance dose of 150 mg of diazoxide choline once daily;
wherein the subject experiences one or more adverse reactions during treatment with diazoxide choline.

Embodiment IV-80. The method of embodiment IV-79, wherein the subject weighs from 30 kg to less than 40 kg.

Embodiment IV-81. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks; and
(c) after the second period of at least two weeks, orally administering to the subject a target maintenance dose of 225 mg of diazoxide choline once daily;
wherein the subject experiences one or more adverse reactions during treatment with diazoxide choline.

Embodiment IV-82. The method of embodiment IV-81, wherein the subject weighs from 40 kg to less than 65 kg.

Embodiment IV-83. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks; and
(e) after the fourth period of at least two weeks, orally administering to the subject a target maintenance dose of 375 mg of diazoxide choline once daily;
wherein the subject experiences one or more adverse reactions during treatment with diazoxide choline.

Embodiment IV-84. A method of treating hyperphagia in a subject having Prader-Willi Syndrome, the method comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 375 mg of diazoxide choline once daily;
wherein the subject experiences one or more adverse reactions during treatment with diazoxide choline.

Embodiment IV-85. The method of embodiment IV-83 or IV-84, wherein the subject weighs from 65 kg to less than 100 kg.

Embodiment IV-86. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks; and
(f) after the fifth period of at least two weeks, orally administering to the subject a target maintenance dose of 450 mg of diazoxide choline once daily;
wherein the subject experiences one or more adverse reactions during treatment with diazoxide choline.

Embodiment IV-87. A method of treating hyperphagia in a subject having Prader-Willi Syndrome, the method comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject an initial target dose of 325 mg of diazoxide choline once daily for a period of at least six months; and
   (i) after the period of at least six months, orally administering to the subject the initial target dose of 325 mg of diazoxide choline once daily; or
   (ii) after the period of at least six months, orally administering to the subject a maximum target dose of 450 mg of diazoxide choline once daily;
wherein the subject experiences one or more adverse reactions during treatment with diazoxide choline.

Embodiment IV-88. The method of embodiment IV-86 or IV-87, wherein the subject weighs from 100 kg to less than 135 kg.

Embodiment IV-89. A method of treating hyperphagia in a subject having Prader-Willi Syndrome, the method comprising:
(a) orally administering to the subject 75 mg of diazoxide choline once daily for an initial period of at least two weeks;
(b) after the initial period of at least two weeks, orally administering to the subject 150 mg of diazoxide choline once daily for a second period of at least two weeks;
(c) after the second period of at least two weeks, orally administering to the subject 225 mg of diazoxide choline once daily for a third period of at least two weeks;
(d) after the third period of at least two weeks, orally administering to the subject 300 mg of diazoxide choline once daily for a fourth period of at least two weeks;
(e) after the fourth period of at least two weeks, orally administering to the subject 375 mg of diazoxide choline once daily for a fifth period of at least two weeks;
(f) after the fifth period of at least two weeks, orally administering to the subject 450 mg of diazoxide choline once daily for a sixth period of at least two weeks; and
(g) after the sixth period of at least two weeks, orally administering to the subject a target maintenance dose of 525 mg of diazoxide choline once daily;
wherein the subject experiences one or more adverse reactions during treatment with diazoxide choline.

Embodiment IV-90. A method of treating hyperphagia in a subject having Prader-Willi Syndrome, the method comprising:
(a) orally administering to the subject 75 mg of diazoxide choline once daily for an initial period of at least four weeks;
(b) after the initial period of at least four weeks, orally administering to the subject 150 mg of diazoxide choline once daily for a second period of at least four weeks;
(c) after the second period of at least four weeks, orally administering to the subject 225 mg of diazoxide choline once daily for a third period of at least four weeks;
(d) after the third period of at least four weeks, orally administering to the subject 275 mg of diazoxide choline once daily for a fourth period of at least four weeks;
(e) after the fourth period of at least four weeks, orally administering to the subject 325 mg of diazoxide choline once daily for a fifth period of at least four weeks;
(f) after the fifth period of at least four weeks, orally administering to the subject an initial target dose of 375 mg of diazoxide choline once daily for a period of at least six months; and
   (i) after the period of at least six months, orally administering to the subject the initial target dose of 375 mg of diazoxide choline once daily; or
   (ii) after the period of at least six months, orally administering to the subject a maximum target dose of 525 mg of diazoxide choline once daily;
wherein the subject experiences one or more adverse reactions during treatment with diazoxide choline.

Embodiment IV-91. The method of embodiment IV-89 or IV-90, wherein the subject weighs 135 kg or more.

Embodiment IV-92. The method of any one of embodiments IV-77 to IV-91, wherein the once daily administration of diazoxide choline is paused for a period of time following the development of one or more adverse reactions.

Embodiment IV-93. The method of any one of embodiments IV-77 to IV-91, wherein the once daily administration dose of diazoxide choline is reduced following the development of one or more adverse reactions.

Embodiment IV-94. The method of any one of embodiments IV-77 to IV-93, wherein the subject is concomitantly administered one or more risk mitigating medications following the development of one or more adverse reactions.

Embodiment IV-95. The method of embodiment IV-94, wherein the one or more risk mitigating medications is a glucose-lowering agent or a diuretic.

Embodiment IV-96. The method of any one of embodiments IV-77 to IV-95, wherein the one or more adverse reactions is a cardiovascular, pulmonary, or metabolic/endocrine adverse reaction.

Embodiment IV-97. The method of any one of embodiments IV-77 to IV-96, wherein the one or more adverse reactions is edema, fluid retention, fluid overload, congestive heart failure, reduced cardiac reserve, or heart disease.

Embodiment IV-98. The method of any one of embodiments IV-77 to IV-96, wherein the one or more adverse reactions is asthma, Chronic Obstructive Pulmonary Disease (COPD), obstructive sleep apnea, or central sleep apnea.

Embodiment IV-99. The method of any one of embodiments IV-77 to IV-96, wherein the one or more adverse reactions is obesity, hyperglycemia, elevated fasting glucose, elevated HbA1c, prediabetes, or diabetes.

Embodiment IV-100. The method of any one of embodiments IV-77 to IV-96 or IV-99, wherein the one or more adverse reactions is obesity.

Embodiment IV-101. The method of any one of embodiments IV-77 to IV-96 or IV-99 to IV-100, wherein the one or more adverse reactions is severe obesity.

Embodiment IV-102. The method of any one of embodiments IV-29 to IV-73 or IV-77 to IV-99, wherein the initial period of at least two weeks is two, three, or four weeks.

Embodiment IV-103. The method of any one of embodiments IV-29 to IV-31, IV-36 to IV-73, IV-77 to IV-78, or IV-81 to IV-102, wherein the second period of at least two weeks is two, three, or four weeks.

Embodiment IV-104. The method of any one of embodiments IV-29 to IV-31, IV-38 to IV-73, IV-77 to IV-78, or IV-83 to IV-103, wherein the third period of at least two weeks is two, three, or four weeks.

Embodiment IV-105. The method of any one of embodiments IV-39 to IV-41, IV-43 to IV-45, IV-47 to IV-73, or IV-83 to IV-104, wherein the fourth period of at least two weeks is two, three, or four weeks.

Embodiment IV-106. The method of any one of embodiments IV-43 to IV-45, IV-47 to IV-73, or IV-86 to IV-105, wherein the fifth period of at least two weeks is two, three, or four weeks.

Embodiment IV-107. The method of any one of embodiments IV-47 to IV-73 or IV-89 to IV-106, wherein the sixth period of at least two weeks is two, three, or four weeks.

Embodiment IV-108. The method of any one of embodiments IV-32, IV-34 to IV-35, IV-37, IV-50 to IV-73, or IV-102, wherein the subsequent period of at least four weeks is four, five, six, seven, or eight weeks.

Embodiment IV-109. The method of any one of embodiments IV-29 to IV-73 or IV-77 to IV-108, wherein the target maintenance dose is reached after 12 weeks of treatment.

Embodiment IV-110. The method of any one of embodiments IV-29 to IV-73 or IV-77 to IV-108, wherein the target maintenance dose is reached after 14 weeks of treatment.

Embodiment IV-111. The method of any one of embodiments IV-29 to IV-73 or IV-77 to IV-108, wherein the target maintenance dose is reached after 16 weeks of treatment.

Embodiment IV-112. The method of any one of embodiments IV-29 to IV-73 or IV-77 to IV-108, wherein the target maintenance dose is reached after 20 weeks of treatment.

Embodiment IV-113. The method of any one of embodiments IV-29 to IV-73 or IV-77 to IV-108, wherein the target maintenance dose is reached after 24 weeks of treatment.

Embodiment IV-114. The method of any one of embodiments IV-29 to IV-31, IV-50 to IV-73, IV-77 to IV-78, IV-92 to IV-104, or IV-109 to IV-113, wherein after the target maintenance dose is reached, the amount of diazoxide choline orally administered once daily to the subject is increased by no more than 25 mg every adjustment period, wherein every adjustment period has a duration of at least two weeks, wherein the amount of diazoxide choline orally administered once daily to the subject is no more than 5.8 mg/kg.

Embodiment IV-115. The method of any one of embodiments IV-32 to IV-73 or IV-79 to IV-113, wherein after the target maintenance dose is reached, the amount of diazoxide choline orally administered once daily to the subject is increased by no more than 75 mg every adjustment period, wherein every adjustment period has a duration of at least two weeks, wherein the amount of diazoxide choline orally administered once daily to the subject is no more than 5.8 mg/kg or 525 mg, whichever is lower.

Embodiment IV-116. A method of treating hyperphagia in a subject having Prader-Willi Syndrome, the method comprising:
(a) orally administering to the subject 25 mg of diazoxide choline once daily for an initial period of at least eight weeks;
(b) after the initial period of at least eight weeks, orally administering to the subject 50 mg of diazoxide choline once daily for a sequential period of at least four weeks; and
(c) after the sequential period of at least four weeks, orally administering to the subject a target dose of 75 mg of diazoxide choline once daily;
wherein the subject is concomitantly being administered one or more strong CYP1A2 inhibitors.

Embodiment IV-117. The method of embodiment IV-116, wherein the subject weighs 20 kg to less than 30 kg.

Embodiment IV-118. A method of treating hyperphagia in a subject having Prader-Willi Syndrome, the method comprising:
(a) orally administering to the subject 50 mg of diazoxide choline once daily for an initial period of at least four weeks; and
(b) after the initial period of at least four weeks, orally administering to the subject a target dose of 100 mg of diazoxide choline once daily;
wherein the subject is concomitantly being administered one or more strong CYP1A2 inhibitors.

Embodiment IV-119. The method of embodiment IV-118, wherein the subject weighs 30 kg to less than 40 kg.

Embodiment IV-120. A method of treating hyperphagia in a subject having Prader-Willi Syndrome, the method comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks; and
(c) after the second period of at least four weeks, orally administering to the subject a target dose of 150 mg of diazoxide choline once daily;
wherein the subject is concomitantly being administered one or more strong CYP1A2 inhibitors.

Embodiment IV-121. The method of embodiment IV-120, wherein the subject weighs 40 kg to less than 65 kg.

Embodiment IV-122. A method of treating hyperphagia in a subject having Prader-Willi Syndrome, the method comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject an initial target dose of 200 mg of diazoxide choline once daily for a period of at least six months; and
   (i) after the period of at least six months, orally administering to the subject the initial target dose of 200 mg of diazoxide choline once daily; or
   (ii) after the period of at least six months, orally administering to the subject a maximum target dose of 250 mg of diazoxide choline once daily;
wherein the subject is concomitantly being administered one or more strong CYP1A2 inhibitors.

Embodiment IV-123. The method of embodiment IV-122, wherein the subject weighs 65 kg to less than 100 kg.

Embodiment IV-124. A method of treating hyperphagia in a subject having Prader-Willi Syndrome, the method comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject 200 mg of diazoxide choline once daily for a fourth period of at least four weeks;
(e) after the fourth period of at least four weeks, orally administering to the subject an initial target dose of 225 mg of diazoxide choline once daily for a period of at least six months; and
   (i) after the period of at least six months, orally administering to the subject the initial target dose of 225 mg of diazoxide choline once daily; or
   (ii) after the period of at least six months, orally administering to the subject a maximum target dose of 300 mg of diazoxide choline once daily;
wherein the subject is concomitantly being administered one or more strong CYP1A2 inhibitors.

Embodiment IV-125. The method of embodiment IV-124, wherein the subject weighs 100 kg to less than 135 kg.

Embodiment IV-126. A method of treating hyperphagia in a subject having Prader-Willi Syndrome, the method comprising:
(a) orally administering to the subject 50 mg of diazoxide choline once daily for an initial period of at least four weeks;
(b) after the initial period of at least four weeks, orally administering to the subject 100 mg of diazoxide choline once daily for a second period of at least four weeks;
(c) after the second period of at least four weeks, orally administering to the subject 150 mg of diazoxide choline once daily for a third period of at least four weeks;
(d) after the third period of at least four weeks, orally administering to the subject 200 mg of diazoxide choline once daily for a fourth period of at least four weeks;
(e) after the fourth period of at least four weeks, orally administering to the subject 225 mg of diazoxide choline once daily for a fifth period of at least four weeks;
(f) after the fifth period of at least four weeks, orally administering to the subject an initial target dose of 275 mg of diazoxide choline once daily for a period of at least six months; and
   (i) after the period of at least six months, orally administering to the subject the initial target dose of 275 mg of diazoxide choline once daily; or
   (ii) after the period of at least six months, orally administering to the subject a maximum target dose of 325 mg of diazoxide choline once daily;
wherein the subject is concomitantly being administered one or more strong CYP1A2 inhibitors.

Embodiment IV-127. The method of embodiment IV-126, wherein the subject weighs 135 kg or more.

Embodiment IV-128. The method of any one of embodiments IV-116 to IV-127, wherein the one or more strong CYP1A2 inhibitors is amiodarone, ciprofloxacin, or fluvoxamine.

Embodiment IV-129. The method of any one of embodiments IV-116 to IV-128, wherein the strong CYP1A2 inhibitor is fluvoxamine.

Embodiment IV-130. The method of any one of embodiments IV-116 to IV-117 or IV-128 to IV-129, wherein the initial period of at least eight weeks is eight, nine, or ten weeks.

Embodiment IV-131. The method of any one of embodiments IV-116 to IV-117 or IV-128 to IV-130, wherein the initial period of at least eight weeks is eight weeks.

Embodiment IV-132. The method of any one of embodiments IV-116 to IV-117 or IV-128 to IV-129, wherein the sequential period of at least four weeks is four, five, or six weeks.

Embodiment IV-133. The method of any one of embodiments IV-116 to IV-117, IV-128 to IV-129, or IV-132, wherein the sequential period of at least four weeks is four weeks.

Embodiment IV-134. The method of any one of embodiments IV-40 to IV-41, IV-44 to IV-45, IV-48 to IV-73, IV-84 to IV-85, IV-87 to IV-88, IV-90 to IV-101, or IV-118 to IV-129, wherein the initial period of at least four weeks is four, five, or six weeks.

Embodiment IV-135. The method of any one of embodiments IV-40 to IV-41, IV-44 to IV-45, IV-48 to IV-73, IV-84 to IV-85, IV-87 to IV-88, IV-90 to IV-101, IV-118 to IV-129, or IV-134, wherein the initial period of at least four weeks is four weeks.

Embodiment IV-136. The method of any one of embodiments IV-40 to IV-41, IV-44 to IV-45, IV-48 to IV-73, IV-84 to IV-85, IV-87 to IV-88, IV-90 to IV-101, IV-120 to IV-129, or IV-134 to IV-135, wherein the second period of at least four weeks is four, five, or six weeks.

Embodiment IV-137. The method of any one of embodiments IV-40 to IV-41, IV-44 to IV-45, IV-48 to IV-73, IV-84 to IV-85, IV-87 to IV-88, IV-90 to IV-101, IV-120 to IV-129, or IV-134 to IV-136, wherein the second period of at least four weeks is four weeks.

Embodiment IV-138. The method of any one of embodiments IV-40 to IV-41, IV-44 to IV-45, IV-48 to IV-73, IV-84 to IV-85, IV-87 to IV-88, IV-90 to IV-101, IV-122 to IV-129, or IV-134 to IV-137, wherein the third period of at least four weeks is four, five, or six weeks.

Embodiment IV-139. The method of any one of embodiments IV-40 to IV-41, IV-44 to IV-45, IV-48 to IV-73, IV-84 to IV-85, IV-87 to IV-88, IV-90 to IV-101, IV-122 to IV-129, or IV-134 to IV-138, wherein the third period of at least four weeks is four weeks.

Embodiment IV-140. The method of any one of embodiments IV-44 to IV-45, IV-48 to IV-73, IV-87 to IV-88, IV-90 to IV-101, IV-124 to IV-129, or IV-134 to IV-139, wherein the fourth period of at least four weeks is four, five, or six weeks.

Embodiment IV-141. The method of any one of embodiments IV-44 to IV-45, IV-48 to IV-73, IV-87 to IV-88, IV-90 to IV-101, IV-124 to IV-129, or IV-134 to IV-140, wherein the fourth period of at least four weeks is four weeks.

Embodiment IV-142. The method of any one of embodiments IV-48 to IV-73, IV-90 to IV-101, IV-126 to IV-129, or IV-134 to IV-141, wherein the fifth period of at least four weeks is four, five, or six weeks.

Embodiment IV-143. The method of any one of embodiments IV-48 to IV-73, IV-90 to IV-101, IV-126 to IV-129, or IV-134 to IV-142, wherein the fifth period of at least four weeks is four weeks.

Embodiment IV-144. The method of any one of embodiments IV-40 to IV-41, IV-44 to IV-45, IV-48 to IV-73, IV-84 to IV-85, IV-87 to IV-88, IV-90 to IV-101, IV-122 to IV-129, or IV-134 to IV-143, wherein the period of at least six months is six, seven, eight, nine, ten, eleven, or twelve months.

Embodiment IV-145. The method of any one of embodiments IV-40 to IV-41, IV-44 to IV-45, IV-48 to IV-73, IV-84 to IV-85, IV-87 to IV-88, IV-90 to IV-101, IV-122 to IV-129, or IV-134 to IV-144, wherein the period of at least six months is six months.

Embodiment V-1. The method, use, or compound for use of any one of embodiments I-1 to I-99, II-1 to II-205, III-1 to III-77, or IV-1 to IV-145, wherein the subject has mild hyperphagia.

Embodiment V-2. The method, use, or compound for use of any one of embodiments I-1 to I-99, II-1 to II-205, III-1 to III-77, or IV-1 to IV-145, wherein the subject has moderate hyperphagia.

Embodiment V-3. The method, use, or compound for use of any one of embodiments I-1 to I-99, II-1 to II-205, III-1 to III-77, or IV-1 to IV-145, wherein the subject has severe hyperphagia.

Embodiment V-4. The method, use, or compound for use of any one of embodiments I-1 to I-99, II-1 to II-205, III-1 to III-77, or IV-1 to IV-145, wherein the subject has moderate to severe hyperphagia.

Embodiment V-5. The method, use, or compound for use of any one of embodiments I-1 to I-99, II-1 to II-205, III-1 to III-77, IV-1 to IV-145, or V-1 to V-4, wherein said method, use, or compound for use comprises administration of a corresponding amount of diazoxide free form instead of diazoxide choline.

Embodiment V-6. The method, use, or compound for use of any one of embodiments I-1 to I-99, II-1 to II-205, III-1 to III-77, IV-1 to IV-145, or V-1 to V-4, wherein said method, use, or compound for use comprises administration of a corresponding amount of an alternative salt form of diazoxide instead of diazoxide choline.

Embodiment V-7. The method, use, or compound for use of any one of embodiments I-1 to I-99, II-1 to II-205, III-1 to III-77, IV-1 to IV-145, or V-1 to V-6, wherein the one or more strong OAT1 inhibitors is selected from the group consisting of benzylpenicillin, methotrexate, and pravastatin.

Embodiment V-8. The method, use, or compound for use of any one of embodiments I-1 to I-99, II-1 to II-205, III-1 to III-77, IV-1 to IV-145, or V-1 to V-6, wherein the one or more strong OAT3 inhibitors is selected from the group consisting of benzylpenicillin, methotrexate, and pravastatin.

### EXAMPLES

The following specific examples are to be construed as merely illustrative, and not limitative of the remainder of the disclosure in any way whatsoever.

### Example 1: Metabolic Profiling with Human Liver Microsomes and CYP Isoforms (Identification of CYP Enzymes Responsible for Metabolism of Diazoxide Choline)

Diazoxide choline was prepared as 10 mM stock solutions in dimethyl sulfoxide. Incubation reactions, conducted in duplicate, consisted of 0.5 mg microsomal protein or 12.5 pmol human CYP isoform, 100 mM potassium phosphate buffer, pH 7.4, 100 M test compound, and NADPH regenerating system (1.3 mM NADP+, 3.3 mM glucose-6-phosphate, 0.4 U/mL glucose-6-phosphate dehydrogenase, and 3.3 mM magnesium chloride) in a total volume of 0.5 mL. The reaction mixture was prewarmed for 15 min at 37 °C prior to the addition of the catalyst. Ice-cold enzyme was then added to initiate the reaction. After 4 h incubation in a shaking water bath at 37 °C, the reactions were terminated by the addition of an equal volume of methanol.

After incubation on ice for 15 min, samples were centrifuged (3,000 g, 15 min, 4 °C) to remove precipitated protein.

In all HPLC traces recorded for samples from microsomal and CYP incubations, a number of peaks eluting at the beginning of the gradient (before approximately 6 min) were not related to test compound and were caused by components of the biocatalytic system (microsomes, CYP, or cofactor regeneration system).

Therefore, chromatograms in this report were expanded for clarity to show only the relevant part of HPLC gradient, approximately between 7 and 11 min. Each reaction sample was subjected to a thorough LC/MS analysis to identify potential metabolites and determine their molecular weights.

After four hours of incubation with microsomes, approximately 10% of the test compound was metabolized. Two monooxygenated metabolites of diazoxide choline, M1 and M2, were detected in incubations with human liver microsomes. Based on the size of corresponding UV peaks, these metabolites were formed in amounts of less than 5% of the initial parent compound. One of these metabolites, possibly the more polar M1, may correspond to 3-hydroxy-7-chloro-1,2,4-benzothiadiazine-1,1-dioxide, previously identified in vivo after oral administration of diazoxide choline in humans and animals.

Formation of monooxygenated polar metabolite M1, the primary metabolite of diazoxide is catalyzed by several cytochrome P450 isoforms, CYP1A2 being the major one, with minor contribution from CYP3A4.

### Example 2: Physiologically Based Pharmacokinetic (PBPK) Model for DCCR

This example utilizes a PBPK model based on the available in vitro and clinical PK data to assess (1) the DDI liability of DCCR as a victim of CYP1A2 and CYP3A4-mediated metabolism in healthy subjects; (2) the impact of administration of DCCR on the exposure (AUC_{0-INF} and Cₘₐₓ) of the CYP1A2, OAT3, MATE1, or BCRP substrate (caffeine, methotrexate, metformin, or rosuvastatin) in healthy subjects; and (3) the exposure of DCCR in patients with renal or hepatic impairment.

### Parameter Estimation:

It is not always possible to recover observed drug plasma concentration-time profiles accurately purely from the ‘bottom-up’, using physicochemical, in vitro permeability and metabolism/transport data. To accommodate inherent uncertainty, the parameter estimation module within the Simcyp Simulator may be used to optimise key parameter values. This provides a link between the ‘bottom-up’ and ‘top-down’ pharmacokinetic modelling paradigms by utilising available in vivo data to modify parameters based purely on in vitro studies. The simulations then move forward more effectively to predict the impact of specific perturbations of drug kinetics as a consequence of drug-drug interaction. The parameter estimation module was used to estimate dissolution rate profile using Weibull function (α=4.34 and β=0.84) for DCCR.

### PBPK Model Input Parameters for DCCR:

All final parameters used for the simulation of diazoxide kinetics are listed in Table 6.

TABLE 6
Final input physiochemical, binding, absorption, distribution,
elimination and interaction parameters for DCCR.

| | | |
|---|---|---|
| PARAMETER | Value | Reference |

Physicochemical and Binding Parameters

| | | |
|---|---|---|
| Molecular Weight (g/mol) | 230.67 (diazoxide) | PubChem, accessed on 30 Jul. 2021 |
| Log P | 1.2 | PubChem, accessed on 30 Jul. 2021 |
| Compound type | Monoprotic acid | ACDLab (ACD/Percepta 14.51.0 (Build 3382), |
| | | accessed on 6 Sep. 2021 |
| pKa | 8.74 | PubChem, accessed on 30 Jul. 2021 |
| B:P | 0.75 | Report: BEFA-0003-DV-LA_RBC |
| | | Partitioning_Final_7 Oct. 2021 |
| fu | 0.084 | Report: SLN-P5003_8 Apr. 2020 Equilibrium |
| | | protein binding assay |
| Main binding protein | Human Serum | Sellers and Koch-Weser, 1973 |
| | Albumin | |

Absorption model - ADAM model

| | | |
|---|---|---|
| fu_{gut} | 1 | Assumed |
| Caco-2 Pₐₚₚ (×10⁻⁶ cm/s) at pH | 6.69 | Report: Caco-2 cells data from Permeability |
| 7.4:7.4 | | Report 27th_Feb 2020 AS |
| Calibrator Pₐₚₚ (×10⁻⁶ cm/s) | 0.123 (Atenolol) | Report: Caco-2 cells data from Permeability |
| | | Report 27th_Feb 2020 AS |
| P_{eff, man} (pred) (×10⁻⁴ cm/s) | 3.88974 | Predicted |
| Formulation type | Controlled Release | |
| | (Monolithic System) | |
| Dissolution profile | Weibull function | |
| alpha | 4.34 | Optimised to recover the observed PK profile |
| beta | 0.84 | after single oral dose of 300 mg DCCR (C605 |
| | | control phase) |

Distribution model - full PBPK model

| | | |
|---|---|---|
| V_{SS} (L/kg) | 0.20 | Method 2 |
| Kₚ scalar | 1.20 | Optimised to recover the observed volume of |
| | | distribution following a single intravenous dose |
| | | of diazoxide (Sadee et al., 1973) |

Elimination parameters

| | | |
|---|---|---|
| CL(L/h) | 0.488 | Sellers and Koch-Weser, 1969; NCA for Sellers |
| | | and Koch-Weser 1969 study |
| fm_{CYP1A2} | 0.54 | |
| fm_{CYP3A4} | 0.13 | |
| CYP1A2 CLᵢₙₜ (μl/min/pmol) | 0.0199 | Retrograde model using CL and % hepatic |
| | | metabolism CL of CYP1A2 = 80% |
| CYP3A4 CLᵢₙₜ (μl/min/pmol) | 0.00169 | Retrograde model using CL and % hepatic |
| | | metabolism CL of CYP3A4 = 20% |
| CL_{R} (L/h) | 0.151 | Applied the fraction of diazoxide excreted |
| | | unchanged in urine (31%, Pruitt et al., 1974) to |
| | | CL |

Interaction parameters

| | | |
|---|---|---|
| CYP1A2 Kᵢ (μM) | 80.5 | Report_BEFA-0005-DV-TB_CYP |
| | | TDI_Final_7 Oct. 2021 |
| fu_{mic} | 1 | Assumed/measured (Report_BEFA-0002-DV- |
| | | EB_Microsomal Binding_Final_7 Oct. 2021) |
| BCRP Kᵢ (μM) | 157.79 | Report: Soleno-03_ECTD_Report_final_signed |
| fu_{inc} | 1 | Assumed |
| MATE Kᵢ (μM) | 273.89 | Report: Soleno-03_ECTD_Report_final_signed |
| fu_{inc} | 1 | Assumed |
| OAT1 Kᵢ (μM) | 3.41 | Report: Soleno-03_ECTD_Report_final_signed |
| fu_{inc} | 1 | Assumed |
| OAT3 Kᵢ (μM) | 2.28 | Report: Soleno-03_ECTD_Report_final_signed |
| fu_{inc} | 1 | Assumed |
| CYP1A2 Kₐₚₚ (μM) | 45.77 | Report: 212428_20300474_Soleno_CYP |
| | | Inhibition |
| CYP1A2 k_{inact} (1/h) | 0.924 | Report: 212428_20300474_Soleno_CYP |
| | | Inhibition |
| fu_{mic} | 1 | Assumed/measured (Report_BEFA-0002-DV- |
| | | EB_Microsomal Binding_Final_7 Oct. 2021) |

### Model Development:

### Simulation of a Single Oral Dose of 300 mg DCCR in Healthy Subjects

The trial design used for the simulation of the plasma concentration-time profiles of diazoxide in healthy subjects was based on the Clinical Study C605 control phase. In the study, 12 healthy subjects aged 20 to 54 years (17% female) received a single oral dose of 300 mg DCCR. For the simulations, 10 virtual trials of 12 subjects (17% female) aged 20 to 54 years were generated to assess variability across groups. The simulated concentration time profiles of diazoxide were compared to the observed data.

### Model Verification:

### Simulation of Multiple Oral Doses of 290 mg DCCR QD in Healthy Subjects

The trial design used for simulation of the plasma concentration-time profiles of multiple doses of DCCR in healthy subjects was based on the Clinical Study PK015. In the study, 9 healthy subjects aged 20 to 59 years (11% female) received 290 mg QD doses of DCCR orally for 10 days. For the simulations, 10 virtual trials of 9 subjects aged 20 to 59 years (11% female) were generated to assess variability across groups. The simulated concentration time profiles of diazoxide were compared to the observed data.

### Simulation of Multiple Oral Doses of 435 mg DCCR QD in Healthy Subjects

The trial design used for simulation of the plasma concentration-time profiles of multiple doses DCCR in healthy subjects was based on the Clinical Study PK008. In the study, 12 healthy subjects aged 18 to 59 years (25% female) received 435 mg QD doses of DCCR orally for 16 days. For the simulations, 10 virtual trials of 12 subjects aged 18 to 59 years (25% female) were generated to assess variability across groups. The simulated concentration time profiles of diazoxide were compared to the observed data.

### Model Application—As a Victim:

Simulation of a Single Oral Dose of 290 mg DCCR Administered with Itraconazole in Healthy Subjects

Ten virtual trials of 10 healthy subjects (50% female) aged 20 to 50 years receiving a single oral dose of 290 mg DCCR in the absence of itraconazole and on the 7^{th} day of 14 days of dosing of itraconazole (200 mg BID for the first day followed by QD for 13 days) were generated. The default Simcyp compound library file for itraconazole (SV-Itraconaozle_Fasted Soln) was used. Performance verification of itraconazole is provided in Error!Reference source not found.

Simulation of a Single Oral Dose of 290 mg DCCR Administered with Rifampicin in Healthy Subjects

Ten virtual trials of 10 healthy subjects (50% female) aged 20 to 50 years receiving a single oral dose of 290 mg DCCR in the absence of rifampicin and on the 7^{th} day of 14 days of dosing of rifampicin (600 mg QD) were generated. The default Simcyp compound library file for rifampicin (SV-Rifampicin_MD) was used. Performance verification of rifampicin is provided in Error!Reference source not found.

Simulation of Multiple Oral Doses of 435 mg DCCR QD Administered with Itraconazole in Healthy Subjects

Ten virtual trials of 10 healthy subjects (25% female) aged 20 to 50 years receiving multiple oral doses of DCCR (435 mg QD; 14 days) in the absence of itraconazole and co-administered with itraconazole (200 mg BID for the first 2 days followed by QD for 18 days) were generated. The default Simcyp compound library file for itraconazole (SV-Itraconaozle_Fasted Soln) was used.

Simulation of Multiple Oral Doses of 435 mg DCCR QD Administered with Rifampicin in Healthy Subjects

Ten virtual trials of 10 healthy subjects (25% female) aged 20 to 50 years receiving multiple oral doses of DCCR (435 mg QD; 14 days) in the absence of rifampicin and co-administered with rifampicin (600 mg QD; 20 days) were generated. The default Simcyp compound library file for rifampicin (Sim-Rifampicin-MD) was used.

### Simulation of a Single Oral Dose of 290 mg DCCR in Smokers

Ten virtual trials of 10 healthy subjects (50% female) aged 20 to 50 years receiving a single oral dose of 290 mg DCCR in smokers were generated. smoking-induced changes in hepatic CYP1A2 abundance were extrapolated from reported in vivo caffeine clearance data for sub-groups of a smoking population that were categorized according to their daily cigarette consumption (Plowchalk and Rowland, 2012). The hepatic CYP1A2 abundance in Sim-HV was adjusted accordingly in the simulations.

### Model Application—As a Perpetrator:

Victim drugs were administered as a single dose with and without co-administration with DCCR. All simulations were performed under steady-state conditions for DCCR.

Simulation of a Single Oral Dose of 150 mg Caffeine Administered with DCCR in Healthy Subjects

Ten virtual trials of 10 healthy subjects (50% female) aged 20 to 50 years receiving a single oral dose of 150 mg caffeine in the absence of DCCR and on the 14^{th} day of 23 days of dosing of DCCR (435 mg QD) were generated. The default compound library file for caffeine (Sim-Caffeine) was used. Performance verification of the caffeine model is provided in Error!Reference source not found.

Simulation of a Single Oral Dose of 290 mg DCCR Administered with Itraconazole in Healthy Subjects

The simulated geometric mean AUC_{0-INF} and Cₘₐₓ values and corresponding GMRs for DCCR in the absence and presence of itraconazole are listed in Table 7.

TABLE 7
Simulated diazoxide geometric mean AUC_{0-INF} and Cₘₐₓ values and
corresponding GMRs following a single oral dose of DCCR in the
absence and presence of itraconazole in healthy subjects.

| | DCCR | | DCCR + Itraconazole | | | |
|---|---|---|---|---|---|---|
| | AUC_{0-INF} | Cₘₐₓ | AUC_{0-INF} | Cₘₐₓ | GMR | |
| | (h · mg/L) | (mg/L) | (h · mg/L) | (mg/L) | AUC_{0-INF} | Cₘₐₓ |
| Geometric Mean | 434.43 | 8.42 | 536.51 | 8.77 | 1.23 | 1.04 |
| 90% CI - Lower | 408.97 | 8.15 | 502.31 | 8.49 | 1.22 | 1.04 |
| 90% CI - Upper | 461.47 | 8.69 | 573.04 | 9.07 | 1.25 | 1.05 |
| 5^{th} Percentile | 227.11 | 5.86 | 271.70 | 6.25 | 1.10 | 1.02 |
| 95^{th} Percentile | 731.55 | 11.51 | 924.62 | 12.38 | 1.43 | 1.08 |

GMR: geometric mean ratio; CI: Confidence Interval; Source simulated data: DDI with Itraconzole_Fasted Solution_DCCR 290 mg SD_Sim-HV

Simulation of a Single Oral Dose of 290 mg DCCR Administered with Rifampicin in Healthy Subjects

The simulated geometric mean AUC_{0-INF} and Cₘₐₓ values and corresponding GMRs for DCCR in the absence and presence of rifampicin are listed in Table 8.

TABLE 8
Simulated diazoxide geometric mean AUC_{0-INF} and Cₘₐₓ values and
corresponding GMRs following a single oral dose of DCCR in the
absence and presence of rifampicin in healthy subjects.

| | DCCR | | DCCR + Rifampicin | | | |
|---|---|---|---|---|---|---|
| | AUC_{0-INF} | Cₘₐₓ | AUC_{0-INF} | Cₘₐₓ | GMR | |
| | (h · mg/L) | (mg/L) | (h · mg/L) | (mg/L) | AUC_{0-INF} | Cₘₐₓ |
| Geometric Mean | 430.01 | 8.32 | 195.98 | 6.63 | 0.46 | 0.80 |
| 90% CI - Lower | 404.80 | 8.05 | 181.18 | 6.38 | 0.43 | 0.78 |
| 90% CI - Upper | 456.78 | 8.61 | 211.99 | 6.90 | 0.48 | 0.81 |
| 5^{th} Percentile | 235.42 | 5.75 | 92.67 | 4.26 | 0.28 | 0.68 |
| 95^{th} Percentile | 699.37 | 10.93 | 416.26 | 9.51 | 0.67 | 0.92 |

GMR: geometric mean ratio; CI: Confidence Interval; Source simulated data: DDI with rifampicin_DCCR 290 mg SD_Sim-HV

Simulation of Multiple Oral Doses of 435 mg DCCR QD Administered with Itraconazole in Healthy Subjects

The simulated geometric mean AUCtau and Cₘₐₓ values and corresponding GMRs for DCCR in the presence and absence of itraconazole are listed in Table 9.

TABLE 9
Simulated diazoxide geometric mean AUCₜₐᵤ and Cₘₐₓ values and
corresponding GMRs following multiple oral doses of DCCR in
the absence and presence of itraconazole in healthy subjects.

| | DCCR | | DCCR + Itraconazole | | | |
|---|---|---|---|---|---|---|
| | AUCₜₐᵤ | Cₘₐₓ | AUCₜₐᵤ | Cₘₐₓ | GMR | |
| | (h · mg/L) | (mg/L) | (h · mg/L) | (mg/L) | AUCₜₐᵤ | Cₘₐₓ |
| Geometric Mean | 955.02 | 42.77 | 1286.52 | 56.52 | 1.35 | 1.32 |
| 90% CI - Lower | 912.61 | 40.98 | 1229.15 | 54.10 | 1.32 | 1.30 |
| 90% CI - Upper | 999.40 | 44.65 | 1346.57 | 59.05 | 1.37 | 1.34 |
| 5^{th} Percentile | 588.99 | 28.12 | 836.54 | 37.64 | 1.12 | 1.11 |
| 95^{th} Percentile | 1404.74 | 62.09 | 1918.61 | 82.18 | 1.62 | 1.57 |

GMR: geometric mean ratio; CI: Confidence Interval; Source simulated data: DDI with Itraconazole_Fasted Solution_DCCR 435 mg MDx14D_Sim-HV

Simulation of Multiple Oral Doses of 435 mg DCCR QD Administered with Rifampicin in Healthy Subjects

The simulated geometric mean AUCtau and Cₘₐₓ values and corresponding GMRs for DCCR in the presence and absence of rifampicin are listed in Table 10.

TABLE 10
Simulated diazoxide geometric mean AUCₜₐᵤ and Cₘₐₓ values and
corresponding GMRs for following multiple oral doses of DCCR
in the absence and presence of rifampicin in healthy subjects.

| | DCCR | | DCCR + Rifampicin | | | |
|---|---|---|---|---|---|---|
| | AUCₜₐᵤ | Cₘₐₓ | AUCₜₐᵤ | Cₘₐₓ | | |
| | (h · | (mg/ | (h · | (mg/ | GMR | |
| | mg/L) | L) | mg/L) | L) | AUCₜₐᵤ | Cₘₐₓ |
| Geometric Mean | 949.64 | 42.47 | 393.16 | 19.74 | 0.41 | 0.46 |
| 90% CI - Lower | 909.18 | 40.76 | 362.25 | 18.42 | 0.39 | 0.44 |
| 90% CI - Upper | 991.90 | 44.25 | 426.71 | 21.15 | 0.44 | 0.49 |
| 5^{th} Percentile | 576.77 | 25.96 | 179.03 | 10.77 | 0.24 | 0.31 |
| 95^{th} Percentile | 1316.74 | 58.32 | 834.49 | 39.16 | 0.69 | 0.72 |

GMR: geometric mean ratio;
CI: Confidence Interval;
Source simulated data: DDI with rifampicin_DCCR 435 mg MDx14D_Sim-HV

Simulation of a Single Oral Dose of 150 mg Caffeine Administered with DCCR in Healthy Subjects

The simulated geometric mean AUC_{0-INF} and Cₘₐₓ values and corresponding GMRs for caffeine in the presence and absence of DCCR are listed in Table 11.

TABLE 11
Simulated caffeine geometric mean AUC_{0-INF} and Cₘₐₓ values
and corresponding GMRs following caffeine administration
in the absence and presence of DCCR in healthy subjects.

| | Caffeine | | DCCR + Caffeine | | | |
|---|---|---|---|---|---|---|
| | AUC_{0-INF} | Cₘₐₓ | AUC_{0-INF} | Cₘₐₓ | GMR | |
| | (h · mg/L) | (mg/L) | (h · mg/L) | (mg/L) | AUC_{0-INF} | Cₘₐₓ |
| Geometric Mean | 25.00 | 3.39 | 292.97 | 4.39 | 11.81 | 1.29 |
| 90% CI - Lower | 22.39 | 3.23 | 258.66 | 4.21 | 10.93 | 1.27 |
| 90% CI - Upper | 27.92 | 3.56 | 331.84 | 4.57 | 12.77 | 1.32 |
| 5^{th} Percentile | 8.75 | 2.18 | 75.20 | 2.90 | 5.32 | 1.11 |
| 95^{th} Percentile | 71.93 | 5.48 | 754.69 | 6.37 | 24.57 | 1.60 |

GMR: geometric mean ratio;
CI: Confidence Interval;
Source simulated data: DDI with caffeine_DCCR 435 mg MD_Sim-HV

### Example 3: Pharmacokinetics of Diazoxide Choline—Administered with and without a Strong CYP1A2 Inhibitor

This example delineates a clinical study evaluating the pharmacokinetics of a Diazoxide Choline Controlled Release (DCCR) formulation administered with or without a strong CYP1A2 inhibitor, fluvoxamine.

Clinical study C605 was a single-center, open-label, fixed-sequence, two-period crossover study conducted in 12 healthy male or female subjects. The study included a screening period, two open-label dosing periods, and an end-of-study (EOS) or early withdrawal assessment visit. Subjects were administered a single dose of DCCR alone at the start of Dosing Period 1 and in combination with fluvoxamine during Dosing Period 2. The majority of subjects were male (10 subjects [83.3%]); white (7 subjects [58.3%]); and not Hispanic or Latino (10 subjects [83.3%]). The mean age was 39.4 years (range: 20 to 54 years), mean body weight was 81.81 kg, and mean BMI was 25.93 kg/m2. Demographic and baseline characteristics are presented in Table 12.

TABLE 12
Summary of Subject Demographics and Baseline
Characteristics (Safety Population)

| | Overall | |
|---|---|---|
| | (N = 12) | |
| Age (years) | | |
| Mean (SD) | 39.4 | (10.95) |
| Minimum, maximum | 20, 54 | |
| Sex, No. (%) | | |
| Male | 10 | (83.3) |
| Female | 2 | (16.7) |
| Post-menopausal^{a} | 1 | (50.0) |
| Potentially able to bear children^{a} | 1 | (50.0) |
| Race, No. (%) | | |
| White | 7 | (58.3) |
| Black or African American | 4 | (33.3) |
| Multiracial | 1 | (8.3) |
| Ethnicity, No. (%) | | |
| Not Hispanic or Latino | 10 | (83.3) |
| Hispanic or Latino | 2 | (16.7) |
| Height (cm) | | |
| Mean (SD) | 177.79 | (7.271) |
| Minimum, maximum | 161.7, 190.4 | |
| Weight (kg) | | |
| Mean (SD) | 81.81 | (8.930) |
| Minimum, maximum | 70.1, 102.5 | |
| Body mass index (kg/m²) | | |
| Mean (SD) | 25.93 | (2.745) |

| | |
|---|---|
| Minimum, maximum | 22.3, 31.3 |

Abbreviations: DCCR = diazoxide choline extended-release tablet; Q12H = every 12 hours; Q24H = every 24 hours.
Note:
Single sequence crossover of interest: DCCR alone: single dose of 300 mg DCCR administered on Day 1; Fluvoxamine alone: 50 mg fluvoxamine administered Q24H on Days 8 and 9, Q12H on Days 10 to 13; DCCR with fluvoxamine: single dose of 300 mg DCCR administered on Day 14 with 50 mg fluvoxamine administered Q12H on Days 14 to 20 and 50 mg fluvoxamine administered Q24H on Days 21 and 22. Percentages were based on the number of subjects in the Safety population.
^{a}Percentages were based on the number of female subjects in the safety population.

### Period 1 (Day 1 Through Day 7):

On Day 1, 300 mg of DCCR was administered as 2×150 mg DCCR tablets after an overnight fast of at least 10 hours. Pharmacokinetic samples were collected up to 144 hours after dosing.

### Period 2 (Day 8 Through Day 22):

Subjects received a regimen of 50 mg fluvoxamine every 24 hours (Q24H) on Day 8 and Day 9. On Day 10, subjects began a regimen of 50 mg fluvoxamine every 12 hours (Q12H) up to Day 20. On Day 21, subjects began a taper regimen of 50 mg fluvoxamine Q24H up to Day 22. On Day 14, 300 mg DCCR was administered as 2×150 mg DCCR tablets after an overnight fast of at least 10 hours, and 50 mg fluvoxamine was given 30 minutes after DCCR administration.

All fluvoxamine morning doses were administered 30 minutes following the start of a standard breakfast, except for Day 14, when 50 mg fluvoxamine was given 30 minutes after 300 mg of DCCR. All fluvoxamine evening doses were given approximately 12 hours following the morning fluvoxamine dose. Pharmacokinetic blood samples were collected up to 168 hours (+5 minutes) after dosing on Day 14.

### Trial Details:

Subjects remained confined in the clinic from check-in on Day 0 through the EOS visit on Day 23 (or early termination).

Blood samples for PK analysis of diazoxide were collected at the following time points:
Day 1: Predose (<15 minutes) and at 1, 2, 4, 6, 8, 12, 16, 20, 24, 32, 40, 48, 72, 96, 120, and 144 hours (±5 minutes) post-dose
Day 14 Predose (<15 minutes) and at 1, 2, 4, 6, 8, 12, 16, 20, 24, 32, 40, 48, 72, 96, 120, 144, and 168 hours (±5 minutes) post-dose

The following PK parameters of diazoxide were determined after dosing DCCR alone and with fluvoxamine: area under the plasma concentration versus time curve (AUC) from time 0 to the last quantifiable concentration (AUC₀₋ₜ), AUC from time 0 extrapolated to infinity (AUC_{0-inf}), maximum observed plasma concentration (Cₘₐₓ), time to maximum observed plasma concentration (Tₘₐₓ), percentage of the AUC extrapolated for calculation of AUC_{0-inf} (% AUCₑₓₜ), apparent first order rate constant associated with the terminal portion of the concentration-time curve (λ_{z}), terminal phase half-life (t_{1/2}), apparent total body clearance (CL/F; diazoxide only), and apparent volume of distribution (V_{z}/F; diazoxide only). The primary diazoxide PK parameters for inferential statistics were: AUC₀₋ₜ, AUC_{0-inf}, and Cₘₐₓ.

The inferential statistical analysis was based on the PK parameter population. Following natural logarithmic transformation, PK parameters AUC₀₋ₜ, AUC_{0-inf}, and Cₘₐₓ of diazoxide and metabolites (MI and possibly MII, if quantifiable) were analyzed using mixed effects model with treatment as fixed effects and subject as the random effect. The geometric least squares (LS) mean difference was calculated and 90% confidence intervals (CIs) of the difference in the geometric LS mean, consistent with the two one-sided tests, was provided. Geometric LS means were calculated by back transformation (exponent) of LS means and the ratio of geometric LS means were calculated by back transformation (exponent) of LS mean difference. The 90% CI for the geometric LS means ratio was calculated by back transformation (exponent) of 90% CI for the LS mean difference.

Geometric CV % was calculated using the formula: CV %=100*Sqrt(exp(MSE)−1), where MSE was mean square error.

The comparison of interest was DCCR with fluvoxamine (test) versus DCCR alone (reference).

### Pharmacokinetic Results:

Following a single oral dose of 300 mg of DCCR alone and 300 mg DCCR+50 mg fluvoxamine Q12H, the median Tₘₐₓ for diazoxide were 12.00 and 23.85 hours, respectively.

For diazoxide, the estimated geometric mean (geometric CV %) t was 28.6 (27.4%) and 42.3 (41.0%) hours for 300 mg of DCCR alone and 300 mg DCCR+50 mg fluvoxamine Q12H, respectively.

Following administration of a single oral dose of DCCR with fluvoxamine (test), systemic exposure (Cₘₐₓ, AUC₀₋ₜ, and AUC_{0-inf}) of diazoxide was higher than that observed following administration of DCCR alone (reference).

Following a single oral dose of 300 mg of DCCR alone and 300 mg DCCR+50 mg fluvoxamine Q12H, the geometric mean (geometric CV %) diazoxide Cₘₐₓ values were 7.99 (12.2%) and 9.42 (15.1%) pg/mL, respectively. Overall geometric mean (geometric CV %) AUC₀₋ₜ for diazoxide was 452 (30.4%) and 723 (30.1%) h·μg/mL and the geometric mean (geometric CV %) AUC_{0-inf} was 489 (31.6%) and 732 (32.8%) h·μg/mL for 300 mg of DCCR alone and 300 mg DCCR+50 mg fluvoxamine Q12H, respectively. Geometric LS mean ratios (DCCR with fluvoxamine versus DCCR alone) for Cₘₐₓ, AUC₀₋ₜ, and AUC_{0-inf} were 117.54%, 158.66%, and 160.01% respectively. The 90% CIs around the ratio (DCCR with fluvoxamine versus DCCR alone) for Cₘₐₓ, AUC₀₋ₜ, and AUC_{0-inf} were (109.03 to 126.72), (141.11 to 178.40), and (137.54 to 186.16), respectively; all excluded 100%.

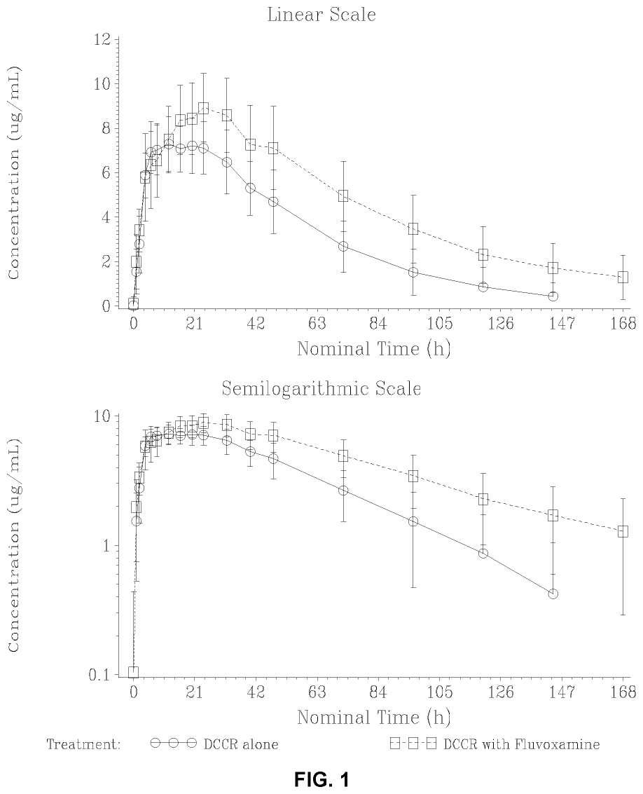

FIG. 1 depicts the change in mean plasma concentration of diazoxide over time (168 hours) for the pharmacokinetic concentration population on both a linear and semilogarithmic scale. The “DCCR alone” subjects (n=12) were administered a single dose of 300 mg DCCR on Day 1 and had at least one measurable plasma concentration of diazoxide, whereas the “DCCR with Fluvoxamine” subjects (n=10) were administered a single dose of 300 mg DCCR on Day 14 with 50 mg fluvoxamine administered Q24H on Days 8 and 9, Q12H on Days 10 to 20, and Q24H on Days 21 and 22, and had at least one measurable plasma concentration of diazoxide. Subjects 1006 and 1020 only had plasma concentrations in Period 1 (DCCR alone) since they were not dosed in Period 2 (DCCR with fluvoxamine). All values below the limit of quantification (0.5 μg/mL) were taken as zero for calculation of summary statistics. Concentrations collected outside the predefined collection window have been excluded.

TABLE 13
Geometric Mean (Geometric CV %) Plasma Pharmacokinetic Parameters
of Diazoxide (Pharmacokinetic Parameter Population)

| | Treatment | | | |
|---|---|---|---|---|
| | DCCR | | DCCR with | |
| | Alone | | Fluvoxamine | |
| Parameter (unit) | (N = 12) | | (N = 10) | |
| AUC_{0-inf} (μg · h/mL)^{a} | 489 | (31.6) | 732 | (32.8) |
| AUC₀₋ₜ (μg · h/mL) | 452 | (30.4) | 723 | (30.1) |
| Cₘₐₓ (μg/mL) | 7.99 | (12.2) | 9.42 | (15.1) |
| Tₘₐₓ (h)^{b} | 12.00 | (4.00-32.08) | 23.85 | (4.00-32.00) |
| t_{1/2} (h) | 28.6 | (27.4) | 42.3 | (41.0) |
| CL/F (L/h) | 0.613 | (31.6) | 0.410 | (32.8) |
| V_{z}/F (L) | 25.3 | (13.9) | 21.6 | (16.1) |

Abbreviations: CV = coefficient of variation; DCCR = diazoxide choline extended-release tablet; h = hours.
Note:
DCCR alone: single dose of 300 mg DCCR administered on Day 1; DCCR with fluvoxamine: single dose of 300 mg DCCR administered on Day 14 with 50 mg fluvoxamine administered Q24H on Days 8 and 9, Q12H on Days 10 to 20 and Q24H on Days 21 and 22.
Subjects 1006 and 1020 only present plasma concentrations on Period 1 (DCCR alone) since they were not dosed in Period 2.
AUC_{0-inf}, CL/F, and V_{z}/F values were excluded where % AUCext >20%. For these parameters n = 8 for DCCR with fluvoxamine.
For Tₘₐₓ, the median (minimum, maximum) values are presented.

### Statistical Analysis of Plasma Pharmacokinetic Parameters of Diazoxide

Statistical analysis of AUCs and Cₘₐₓ plasma parameters of DCCR with fluvoxamine compared to DCCR alone are summarized for diazoxide and MI in Table 14.

TABLE 14
Statistical Analysis of Plasma Pharmacokinetic Parameters
of Diazoxide (Pharmacokinetic Parameter Population)

| | | | | | Ratio (%) of | |
|---|---|---|---|---|---|---|
| Pharmacokinetic | | | Geometric | Treatment | Geometric LS | 90% CI of the |
| Parameter (units) | Treatment | N | LS Means | Comparison | Means | Ratio |
| AUC₀₋ₜ (μg · h/mL) | DCCR with | 10 | 716 | DCCR with | 158.66 | 141.11-178.40 |
| | Fluvoxamine | | | Fluvoxamine/ | | |
| | DCCR alone | 12 | 452 | DCCR alone | | |
| AUC_{0-inf} (μg · h/mL) | DCCR with | 8 | 783 | DCCR with | 160.01 | 137.54-186.16 |
| | Fluvoxamine | | | Fluvoxamine/ | | |
| | DCCR alone | 12 | 489 | DCCR alone | | |
| Cₘₐₓ (μg/mL) | DCCR with | 10 | 9.39 | DCCR with | 117.54 | 109.03-126.72 |
| | Fluvoxamine | | | Fluvoxamine/ | | |
| | DCCR alone | 12 | 7.99 | DCCR alone | | |

Abbreviations: CI, confidence interval; DCCR = diazoxide choline extended-release tablet; LS, least squares; Q12H = every 12 hours; Q24H = every 24 hours.
Note:
Reference: DCCR alone: single dose of 300 mg DCCR administered on Day 1; Test: DCCR with fluvoxamine: single dose of 300 mg DCCR administered on Day 14 with 50 mg fluvoxamine administered Q24H on Days 8 and 9, Q12H on Days 10 to 20 and Q24H on Days 21 and 22.
A linear mixed model was fitted to ln-transformed data with treatment as fixed effect and subject as the random effect; results are presented as percentages.
AUC_{0-inf} values were excluded where % AUCₑₓₜ >20%.
Subjects 1006 and 1020 only present plasma concentrations on Period 1 since they were not dosed in Period 2.

Conclusions from the Study:

For diazoxide, systemic exposure (Cₘₐₓ, AUC₀₋ₜ, and AUC_{0-inf}) was higher for DCCR with fluvoxamine (test) than that observed for DCCR alone (reference). The Cₘₐₓ, AUC₀₋ₜ, and AUC_{0-inf} were approximately 17%, 59%, and 60% higher for DCCR with fluvoxamine compared with DCCR alone. The 90% CIs around the ratio for Cₘₐₓ, AUC₀₋ₜ, and AUC_{0-inf} excluded 100%, indicating that the difference between treatments was statistically significant in these parameters. The higher systemic exposure (Cₘₐₓ, AUC₀₋ₜ, and AUC_{0-inf}) to diazoxide observed for DCCR with fluvoxamine (test) compared to DCCR alone (reference) was likely due to fluvoxamine being a strong CYP1A2 inhibitor.

As assessed from the geometric CV %, between-subject variability in exposure to diazoxide was 15.1% and 12.2% for Cₘₐₓ, 30.1% and 30.4% for AUC₀₋ₜ, and 32.8% and 31.6% for AUC_{0-inf} for DCCR with fluvoxamine and DCCR alone, respectively. The data suggests that between-subject variability of peak and overall exposures to diazoxide is similar following administration of DCCR with fluvoxamine compared to DCCR alone.

### Example 4: Use of Diazoxide Choline in the Treatment of Subjects with High Fasting Triglyceride Levels

This example describes the method by which diazoxide choline may be used to treat subjects with high fasting triglyceride levels. Such as with regard to the subjects enrolled in clinical study CT013.

Clinical study CT013 (NCT01211847) was a multi-center, randomized, double-blind, placebo-controlled study assessing the efficacy, safety and tolerability of Diazoxide Choline Controlled Release (DCCR) tablets in subjects without diabetes mellitus having very high fasting triglyceride levels in the range of 480-1500 mg/dL, with double-blind DCCR-fenofibrate combination extension. It was conducted at 30 sites in the US.

This study consisted of an up to 2-week Screening Period (Day −56 to Day −43), a 6-week Run-in/Washout Period (Day −42 to Day 0), and an 18-week Double-Blind Treatment Period (Day 1 to Day 126). The Double-Blind Treatment Period consisted of a 12-week Placebo-Controlled Treatment Period (Day 1 to Day 84) followed by a 6-week Combination Treatment Period (Day 85 to Day 126). At Baseline (Day 0), eligible subjects were assigned randomly in a 1:1 ratio to receive (starting from Day 1) DCCR 290 mg or Placebo. Randomization was stratified by statin status (Statin-treated or Statin-naive) and gender.

Statin-treated subjects were treated with open-label Lipitor® 20 mg from the start of the Run-in/Washout Period. Statin-naive subjects did not use lipid-lowering medication during the Run-in/Washout Period. During the double-blind treatment period, statin-naive subjects were assigned to take either DCCR or Placebo during the 12-week Placebo-Controlled Treatment Period, followed by the 6-week Combination Treatment Period, during which, in addition to DCCR or Placebo, Statin-naive subjects took open-label Trilipix® 135 mg for 6 weeks. Statin-treated subjects continued to receive treatment with Lipitor® 20 mg during the Double-Blind Treatment Period, and either DCCR or Placebo was assigned for a total of 18 weeks; Trilipix® 135 mg was not given to Statin-treated subjects. The Double-Blind Treatment Period for all subjects was initiated with a 2-step, 20-day titration of DCCR or Placebo to reach the final treatment dose of 290 mg DCCR or matching Placebo. Dose titration started at 145 mg DCCR or matching Placebo, with a dose increment of 72.5 mg DCCR or matching Placebo for each 10-day interval.

The planned enrollment was 44 subjects. This included 21 statin-naive subjects and 23 statin-treated subjects. Ultimately, 40 subjects completed the Placebo-Controlled Treatment Period, with 22 subjects receiving DCCR and 18 receiving Placebo. The demographics of the DCCR-treated subjects enrolled in the study are provided in Table 15 below.

TABLE 15
DCCR-Treated Subject Demographics.

| | Statin-naïve | | Statin-treated | | Overall | |
|---|---|---|---|---|---|---|
| | (N = 11) | | (N = 11) | | (N = 22) | |
| Age (years) | | | | | | |
| n | 11 | | 11 | | 22 | |
| Mean (SD) | 46.8 | (10.0) | 47.5 | (10.9) | 47.1 | (10.2) |
| Minimum-Maximum | 28-62 | | 25-68 | | 25-68 | |
| Age group 1 (n, %) | | | | | | |
| <50 years | 5 | (45.5) | 6 | (54.5) | 11 | (50.0) |
| ³50 years | 6 | (54.5) | 5 | (45.5) | 11 | (50.0) |
| Age group 2 (n, %) | | | | | | |
| <65 years | 11 | (100.0) | 10 | (90.9) | 21 | (95.5) |
| ³65 years | 0 | (0.0) | 1 | (9.1) | 1 | (4.5) |
| Sex (n, %) | | | | | | |
| Male | 9 | (81.8) | 8 | (72.2) | 17 | (77.3) |
| Female | 2 | (18.2) | 3 | (27.3) | 5 | (22.7) |
| Race (n, %) | | | | | | |
| Caucasian | 9 | (81.8) | 10 | (90.9) | 19 | (86.4) |
| Black | 0 | (0.0) | 1 | (9.1) | 1 | (4.5) |
| Asian | 1 | (9.1) | 0 | (0.0) | 1 | (4.5) |
| American Indian | 1 | (9.1) | 0 | (0.0) | 1 | (4.5) |
| Ethnicity (n, %) | | | | | | |
| Hispanic or Latino | 1 | (9.1) | 7 | (63.6) | 8 | (36.4) |
| Not Hispanic or Latino | 10 | (90.9) | 4 | (36.4) | 14 | (63.6) |
| Height (cm) | | | | | | |
| n | 11 | | 11 | | 22 | |
| Mean (SD) | 175.5 | (10.2 | 173.2 | (9.3) | 174.3 | (9.6) |
| Minimum-maximum | 153-188 | | 156-186 | | 153-188 | |
| Body mass | | | | | | |
| index (kg/m²) | | | | | | |
| n | 11 | | 11 | | 22 | |
| Mean (SD) | 31.2 | (5.7) | 30.5 | (4.5) | 30.8 | (5.0) |
| Minimum-maximum | 24.6-41.1 | | 23.7-38.9 | | 23.7-41.1 | |
| Weight (kg) | | | | | | |
| n | 11 | | 11 | | 22 | |
| Mean (SD) | 96.9 | (24.6) | 91.6 | (16.6) | 94.3 | (20.7) |

| | | | |
|---|---|---|---|
| Minimum-maximum | 71.8-145.1 | 69.4-123.1 | 69.4-145.1 |

As shown above, the DCCR-treated subjects enrolled in this study ranged from 69.4-145.1 kg, each of whom received a final treatment dose of 290 mg DCCR. This corresponds to receiving between about 1.9-4.2 mg/kg/day of diazoxide choline.

### Effect on Triglyceride Levels:

Triglyceride levels of the DCCR-treated subject population were found to significantly decrease following the 18-week Double-Blind Treatment Period. Triglyceride levels for individual DCCR-treated subjects are presented below in Table 16.

TABLE 16
Triglyceride Levels of DCCR-Treated Subjects.

| | | | | Baseline | End | | |
|---|---|---|---|---|---|---|---|
| | | DCCR | | Triglyceride | Triglyceride | Relative | |
| Subject | Weight | Dose | Statin | Level | Level | Change | Percent |
| Identifier | (kg) | (mg/kg) | Status | (mg/dL) | (mg/dL) | (mg/dL) | Change |
| 103-011 | 100.9 | 2.87 | Naive | 522 | 86 | −436 | −83.5% |
| 105-005 | 119.9 | 2.42 | Naive | 503 | 412 | −91 | −18.1% |
| 106-002 | 105.6 | 2.75 | Naive | 949 | 167 | −782 | −82.4% |
| 117-001 | 83 | 3.49 | Naive | 819 | 348 | −471 | −57.5% |
| 117-002 | 145.1 | 2.00 | Naive | 606 | 85 | −521 | −86.0% |
| 122-003 | 81.6 | 3.55 | Naive | 1132 | 210 | −922 | −81.4% |
| 128-001 | 80.4 | 3.61 | Naive | 756 | 575 | −181 | −23.9% |
| 128-002 | 76.5 | 3.79 | Naive | 593 | 354 | −239 | −40.3% |
| 128-005 | 74.8 | 3.88 | Naive | 647 | 92 | −555 | −85.8% |
| 101-010 | 99.8 | 2.91 | Treated | 524 | 642 | 118 | 22.5% |
| 113-006 | 123.1 | 2.36 | Treated | 1364 | 505 | −859 | −63.0% |
| 114-007 | 72.7 | 3.99 | Treated | 639 | 472 | −167 | −26.1% |
| 124-001 | 71.6 | 4.05 | Treated | 842 | 593 | −249 | −29.6% |
| 124-002 | 90.9 | 3.19 | Treated | 608 | 175 | −433 | −71.2% |
| 124-007 | 101.9 | 2.85 | Treated | 576 | 495 | −81 | −14.1% |
| 124-013 | 88 | 3.30 | Treated | 482 | 105 | −377 | −78.2% |
| 130-016 | 106.3 | 2.73 | Treated | 667 | 261 | −406 | −60.9% |

### Effect on Non-HDL Cholesterol Levels:

Non-HDL cholesterol levels of the DCCR-treated subject population were also found to significantly decrease following the 18-week Double-Blind Treatment Period. Non-HDL cholesterol levels for individual DCCR-treated subjects are presented below in Table 17.

TABLE 17
Non-HDL Cholesterol Levels of DCCR-Treated Subjects.

| | | DCCR | | Baseline Non-HDL | End Non-HDL | Relative | |
|---|---|---|---|---|---|---|---|
| Subject | Weight | Dose | Statin | Cholesterol | Cholesterol | Change | Percent |
| Identifier | (kg) | (mg/kg) | Status | (mg/dL) | (mg/dL) | (mg/dL) | Change |
| 103-011 | 100.9 | 2.87 | Naive | 159 | 123 | −36 | −22.6% |
| 105-005 | 119.9 | 2.42 | Naive | 213 | 236 | 23 | 10.8% |
| 106-002 | 105.6 | 2.75 | Naive | 423 | 221 | −202 | −47.8% |
| 117-001 | 83 | 3.49 | Naive | 369 | 213 | −156 | −42.3% |
| 117-002 | 145.1 | 2.00 | Naive | 290 | 69 | −221 | −76.2% |
| 122-003 | 81.6 | 3.55 | Naive | 248 | 110 | −138 | −55.6% |
| 128-001 | 80.4 | 3.61 | Naive | 213 | 209 | −4 | −1.9% |
| 128-002 | 76.5 | 3.79 | Naive | 214 | 231 | 17 | 7.9% |
| 128-005 | 74.8 | 3.88 | Naive | 312 | 173 | −139 | −44.6% |
| 101-010 | 99.8 | 2.91 | Treated | 150 | 210 | 60 | 40.0% |
| 113-006 | 123.1 | 2.36 | Treated | 199 | 142 | −57 | −28.6% |
| 114-007 | 72.7 | 3.99 | Treated | 199 | 152 | −47 | −23.6% |
| 124-001 | 71.6 | 4.05 | Treated | 141 | 119 | −22 | −15.6% |
| 124-002 | 90.9 | 3.19 | Treated | 220 | 183 | −37 | −16.8% |
| 124-007 | 101.9 | 2.85 | Treated | 116 | 114 | −2 | −1.7% |
| 124-013 | 88 | 3.30 | Treated | 338 | 151 | −187 | −55.3% |
| 130-016 | 106.3 | 2.73 | Treated | 146 | 120 | −26 | −17.8% |

### Effect on Fasting Insulin Levels:

Fasting insulin levels of the DCCR-treated subject population were also found to significantly decrease following the 18-week Double-Blind Treatment Period. Fasting insulin levels for individual DCCR-treated subjects are presented below in Table 18.

TABLE 18
Fasting Insulin Levels of DCCR-Treated Subjects.

| | | DCCR | | Baseline Fasting | End Fasting | Relative | |
|---|---|---|---|---|---|---|---|
| Subject | Weight | Dose | Statin | Insulin Level | Insulin Level | Change | Percent |
| Identifier | (kg) | (mg/kg) | Status | (μIU/mL) | (μIU/mL) | (μIU/mL) | Change |
| 103-011 | 100.9 | 2.87 | Naive | 69.8 | 2.6 | −67.2 | −96.3% |
| 101-010 | 99.8 | 2.91 | Treated | 83 | 26.2 | −56.8 | −68.4% |
| 113-006 | 123.1 | 2.36 | Treated | 42.9 | 19.1 | −23.8 | −55.5% |
| 124-007 | 101.9 | 2.85 | Treated | 23.7 | 8.1 | −15.6 | −65.8% |
| 124-009 | 98.8 | 2.94 | Treated | 40.2 | 14.1 | −26.1 | −64.9% |
| 124-012 | 84.7 | 3.42 | Treated | 22.7 | 9.9 | −12.8 | −56.4% |

### Effect on Insulin Resistance:

Insulin Resistance of the DCCR-treated subject population was also found to significantly decrease following the 18-week Double-Blind Treatment Period. Insulin Resistance was assessed via the Homeostatic Model Assessment of Insulin Resistance (HOMA-IR) score, which is depicted for individual DCCR-treated subjects below in Table 19.

TABLE 19
HOMA-IR Scores of DCCR-Treated Subjects.

| | | DCCR | | Baseline | End | | |
|---|---|---|---|---|---|---|---|
| Subject | Weight | Dose | Statin | HOMA-IR | HOMA-IR | Relative | Percent |
| Identifier | (kg) | (mg/kg) | Status | Score | Score | Change | Change |
| 103-011 | 100.9 | 2.87 | Naive | 12.72 | 0.54 | −12.18 | −95.8% |
| 101-010 | 99.8 | 2.91 | Treated | 26.56 | 8.03 | −18.53 | −69.8% |
| 113-006 | 123.1 | 2.36 | Treated | 7.44 | 4.5 | −2.94 | −39.5% |
| 124-007 | 101.9 | 2.85 | Treated | 7.37 | 2.05 | −5.32 | −72.2% |
| 124-009 | 98.8 | 2.94 | Treated | 13.79 | 3.7 | −10.09 | −73.2% |
| 124-012 | 84.7 | 3.42 | Treated | 5.85 | 2.6 | −3.25 | −55.6% |
| 130-016 | 106.3 | 2.73 | Treated | 4.52 | 2.5 | −2.02 | −44.7% |

### Example 5: Use of Diazoxide Choline in the Treatment of Obese Subjects

This example describes the method by which diazoxide choline may be used to treat obese subjects. For example, as discussed below with regard to the subjects enrolled in clinical study TR002.

Clinical study TR002 was a multicenter, randomized, open-label, sequential ascending repeated dose study using 6 dose regimens of diazoxide choline conducted in 72 obese subjects. For Groups 1-6, the duration of the treatment period was 14 days, with 2 subjects in each group serving as untreated controls and receiving no drug treatment. For Group 7, all subjects received drug treatment with increasing doses of diazoxide choline during the first 28 days of the treatment period before reaching the target dose, and the duration of the treatment period was 49 days (Days −1 to 48). There was a post-treatment follow-up period of 7 days in all groups. During the entire study (from Baseline to the Follow-Up Visit) a mild hypocaloric diet was prescribed to all subjects. The energy requirement was calculated by a dietitian based on the estimated basal metabolism rate using the Harris-Benedict equation and the physical activity of each subject at the Screening Visit. From this amount of energy, 600 kcal/day was subtracted to obtain the recommended hypocaloric diet for each subject.

In this study, subjects were male or female, 18-65 years old, and had a Body Mass Index (BMI) of either (a) 27-30 kg/m² (with untreated hypertension and/or dyslipidemia), or (b) 30-45 kg/m² (with or without comorbidities). All subjects exhibited the ability to follow verbal and written instructions. An informed consent form was signed by each subject. The demographics of the subjects enrolled in this study are provided in Tables 20-21 below.

TABLE 20
Subject Demographics for Groups 1-6.

| | Overall | |
|---|---|---|
| Demographic Variable | N = 59 | |
| Mean age (range) in years | 41.4 | (21 to 64) |
| Mean weight (range) in kg | 105.5 | (74.4 to 150.5) |
| Mean height (range) in cm | 169.3 | (149.0 to 188.8) |
| Mean BMI (range) in kg/m² | 36.7 | (30.4 to 44.0) |
| Gender [n(%)] | | |
| Male | 29 | (49.2%) |
| Female | 30 | (50.8%) |
| Ethnicity [n(%)] | | |
| Hispanic or Latino | 6 | (10.2%) |
| Race [n(%)] | | |
| White | 45 | (76.3%) |
| Black | 10 | (16.9%) |
| Asian | 1 | (1.7%) |
| Native Hawaiian or | 1 | (1.7%) |
| Other Pacific Islander | | |
| American Indian/ | 1 | (1.7%) |
| Hispanic | | |
| Mexican | 1 | (1.7%) |

TABLE 21
Subject Demographics for Group 7.

| | Overall | |
|---|---|---|
| Demographic Variable | N = 12 | |
| Mean age (range) in years | 40.4 | (18 to 57) |
| Mean weight (range) in kg | 100.6 | (79.1 to 154.8) |
| Mean height (range) in cm | 167.5 | (155.2 to 193.0) |
| Mean BMI (range) in kg/m² | 35.5 | (31.5 to 42.5) |
| Gender [n(%)] | | |
| Male | 5 | (41.7%) |
| Female | 7 | (58.3%) |
| Ethnicity [n(%)] | | |
| Hispanic or Latino | 0 | |
| Race [n(%)] | | |
| White | 11 | (91.7%) |
| Native Hawaiian or | 1 | (8.3%) |

Other Pacific Islander

### Effect on Weight:

The weight of the subject population was found to significantly decrease following treatment. The weights of individual subjects are presented below in Table 22.

TABLE 22
Weight of Subjects.

| | | | | Base- | | | |
|---|---|---|---|---|---|---|---|
| Subject | BMI | DCCR | DCCR | line | End | Relative | |
| Iden- | (kg/ | Dose | Dose | Weight | Weight | Change | Percent |
| tifier | m²) | (mg) | (mg/kg) | (kg) | (kg) | (kg) | Change |
| 61 | 41.6 | 507.5 | 3.26 | 155.6 | 140.5 | −15.1 | −9.7% |
| 62 | 32.4 | 362.5 | 4.09 | 88.6 | 85.5 | −3.1 | −3.5% |
| 64 | 36.5 | 507.5 | 4.45 | 114.1 | 103.4 | −10.7 | −9.4% |
| 65 | 32.7 | 507.5 | 6.46 | 78.6 | 75.2 | −3.4 | −4.3% |
| 66 | 32.9 | 507.5 | 5.70 | 89.1 | 85.2 | −3.9 | −4.4% |
| 68 | 32.2 | 507.5 | 5.97 | 85.0 | 79.1 | −5.9 | −6.9% |
| 70 | 42.5 | 507.5 | 4.06 | 125.0 | 118.9 | −6.1 | −4.9% |
| 71 | 32.9 | 507.5 | 5.70 | 89.1 | 84.1 | −5 | −5.6% |
| 72 | 36.3 | 507.5 | 5.39 | 94.1 | 90.2 | −3.9 | −4.1% |

### Effect on Fasting Glucose Levels:

The fasting glucose levels of the subject population were found to significantly increase following treatment. The fasting glucose levels of individual subjects are presented below in Table 23.

TABLE 23
Fasting Glucose Levels of Subjects.

| | | | | | Baseline | | | |
|---|---|---|---|---|---|---|---|---|
| | DCCR | Baseline | DCCR | Treatment | Fasting | End Fasting | Relative | |
| Subject | Dose | Weight | Dose | Duration | Glucose | Glucose | Change | Percent |
| Identifier | (mg) | (kg) | (mg/kg) | (days) | (mg/dL) | (mg/dL) | (mg/dL) | Change |
| 59 | 435 | 112.0 | 3.88 | 14 | 92 | 131 | 39 | 42.4% |
| 64 | 507.5 | 114.1 | 4.45 | 49 | 106 | 132 | 26 | 24.5% |
| 65 | 507.5 | 78.6 | 6.46 | 49 | 113 | 152 | 39 | 34.5% |
| 71 | 507.5 | 89.1 | 5.70 | 49 | 104 | 129 | 25 | 24.0% |

### Effect on Fasting Insulin Levels:

The fasting insulin levels of the subject population were found to significantly decrease following treatment. The fasting insulin levels of individual subjects are presented below in Table 24.

TABLE 24
Fasting Insulin Levels of Subjects.

| | | | | | Baseline | | | |
|---|---|---|---|---|---|---|---|---|
| | DCCR | Baseline | DCCR | Treatment | Fasting | End Fasting | Relative | |
| Subject | Dose | Weight | Dose | Duration | Insulin | Insulin | Change | Percent |
| Identifier | (mg) | (kg) | (mg/kg) | (days) | (μIU/mL) | (μIU/mL) | (μIU/mL) | Change |
| 3 | 145 | 123.8 | 1.17 | 14 | 35.7 | 16.6 | −19.1 | −53.5% |
| 5 | 145 | 95.0 | 1.53 | 14 | 23.2 | 6.2 | −17 | −73.3% |
| 17 | 290 | 107.0 | 2.71 | 14 | 24.7 | 22.8 | −1.9 | −7.7% |
| 56 | 435 | 135.2 | 3.22 | 14 | 24.7 | 11.6 | −13.1 | −53.0% |

### Effect on Insulin Resistance:

The insulin resistance of the subject population was found to significantly decrease following treatment. Insulin Resistance was assessed via the Homeostatic Model Assessment of Insulin Resistance (HOMA-IR) score, which is depicted for individual subjects below in Table 25.

TABLE 25
HOMA-IR Scores of Subjects.

| | DCCR | Baseline | DCCR | Treatment | Baseline | End | | |
|---|---|---|---|---|---|---|---|---|
| Subject | Dose | Weight | Dose | Duration | HOMA-IR | HOMA-IR | Relative | Percent |
| Identifier | (mg) | (kg) | (mg/kg) | (days) | Score | Score | Change | Change |
| 61 | 507.5 | 155.6 | 3.26 | 49 | 5.1 | 2.3 | −2.8 | −54.9% |
| 62 | 362.5 | 88.6 | 4.09 | 49 | 4.9 | 4.1 | −0.8 | −16.3% |
| 64 | 507.5 | 114.1 | 4.45 | 49 | 5.2 | 2.2 | −3 | −57.7% |
| 65 | 435 | 78.6 | 5.53 | 49 | 4.7 | 4.4 | −0.3 | −6.4% |
| 68 | 507.5 | 85 | 5.97 | 49 | 6.0 | 3.4 | −2.6 | −43.3% |

### Example 6: Use of Diazoxide Choline in the Treatment of Subjects with High Fasting Triglyceride Levels

This example describes the method by which diazoxide choline may be used to treat subjects with high fasting triglyceride levels, specifically with regard to the subjects enrolled in clinical study PC007.

Clinical study PC007 (NCT00696475) was a multi-center, randomized, double blind, placebo controlled, parallel group study. Subjects were randomized in a 1:1:1:1 ratio to treatment with placebo or DCCR tablet in dosages equivalent to DCCR 290 mg/day, 435 mg/day, or 580 mg/day. Subjects assigned to the 435 mg/day or 580 mg/day groups were to receive 290 mg/day for the first 7 days of treatment and an increase of 145 mg/day after every 7 days until reaching their assigned dosage at Day 8 or Day 15, respectively. Subjects were treated for a total of 56 days.

In this study, subjects were ambulatory men and women that were 18-75 years of age, inclusive, with triglyceride levels in the range of 200-650 mg/dL. The demographics of the subject population in this study are provided in Table 26 below.

TABLE 26
Summary of Subject Demographics.

| | DCCR | | DCCR | | DCCR | |
|---|---|---|---|---|---|---|
| Demographic | 290 mg | | 435 mg | | 580 mg | |
| Variable | (N = 22) | | (N = 23) | | (N = 23) | |
| Age (yrs) | | | | | | |
| n | 22 | | 23 | | 23 | |
| Mean (SD) | 56.9 | (9.0) | 54.7 | (9.9) | 56.0 | (8.2) |
| Range | 37-73 | | 35-74 | | 42-71 | |
| Sex | | | | | | |
| Male | 11 | (50.0) | 17 | (73.9) | 16 | (69.6) |
| Female | 11 | (50.0) | 6 | (26.1) | 7 | (30.4) |
| Race | | | | | | |
| Caucasian | 20 | (90.9) | 20 | (87.0) | 22 | (95.7) |
| Black | 1 | (4.5) | 2 | (8.7) | 0 | (0.0) |
| Asian | 1 | (4.5) | 1 | (4.3) | 1 | (4.3) |
| Ethnicity | | | | | | |
| Hispanic/Latino | 5 | (22.7) | 3 | (13.0) | 2 | (8.7) |
| Not Hispanic/Latino | 17 | (77.3) | 20 | (87.0) | 21 | (91.3) |

### Effect on Fasting Glucose Levels:

The fasting glucose levels of the subject population were found to significantly increase following treatment. The fasting glucose levels of individual subjects are presented below in Table 27.

TABLE 27
Fasting Glucose Levels of Subjects.

| | | | | Baseline | | | |
|---|---|---|---|---|---|---|---|
| | DCCR | Baseline | DCCR | Fasting | End Fasting | Relative | |
| Subject | Dose | Weight | Dose | Glucose | Glucose | Change | Percent |
| Identifier | (mg) | (kg) | (mg/kg) | (mg/dL) | (mg/dL) | (mg/dL) | Change |
| 003-001 | 290 | 102.7 | 2.82 | 106 | 122 | 16 | 15.1% |
| 004-005 | 290 | 102.5 | 2.83 | 105 | 125 | 20 | 19.0% |
| 014-005 | 290 | 52.1 | 5.57 | 104 | 129 | 25 | 24.0% |
| 001-014 | 435 | 92.6 | 4.70 | 95 | 126 | 31 | 32.6% |
| 002-005 | 580 | 70.2 | 8.26 | 89 | 122 | 33 | 37.1% |
| 006-020 | 580 | 73.7 | 7.87 | 99 | 153 | 54 | 54.5% |
| 008-003 | 580 | 70.6 | 8.22 | 98 | 120 | 22 | 22.4% |
| 008-020 | 580 | 79.2 | 7.32 | 108 | 143 | 35 | 32.4% |
| 008-036 | 580 | 73.6 | 7.88 | 90 | 124 | 34 | 37.8% |
| 011-024 | 580 | 116.7 | 4.97 | 112 | 131 | 19 | 17.0% |
| 012-010 | 580 | 117.3 | 4.94 | 93 | 139 | 46 | 49.5% |
| 014-003 | 580 | 55.0 | 10.55 | 114 | 128 | 14 | 12.3% |

### Effect on Fasting Insulin Levels:

The fasting insulin levels of the subject population were found to significantly decrease following treatment. The fasting insulin levels of individual subjects are presented below in Table 28.

TABLE 28
Fasting Insulin Levels of Subjects.

| | | | | Baseline | | | |
|---|---|---|---|---|---|---|---|
| | DCCR | Baseline | DCCR | Fasting | End Fasting | Relative | |
| Subject | Dose | Weight | Dose | Insulin | Insulin | Change | Percent |
| Identifier | (mg) | (kg) | (mg/kg) | (μIU/mL) | (μIU/mL) | (μIU/mL) | Change |
| 008-004 | 290 | 76.0 | 3.82 | 56 | 27.8 | 20.4 | −7.4 |
| 004-002 | 435 | 86.7 | 5.02 | 56 | 33.7 | 14.8 | −18.9 |
| 012-005 | 580 | 98.6 | 5.88 | 56 | 31.8 | 15.3 | −16.5 |
| 012-010 | 580 | 117.3 | 4.94 | 56 | 28.9 | 13.1 | −15.8 |

### Example 7: Use of Diazoxide Choline in the Treatment of Severely Obese Subjects

This example describes the method by which diazoxide choline may be used to treat severely obese subjects. For example, as discussed below with regard to the subjects in clinical study C632.

Clinical study C632 is a multicenter, open-label study using 2 dose regimens (each with 6 distinct weight-based titration schedules) of diazoxide choline, with up to 30 severely obese subjects.

Participants not taking one or more strong CYP1A2 inhibitors titrate to the lowest diazoxide choline dose within a target range, as tolerated for up to 24 weeks (as shown in Table 29). Participants should remain on their initial target dose until the End of Treatment Visit (Week 26). The maximum allowed dosage is 5.8 mg/kg/day or 525 mg/day (whichever is lower).

TABLE 29
Diazoxide Choline Titration Regimen (by Weight Group) for Participants
not Taking One or More Strong CYP1A2 Inhibitors

| | Step 1 | Step 2 | | Step 3 | | Step 4 | Step 5 | Step 6 | Initial | Max |
|---|---|---|---|---|---|---|---|---|---|---|
| | Weeks | Weeks | | Weeks | | Weeks | Weeks | Weeks | Target | Target |
| Weight | 1-4 | 5-8 | | 9-12 | | 12-16 | 17-20 | 20-24 | Dose | Dose |
| 20 to <30 kg | 25 mg | 50 | mg | 75 | mg | 100 mg | — | — | 100 mg | 100 mg |
| 30 to <40 kg | 75 mg | 150 | mg | — | | — | — | — | 150 mg | 150 mg |
| 40 to <65 kg | 75 mg | 150 | mg | 225 | mg | — | — | — | 225 mg | 225 mg |
| 65 to <100 kg | 75 mg | 150 | mg | 225 | mg | 275 mg | — | | 275 mg | 375 mg |
| 100 to <135 kg | 75 mg | 150 | mg | 225 | mg | 275 mg | 325 mg | — | 325 mg | 450 mg |
| ≥135 kg | 75 mg | 150 | mg | 225 | mg | 275 mg | 325 mg | 375 mg | 375 mg | 525 mg |

Participants taking one or more strong CYP1A2 inhibitors titrate to the lowest diazoxide choline dose within a target range, as tolerated for up to 24 weeks (as shown in Table 30). Participants should remain on their initial target dose until the End of Treatment Visit (Week 26) The maximum allowed dosage for participants taking strong CYP1A2 inhibitors is 3.6 mg/kg/day or 325 mg/day (whichever is lower).

TABLE 30
Diazoxide Choline Titration Regimen (by Weight Group) for
Participants Taking One or More Strong CYP1A2 Inhibitors

| | Step 1 | Step 2 | | Step 3 | | Step 4 | | Step 5 | Step 6 | Initial | | Max | |
|---|---|---|---|---|---|---|---|---|---|---|---|---|---|
| | Weeks | Weeks | | Weeks | | Weeks | | Weeks | Weeks | Target | | Target | |
| Weight | 1-4 | 5-8 | | 9-12 | | 12-16 | | 17-20 | 20-24 | Dose | | Dose | |
| 20 to <30 kg | 25 mg | 25 | mg | 50 | mg | 75 | g | — | — | 75 | mg | 75 | mg |
| 30 to <40 kg | 50 mg | 100 | mg | — | | — | | — | — | 100 | mg | 100 | mg |
| 40 to <65 kg | 50 mg | 100 | mg | 150 | mg | — | | — | — | 150 | mg | 150 | mg |
| 65 to <100 kg | 50 mg | 100 | mg | 150 | mg | 200 | mg | | — | 200 | mg | 250 | mg |
| 100 to <135 kg | 50 mg | 100 | mg | 150 | mg | 200 | mg | 225 mg | | 225 | mg | 300 | mg |
| ≥135 kg | 50 mg | 100 | mg | 150 | mg | 200 | mg | 225 mg | 275mg | 275 | mg | 325 | mg |

Participants in this study must meet all of the following inclusion criteria to be eligible for this trial: 1) Provide voluntary, written informed consent (parent(s)/legal guardian(s) of participant) or written assent (participants, as appropriate) at Visit 1; 2) Male or female with PWS who has hyperphagia and is ≥4 years of age at Visit 1; 3) Documented history of genetically confirmed PWS; 4) Has not received diazoxide choline within 3 months of Visit 1 (Note: patients previously treated with diazoxide choline who discontinued, may be allowed to participate provided they meet all eligibility criteria and none of the exclusion criteria); 5) Severe obesity (Class 2 or Class 3 as defined in Table 31 below); 6) Female participants of child-bearing potential must have a negative pregnancy test at Visit 1 and Visit 2 and agree to remain abstinent or use a highly-effective contraceptive during the trial and for 90 days after trial participation; 7) Male participants with female partners of child bearing potential must agree to a barrier method of birth control if they become sexually active during the trial. Male participants who are sexually active must use birth control during the trial and for 90 days after trial participation. Male participants must not donate sperm during and for 90 days following their participation in the trial; and 8) Participant and/or caregiver is willing and able to comply with the protocol-required procedures and visits.

TABLE 31
Obesity Classification

| BMI (kg/m²) | Obesity Class |
|---|---|
| Children and adolescents | |
| 95th to less than 120% of the 95th percentile | 1 |
| 120% to less than 140% of the 95th percentile | 2 |
| 140% or greater of the 95th percentile. | 3 |
| Adults | |
| 30.0-34.9 | 1 |
| 35.0-39.9 | 2 |
| ≥40 | 3 |

Obesity Classification in Children and Adolescents (Hampl, 2023)
Obesity Classification in Adults (CDC, 2024)
Growth charts available at http://www.cdc.gov/growthcharts

Individuals who provide voluntary informed consent/assent undergo Screening procedures to determine trial eligibility at Visit 1 (Study Days −42 to −2) as described above. Visit 1 may occur over more than 1 day to facilitate the required Screening procedures and assessments and review of results. If a repeat test is needed or washout of a disallowed concomitant medication is required prior to enrollment, the Screening Period may be extended with approval by the Medical Monitor. Participants who meet the eligibility criteria return to the clinical site at Visit 2. Participants who are confirmed eligible are enrolled and complete predose study assessments.

Participants titrate for up to 24 weeks until they reach their initial weight-based target dose (see Tables 29-30 above). Participants should remain on their initial target dose until the End of Treatment Visit (Week 26). Dose adjustments (dose reductions and/or dose interruptions) may be made at any time during the trial for safety or tolerability. Dose increases for efficacy may be made within the target weight-based dose range described in Tables 29-30 above and the VYKAT XR United States Prescribing Information (USPI) only after the participant has reached and have been on their initial target dose for a minimum of 2 weeks and only after consultation between the Investigator and Medical Monitor. Dose increases for efficacy are to be made in 25 mg to 75 mg increments. All participants have a safety follow-up telemedicine visit 2 weeks after their last trial dose. The End of Trial is defined as completion the safety follow-up visit.

1.-145. (canceled)

146. A method of treating hyperphagia in a patient having Prader-Willi Syndrome, comprising administering to the patient by re-titration a therapeutically effective amount of diazoxide, or a pharmaceutically acceptable salt thereof, after a temporary interruption in the administration of the therapeutically effective amount of diazoxide, or a pharmaceutically acceptable salt thereof.

147. The method of claim 146, wherein the temporary interruption is for 7 days, 10 days, or 14 days.

148. The method of claim 146, wherein the temporary interruption is for 7 days or more.

149. The method of claim 146, wherein the temporary interruption is due to an adverse event.

150. The method of claim 149, wherein the adverse event is clinically significant fasting glucose or HbA1c elevation.

151. The method of claim 149, wherein the adverse event is clinically significant fluid overload.

152. The method of claim 146, wherein the diazoxide, or a pharmaceutically acceptable salt thereof, is re-titrated by administering dosage amounts corresponding to the dosage of diazoxide choline in the below dosing schedule:

| | | Titration | | Titration | | Titration | | Target |
|---|---|---|---|---|---|---|---|---|
| Patient | | Step 1 | | Step 2 | | Step 3 | | Maintenance |
| Weight | | (Weeks | | (Weeks | | (Weeks | | Dose (Week 7 |
| Range | | 1 and 2) | | 3 and 4) | | 5 and 6) | | and After) |
| ≥20 to <30 | kg | 25 | mg/day | 50 | mg/day | 75 | mg/day | 100 mg/day |
| ≥30 to <40 | kg | 75 | mg/day | 150 | mg/day | 150 | mg/day | 150 mg/day |
| ≥40 to <65 | kg | 75 | mg/day | 150 | mg/day | 225 | mg/day | 225 mg/day |
| ≥65 to <100 | kg | 150 | mg/day | 225 | mg/day | 300 | mg/day | 375 mg/day |
| ≥100 to <135 | kg | 150 | mg/day | 300 | mg/day | 375 | mg/day | 450 mg/day |
| ≥135 | kg | 150 | mg/day | 300 | mg/day | 450 | mg/day | 525 mg/day |

153. The method of claim 146, wherein the diazoxide, or a pharmaceutically acceptable salt thereof, is re-titrated at a dosage amount that is reduced relative to the amount that would be administered to the patient if the patient had not experienced the adverse event.

154. The method of claim 146, wherein the diazoxide, or a pharmaceutically acceptable salt thereof, is re-titrated at a dosage amount that is reduced relative to the dosage amount corresponding to the dosages of diazoxide choline in the below dosing schedule:

| | | Titration | | Titration | | Titration | | Target |
|---|---|---|---|---|---|---|---|---|
| Patient | | Step 1 | | Step 2 | | Step 3 | | Maintenance |
| Weight | | (Weeks | | (Weeks | | (Weeks | | Dose (Week 7 |
| Range | | 1 and 2) | | 3 and 4) | | 5 and 6) | | and After) |
| ≥20 to <30 | kg | 25 | mg/day | 50 | mg/day | 75 | mg/day | 100 mg/day |
| ≥30 to <40 | kg | 75 | mg/day | 150 | mg/day | 150 | mg/day | 150 mg/day |
| ≥40 to <65 | kg | 75 | mg/day | 150 | mg/day | 225 | mg/day | 225 mg/day |
| ≥65 to <100 | kg | 150 | mg/day | 225 | mg/day | 300 | mg/day | 375 mg/day |
| ≥100 to <135 | kg | 150 | mg/day | 300 | mg/day | 375 | mg/day | 450 mg/day |
| ≥135 | kg | 150 | mg/day | 300 | mg/day | 450 | mg/day | 525 mg/day |

155. The method of claim 146, wherein a pharmaceutically acceptable salt of diazoxide is administered.

156. The method of claim 155, wherein the pharmaceutically acceptable salt of diazoxide is diazoxide choline.

157. The method of claim 146, wherein diazoxide free form is administered.

158. A method of treating hyperphagia in a patient having Prader-Willi Syndrome, comprising resuming administration to the patient of a therapeutically effective amount of diazoxide, or a pharmaceutically acceptable salt thereof, after a temporary interruption in the administration of the diazoxide, or a pharmaceutically acceptable salt thereof,
wherein the dosage amount of diazoxide, or a pharmaceutically acceptable salt thereof, administered upon resuming treatment is the previous once daily dose of diazoxide choline administered to the patient prior to the adverse event.

159. The method of claim 158, wherein the interruption in treatment lasted less than 7 days.

160. The method of claim 158, wherein the interruption in treatment is due to an adverse event.

161. The method of claim 160, wherein the adverse event is clinically significant fasting glucose or HbA1c elevation.

162. The method of claim 160, wherein the adverse event is clinically significant fluid overload.

163. The method of claim 158, wherein a pharmaceutically acceptable salt of diazoxide is administered.

164. The method of claim 163, wherein the pharmaceutically acceptable salt of diazoxide is diazoxide choline.

165. The method of claim 158, wherein diazoxide free form is administered.
